(19) United States
(12) Patent Application Publication
(10) Pub. No.: US 2026/0152514 A1
(43) Pub. Date: Jun. 4, 2026

(54) RAS INHIBITORS

(71) Applicant: Revolution Medicines, Inc., Redwood City, CA (US)

(72) Inventors: Jinyu LIU, Union City, CA (US); Kristof POTA, San Carlos, CA (US); Jingwei YIN, Redwood City, CA (US); James CREGG, Belmont, CA (US); Adrian L. GILL, Atherton, CA (US)

(21) Appl. No.: 19/460,508

(22) Filed: Jan. 27, 2026

Related U.S. Application Data
(63) Continuation of application No. PCT/US2025/051790, filed on Oct. 21, 2025.
(60) Provisional application No. 63/709,587, filed on Oct. 21, 2024.
(60) Provisional application No. 63/897,138, filed on Oct. 10, 2025.

(51) Int. Cl. C07D 519/00; A61K 31/504; A61K 31/506; A61K 31/519; A61K 31/5377; A61K 31/5383; A61K 31/5386; A61K 31/55; A61K 45/06; C07D 498/22; C07D 513/22

(52) U.S. Cl. CPC C07D 519/00; A61K 31/504; A61K 31/506; A61K 31/519; A61K 31/5377; A61K 31/5383; A61K 31/5386; A61K 31/55; A61K 45/06; C07D 498/22; C07D 513/22

(57) ABSTRACT

The disclosure features macrocyclic compounds and pharmaceutical compositions thereof, capable of inhibiting Ras proteins, and their uses in the treatment of cancers.

### BACKGROUND

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by the various Ras mutations.

### SUMMARY OF INVENTION

Provided herein are Ras inhibitors and compounds useful for studying Ras inhibition. The approach described herein entails formation of a high affinity three-component complex, or conjugate, between a synthetic ligand and two intracellular proteins which do not interact under normal physiological conditions: the target protein of interest (e.g., Ras) and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, is that they form is by steric occlusion of the interaction site between Ras and downstream effector molecules, such as RAF and PI3K, which are required for propagating the oncogenic signal.

As such, in one aspect, the disclosure features a compound, or pharmaceutically acceptable salt thereof, of structural Formula I:

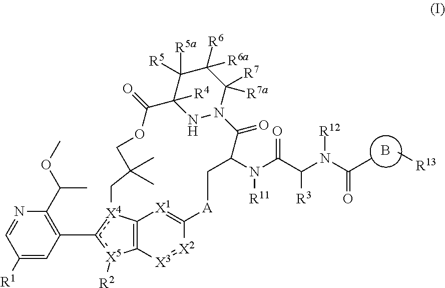

or a pharmaceutically acceptable salt thereof, wherein:
A is optionally substituted 3- to 6-membered cycloalkylene, optionally substituted 4- to 6-membered heterocycloalkylene, optionally substituted C₆₋₁₀ arylene, or optionally substituted 5- to 10-membered heteroarylene;
ring B is optionally substituted 4- to 11-membered heterocycloalkylene, wherein 1 or 2 ring carbon atoms of the 4- to 11-membered heterocycloalkylene are optionally replaced by a member independently selected from C(═O) and SO₂;
X¹, X² and X³ are each independently N or CR⁸, wherein each R⁸ is independently hydrogen, halogen, CN, or C₁₋₆ alkyl;
X⁴ is N and X⁵ is C; or
X⁵ is N and X⁴ is C;
[symbol] is either a single bond or a double bond to maintain the aromaticity of the 5-membered ring containing X⁴ and X⁵;
R¹ is hydrogen, optionally substituted C₁₋₆ alkyl, optionally substituted C₂₋₆ alkenyl, optionally substituted C₂-6 alkynyl, optionally substituted C₁₋₆ heteroalkyl, optionally substituted 3- to 6-membered cycloalkyl, optionally substituted 3- to 6-membered cycloalkenyl, optionally substituted 4- to 14-membered heterocycloalkyl, optionally substituted 6- to 10-membered aryl, or optionally substituted 5- to 10-membered heteroaryl;
R² is optionally substituted C₁₋₆ alkyl, optionally substituted C₂₋₆ alkynyl, optionally substituted C₁₋₆ heteroalkyl, optionally substituted 3- to 6-membered cycloalkyl, or optionally substituted 4- to 14-membered heterocycloalkyl;
R³ is hydrogen, optionally substituted C₁₋₆ alkyl, optionally substituted C₁₋₃ heteroalkyl, optionally substituted C₃₋₆ cycloalkyl, or optionally substituted 4- to 6-membered heterocycloalkyl;
or R² and R³ together with the atoms to which they are attached form a fused C₄₋₁₀ cycloalkyl or a fused 4- to 10-membered heterocycloalkyl having 1, 2 or 3 ring heteroatoms selected from N, O, and S, wherein the fused C₄₋₁₀ cycloalkyl and heterocycloalkyl are each optionally substituted;
R⁴, R⁵, R^{5a}, R⁶, R^{6a}, R⁷ and R^{7a} are each independently hydrogen, halogen, or C₁₋₆ alkyl;
or R⁵ and R⁷ are taken together to form methylene or ethylene, which is covalently bound to the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form methylene or ethylene, which is covalently bound to the carbon atoms to which they are attached, and R⁵, R^{5a}, R^{6a}, R⁷ and R^{7a} are each independently hydrogen, halogen, or C₁₋₆ alkyl;
or R⁴ and R⁷ are taken together to form methylene or ethylene, which is covalently bound to the carbon atoms to which they are attached, R⁵, R^{5a}, R⁶, R^{6a}, and R^{7a} are each independently hydrogen, halogen, or C₁₋₆ alkyl;
or R⁵ and R⁶ taken together with the atoms to which they are attached form optionally substituted fused C₃₋₆ cycloalkyl or optionally substituted 4- to 6-membered fused heterocycloalkyl;
or R⁶ and R⁷ taken together with the atoms to which they are attached form optionally substituted fused C₃₋₆ cycloalkyl or optionally substituted 4- to 6-membered fused heterocycloalkyl;
or R⁵ and R^{5a} taken together with the atom to which they are attached form optionally substituted C₃₋₆ cycloalkyl or optionally substituted 4- to 6-membered heterocycloalkyl;
or R⁶ and R^{6a} taken together with the atom to which they are attached form optionally substituted C₃₋₆ cycloalkyl or optionally substituted 4- to 6-membered heterocycloalkyl;
or R⁷ and R^{7a} taken together with the atom to which they are attached form an optionally C₃₋₆ cycloalkyl or optionally substituted 4- to 6-membered heterocycloalkyl;
or R⁵ and R⁶ are taken together to form a bond;
R¹¹ is hydrogen or optionally substituted C₁₋₆ alkyl;
R¹² is hydrogen or optionally substituted C₁₋₆ alkyl; and
R¹³ is H, optionally substituted C₁₋₆ alkyl, —C(O)—R¹⁴, —C(O)OR¹⁴, —C(O)NHR¹⁴, —C(O)NR¹⁴R¹⁴, —SO₂R¹⁴, —SO₂NHR¹⁴, —SO₂NR¹⁴R¹⁴, wherein each R¹⁴ is independently optionally substituted C₁₋₆ alkyl, optionally substituted C₆₋₁₀ aryl, optionally substituted C₃₋₆ cycloalkyl, optionally substituted 5-6 membered heteroaryl, optionally substituted 4-14 membered heterocycloalkyl, optionally substituted C₆₋₁₀ aryl-C₁₋₆ alkyl-, optionally substituted C₃₋₆ cycloalkyl-C₁₋₆ alkyl-, optionally substituted 5-6 membered heteroaryl-C₁₋₆ alkyl-, or optionally substituted 4-14 membered heterocycloalkyl-C₁₋₆ alkyl.

Also provided are pharmaceutical compositions comprising a compound of Formula I, or a pharmaceutically acceptable salt or stereoisomer thereof, and a pharmaceutically acceptable excipient.

Also provided are pharmaceutical compositions comprising a compound of Formula II:

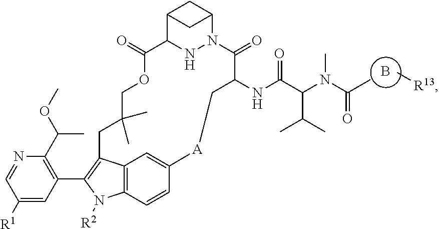

or a pharmaceutically acceptable salt or stereoisomer thereof, and a pharmaceutically acceptable excipient.

Also provided are pharmaceutical compositions comprising a compound of Formula III:

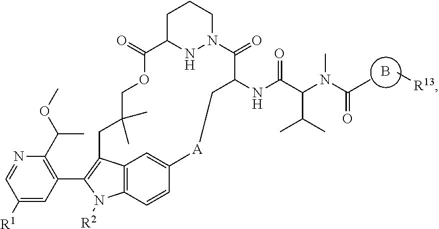

or a pharmaceutically acceptable salt or stereoisomer thereof, and a pharmaceutically acceptable excipient.

Also provided are pharmaceutical compositions comprising a compound of Table 1, or a pharmaceutically acceptable salt or stereoisomer thereof, and a pharmaceutically acceptable excipient.

Also provided is a method of treating cancer in a subject in need thereof, the method comprising administering to the subject a therapeutically effective amount of a compound of the present invention, or a pharmaceutically acceptable salt thereof.

In some embodiments, a method is provided of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I or Formula I′ and subformulae thereof, for example, a compound of Table 1 as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Accordingly, unless explicitly excluded, the term “compound” encompasses all such stereoisomers. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. The terms “C₁-C₆ alkyl” and “C₁₋₆ alkyl” are used interchangeably. For example, the term “C₁-C₆ alkyl” or “C₁₋₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted,” whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; oxo; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4- to 8-membered saturated or unsaturated heterocycloalkyl (e.g., pyridyl); 3- to 8-membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R⁰)—S(O)₂—R^{∘}; —C(NCN)NR^{∘}₂; —(CH₂)₀₋₄C(O)SR^{∘}; —(CH₂)₀₋₄C(O)oSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ alkyl, C₂₋₆ alkenyl, C₂₋₆ alkynyl, C₃₋₁₀ cycloalkyl, C₆₋₁₀ aryl, 5- to 14-membered heteroaryl having 1-4 ring heteroatoms selected from O, N and S, 4- to 14-membered heterocycloalkyl having 1-4 ring heteroatoms selected from O, N, S, SO and SO₂, wherein the nitrogen is optionally oxidized, C₃₋₁₀ cycloalkyl-C₁₋₆ alkyl-, C₆₋₁₀ aryl-C₁₋₆ alkyl-, (5-14-membered heteroaryl)-C₁₋₆ alkyl, (4-14 membered heterocycloalkyl)-C₁₋₆ alkyl-, —CH₂Ph, —O(CH₂)₀₋₁Ph, —CH₂-(5- to 6-membered heteroaryl ring), —CH₂-(5- to 10-membered heteroaryl ring), 3- to 15-membered saturated, partially unsaturated, or aryl ring having 0-4 heteroatoms independently selected from nitrogen, oxygen, sulfur, SO or SO₂, wherein the nitrogen is optionally oxidized; or a 3- to 6-membered saturated, partially unsaturated, or aryl ring having 0-4 heteroatoms independently selected from nitrogen, oxygen, or sulfur, wherein the nitrogen is optionally oxidized; or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

R^{∘} (or the ring formed by taking two independent occurrences of R^{∘} together with their intervening atoms), may be substituted with 1, 2, 3, 4, or 5 substituents independently selected from halogen, oxo, CN, N₃, C₁₋₆ alkyl, C₂₋₆ alkenyl, C₂₋₆ alkynyl, —(CH₂)₀₋₂R′, -(haloR′), —(CH₂)₀₋₂OH, —(CH₂)₀₋₂OR′, —(CH₂)₀₋₂CH(OR′)₂; —O(haloR′), —CN, —N₃, —(CH₂)₀₋₂C(O)R′, —(CH₂)₀₋₂C(O)OH, —(CH₂)₀₋₂C(O)OR′, —(CH₂)₀₋₂SR′, —(CH₂)₀₋₂SH, —(CH₂)₀₋₂NH₂, —(CH₂)₀₋₂NHR′, —(CH₂)₀₋₂NR′2, —NO₂, —SiR′₃, —OSiR′₃, —C(O)SR′, —(C₁₋₄ straight or branched alkylene)C(O)OR′, —SSR′, —(CH₂)₀₋₄N(R′)₂; —(CH₂)₀₋₄N(R′)C(O)R′; —N(R′)C(S)R′; —(CH₂)₀₋₄N(R′)C(O)NR′₂; —N(R′)C(S)NR′₂; —(CH₂)₀₋₄N(R′)C(O)OR′; —N(R′)N(R′)C(O)R′; —N(R′)N(R′)C(O)NR′₂; —N(R′)N(R′)C(O)OR′; —(CH₂)₀₋₄C(O)R′; —C(S)R′; —(CH₂)₀₋₄C(O)OR′; —(CH₂)₀₋₄—C(O)—N(R′)₂; —(CH₂)₀₋₄—C(O)—N(R^{∘})—S(O)₂—R′; —C(NCN)NR′₂; —(CH₂)₀₋₄C(O)SR′; —(CH₂)₀₋₄C(O)OSiR′₃; —(CH₂)₀₋₄OC(O)R′; —OC(O)(CH₂)₀₋₄SR′; —SC(S)SR′; —(CH₂)₀₋₄SC(O)R′; —(CH₂)₀₋₄C(O)NR′₂; —C(S)NR′₂; —C(S)SR^{∘}; —(CH₂)₀₋₄OC(O)NR′₂; —C(O)N(OR′)R′; —C(O)C(O)R′; —C(O)CH₂C(O)R′; —C(NOR′)R′; —(CH₂)₀₋₄SSR′; —(CH₂)₀₋₄S(O)₂R′; —(CH₂)₀₋₄S(O)₂₀R′; —(CH₂)₀₋₄OS(O)₂R′; —S(O)₂NR′₂; —(CH₂)₀₋₄S(O)R′; —N(R′)S(O)₂NR′₂; —N(R′)S(O)₂R′; —N(OR′)R′; —C(NOR′)NR′₂; —C(NH)NR′₂; —P(O)₂R′; —P(O)R′₂; —P(O)(OR′)₂; —OP(O)R′₂; —OP(O)(OR′)₂; —OP(O)(OR′)R′; wherein each R′ is where preceded by “halo” is substituted only with one or more halogens, and is each independently selected from C₁₋₆ alkyl, C₁₋₆ haloalkyl, C₂₋₆ alkenyl, C₂₋₆ alkynyl, C₃₋₁₀ cycloalkyl, C₆₋₁₀ aryl, 5- to 14-membered heteroaryl having 1-4 ring heteroatoms selected from O, N and S, 4- to 14-membered heterocycloalkyl having 1-4 ring heteroatoms selected from O, N, S, SO and SO₂, wherein the nitrogen is optionally oxidized, C₃₋₁₀ cycloalkyl-C₁₋₆ alkyl-, C-10 aryl-C₁₋₆ alkyl-, (5-14-membered heteroaryl)-C₁₋₆ alkyl, (4-14 membered heterocycloalkyl)-C₁₋₆ alkyl-C₁₋₄ aliphatic, —CH₂Ph, —O(CH₂)₀₋₁Ph, a 3- to 8-membered saturated, partially unsaturated, or aryl ring having 0-4 heteroatoms independently selected from nitrogen, oxygen, or sulfur, or a 5- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S. At each occurrence, each R′ is optionally substituted with 1, 2, 3, 4 or 5 independently selected R″ substituents.

Each R″ is independently selected from C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₆ alkyl-, (4-14 membered heterocycloalkyl)-C₁₋₆ alkyl-, halogen, CN, OR^{a}, SR^{a}, NHOR^{a}, C(O)R^{a}, C(O)NR^{a}R^{a}, C(O)OR^{a}, C(O)NR^{a}S(O)₂R^{a}, OC(O)R^{a}, OC(O)NR^{a}R^{a}, NHR^{a}, NR^{a}R^{a}, NR^{a}C(O)R^{a}, NR^{a}C(═NR^{a})R^{a}, NR^{a}C(O)OR^{a}, NR^{a}C(O)NR^{a}R^{a}, C(═NR^{a})R^{a}, C(═NOH)R^{a}, C(═NOH)NR^{a}, C(═NCN)NR^{a}R^{a}, NR^{a}C(═NCN)NR^{a}R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, S(O)₂NR^{a}C(O)R^{a}, —P(O)R^{a}R^{a}, —P(O)(OR^{a})(OR^{a}), —B(OH)₂, —B(OR^{a})₂ and S(O)₂NR^{a}R^{a}, wherein each R″ is optionally substituted with 1, 2, or 3 independently selected R^{b} substituents.

Each R^{a} is independently selected from H, C₁₋₆ alkyl, C₁₋₆ haloalkyl, C₂₋₆ alkenyl, C₂₋₆ alkynyl, phenyl, C₃₋₁₀ cycloalkyl, 5-10 membered heteroaryl, 4-14 membered heterocycloalkyl, phenyl-C₁₋₆ alkyl, C₃₋₁₀ cycloalkyl-C₁₋₆ alkyl, (5-10 membered heteroaryl)-C₁₋₆ alkyl, and (4-14 membered heterocycloalkyl)-C₁₋₆ alkyl, each of which is optionally substituted with 1, 2, or 3 independently selected R^{d} substituents.

Each R^{b} is independently selected from oxo, CN, halogen, NH₂, OH, COOH, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₁₄ cycloalkyl, 5-10 membered heteroaryl, 4-14 membered heterocycloalkyl, phenyl-C₁₋₄ alkyl, C₃₋₁₀ cycloalkyl-C₁₋₄ alkyl, (5-10 membered heteroaryl)-C₁₋₄ alkyl, and (4-14 membered heterocycloalkyl)-C₁₋₄ alkyl, NHOR^{c}, OR^{c}, SR^{c}, C(O)R^{c}, C(O)NR^{c}R^{c}, C(O)OR^{c}, C(O)NR^{c}S(O)₂R^{c}, OC(O)R^{c}, OC(O)NR^{c}R^{c}, C(═NOH)R^{c}, C(═NOH)NR^{c}, C(═NCN)NR^{c}R^{c}, NR^{c}C(═NCN)NR^{c}R^{c}, C(═NR^{c})NR^{c}R^{c}, NR^{c}C(═NR^{c})NR^{c}R^{c}, NHR^{c}, NR^{c}R^{c}, NR^{c}C(O)R^{c}, NR^{c}C(═NR^{c})R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c}, S(O)₂NR^{c}C(O)R^{c}, —P(O)R^{c}R^{c}, —P(O)(OR^{c})(OR^{c}), —B(OH)₂, —B(OR^{c})₂ and S(O)₂NR^{c}R^{c};

Each R^{c} is independently selected from H, C₁₋₆ alkyl, C₁₋₄ haloalkyl, C₂₋₆ alkenyl, C₂₋₆ alkynyl, phenyl, C₃₋₁₀ cycloalkyl, 5-10 membered heteroaryl, 4-10 membered heterocycloalkyl, phenyl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl;
wherein each R^{d} is independently selected from H, C₁₋₆ alkyl, C₁₋₄ haloalkyl, C₂₋₆ alkenyl, C₂₋₆ alkynyl, phenyl, C₃₋₁₀ cycloalkyl, 5-10 membered heteroaryl, 4-10 membered heterocycloalkyl, phenyl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl;
wherein two R′ substituents together with the nitrogen atom to which they are attached can form a 4-, 5-, 6-, 6-, 8-, 9- or 10-membered heterocycloalkyl group optionally substituted with 1, 2, or 3 independently selected R^{e} substituents;
wherein any two R^{a} substituents together with the nitrogen atom to which they are attached can form a 4-, 5-, 6-, 6-, 8-, 9- or 10-membered heterocycloalkyl group optionally substituted with 1, 2, or 3 independently selected R^{e} substituents;
wherein two R^{c} substituents together with the nitrogen atom to which they are attached can form a 4-, 5-, 6-, 6-, 8-, 9- or 10-membered heterocycloalkyl group optionally substituted with 1, 2, or 3 independently selected R^{e} substituents;
wherein each R^{e} is independently selected from C₁₋₆ alkyl, C₁₋₆ alkoxy, C₁₋₆ haloalkyl, C₁₋₆ haloalkoxy, C₂₋₆ alkenyl, C₂₋₆ alkynyl, CN, OH, —COOH, —COOR^{f}, NH₂, —NHR^{f}, —N(R^{f})₂, —C(O)NHR^{f}, —SO₂R^{f}, —NHC(O)R^{f}, —C(O)R^{f}, NR′^{f}S(O)₂R′^{f}, NR′^{f}S(O)₂NR′^{f}R′^{f}, S(O)₂NR′^{f}C(O)R′^{f}, S(O)₂NR′^{f}R^{f}, phenyl, C₃₋₆ cycloalkyl, 4-6 membered heterocycloalkyl, or 5-6 membered heteroaryl;
wherein each R^{f} is independently selected from H, C₁₋₆ alkyl, phenyl, C₃₋₆ cycloalkyl, 4-6 membered heterocycloalkyl, and 5-6 membered heteroaryl; or
wherein two R^{f} substituents together with the nitrogen atom to which they are attached can form a 4-, 5-, 6-, 6-, 8-, 9- or 10-membered heterocycloalkyl group optionally substituted with 1, 2, or 3 independently selected R^{g} substituents.

In some embodiments, R^{g} can be halogen, —CN, —OH, —COOH, —NH₂, —NH—C₁₋₆ alkyl, —N(C₁₋₆ alkyl)₂, C₁₋₆ alkyl, C₁₋₆ alkoxy, C₁₋₆ haloalkyl, C₁₋₆ haloalkyl, C₁₋₆ haloalkoxy, —C(O)NH₂, —C(O)NH—C₁₋₆ alkyl, phenyl, C₃₋₆ cycloalkyl, 4-6 membered heterocycloalkyl, or 5-6 membered heteroaryl.

In some embodiments, divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, a 3- to 8-membered saturated, partially unsaturated, or aryl ring having 0-4 ring heteroatoms independently selected from nitrogen, oxygen, or sulfur, or an unsubstituted 5- to 6-membered saturated, partially unsaturated, or aryl ring having 0-4 ring heteroatoms independently selected from nitrogen, oxygen, or sulfur, wherein the nitrogen is optionally oxidized. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur.

In some embodiments, substituents on the aliphatic group of R* include halogen, —R′, -(haloR′), —OH, —OR′, —O(haloR′), —CN, —C(O)OH, —C(O)OR′, —NH₂, —NHR′, —NR′₂, or —NO₂. In some embodiments, each R′ is unsubstituted or where preceded by “halo” is substituted only with one or more halogens, and is independently C₁₋₄ aliphatic, —CH₂Ph, —O(CH₂)₀₋₁Ph, a 3- to 8-membered saturated, partially unsaturated, or aryl ring having 0-4 heteroatoms independently selected from nitrogen, oxygen, or sulfur, or a 5- to 6-membered saturated, partially unsaturated, or aryl ring having 0-4 heteroatoms independently selected from nitrogen, oxygen, or sulfur.

In some embodiments, substituents on a substitutable nitrogen of an “optionally substituted” group include: —R^{†}, —NR†₂, —C(O)R^{†}, —C(O)OR†, —C(O)C(O)R^{†}, —C(O)CH₂C(O)R^{†}, —S(O)₂R^{†}, —S(O)₂NR†₂, —C(S)NR†₂, —C(NH)NR†₂, or —N(R^{†})S(O)₂R^{†}; wherein each R^{†} is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

In some embodiments, substituents on an aliphatic group of Rt are independently halogen, —R′, -(haloR′), —OH, —OR′, —O(haloR′), —CN, —C(O)OH, —C(O)OR′, —NH₂, —NHR′, —NR′₂, or —NO₂. In some embodiments, each R′ is unsubstituted or where preceded by “halo” is substituted only with one or more halogens, and is independently C₁₋₄ aliphatic, —CH₂Ph, —O(CH₂)₀₋₁Ph, a 3- to 8-membered saturated, partially unsaturated, or aryl ring having 0-4 heteroatoms independently selected from nitrogen, oxygen, or sulfur, or a 5- to 6-membered saturated, partially unsaturated, or aryl ring having 0-4 heteroatoms independently selected from nitrogen, oxygen, or sulfur. Suitable divalent substituents on a saturated carbon atom of Rt include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers.

The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

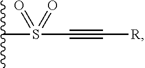

wherein R is any chemically feasible substituent described herein.

The term “amino,” as used herein, represents —N(Rt)₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any carbon ring atom that results in a stable structure and any of the ring atoms can be optionally substituted unless otherwise specified.

The term “arylene,” as used herein, refers to a divalent aryl group. An optionally substituted arylene is an arylene that is optionally substituted as described herein for aryl.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, cyclic hydrocarbon group, which may be bridged, fused or spirocyclic having from three to eight ring carbons, unless otherwise specified, and containing one or more carbon-carbon double bonds.

The term “cycloalkylene,” as used herein, represents a divalent cycloalkyl group. An optionally substituted cycloalkylene is a cycloalkylene that is optionally substituted as described herein for cycloalkyl.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

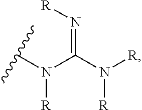

wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic, or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heteroarylene,” as used herein, represents a divalent heteroaryl. An optionally substituted heteroarylene is a heteroarylene that is optionally substituted as described herein for heteroaryl.

The term “heterocycloalkyl,” as used herein, represents a monovalent monocyclic, bicyclic, or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The non-aromatic ring may optionally contain one or more double bonds while still being non-aromatic. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “heterocycloalkylene,” as used herein, represents a divalent heterocycloalkyl group. An optionally substituted heterocycloalkylene is a heterocycloalkylene that is optionally substituted as described herein for heterocycloalkyl.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., one portion of a macrocycle) to a second moiety (e.g., a second portion of the same macrocycle).

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

The term “stereoisomer,” as used herein, refers to all possible different isomeric as well as conformational forms which a compound may possess (e.g., a compound of any formula described herein), in particular all possible stereochemical and conformational isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that non-covalent interactions of a compound of the present invention with Ras and the chaperone protein (e.g., cyclophilin A) contribute to the inhibition of Ras activity. For example, van der Waals, hydrophobic, hydrophilic and hydrogen bond interactions, and combinations thereof, may contribute to the ability of the compounds of the present invention to form complexes and act as Ras inhibitors.

The compounds of the present invention exhibit inhibitory activities across all RAS mutants. In some embodiments, a compound of the present invention inhibits a RAS mutant with one or more mutations at G12X, G13X, and/or Q61X, wherein X represents any naturally occurring amino acid residue. In certain instances, X is A, C, D, V, S, R, H, K, or L amino acid residue.

In certain embodiments, a compound of the present invention inhibits a RAS mutant with one or more mutations at G12X, wherein X represents any naturally occurring amino acid residue. In certain instances, X is A, C, D, V, S or R amino acid residue.

In other embodiments, a compound of the present invention inhibits a RAS mutant with one or more mutations at G13X, wherein X is any naturally occurring amino acid residue. In certain instances, X is A, C, D, V, S or R amino acid residue.

In other embodiments, a compound of the present invention inhibits a RAS mutant with one or more mutations at Q61X, wherein X is any naturally occurring amino acid residue. In certain instances, X is A, C, D, V, S, R, H, K, or L amino acid residue. In other instances, X is H, K, R, or L amino acid residue.

A variety of Ras proteins may be inhibited by a compound of the present invention (e.g., K-Ras, N-Ras, H-Ras, and mutants thereof at positions 12, 13 and 61, such as G12C, G12D, G12V, G12S, G12R, G13C, G13D, Q61H, Q61K, Q61R and Q61L, and others described herein, or a combination thereof). In some embodiments, a compound of the present invention inhibits a G12C, G12D, G12R or G12S mutant of RAS, or a combination thereof. In some embodiments, a compound of the present invention interacts selectively with K-RasG12V versus wildtype and versus other mutants.

Oncogenic RAS mutations increase the proportion of RAS protein in the GTP-bound state. Without wishing to be bound by theory, the inventors postulate that compounds as disclosed herein increase the rate of GTP hydrolysis by oncogenic RAS and/or wild-type RAS, thereby increasing the levels of GDP-bound RAS. The RAS(ON) GTP hydrolysis-promoting compounds disclosed herein may show greater selectivity for RAS^{G12X} relative to RAS^{WT} due to the inherent GAP-deficiency of mutant RAS isoforms. Thus, RAS(ON) GTP hydrolysis-promoting compounds disclosed herein may be useful in the context of RAS^{AMP} (e.g., mutant RAS^{AMP}) due to the catalytic rather than stoichiometric mechanism of target inhibition. Moreover, the unique profile of the RAS(ON) GTP hydrolysis-promoting compounds disclosed herein may lend itself to improved tolerability including in the context of combination therapies, particularly in-pathway combinations. As used herein, a “RAS(ON) GTP hydrolysis-promoting compound” refers to a tri-complex-forming compound that, when bound in a tri-complex (i.e. CYPA-RAS(ON) GTP hydrolysis-promoting compound-RAS(ON) isoform), exhibits a RAS(ON) GTP hydrolysis rate that is greater than the intrinsic hydrolysis rate of the RAS(ON) mutant isoform (RAS^{MUT}) and/or RAS wild-type isoform (RAS^{WT}) in the absence of the compound. In some embodiments, a RAS(ON) GTP hydrolysis-promoting compound exhibits a hydrolysis rate that is greater than 14× the intrinsic hydrolysis rate (a “strong hydrolyzer”). In some embodiments, a RAS(ON) GTP-hydrolysis-promoting compound exhibits a hydrolysis rate that is 5-14× the intrinsic hydrolysis rate (a “moderate hydrolyzer”). In some embodiments, a RAS(ON) GTP hydrolysis-promoting compound exhibits a hydrolysis rate that is greater than 1× the intrinsic hydrolysis rate but less than 5× the intrinsic hydrolysis rate (a “weak hydrolyzer”). In some embodiments, KRAS^{G12V} is the isoform used for determining a strong hydrolyzer, moderate hydrolyzer, or weak hydrolyzer. Methods of measuring hydrolysis are known in the art, such as those described herein. In some embodiments, a RAS(ON) GTP hydrolysis-promoting compound is a RAS(ON) inhibitor. All RAS(ON) GTP hydrolysis-promoting compounds retain a catalytic water in the proximity of the gamma phosphorous of GTP (distance <5 angstroms) and position the delta carbon of the Q61 of RAS(ON) within 8 angstroms of the gamma phosphorous: these parameters may be determined by one of skill in the art. Further description of RAS(ON) GTP hydrolysis-promoting compounds are described herein.

Compounds of the present disclosure may be a RAS(ON) GTP hydrolysis-promoting compounds. A RAS(ON) GTP hydrolysis-promoting compound of the present disclosure forms a high affinity tri-complex with two intracellular proteins which do not interact under normal physiological conditions: RAS and a widely expressed cytosolic chaperone in the cell, cyclophilin A (CypA). In addition, the present disclosure provides non-covalent binding of RAS by a compound of the present invention to promote a catalytically competent orientation of RAS(ON) in which the glutamine 61 (Q61) side chain coordinates a catalytic water to promote nucleophilic attack at the gamma phosphate GTP bound to the RAS protein.

Accordingly, provided herein is a compound of Formula (I):

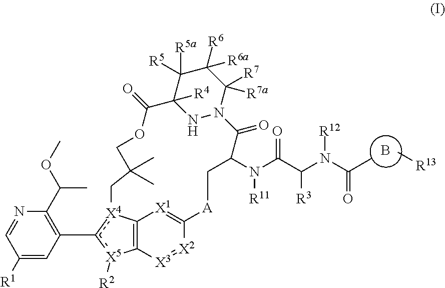

or a pharmaceutically acceptable salt thereof, wherein:
A is optionally substituted 3- to 6-membered cycloalkylene, optionally substituted 4- to 6-membered heterocycloalkylene, optionally substituted C₆₋₁₀ arylene, or optionally substituted 5- to 10-membered heteroarylene;
ring B is optionally substituted 4- to 11-membered heterocycloalkylene, wherein 1 or 2 ring carbon atoms of the 4- to 11-membered heterocycloalkylene are optionally replaced by a member independently selected from C(═O) and SO₂;
X¹, X² and X³ are each independently N or CR⁸, wherein each R⁸ is independently hydrogen, halogen, CN, or C₁₋₆ alkyl;
X⁴ is N and X⁵ is C; or
X⁵ is N and X⁴ is C;
[symbol] is either a single bond or a double bond to maintain the aromaticity of the 5-membered ring containing X⁴ and X⁵;
R¹ is hydrogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted 3- to 6-membered cycloalkyl, optionally substituted 3- to 6-membered cycloalkenyl, optionally substituted 4- to 14-membered heterocycloalkyl, optionally substituted 6- to 10-membered aryl, or optionally substituted 5- to 10-membered heteroaryl;
R² is optionally substituted C₁₋₆ alkyl, optionally substituted C₂₋₆ alkynyl, optionally substituted C₁₋₆ heteroalkyl, optionally substituted 3- to 6-membered cycloalkyl, or optionally substituted 4- to 14-membered heterocycloalkyl;
or R² and R³ together with the atoms to which they are attached form a fused C₄₋₁₀ cycloalkyl or a fused 4- to 10-membered heterocycloalkyl having 1, 2 or 3 ring heteroatoms selected from N, O and S, wherein the fused C₄₋₁₀ cycloalkyl and heterocycloalkyl are each optionally substituted;
R³ is hydrogen, optionally substituted C₁₋₆ alkyl, optionally substituted C₁₋₃ heteroalkyl, optionally substituted C₃₋₆ cycloalkyl, or optionally substituted 4- to 6-membered heterocycloalkyl;
R⁴, R⁵, R^{5a}, R⁶, R^{6a}, R⁷ and R^{7a} are each independently hydrogen, halogen, or C₁₋₆ alkyl;
or R⁵ and R⁷ are taken together to form methylene or ethylene, which is covalently bound to the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form methylene or ethylene, which is covalently bound to the carbon atoms to which they are attached, and R⁵, R^{5a}, R^{6a}, R⁷ and R^{7a} are each independently hydrogen, halogen, or C₁₋₆ alkyl;
or R⁴ and R⁷ are taken together to form methylene or ethylene, which is covalently bound to the carbon atoms to which they are attached, R⁵, R^{5a}, R⁶, R^{6a}, and R^{7a} are each independently hydrogen, halogen, or C₁₋₆ alkyl;
or R⁵ and R⁶ taken together with the atoms to which they are attached form optionally substituted fused C₃₋₆ cycloalkyl or optionally substituted 4- to 6-membered fused heterocycloalkyl;
or R⁶ and R⁷ taken together with the atoms to which they are attached form optionally substituted fused C₃₋₆ cycloalkyl or optionally substituted 4- to 6-membered fused heterocycloalkyl;
or R⁵ and R^{5a} taken together with the atom to which they are attached form optionally substituted C₃₋₆ cycloalkyl or optionally substituted 4- to 6-membered heterocycloalkyl;
or R⁶ and R^{6a} taken together with the atom to which they are attached form optionally substituted C₃₋₆ cycloalkyl or optionally substituted 4- to 6-membered heterocycloalkyl;
or R⁷ and R^{7a} taken together with the atom to which they are attached form an optionally C₃₋₆ cycloalkyl or optionally substituted 4- to 6-membered heterocycloalkyl;
or R⁵ and R⁶ are taken together to form a bond;
R¹¹ is hydrogen or optionally substituted C₁₋₆ alkyl;
R¹² is hydrogen or optionally substituted C₁₋₆ alkyl; and
R¹³ is H, optionally substituted C₁₋₆ alkyl, —C(O)—R¹⁴, —C(O)OR¹⁴, —C(O)NHR¹⁴, —C(O)NR¹⁴R¹⁴, —SO₂R¹⁴, —SO₂NHR¹⁴, —SO₂NR¹⁴R¹⁴, wherein each R¹⁴ is independently optionally substituted C₁₋₆ alkyl, optionally substituted C₆₋₁₀ aryl, optionally substituted C₃₋₆ cycloalkyl, optionally substituted 5-6 membered heteroaryl, optionally substituted 4-14 membered heterocycloalkyl, optionally substituted C₆₋₁₀ aryl-C₁₋₆ alkyl-, optionally substituted C₃₋₆ cycloalkyl-C₁₋₆ alkyl-, optionally substituted 5-6 membered heteroaryl-C₁₋₆ alkyl-, or optionally substituted 4-14 membered heterocycloalkyl-C₁₋₆ alkyl,
or a pharmaceutically acceptable salt thereof.

In some embodiments, the compound is of formula (I), wherein X¹, X² and X³ are each CR⁸; X⁴ is C and X⁵ is N.

In some embodiments, the compound of formula (I) is a compound of formula (I-a):

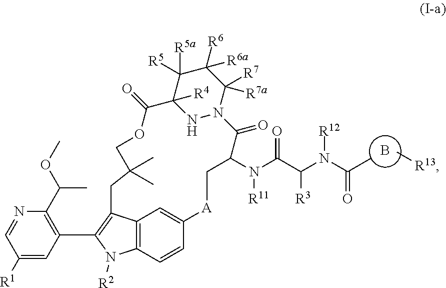

or a pharmaceutically acceptable salt thereof.

In some embodiments, the compound is of formula I or I-a, wherein:
R⁵ and R⁷ are taken together to form methylene or ethylene, which is covalently bound to the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form methylene or ethylene, which is covalently bound to the carbon atoms to which they are attached, and R⁵, R^{5a}, R^{6a}, R⁷ and R^{7a} are each independently hydrogen, halogen, or C₁₋₆ alkyl;
or R⁴ and R⁷ are taken together to form methylene or ethylene, which is covalently bound to the carbon atoms to which they are attached, R⁵, R^{5a}, R⁶, R^{6a}, and R^{7a} are each independently hydrogen, halogen, or C₁₋₆ alkyl;
or R⁵ and R⁶ taken together with the atoms to which they are attached form optionally substituted fused C₃₋₆ cycloalkyl or optionally substituted 4- to 6-membered fused heterocycloalkyl;
or R⁶ and R⁷ taken together with the atoms to which they are attached form optionally substituted fused C₃₋₆ cycloalkyl or optionally substituted 4- to 6-membered fused heterocycloalkyl;
or R⁵ and R^{5a} taken together with the atom to which they are attached form optionally substituted C₃₋₆ cycloalkyl or optionally substituted 4- to 6-membered heterocycloalkyl;
or R⁶ and R^{6a} taken together with the atom to which they are attached form optionally substituted C₃₋₆ cycloalkyl or optionally substituted 4- to 6-membered heterocycloalkyl;
or R⁷ and R^{7a} taken together with the atom to which they are attached form an optionally C₃₋₆ cycloalkyl or optionally substituted 4- to 6-membered heterocycloalkyl;
or R⁵ and R⁶ are taken together to form a bond.

In some embodiments, the compound is of formula I or I-a, or a pharmaceutically acceptable salt thereof, wherein R¹² is H or methyl.

In some embodiments, the compound is of formula I or I-a, or a pharmaceutically acceptable salt thereof, wherein R³ is isopropyl.

In some embodiments, the compound is of formula I or I-a, or a pharmaceutically acceptable salt thereof, wherein R¹¹ is H.

In some embodiments, the compound is of formula I or I-a, or a pharmaceutically acceptable salt thereof, wherein R¹¹ is CH₃.

In some embodiments, the compound is of formula I or I-a, or a pharmaceutically acceptable salt thereof, wherein R¹ is H.

In particular embodiments, the compound is of formula (II):

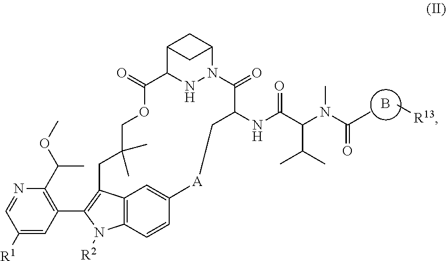

or a pharmaceutically acceptable salt thereof,
wherein
R¹ is hydrogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted 3- to 6-membered cycloalkyl, optionally substituted 3- to 6-membered cycloalkenyl, optionally substituted 4- to 14-membered heterocycloalkyl, optionally substituted 6- to 10-membered aryl, or optionally substituted 5- to 10-membered heteroaryl;
R² is optionally substituted C₁₋₆ alkyl, optionally substituted C₂₋₆ alkynyl, optionally substituted C₁₋₆ heteroalkyl, optionally substituted 3- to 6-membered cycloalkyl, or optionally substituted 4- to 14-membered heterocycloalkyl; and
R¹³ is H, optionally substituted C₁₋₆ alkyl, —C(O)—R¹⁴, —C(O)OR¹⁴, —C(O)NHR¹⁴, —C(O)NR¹⁴R¹⁴, —SO₂R¹⁴, —SO₂NHR¹⁴, —SO₂NR¹⁴R¹⁴, wherein each R¹⁴ is independently optionally substituted C₁₋₆ alkyl, optionally substituted C₆₋₁₀ aryl, optionally substituted C₃₋₆ cycloalkyl, optionally substituted 5-6 membered heteroaryl, optionally substituted 4-14 membered heterocycloalkyl, optionally substituted C₆₋₁₀ aryl-C₁₋₆ alkyl-, optionally substituted C₃₋₆ cycloalkyl-C₁₋₆ alkyl-, optionally substituted 5-6 membered heteroaryl-C₁₋₆ alkyl-, or optionally substituted 4-14 membered heterocycloalkyl-C₁₋₆ alkyl.

In other particular embodiments, the compound is of formula (III):

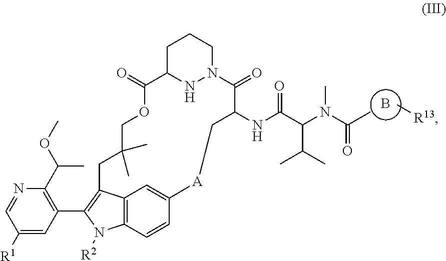

or a pharmaceutically acceptable salt thereof,
wherein
R¹ is hydrogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted 3- to 6-membered cycloalkyl, optionally substituted 3- to 6-membered cycloalkenyl, optionally substituted 4- to 14-membered heterocycloalkyl, optionally substituted 6- to 10-membered aryl, or optionally substituted 5- to 10-membered heteroaryl;
R² is optionally substituted C₁₋₆ alkyl, optionally substituted C₂₋₆ alkynyl, optionally substituted C₁₋₆ heteroalkyl, optionally substituted 3- to 6-membered cycloalkyl, or optionally substituted 4- to 14-membered heterocycloalkyl; and
R¹³ is H, optionally substituted C₁₋₆ alkyl, —C(O)—R¹⁴, —C(O)OR¹⁴, —C(O)NHR¹⁴, —C(O)NR¹⁴R¹⁴, —SO₂R¹⁴, —SO₂NHR¹⁴, —SO₂NR¹⁴R¹⁴, wherein each R¹⁴ is independently optionally substituted C₁₋₆ alkyl, optionally substituted C₆₋₁₀ aryl, optionally substituted C₃₋₆ cycloalkyl, optionally substituted 5-6 membered heteroaryl, optionally substituted 4-14 membered heterocycloalkyl, optionally substituted C₆₋₁₀ aryl-C₁₋₆ alkyl-, optionally substituted C₃₋₆ cycloalkyl-C₁₋₆ alkyl-, optionally substituted 5-6 membered heteroaryl-C₁₋₆ alkyl-, or optionally substituted 4-14 membered heterocycloalkyl-C₁₋₆ alkyl.

In particular embodiments, the compound is of formula (VI):

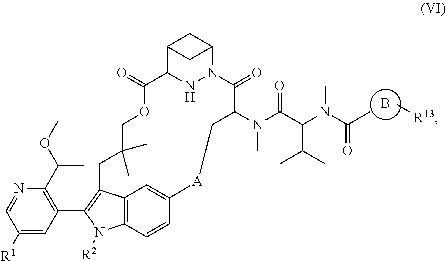

or a pharmaceutically acceptable salt thereof,
wherein
R¹ is hydrogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted 3- to 6-membered cycloalkyl, optionally substituted 3- to 6-membered cycloalkenyl, optionally substituted 4- to 14-membered heterocycloalkyl, optionally substituted 6- to 10-membered aryl, or optionally substituted 5- to 10-membered heteroaryl;
R² is optionally substituted C₁₋₆ alkyl, optionally substituted C₂₋₆ alkynyl, optionally substituted C₁₋₆ heteroalkyl, optionally substituted 3- to 6-membered cycloalkyl, or optionally substituted 4- to 14-membered heterocycloalkyl; and
R¹³ is H, optionally substituted C₁₋₆ alkyl, —C(O)—R¹⁴, —C(O)OR¹⁴, —C(O)NHR¹⁴, —C(O)NR¹⁴R¹⁴, —SO₂R¹⁴, —SO₂NHR¹⁴, —SO₂NR¹⁴R¹⁴, wherein each R¹⁴ is independently optionally substituted C₁₋₆ alkyl, optionally substituted C₆₋₁₀ aryl, optionally substituted C₃₋₆ cycloalkyl, optionally substituted 5-6 membered heteroaryl, optionally substituted 4-14 membered heterocycloalkyl, optionally substituted C₆₋₁₀ aryl-C₁₋₆ alkyl-, optionally substituted C₃₋₆ cycloalkyl-C₁₋₆ alkyl-, optionally substituted 5-6 membered heteroaryl-C₁₋₆ alkyl-, or optionally substituted 4-14 membered heterocycloalkyl-C₁₋₆ alkyl.

In other particular embodiments, the compound is of formula (V):

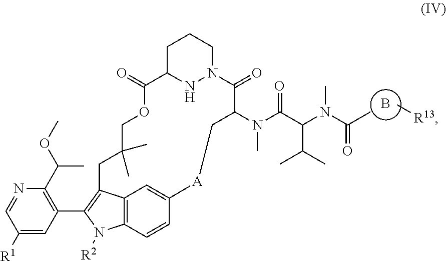

or a pharmaceutically acceptable salt thereof,
wherein
R¹ is hydrogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted 3- to 6-membered cycloalkyl, optionally substituted 3- to 6-membered cycloalkenyl, optionally substituted 4- to 14-membered heterocycloalkyl, optionally substituted 6- to 10-membered aryl, or optionally substituted 5- to 10-membered heteroaryl;
R² is optionally substituted C₁-C₆ alkyl, optionally substituted C₂₋₆ alkynyl, optionally substituted C₁-C₆ heteroalkyl, optionally substituted 3- to 6-membered cycloalkyl, or optionally substituted 4- to 14-membered heterocycloalkyl; and
R¹³ is H, optionally substituted C₁₋₆ alkyl, —C(O)—R¹⁴, —C(O)OR¹⁴, —C(O)NHR¹⁴, —C(O)NR¹⁴R¹⁴, —SO₂R¹⁴, —SO₂NHR¹⁴, —SO₂NR¹⁴R¹⁴, wherein each R¹⁴ is independently optionally substituted C₁₋₆ alkyl, optionally substituted C₆₋₁₀ aryl, optionally substituted C₃₋₆ cycloalkyl, optionally substituted 5-6 membered heteroaryl, optionally substituted 4-14 membered heterocycloalkyl, optionally substituted C₆₋₁₀ aryl-C₁₋₆ alkyl-, optionally substituted C₃₋₆ cycloalkyl-C₁₋₆ alkyl-, optionally substituted 5-6 membered heteroaryl-C₁₋₆ alkyl-, or optionally substituted 4-14 membered heterocycloalkyl-C₁₋₆ alkyl.

In some embodiments, the compound is of formula I, I-a, II, III, VI, or V, or a pharmaceutically acceptable salt thereof, and A is of the formula:

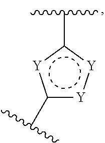

wherein
[symbol] indicates optional double bonds in the ring structure; and
each Y is independently CH, N, O, or S.

In particular embodiments, the compound is of formula II, or a pharmaceutically acceptable salt thereof, and A is

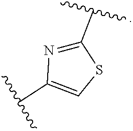 [SMILES: *c1csc(*)n1]

In other particular embodiments, the compound is of formula III, or a pharmaceutically acceptable salt thereof, and A is

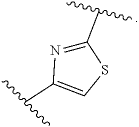 [SMILES: *c1csc(*)n1]

In other particular embodiments, the compound is of formula II, or a pharmaceutically acceptable salt thereof, and A is

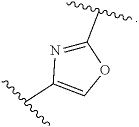 [SMILES: *c1coc(*)n1]

In other particular embodiments, the compound is of formula III, or a pharmaceutically acceptable salt thereof, and A is

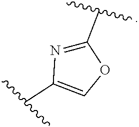 [SMILES: *c1coc(*)n1]

In some embodiments, the compound is of formula I, I-a, II, III, VI, or V, or a pharmaceutically acceptable salt thereof, and A is of the formula:

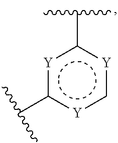

wherein
[symbol] indicates optional double bonds in the ring structure; and
each Y is independently CH or N.

In particular embodiments, the compound is of formula II or III, or a pharmaceutically acceptable salt thereof, and A is

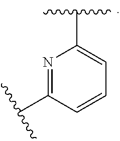 [SMILES: *c1cccc(*)n1]

In some embodiments, the compound is of formula I, I-a, II, III, VI, or V, or a pharmaceutically acceptable salt thereof, and A is of the formula:

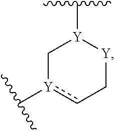

wherein
[symbol] is a single bond or a double bond; and
each Y is independently CH, CH₂, N, NH, or O.

In particular embodiments, the compound is of formula II or III, or a pharmaceutically acceptable salt thereof, and A is

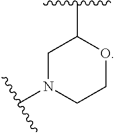 [SMILES: *C1CN(*)CCO1]

In other particular embodiments, the compound is of formula II or III, or a pharmaceutically acceptable salt thereof, and A is

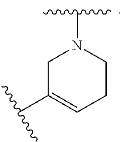 [SMILES: *C1=CCCN(*)C1]

In some embodiments, the compound is of formula I, I-a, II, III, VI, or V, or a pharmaceutically acceptable salt thereof, wherein ring B is optionally substituted 4- to 11-membered fused heterocycloalkyl or optionally substituted 4- to 11-membered spiro heterocycloalkyl or optionally substituted 4- to 6-membered heterocycloalkyl.

In some embodiments, the compound is of formula I, I-a, II, III, VI, or V, or a pharmaceutically acceptable salt thereof, and B is of the formula:

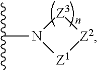

wherein,
n is 1, 2, or 3;
each of Z¹, Z², and Z³ is, independently, C═O, O, NR^{Z}, CH—R^{Z}, C(R^{Z})₂; and
each R^{Z} is independently H, optionally substituted C₁₋₆ alkyl, optionally substituted C₁₋₆ heteroalkyl, optionally substituted 4- to 6-membered cycloalkyl, optionally substituted 4- to 6-membered heterocycloalkyl, or two adjacent Z together form an optionally substituted 4- to 6-membered heterocycloalkyl or optionally substituted 4- to 6-membered cycloalkyl.

In some embodiments B is of the formula:

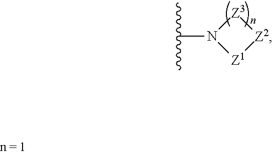

In some embodiments B is of the formula:

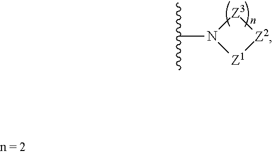

In some embodiments B is of the formula:

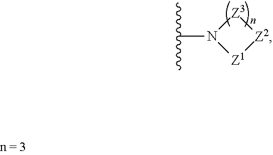

In some embodiments, B is spirocyclic. In particular embodiments, Z¹ is C(R^{Z})₂ and each R^{Z} combine to form an optionally substituted 4-6 membered heterocycloalkyl. In other particular embodiments, Z² is C(R^{Z})₂ and each R^{Z} combine to form an optionally substituted 4- to 6-membered heterocycloalkyl. In specific embodiments, said optionally substituted 4- to 6-membered heterocycloalky is selected from a group consisting of:

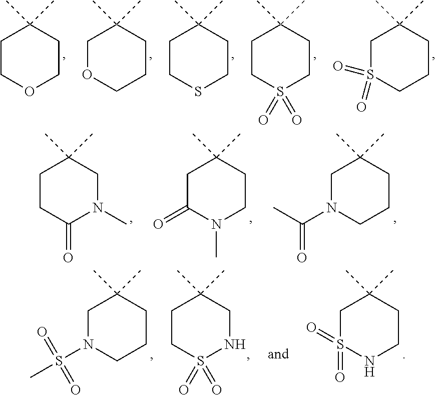

In other specific embodiments, said optionally substituted 4- to 6-membered heterocycloalkyl is selected from a group consisting of:

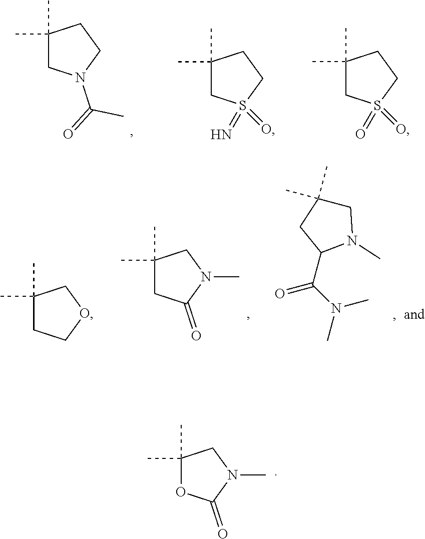

In other specific embodiments, said optionally substituted 4- to 6-membered heterocycloalkyl is selected from a group consisting of:

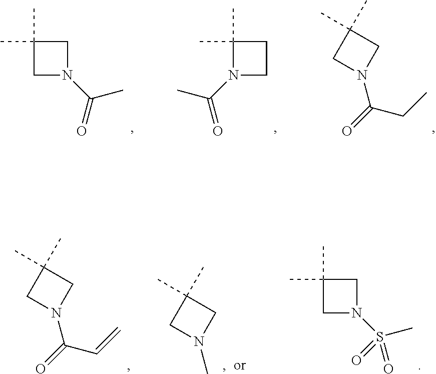

In other particular embodiments, Z¹ is C(R^{Z})₂ and each R^{Z} combine to form an optionally substituted 4- to 6-membered cycloalkyl. In other particular embodiments, Z² is C(R^{Z})₂ and each R^{Z} combine to form an optionally substituted 4- to 6-membered cycloalkyl. In specific embodiments, said optionally substituted 4- to 6-membered cycloalkyl of Z¹ or Z² is selected from a group consisting of:

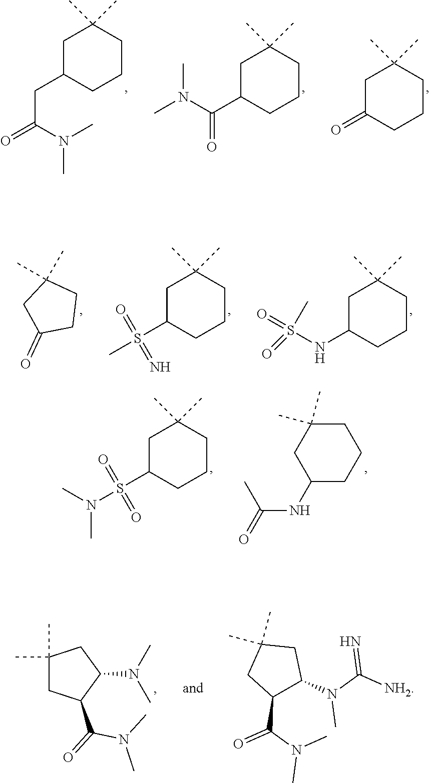

In some embodiments, when Z¹ forms a cyclic structure as described, Z² is C(R^{Z})₂, wherein each R^{Z} is H. In other embodiments, when Z¹ forms a cyclic structure as described, Z² is O. In some embodiments, when Z² forms a cyclic structure as described, Z¹ is C(R^{Z})₂, wherein each R^{Z} is H. In other embodiments, when Z² forms a cyclic structure as described, Z¹ is C═O.

In some embodiments, B is a fused ring system. In particular embodiments, Z¹ and Z² are each CH—R^{Z} and the R^{Z} from Z¹ and the R^{Z} from the an adjacent Z² combine to form an optionally substituted 4- to 6-membered heterocycloalkyl. In specific embodiments, said optionally substituted 4-6 membered heterocycloalkyl is

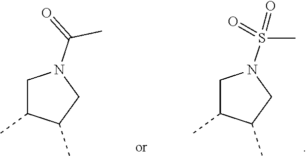

In other particular embodiments, Z² is NR^{Z} and the adjacent Z³ is CH—R^{Z}, and the R^{Z} from Z² and the R^{Z} from the an adjacent Z³ combine to form an optionally substituted 4- to 6-membered heterocycloalkyl. In specific embodiments, said optionally substituted 4- to 6-membered heterocycloalkyl is

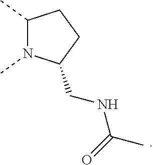

In some embodiments, all Z³ are CH—R^{Z} and R^{Z} is H.

In some embodiments, one Z³ is CH—R^{Z} and R^{Z} is CH₃.

In some embodiments, Z¹ is CH—R^{Z} and R^{Z} is

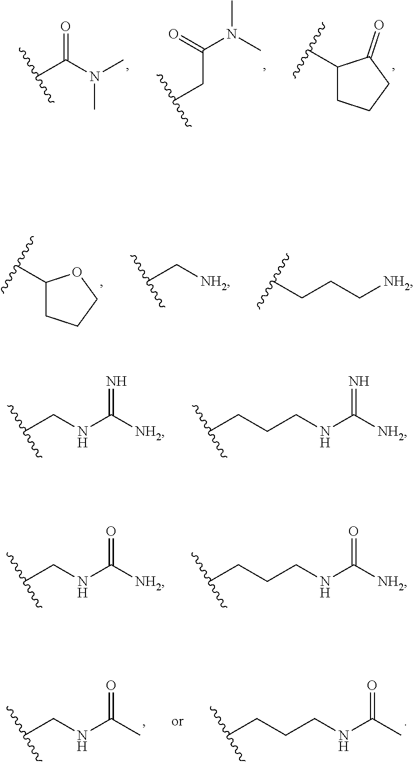

In some embodiments, Z² is CH—R^{Z} and R^{Z} is

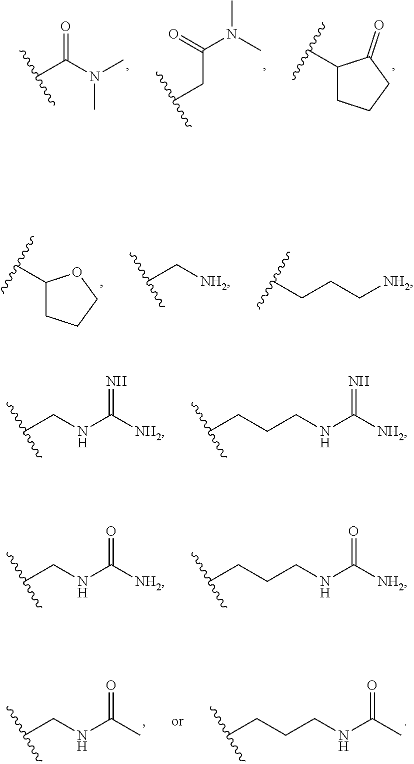

In some embodiments, Z¹ is C(R^{Z})₂, where one R^{Z} is CH₃ and the other R^{Z} is selected from the group consisting of:

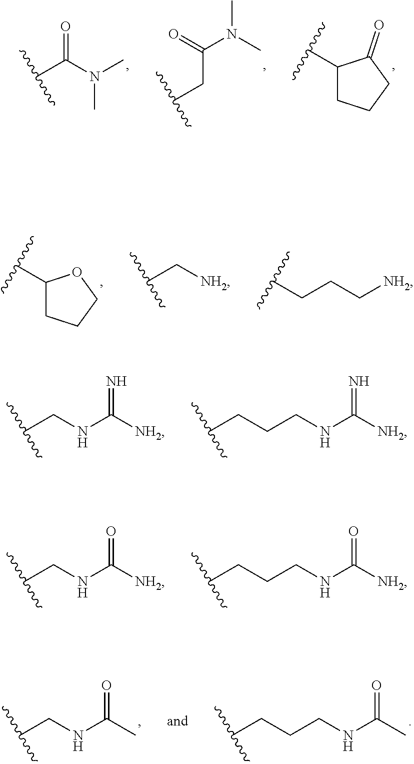

In specific embodiments, one R^{Z} is CH₃ and the other R^{Z} is

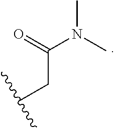 [SMILES: *CC(=O)N(C)C]

In some embodiments, the compound is of formula I, I-a, II, III, VI, or V, or a pharmaceutically acceptable salt thereof, and B—R³ is selected from a group consisting of:

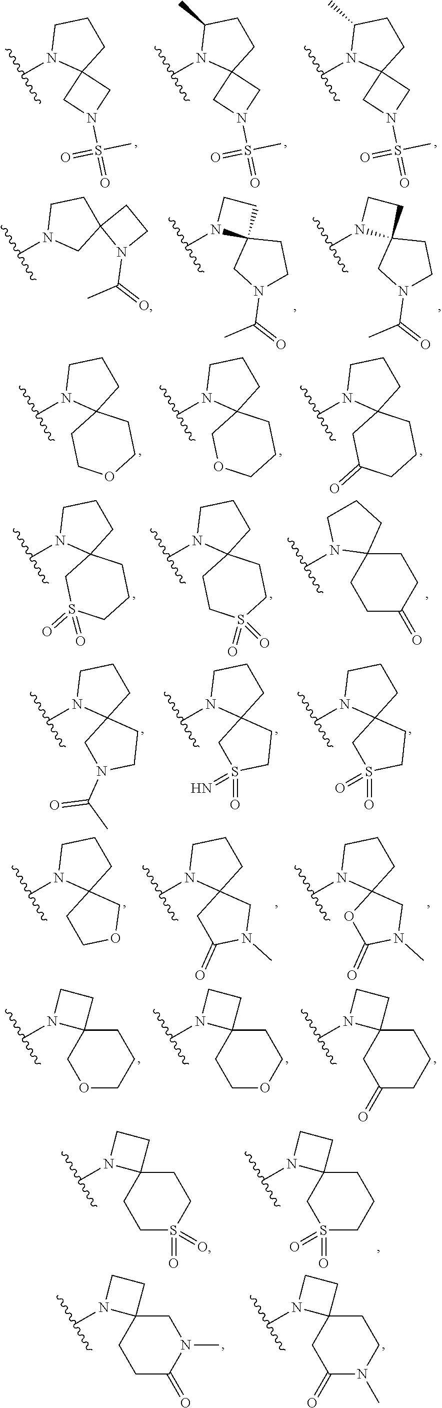

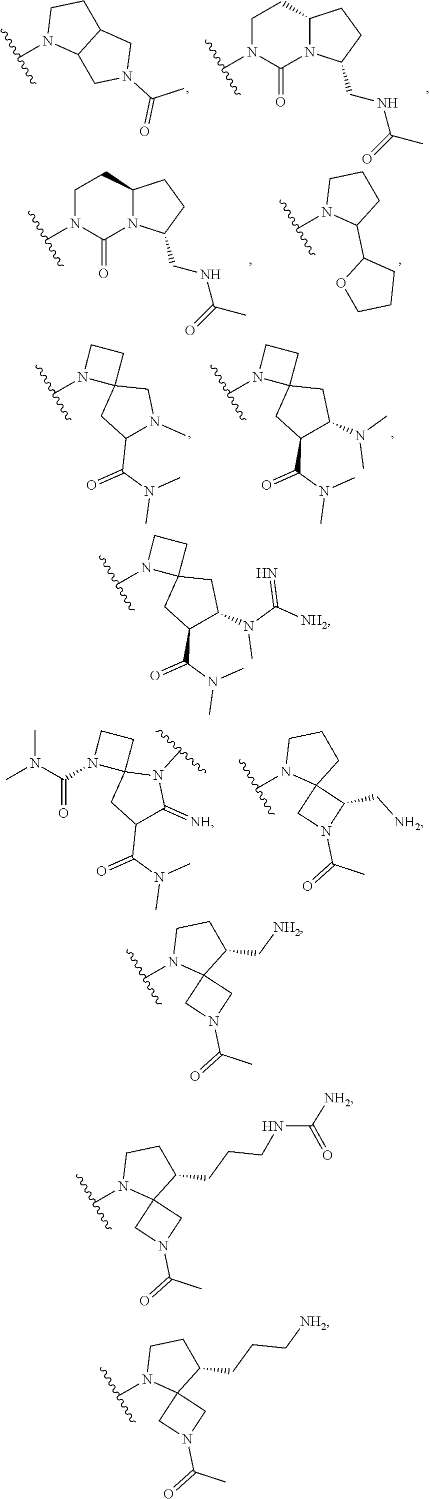

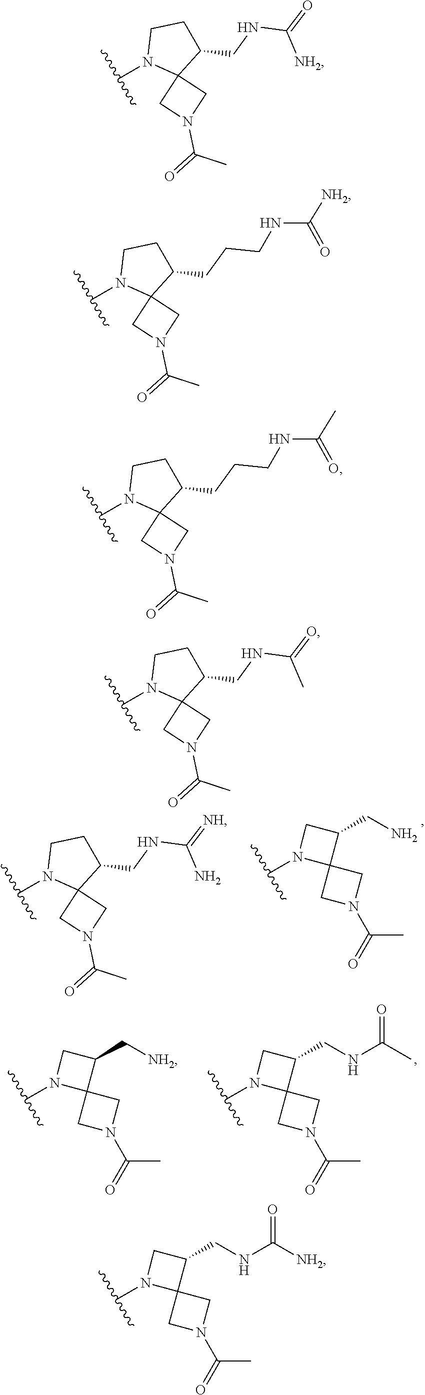

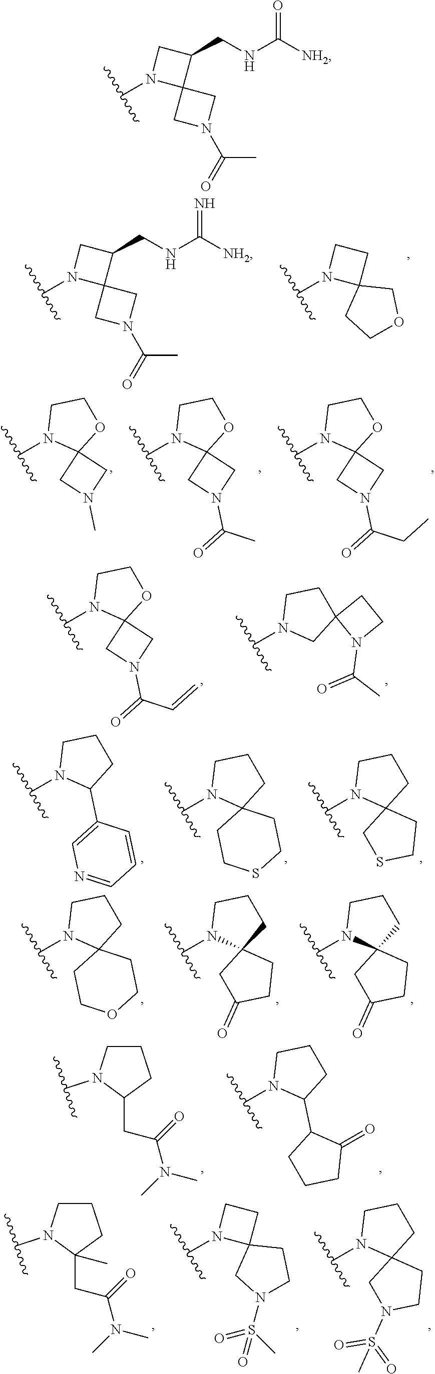

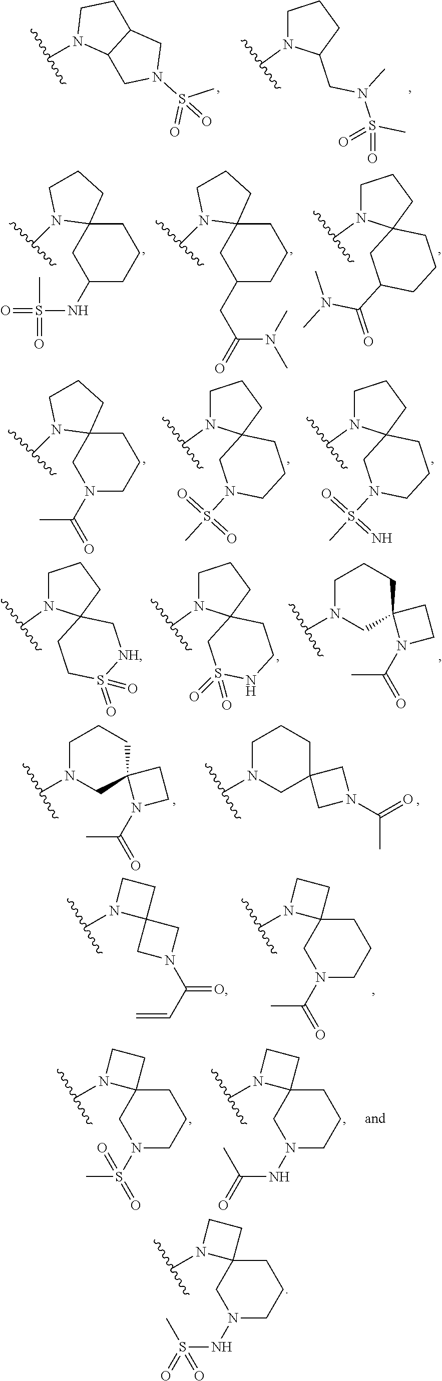

In some embodiments, the compound is of formula I, I-a, II, III, VI, or V, or a pharmaceutically acceptable salt thereof, and B—R³ is selected from a group consisting of:

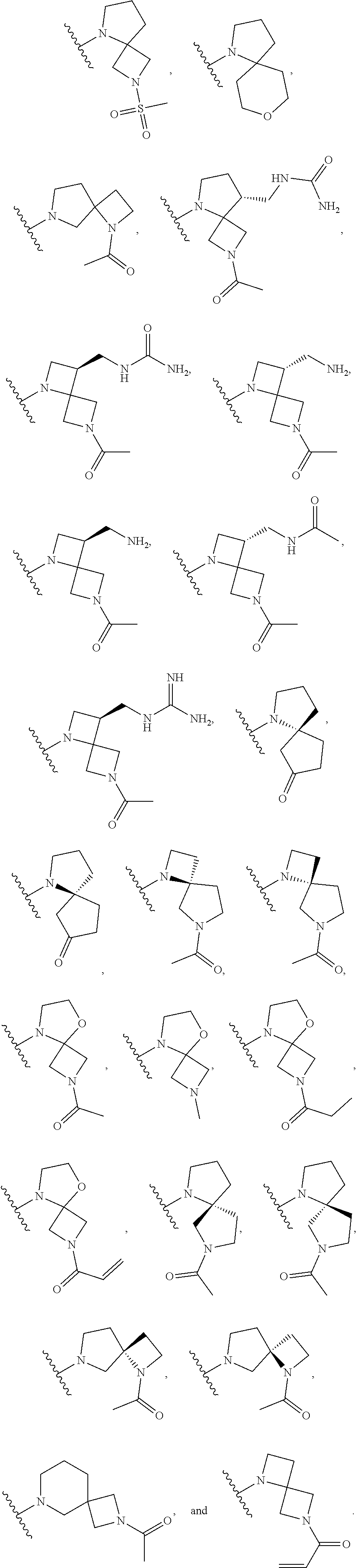

In some embodiments, the compound is of formula I, I-a, II, III, VI, or V, or a pharmaceutically acceptable salt thereof, and B—R³ is selected from a group consisting of:

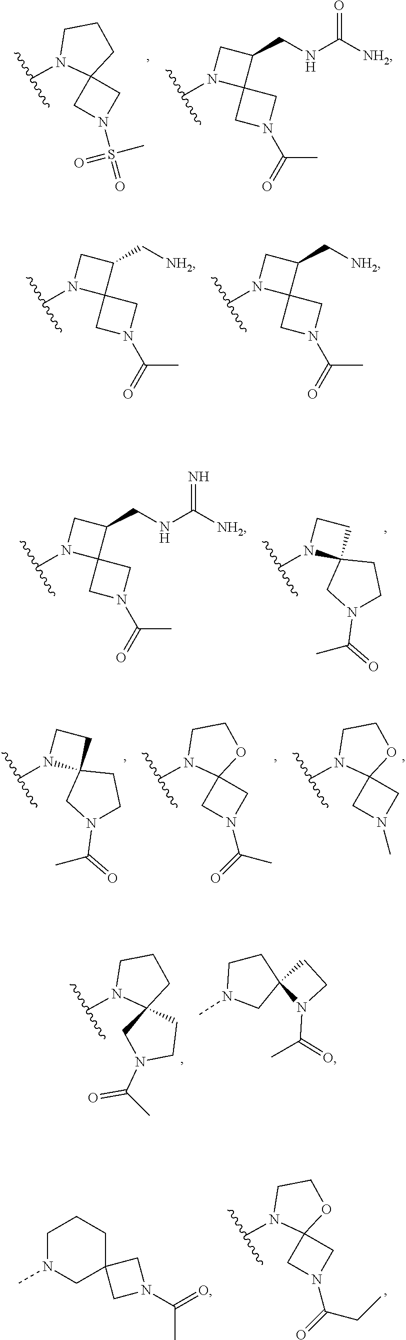

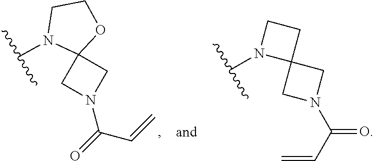 [SMILES: *N1CCOC12CN(C(=O)C=C)C2 ; *N1CCC12CN(C(=O)C=C)C2]

In some embodiments, the compound is of formula II or III, or a pharmaceutically acceptable salt thereof, and R¹ is H.

In some embodiments, R² is optionally substituted 4- to 14-membered heterocycloalkyl.

In some embodiments, the compound is of formula I, I-a, II, III, VI, or V, or a pharmaceutically acceptable salt thereof, and R² is of the formula:

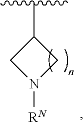

wherein,
N is 1, 2, or 3; and
R^{N} is C₁₋₆ alkyl, 3- to 6-membered cycloalkyl, or 4- to 6-membered heterocycloalkyl.

In particular embodiments, the compound is of formula I, II, or III, or a pharmaceutically acceptable salt thereof, and R² is

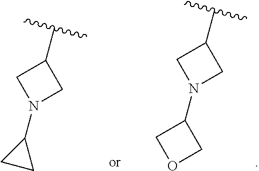 [SMILES: *C1CN(C2CC2)C1 ; *C1CN(C2COC2)C1]

In some embodiments, the compound is of formula I, I-a, II, III, VI, or V, or a pharmaceutically acceptable salt thereof, and R² is optionally substituted C₁-C₆ alkyl. In specific embodiments, R² is

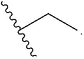 [SMILES: *CC]

In other specific embodiments, R² is

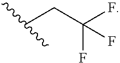 [SMILES: *CC(F)(F)F]

In some embodiments, the compound is of formula I, I-a, II, III, VI, or V, or a pharmaceutically acceptable salt thereof, and R² is optionally substituted 3- to 6-membered cycloalkyl. In specific embodiments, R² is

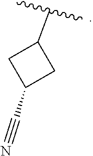

Further provided is a compound selected from compounds A1-A36 in Table 1 or a pharmaceutically acceptable salt thereof. In some embodiments, a compound of the present invention is selected from Table 1, or a pharmaceutically acceptable salt or stereoisomer thereof. In some embodiments, a compound of the present invention is selected from Table 1, or a pharmaceutically acceptable salt or atropisomer thereof.

TABLE 1
Certain Compounds of the Present Invention

| Ex# | Structure |
|---|---|
| A1 | 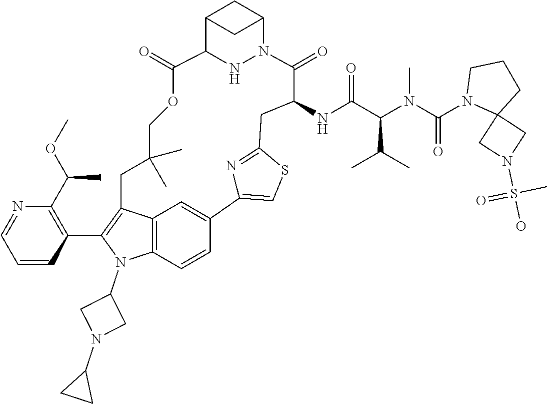 |
| A2 | 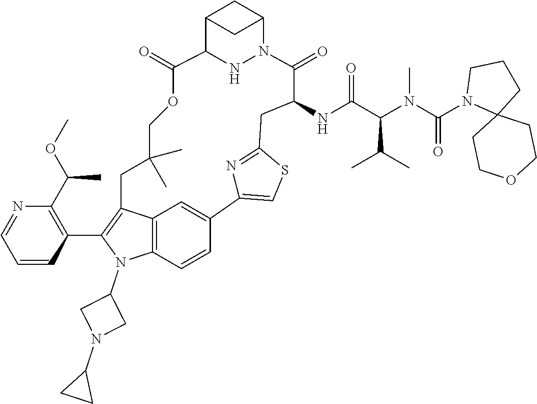 |
| A3 | 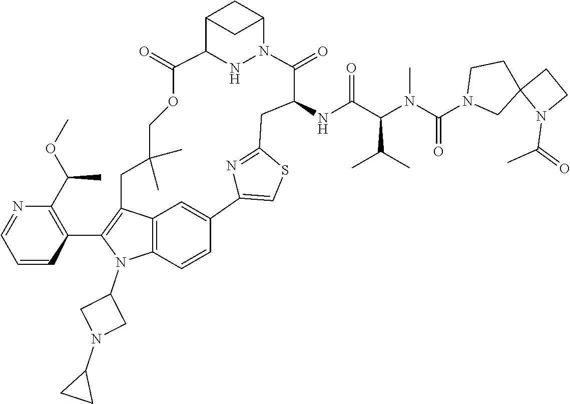 |
| A4 | 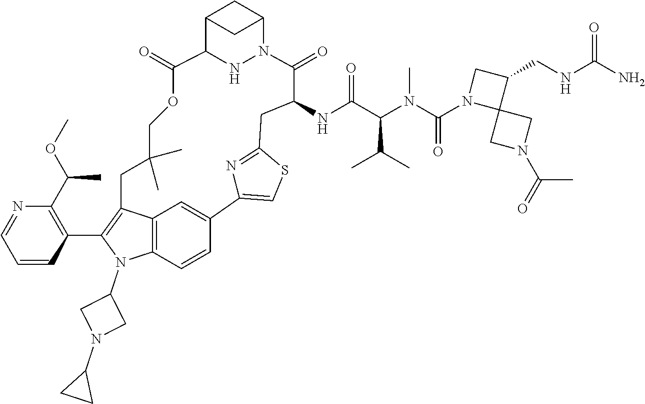 |
| A5 | 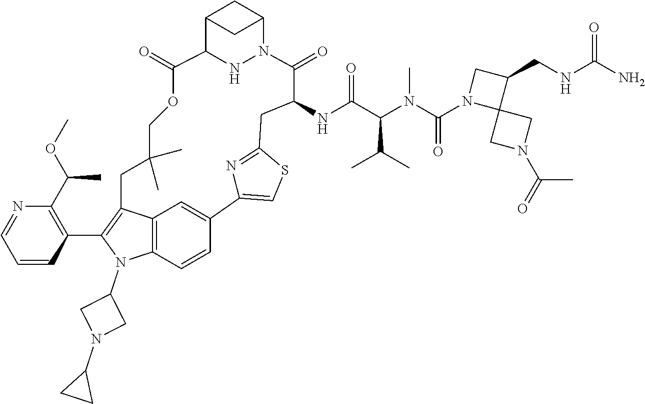 |
| A6 | 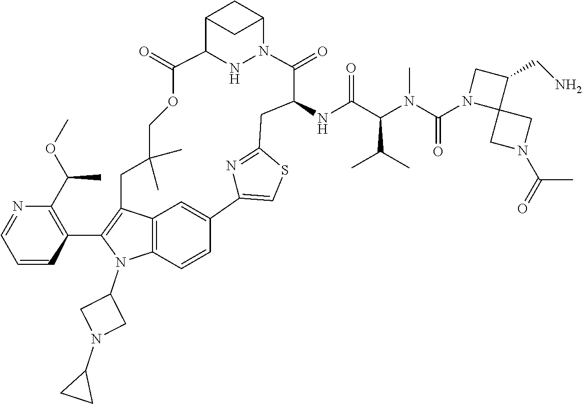 |
| A7 | 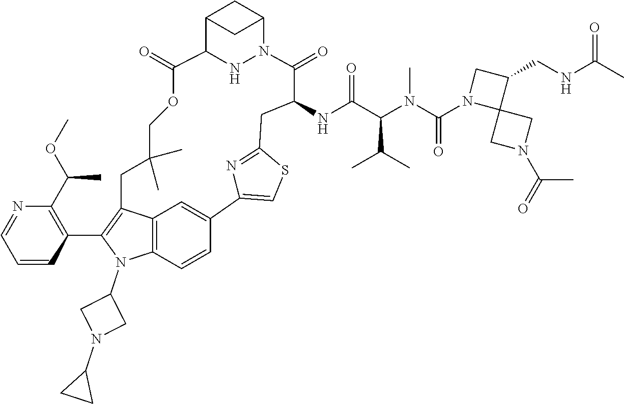 |
| A8 | 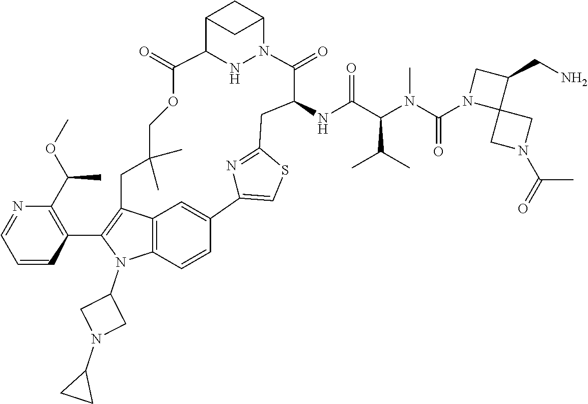 |
| A9 | 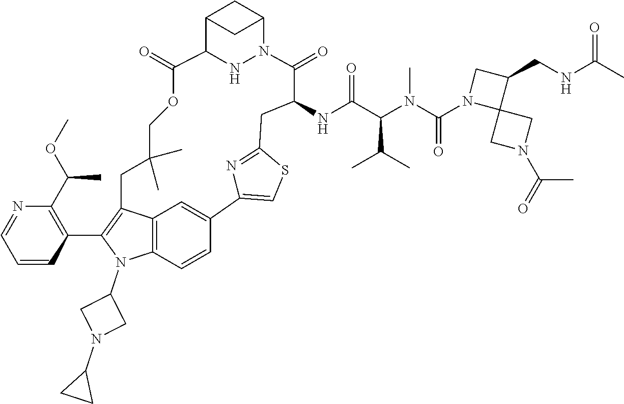 |
| A10 | 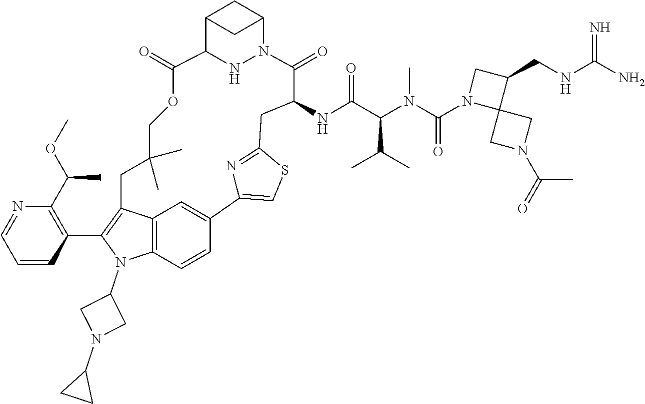 |
| A11 | 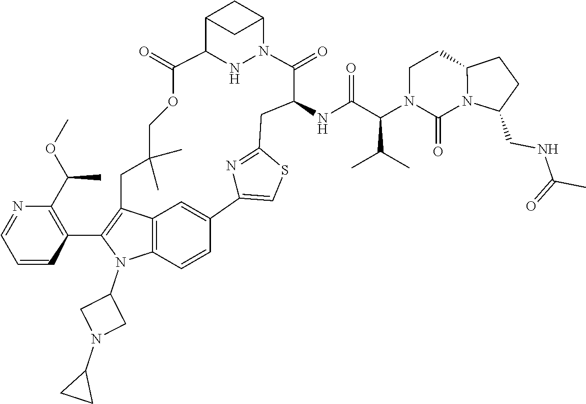 |
| A12 | 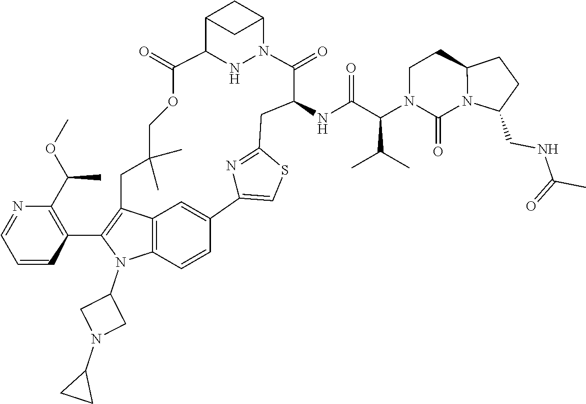 |
| A13 | 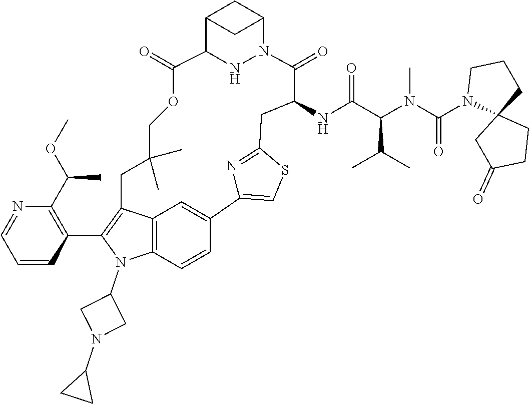 |
| A14 | 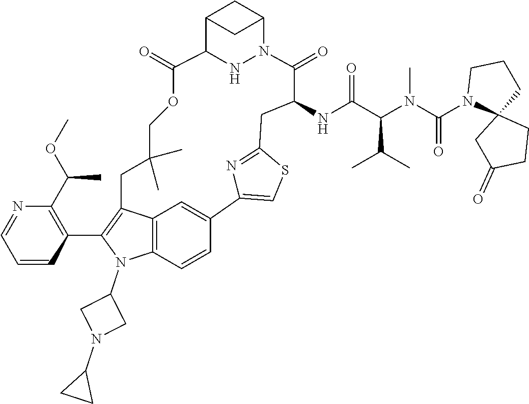 |
| A15 | 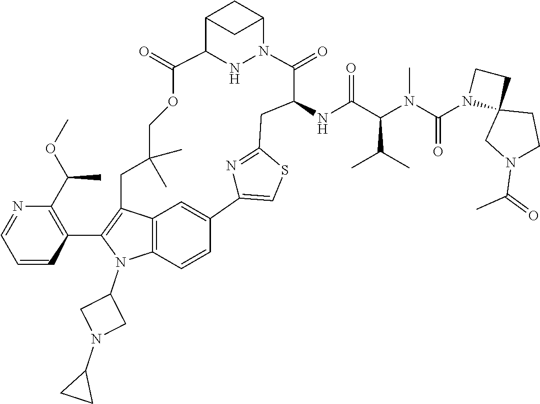 |
| A16 | 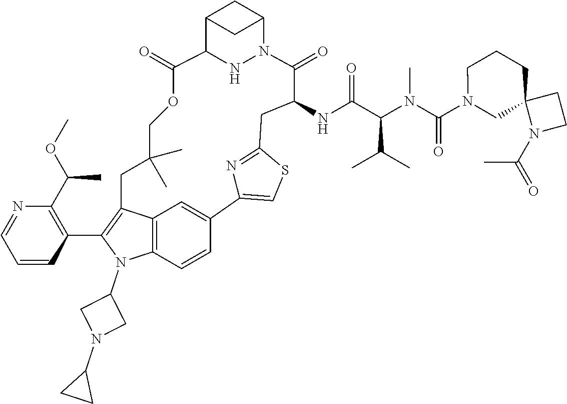 |
| A17 | 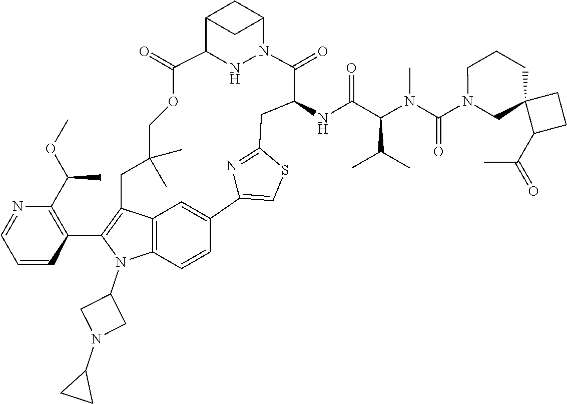 |
| A18 | 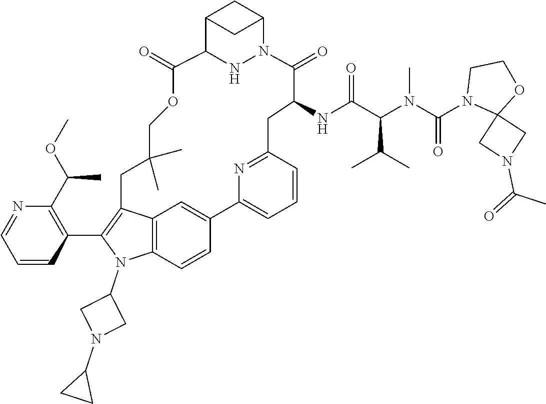 |
| A19 | 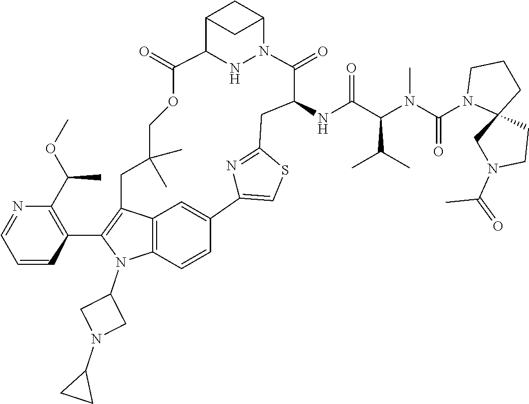 |
| A20 | 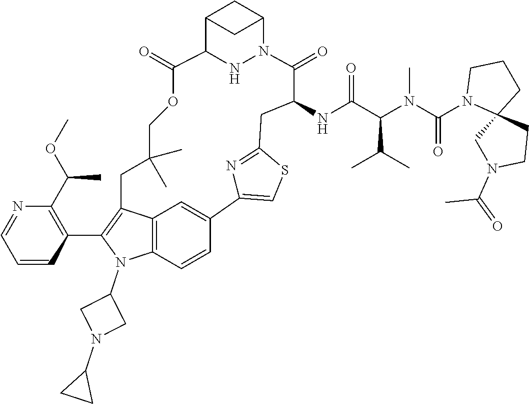 |
| A21 | 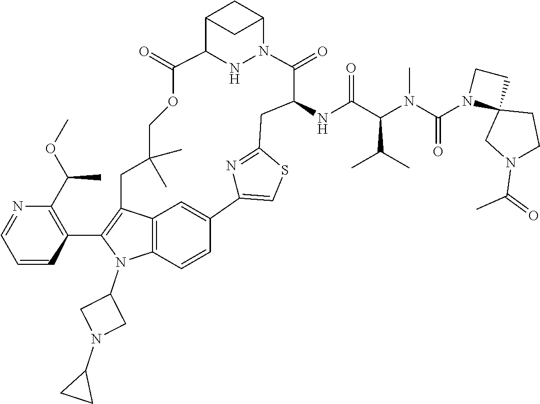 |
| A22 | 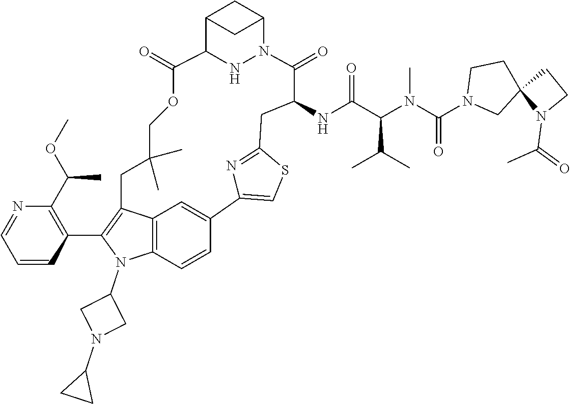 |
| A23 | 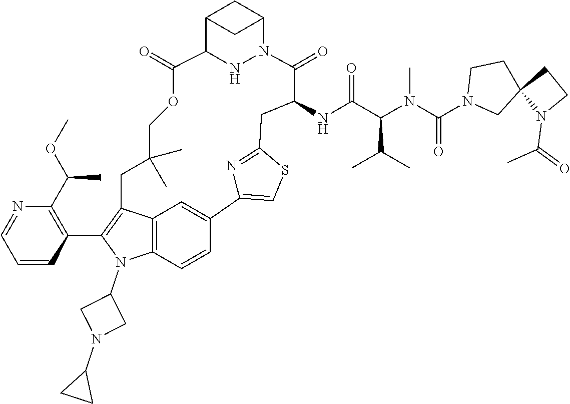 |
| A24 | 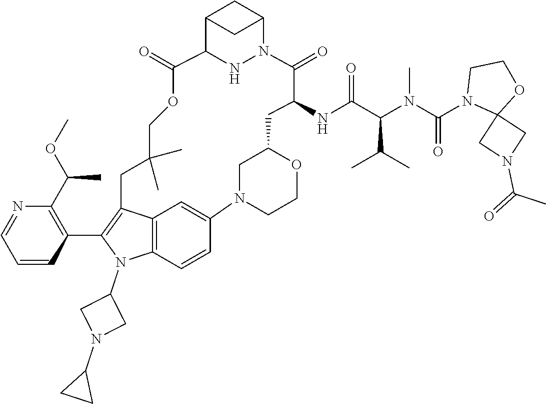 |
| A25 | 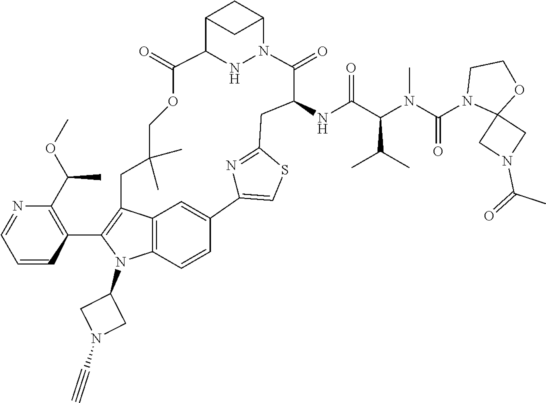 |
| A26 | 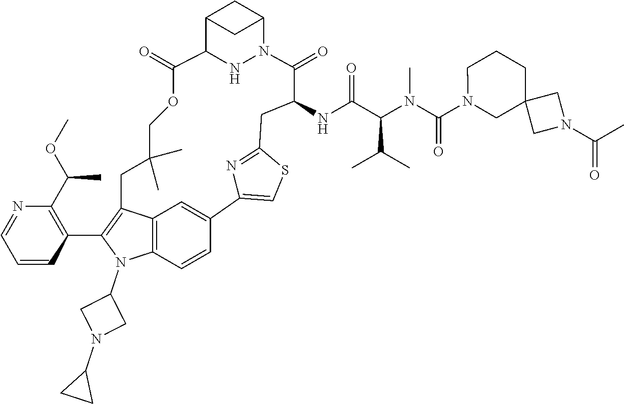 |
| A27 | 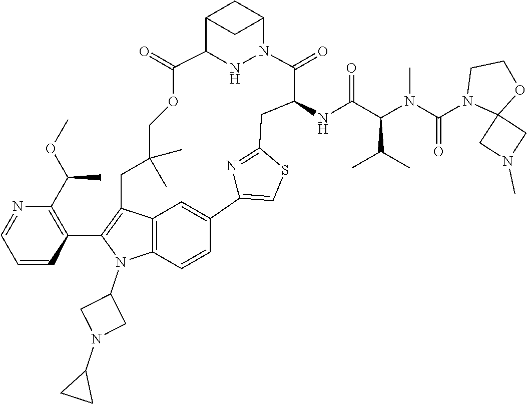 |
| A28 | 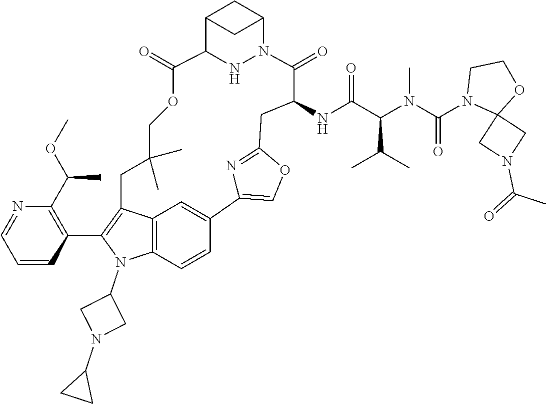 |
| A29 | 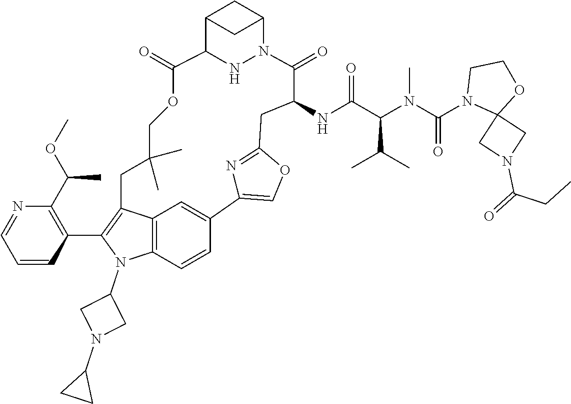 |
| A30 | 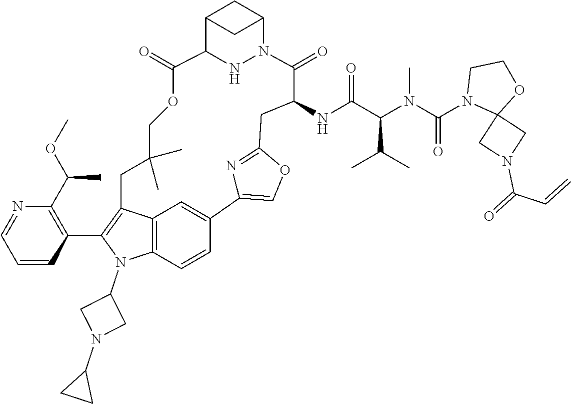 |
| A31 | 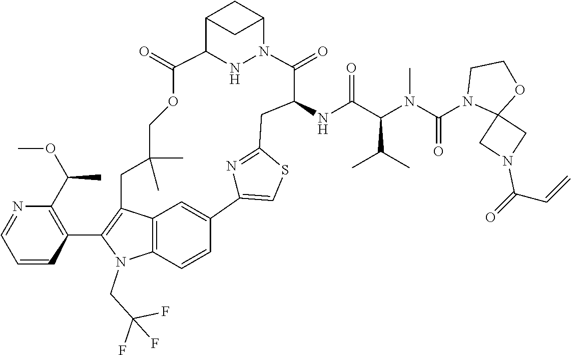 |
| A32 | 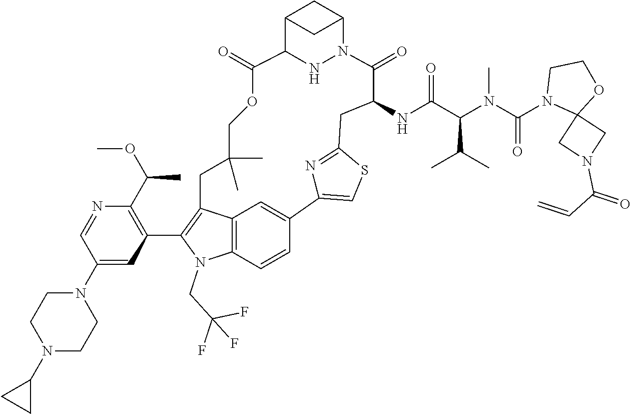 |
| A33 | 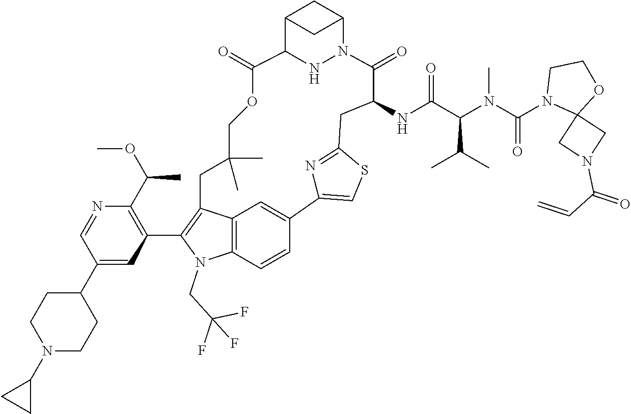 |
| A34 | 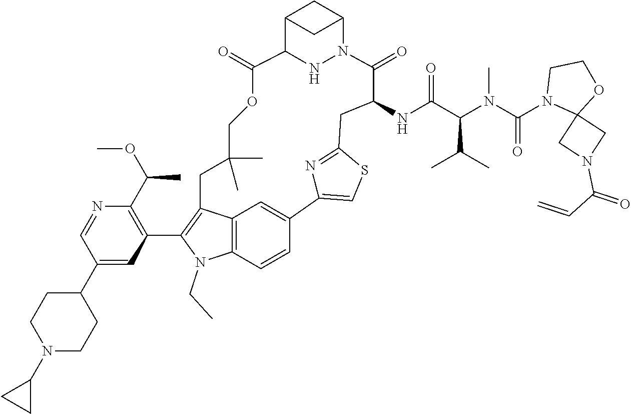 |
| A35 | 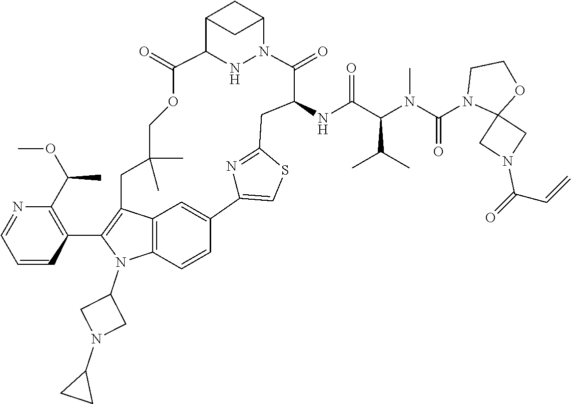 |
| A36 | 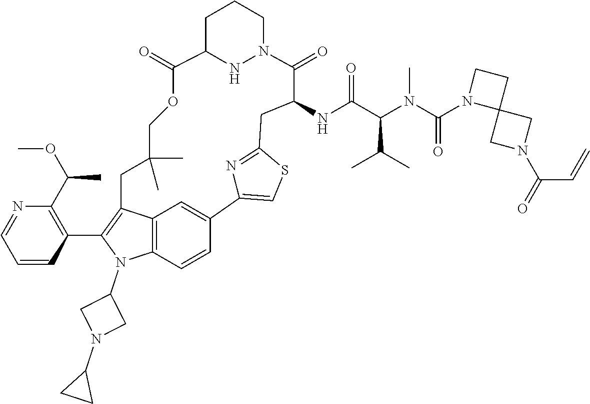 |
| A37 | 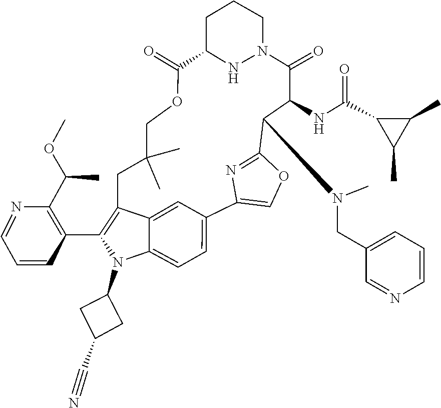 |
| A38 | 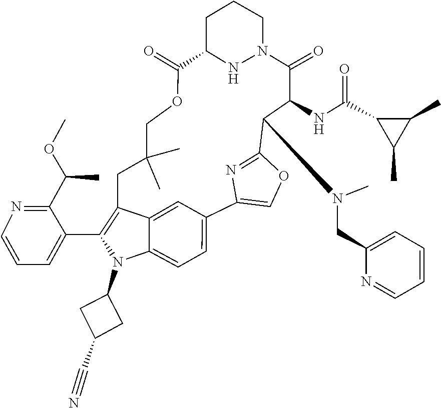 |
| A39 | 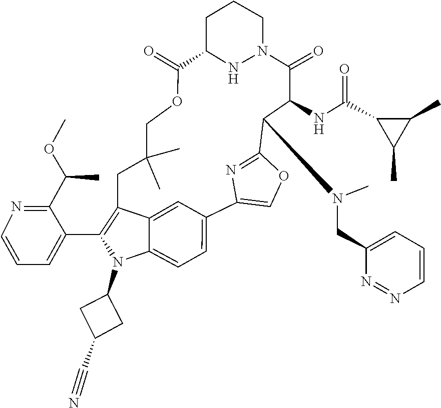 |
| A40 | 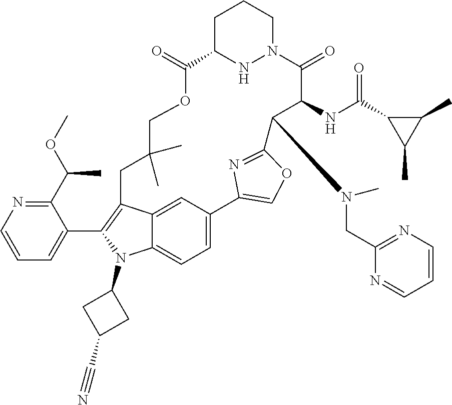 |
| A41 | 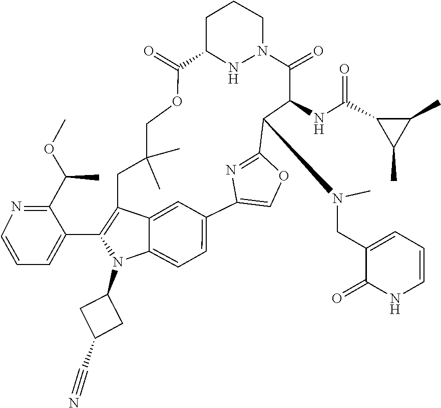 |
| A42 | 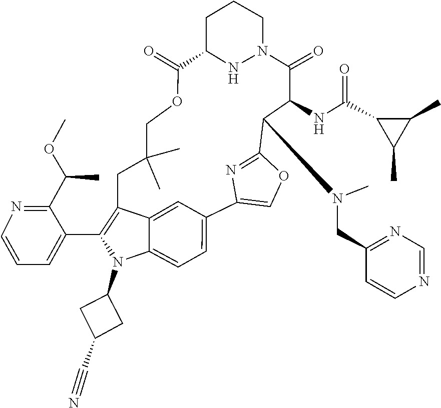 |
| A43 | 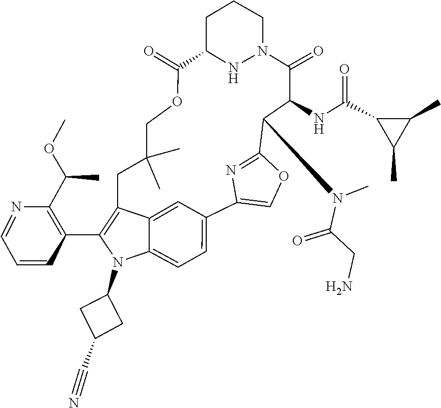 |
| A44 | 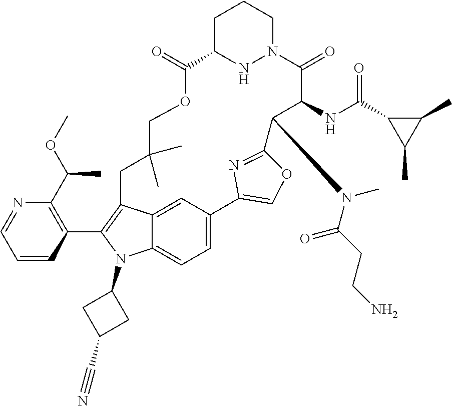 |
| A45 | 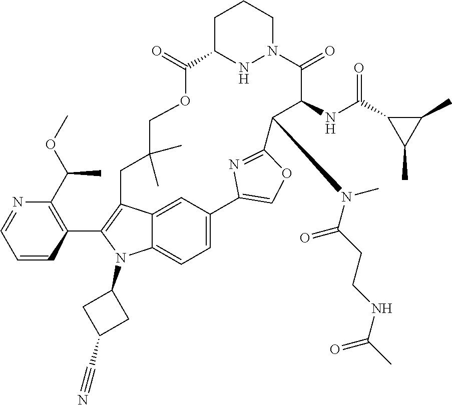 |
| A46 | 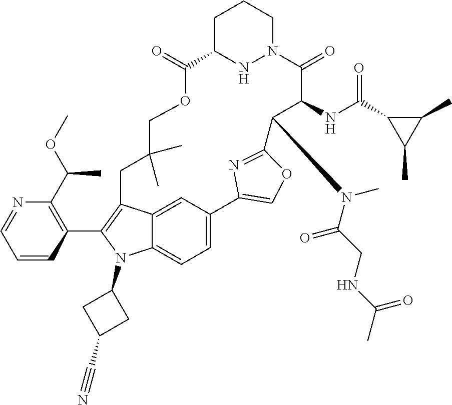 |
| A47 | 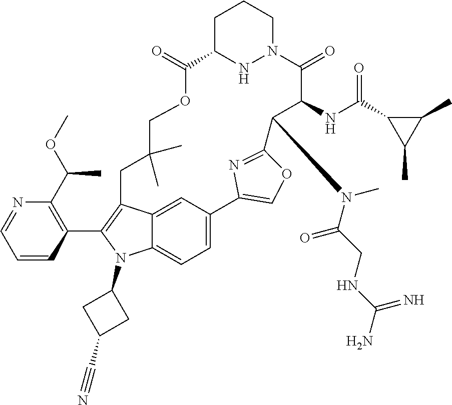 |
| A48 | 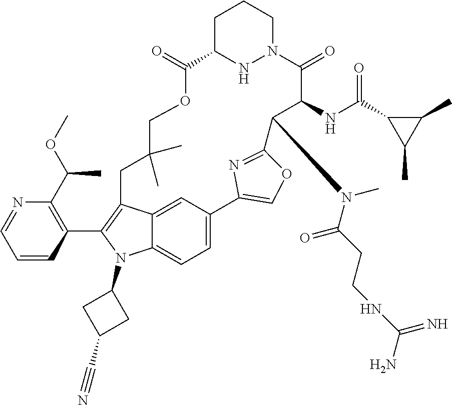 |
| A49 | 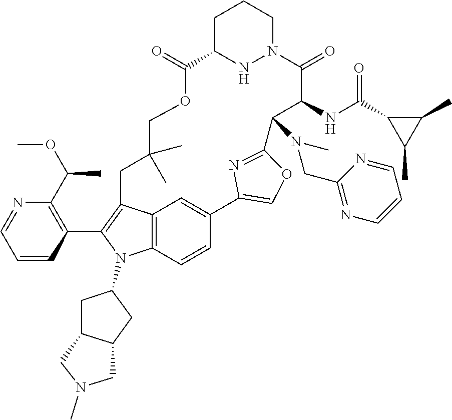 |
| A50 | 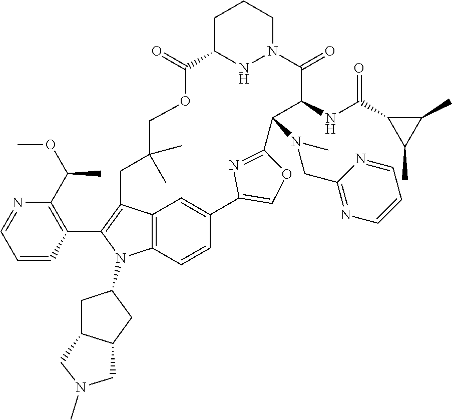 |
| A51 | 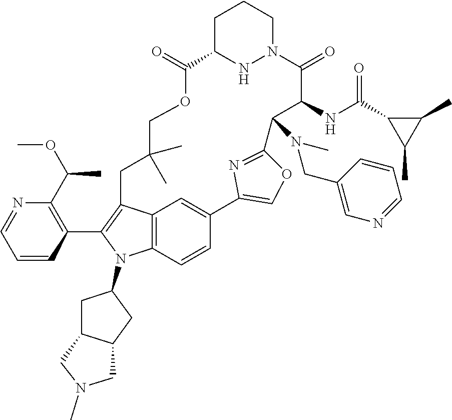 |
| A52 | 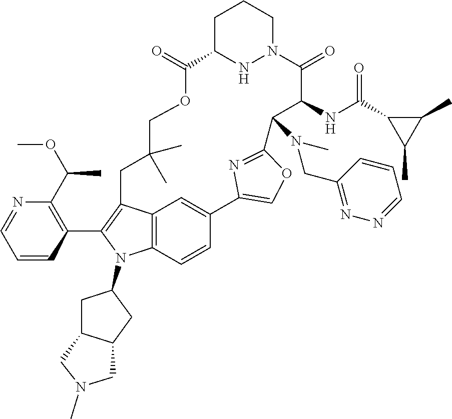 |
| A53 | 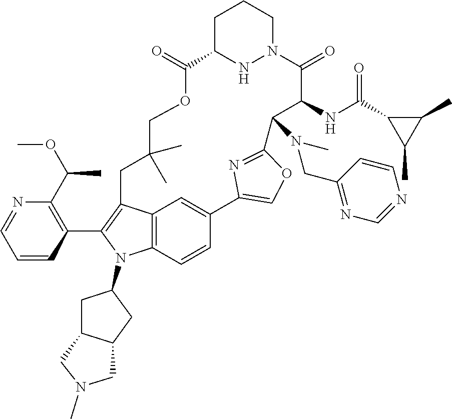 |
| A54 | 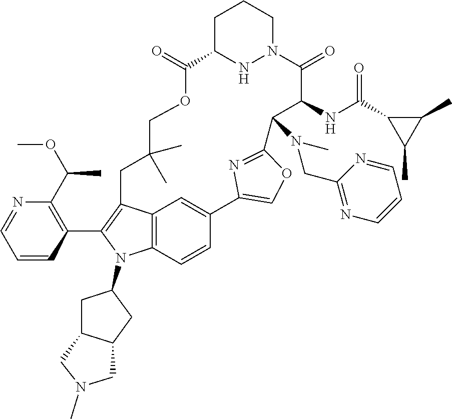 |
| A55 | 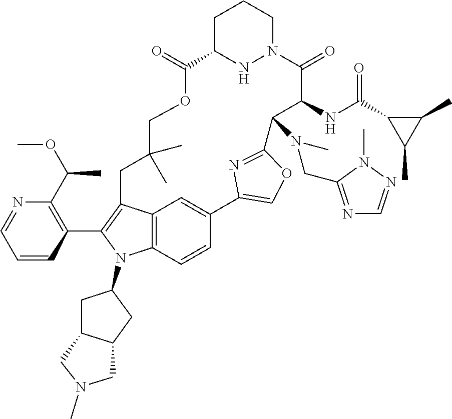 |
| A56 | 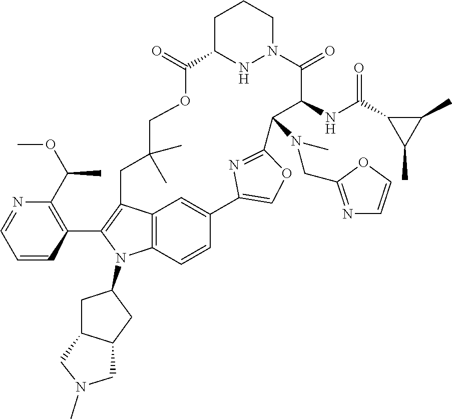 |
| A57 | 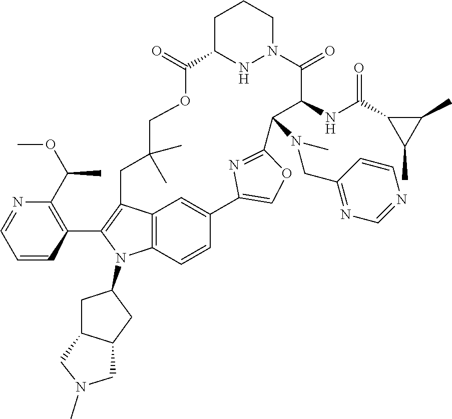 |
| A58 | 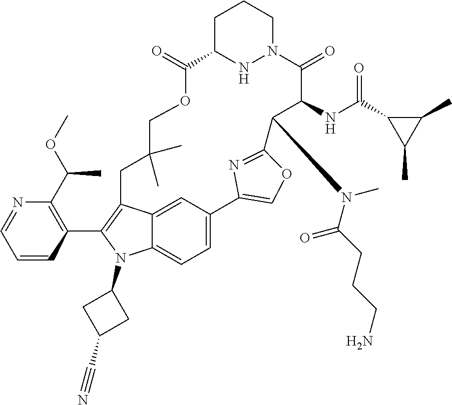 |
| A59 | 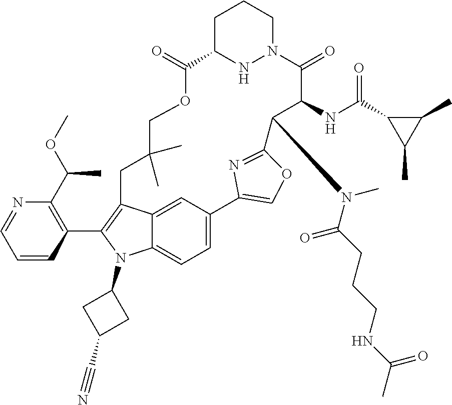 |
| A60 | 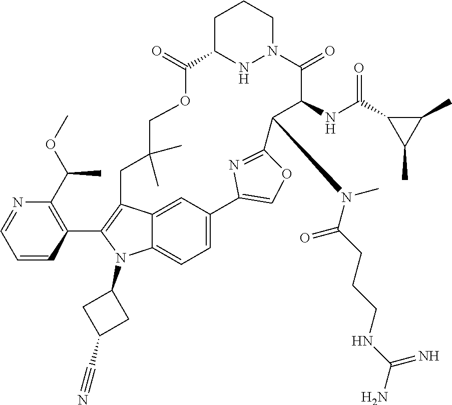 |
| A61 | 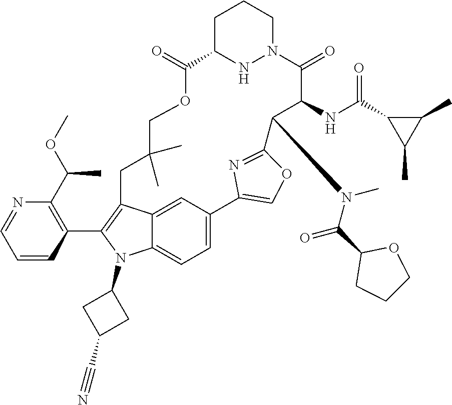 |
| A62 | 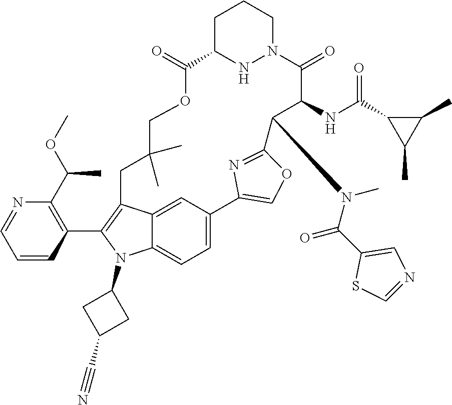 |
| A63 | 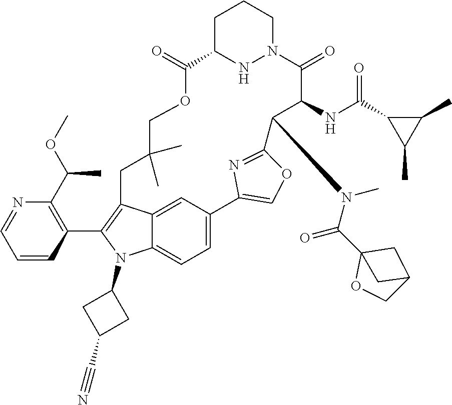 |
| A64 | 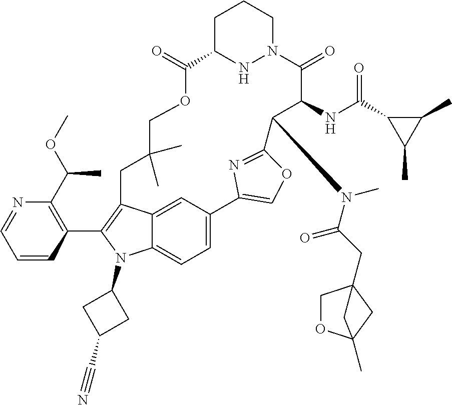 |
| A65 | 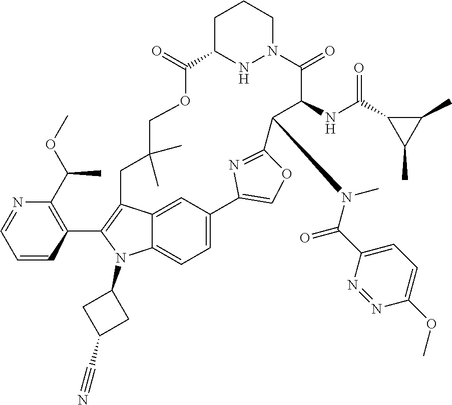 |
| A66 | 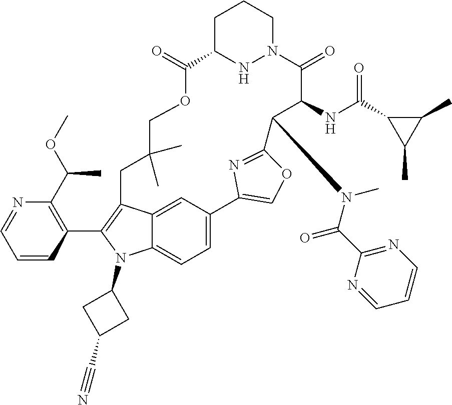 |
| A67 | 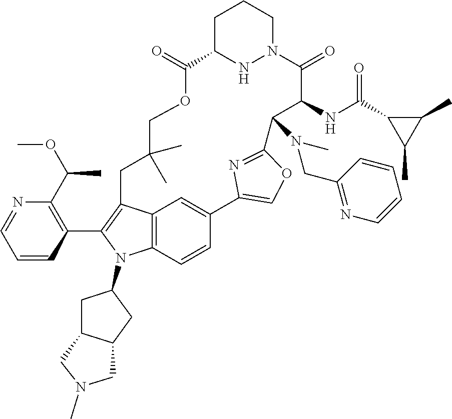 |
| A68 | 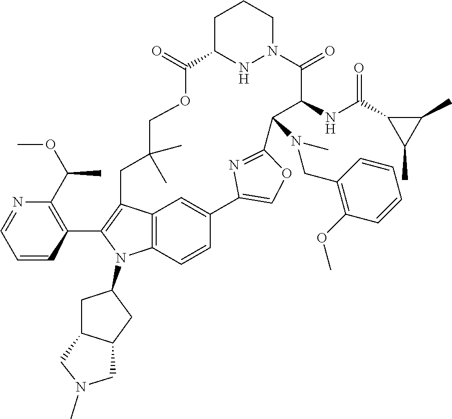 |
| A69 | 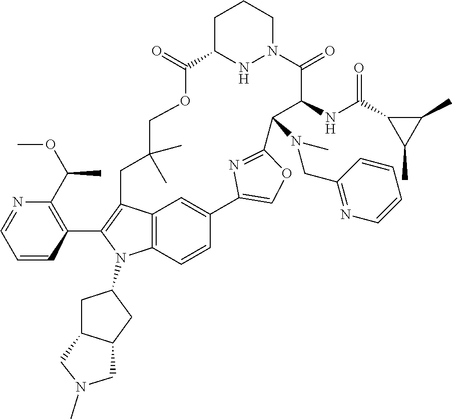 |
| A70 | 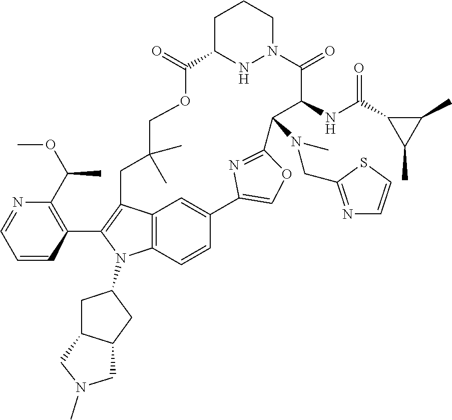 |
| A71 | 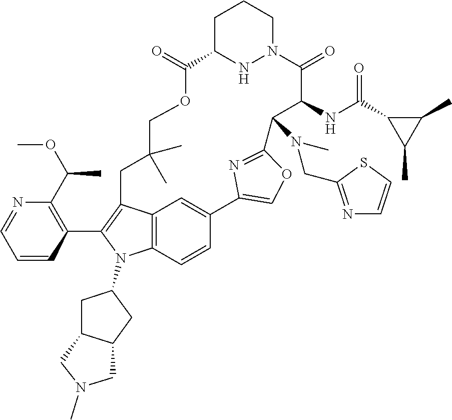 |
| A72 | 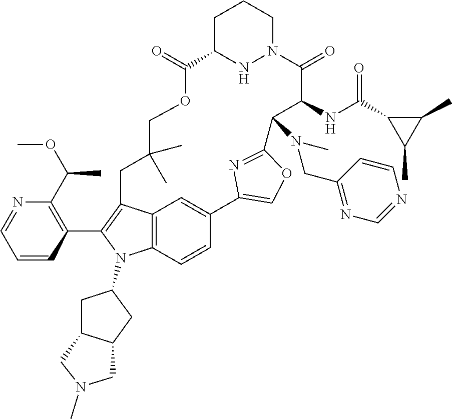 |
| A73 | 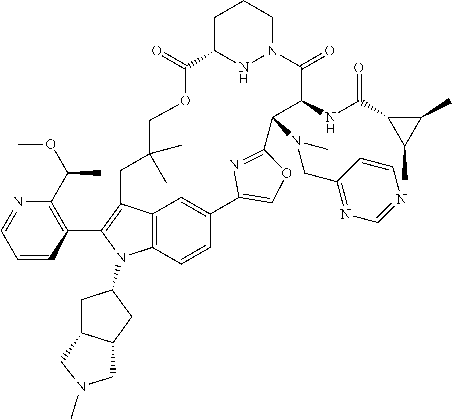 |
| A74 | 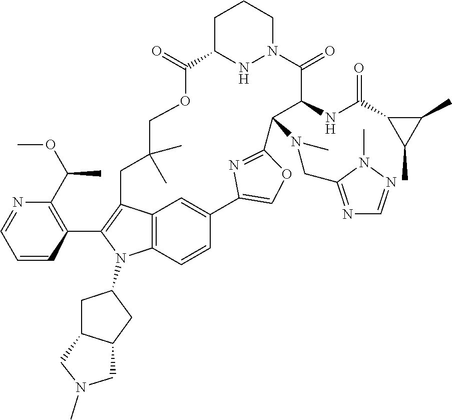 |
| A75 | 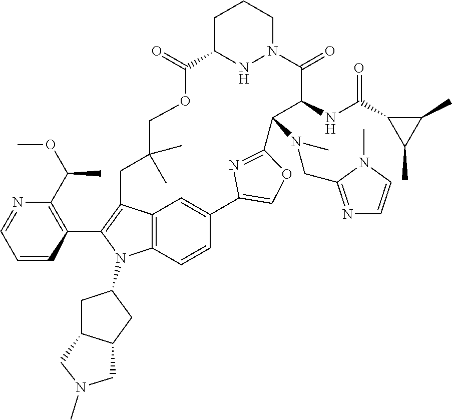 |
| A76 | 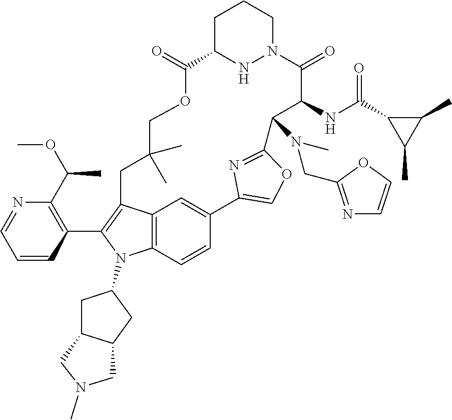 |
| A77 | 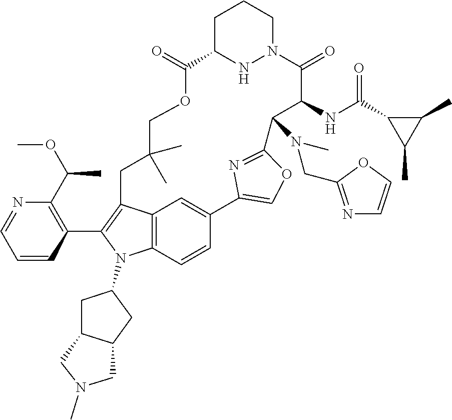 |
| A78 | 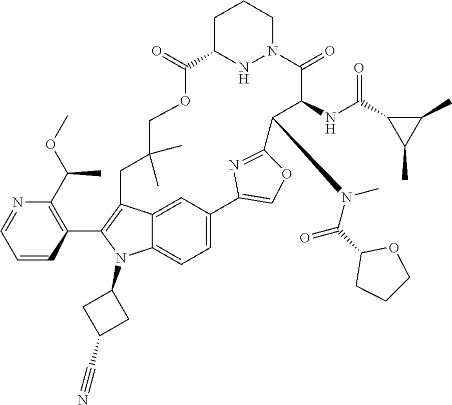 |
| A79 | 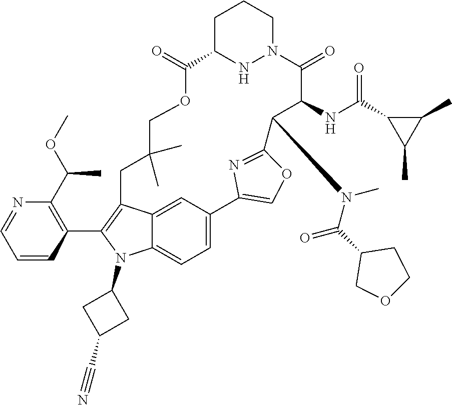 |
| A80 | 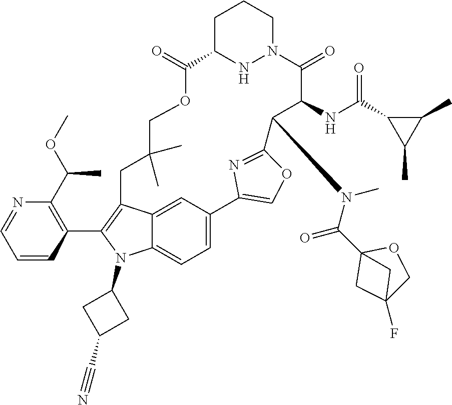 |
| A81 | 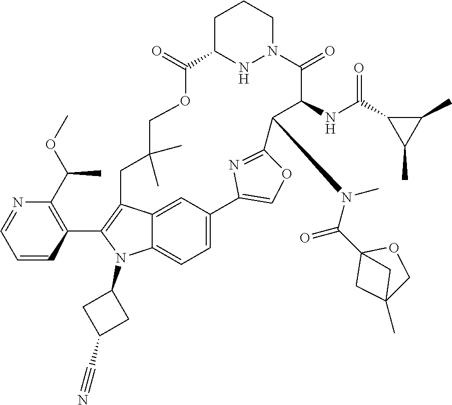 |
| A82 | 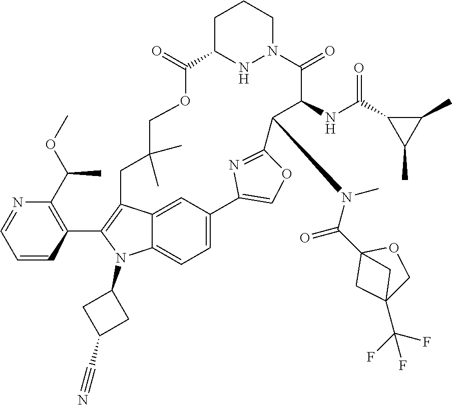 |
| A83 | 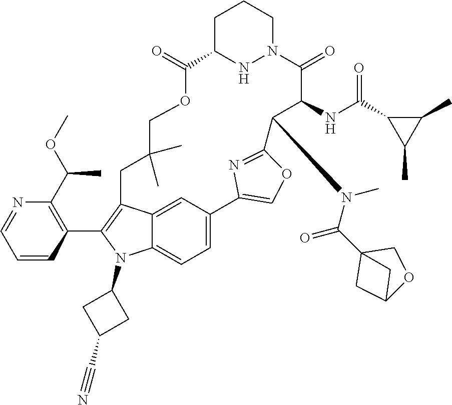 |
| A84 | 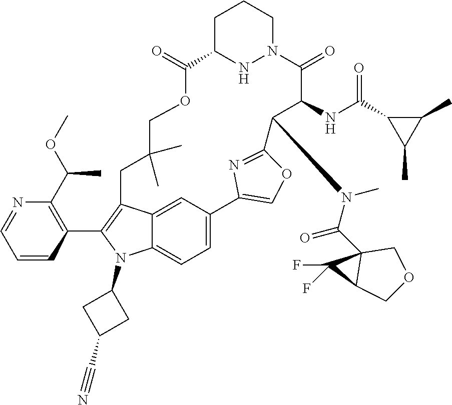 |
| A85 | 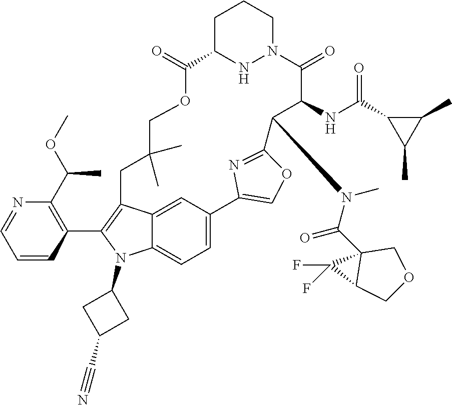 |
| A86 | 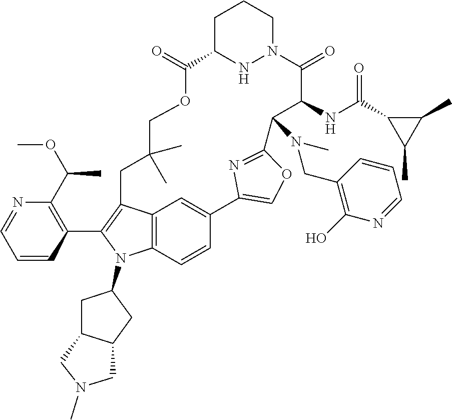 |
| A87 | 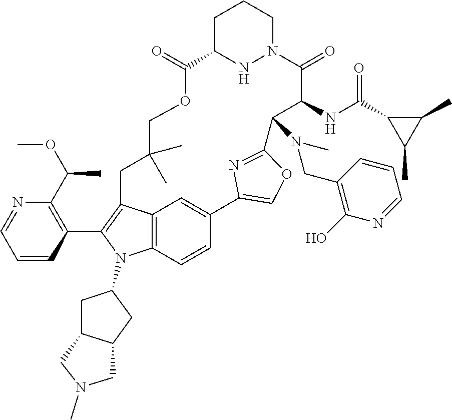 |
| A88 | 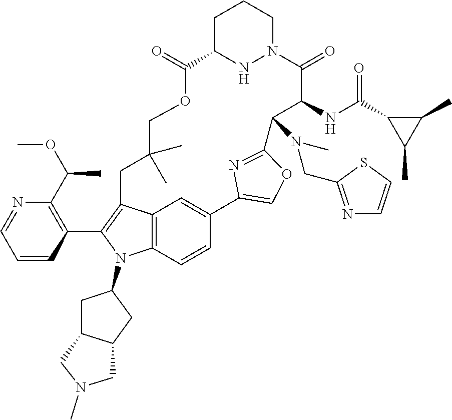 |
| A89 | 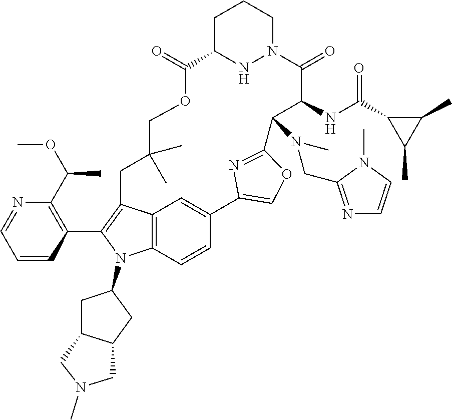 |
| A90 | 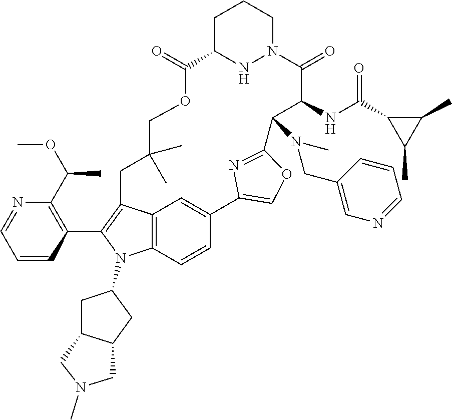 |
| A91 | 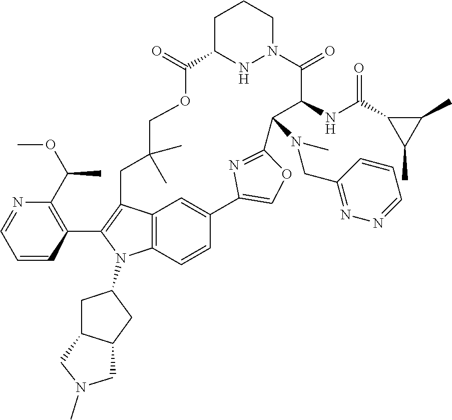 |
| A92 | 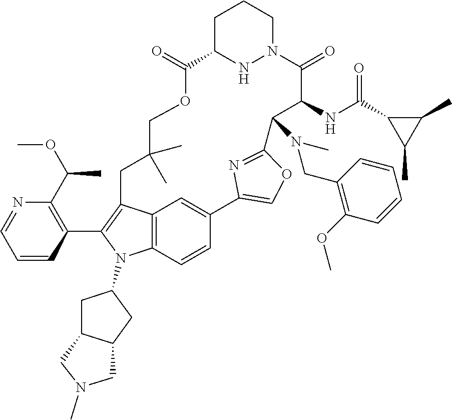 |
| A93 | 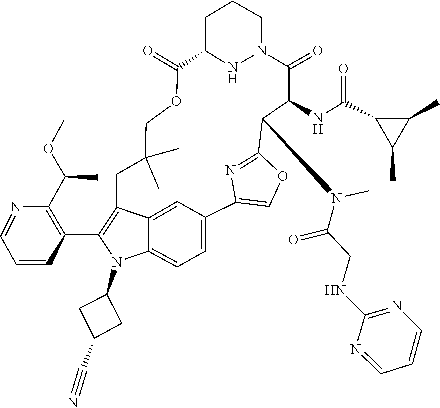 |
| A94 | 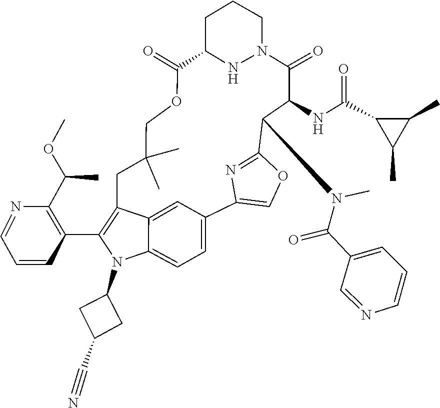 |
| A95 | 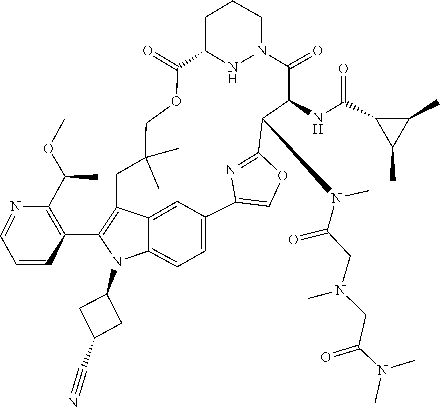 |
| A96 | 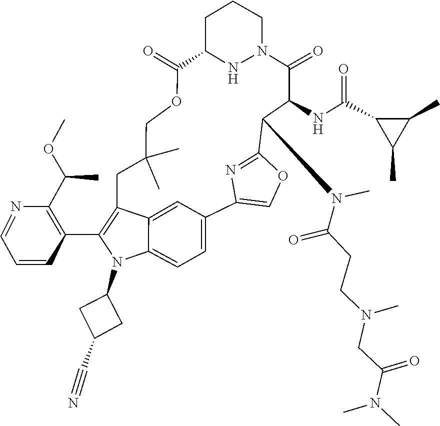 |
| A97 | 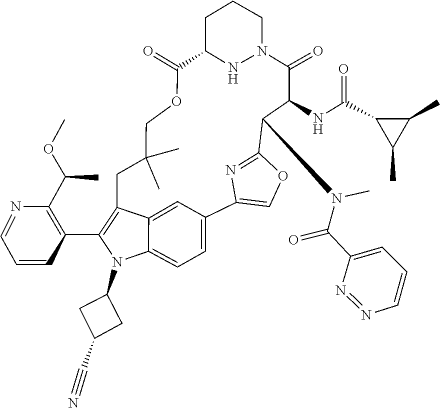 |
| A98 | 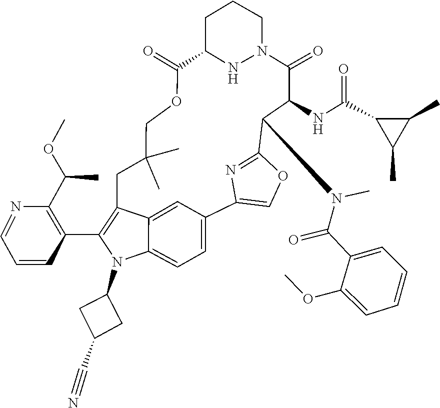 |
| A99 | 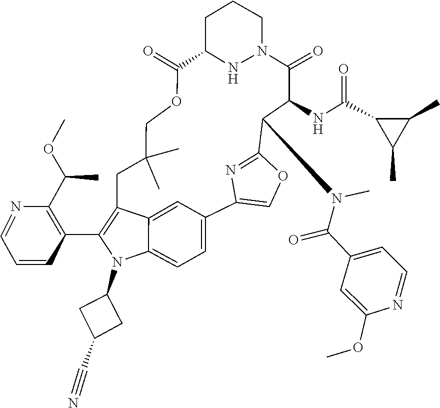 |
| A100 | 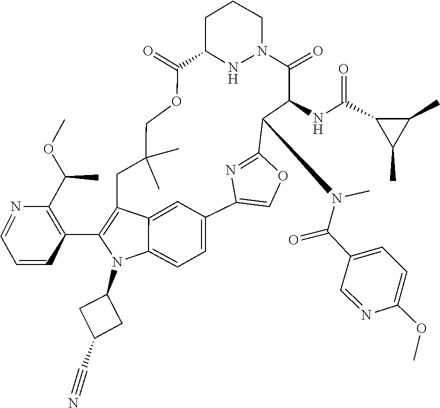 |
| A101 | 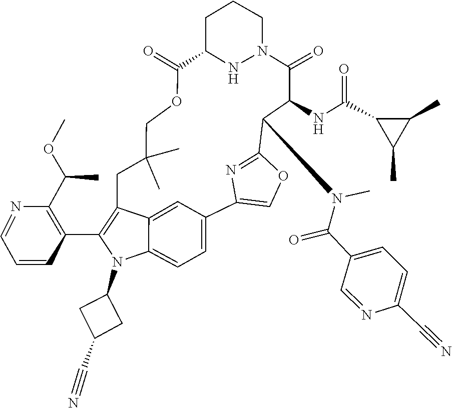 |
| A102 | 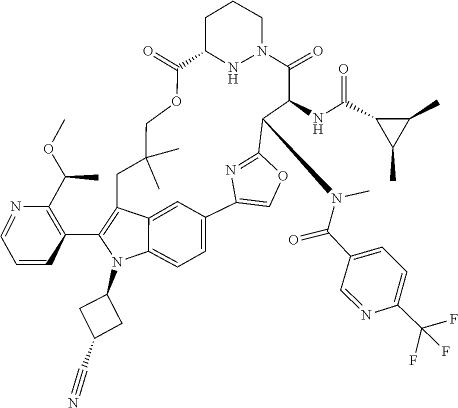 |
| A103 | 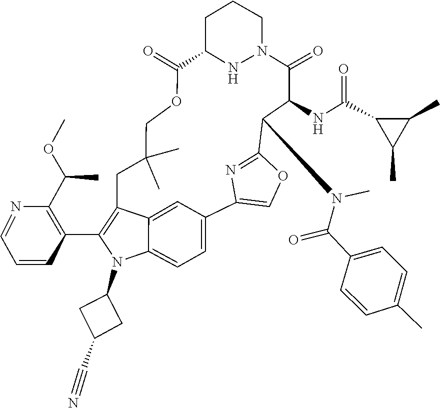 |
| A104 | 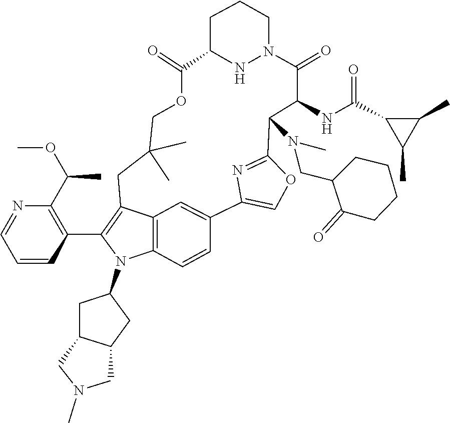 |
| A105 | 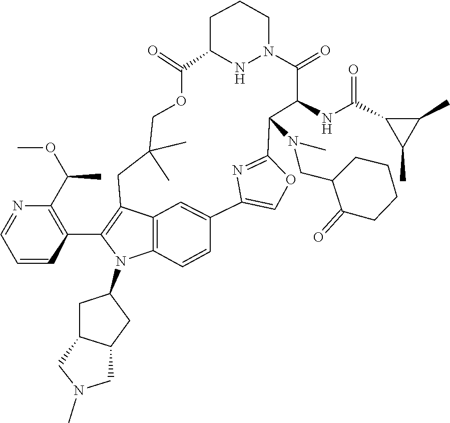 |
| A106 | 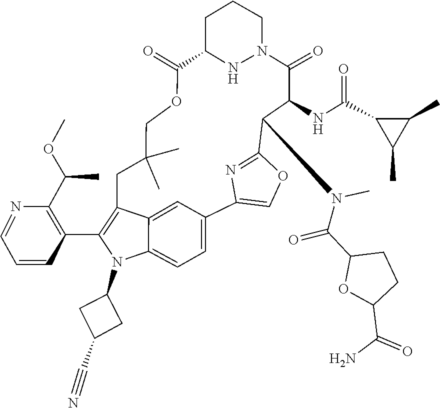 |
| A107 | 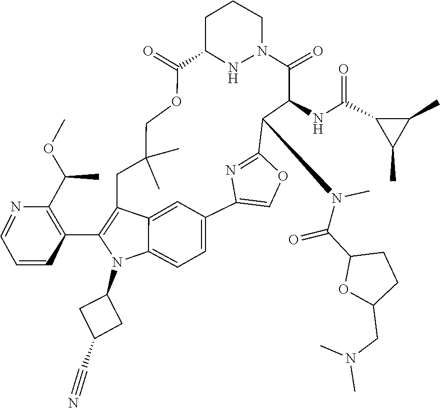 |
| A108 | 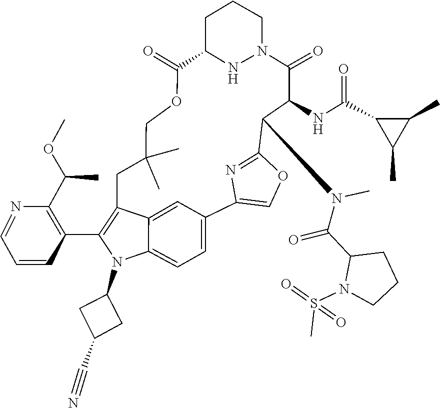 |
| A109 | 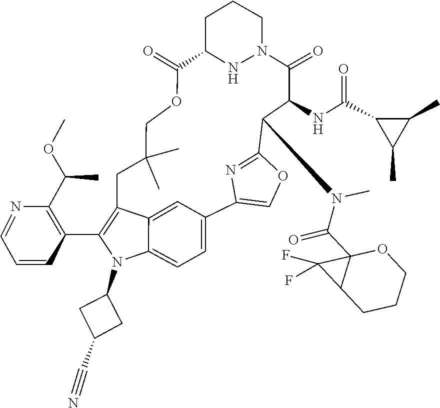 |
| A110 | 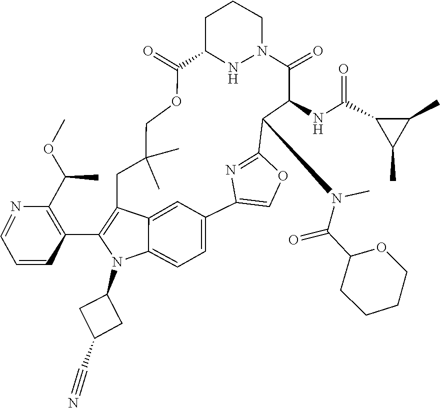 |
| A111 | 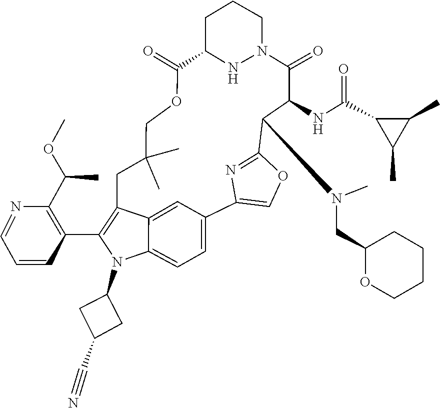 |
| A112 | 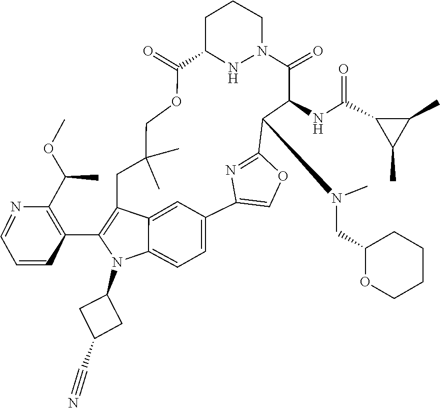 |
| A113 | 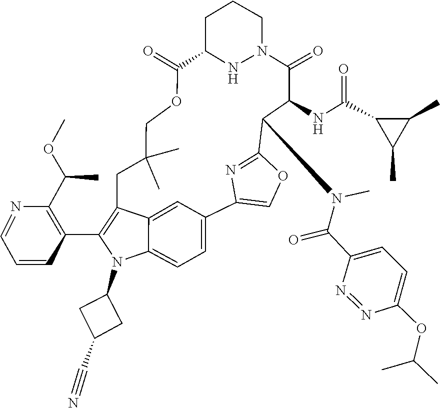 |
| A114 | 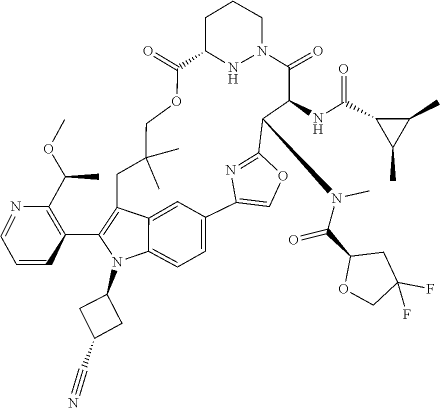 |
| A115 | 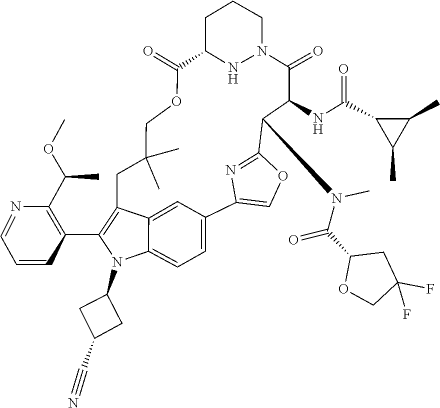 |
| A116 | 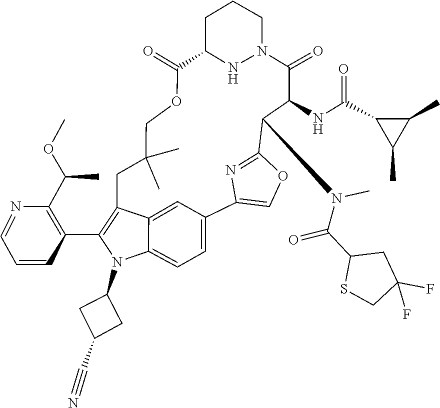 |
| A117 | 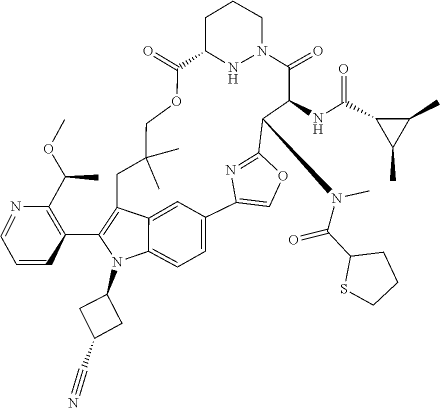 |
| A118 | 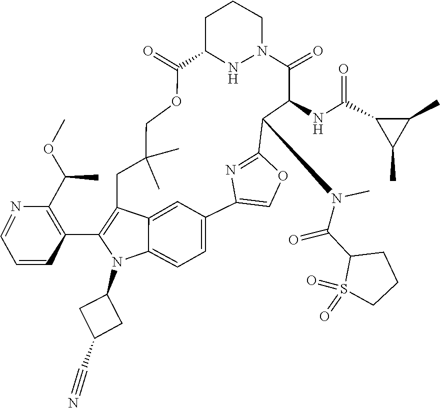 |
| A119 | 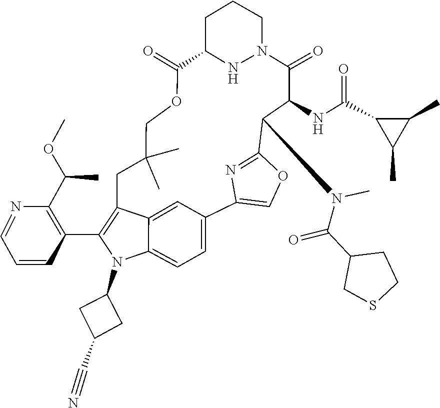 |
| A120 | 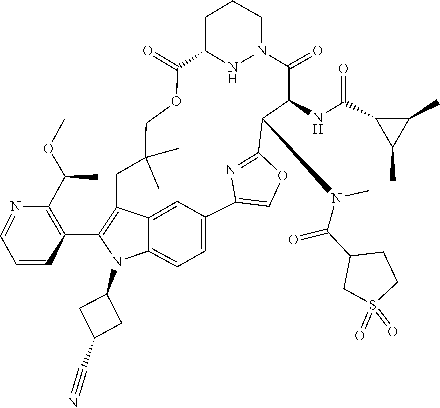 |
| A121 | 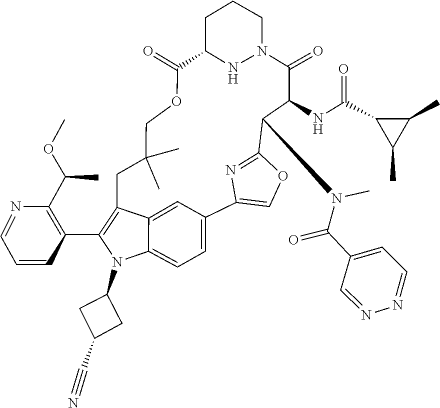 |
| A122 | 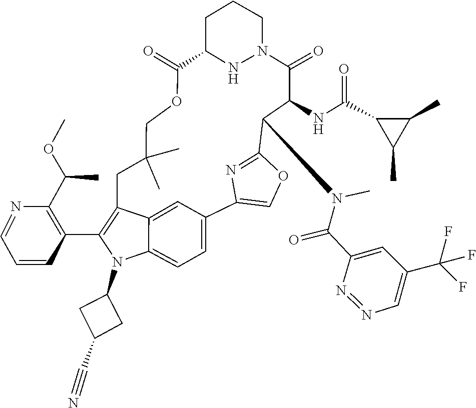 |
| A123 | 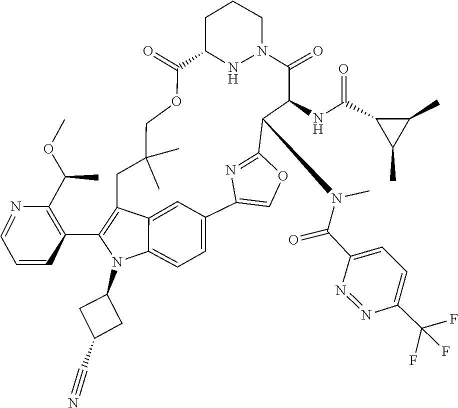 |
| A124 | 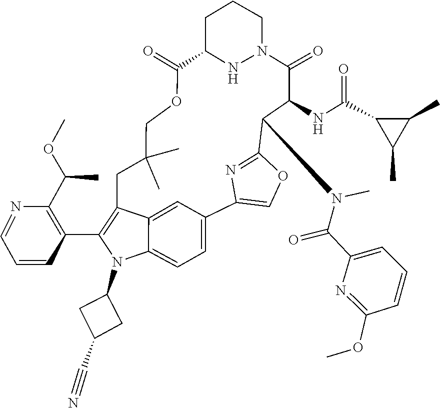 |
| A125 | 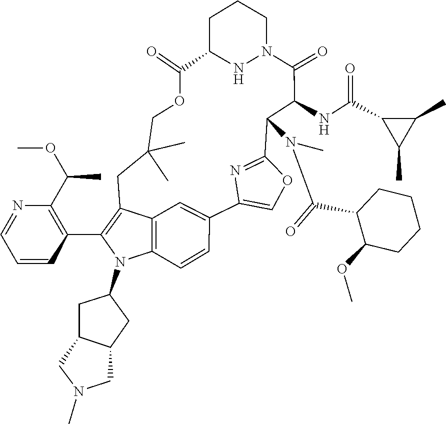 |
| A126 | 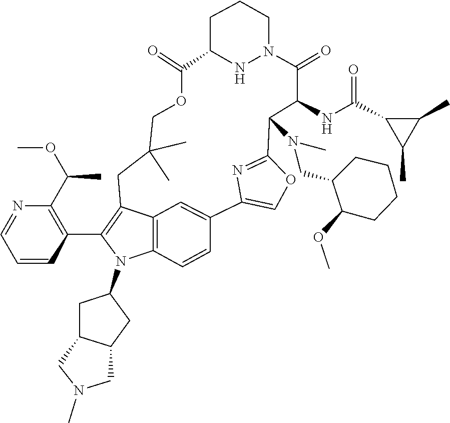 |
| A127 | 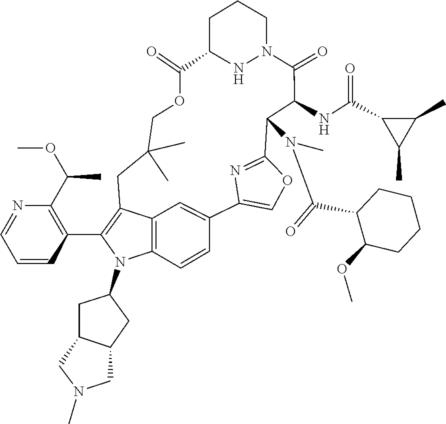 |
| A128 | 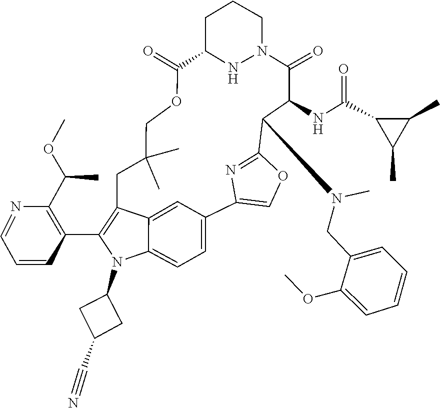 |
| A129 | 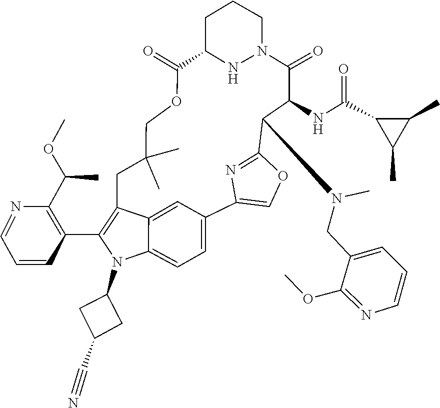 |
| A130 | 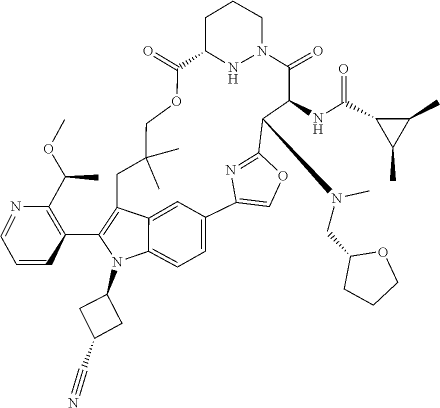 |
| A131 | 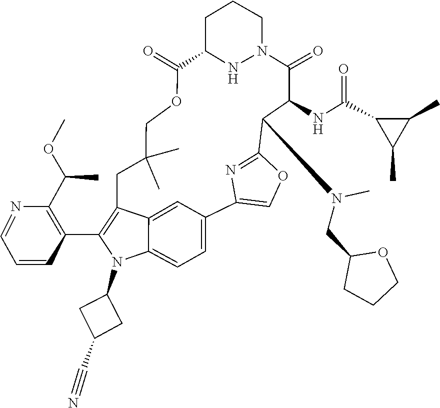 |
| A132 | 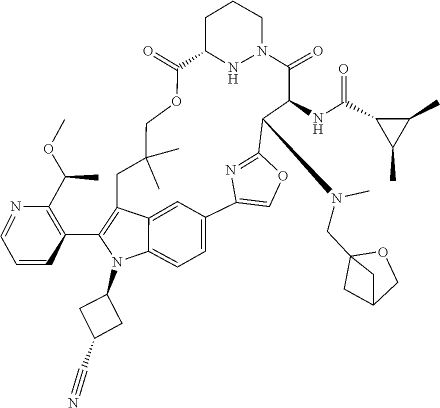 |
| A133 | 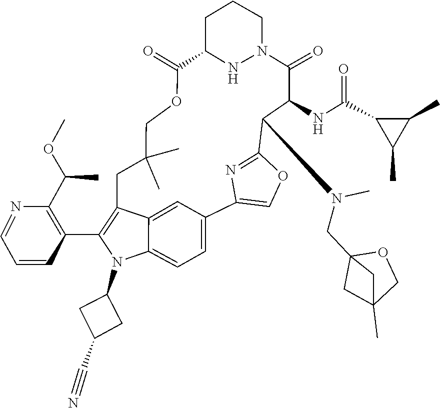 |
| A134 | 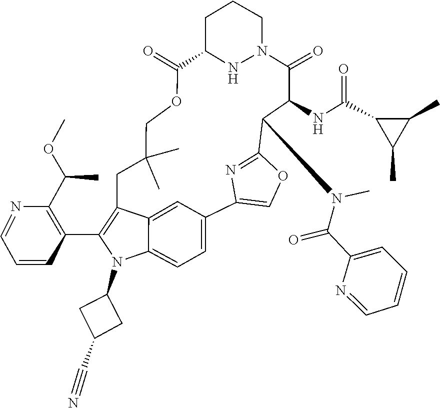 |
| A135 | 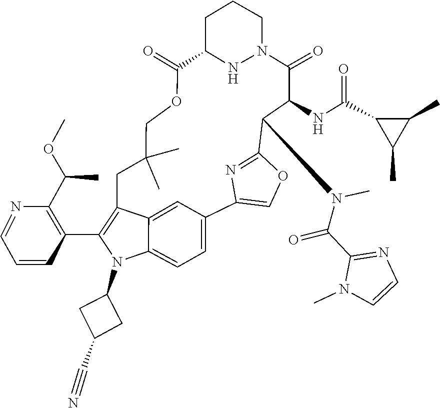 |
| A136 | 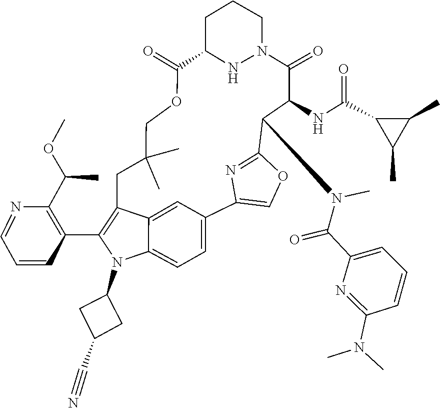 |
| A137 | 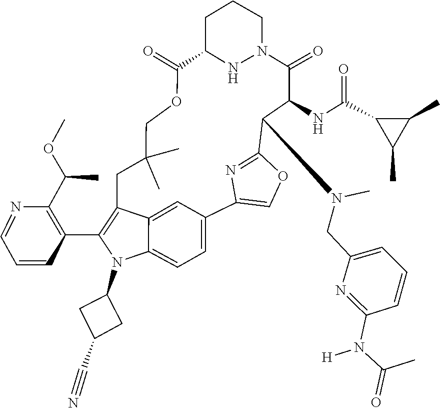 |
| A138 | 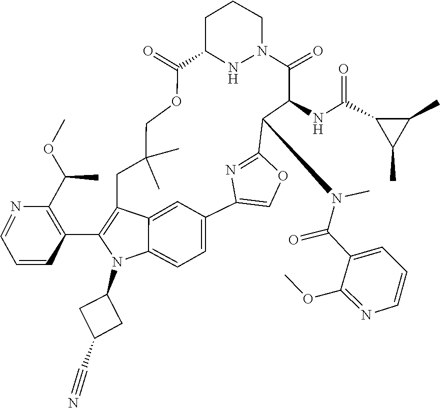 |
| A139 | 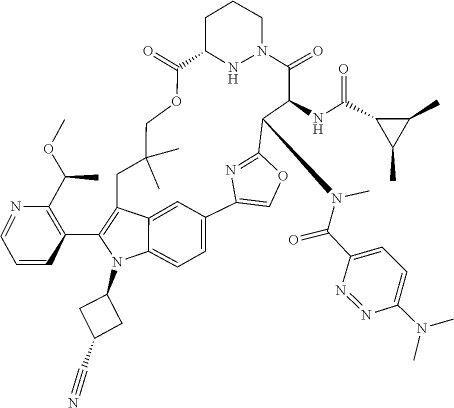 |
| A140 | 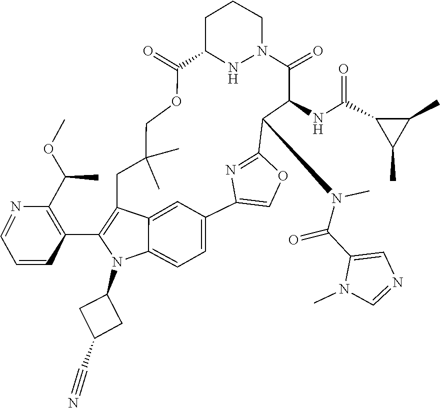 |
| A141 | 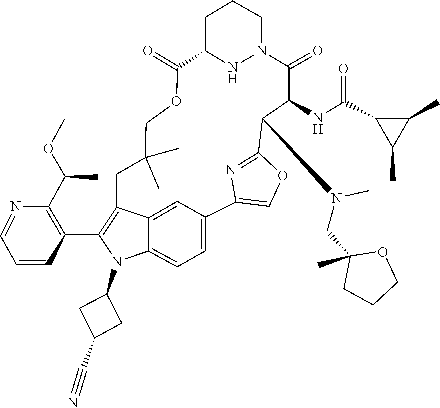 |
| A142 | 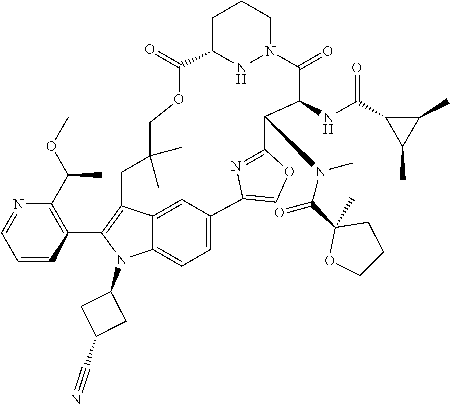 |
| A143 | 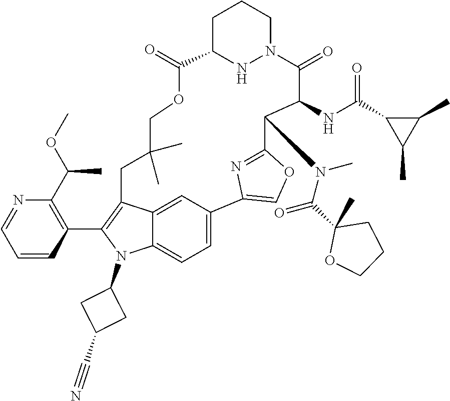 |
| A144 | 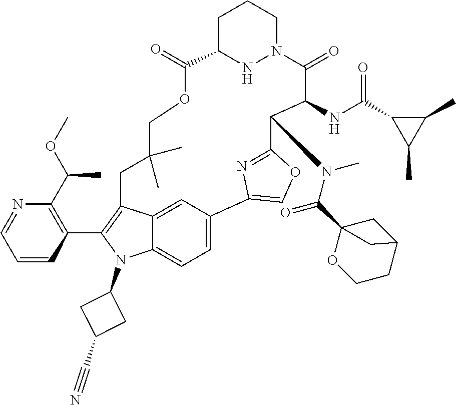 |
| A145 | 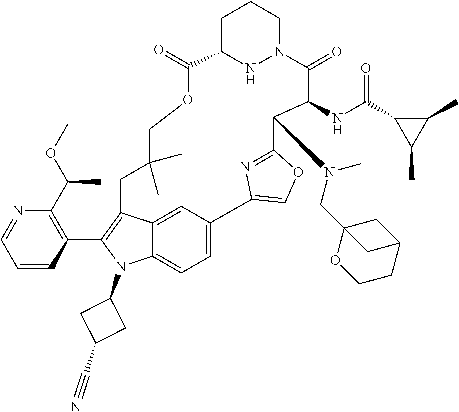 |
| A146 | 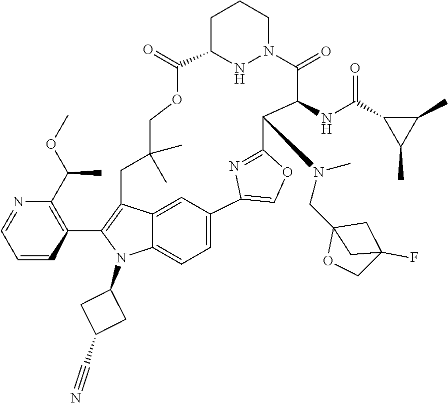 |
| A147 | 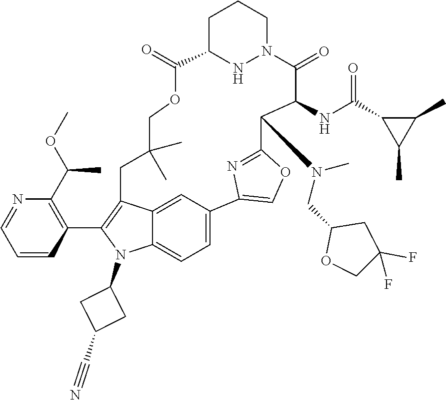 |
| A148 | 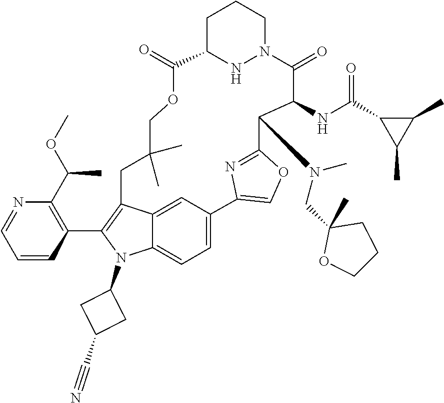 |
| A149 | 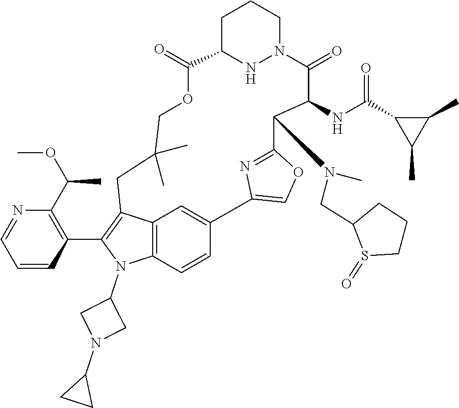 |
| A150 | 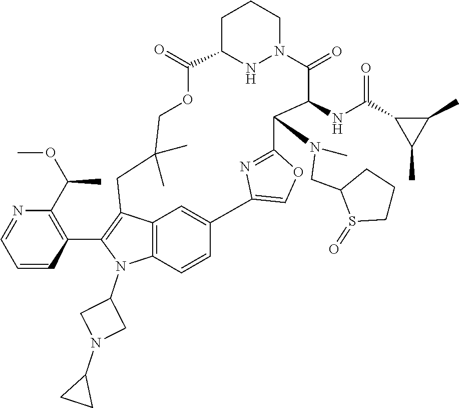 |
| A151 | 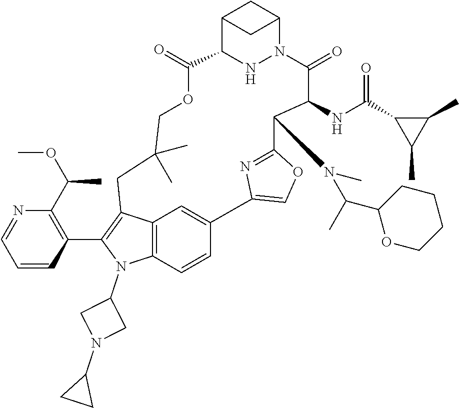 |
| A152 | 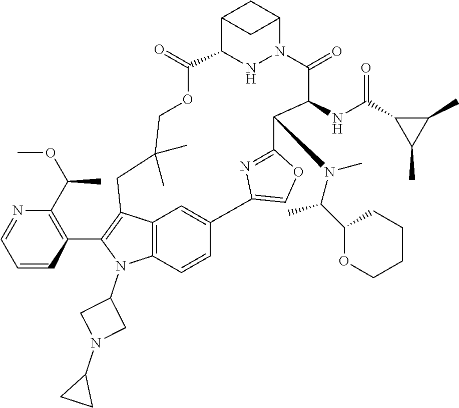 |
| A153 | 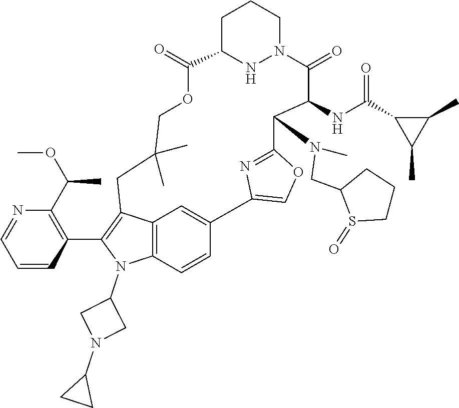 |
| A154 | 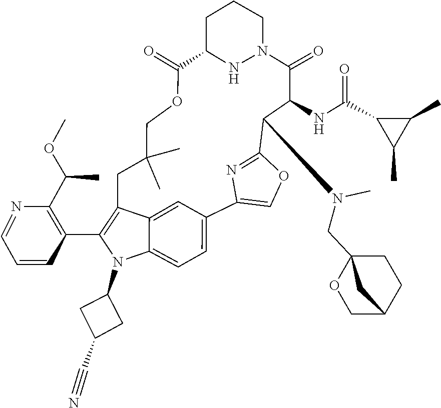 |
| A155 | 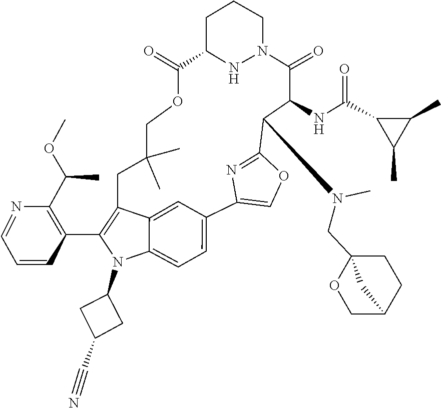 |
| A156 | 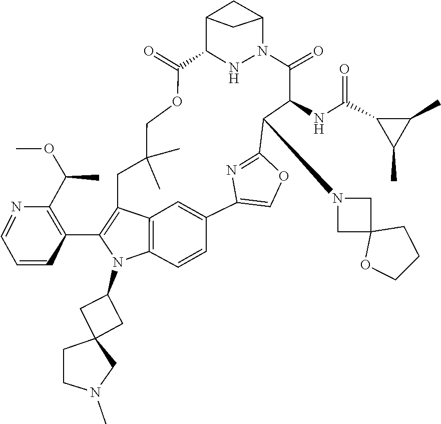 |
| A157 | 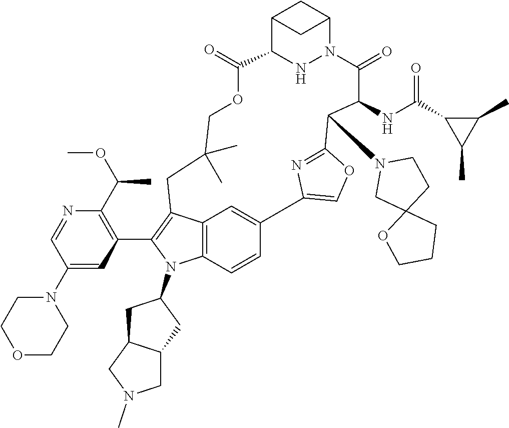 |
| A158 | 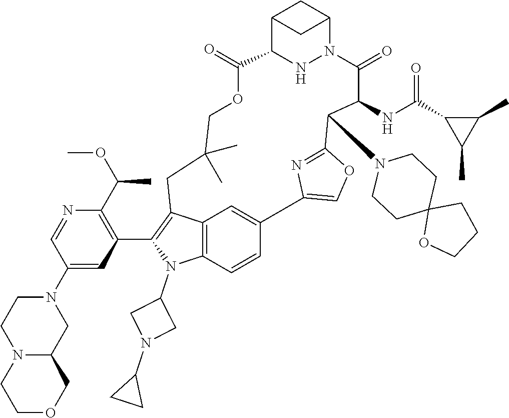 |
| A159 | 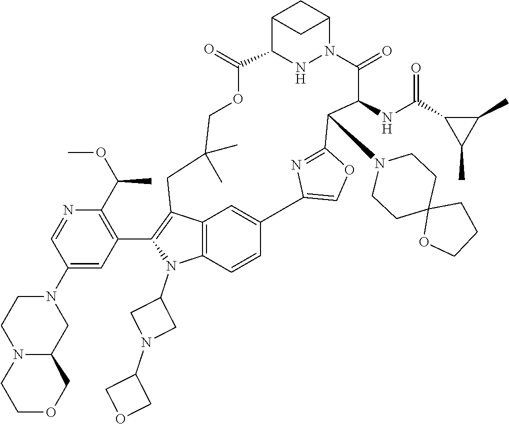 |
| A160 | 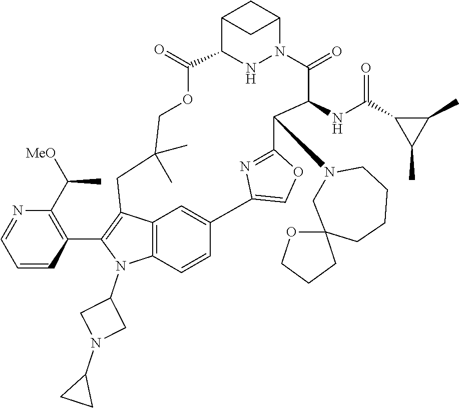 |
| A161 | 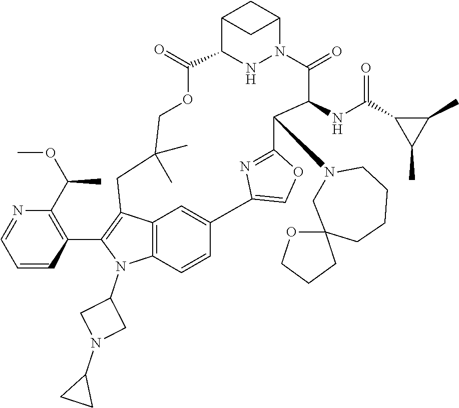 |
| A162 | 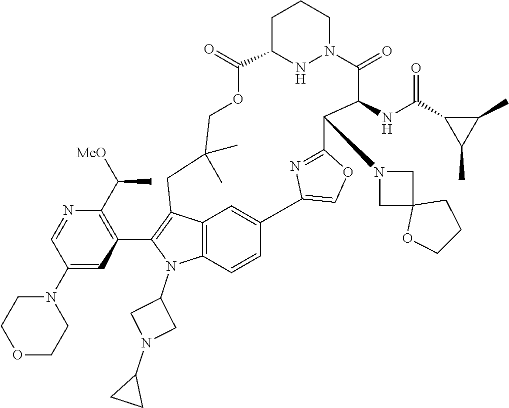 |
| A163 | 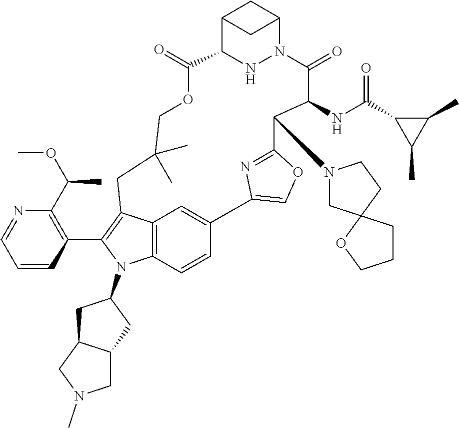 |
| A164 | 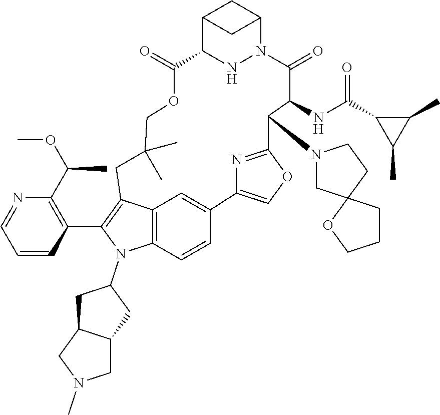 |
| A165 | 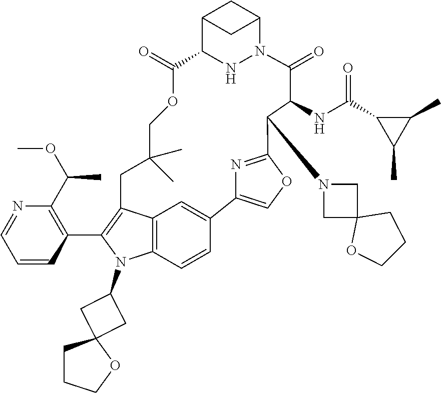 |
| A166 | 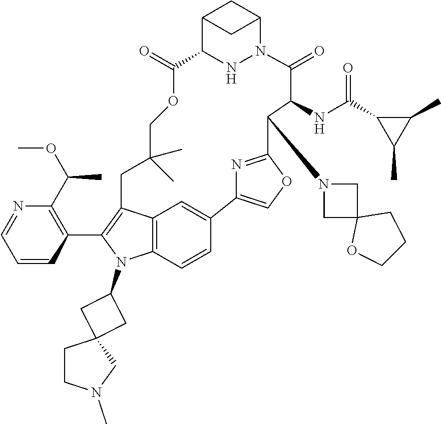 |
| A167 | 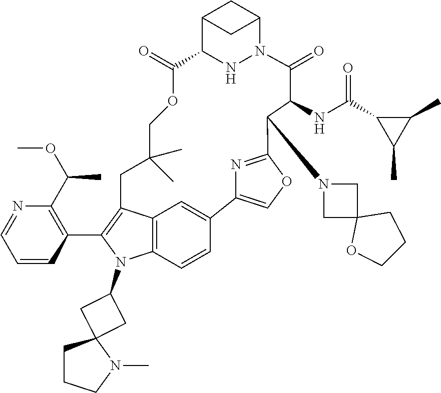 |
| A168 | 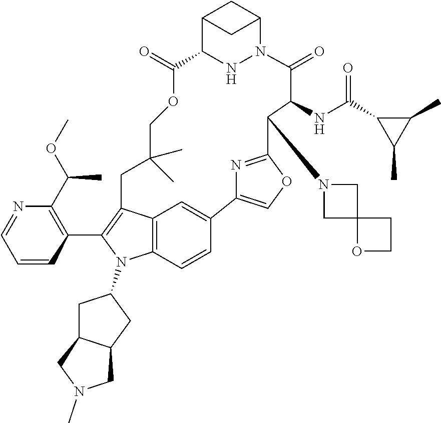 |
| A169 | 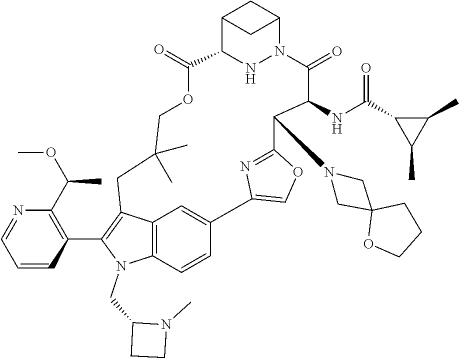 |
| A170 | 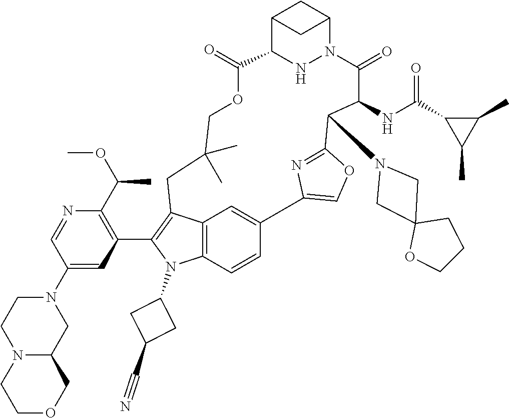 |
| A171 | 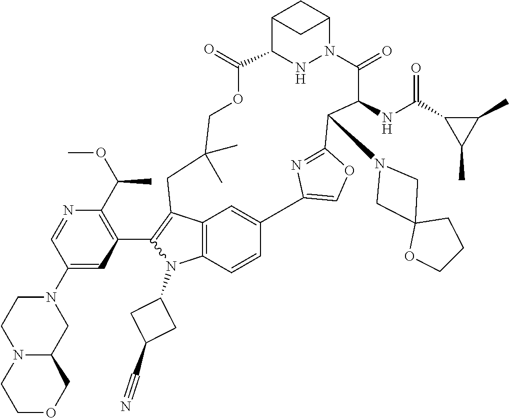 |
| A172 | 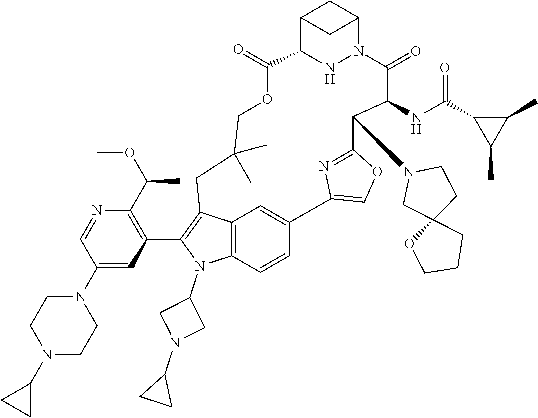 |
| A173 | 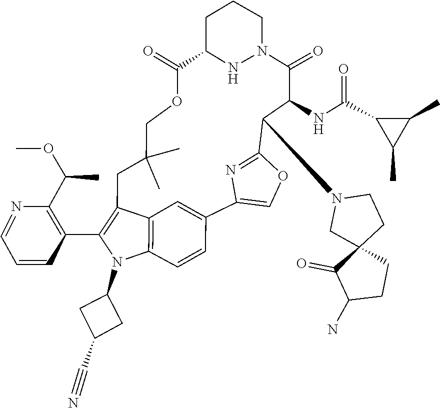 |
| A174 | 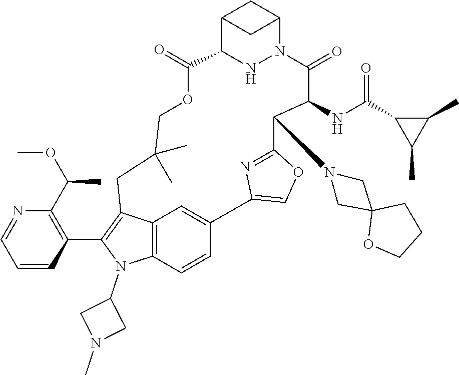 |
| A175 | 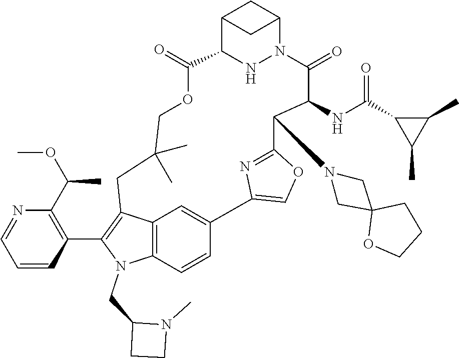 |
| A176 | 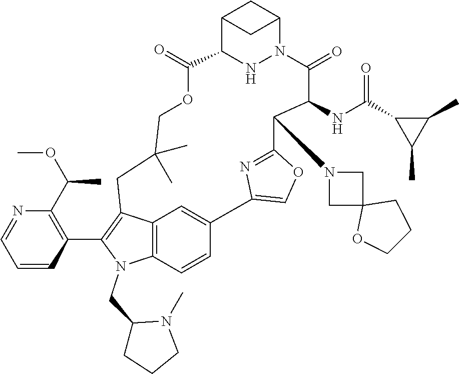 |
| A177 | 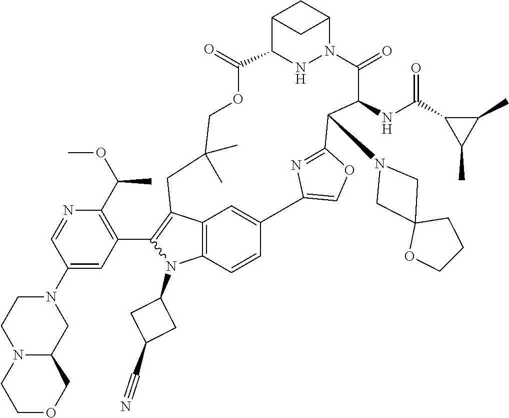 [SMILES: CO[C@@H](C)c1ncc(N2CCN3CCOC[C@@H]3C2)cc1-c1c2c3cc(ccc3n1[C@H]1C[C@@H](C#N)C1)-c1coc(n1)[C@@H](N1CC3(CCCO3)C1)[C@H](NC(=O)[C@H]1[C@H](C)[C@@H]1C)C(=O)N1N[C@H](C(=O)OCC(C)(C)C2)C2CC1C2] |
| A178 | 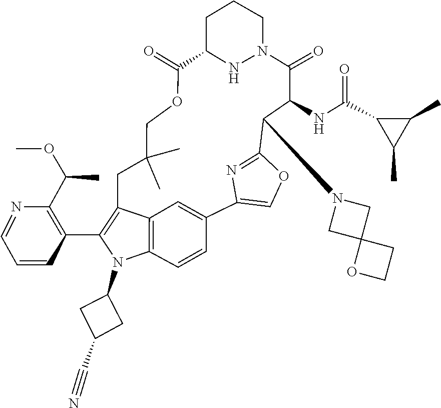 |
| A179 | 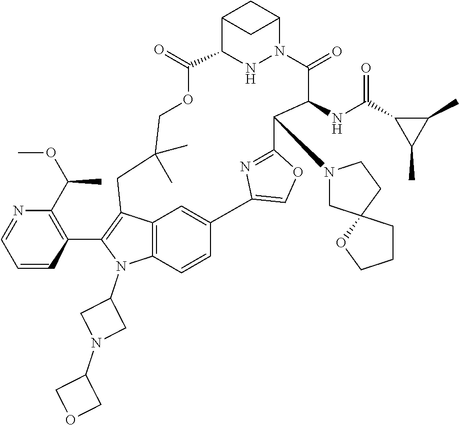 |
| A180 | 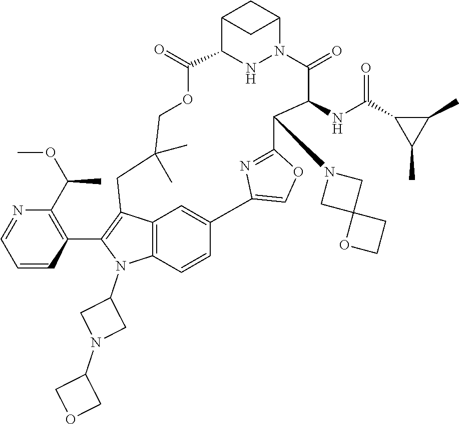 |
| A181 | 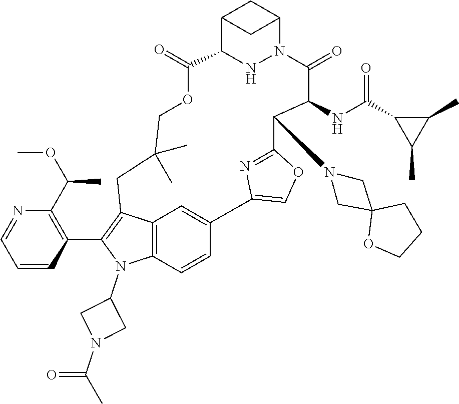 |
| A182 | 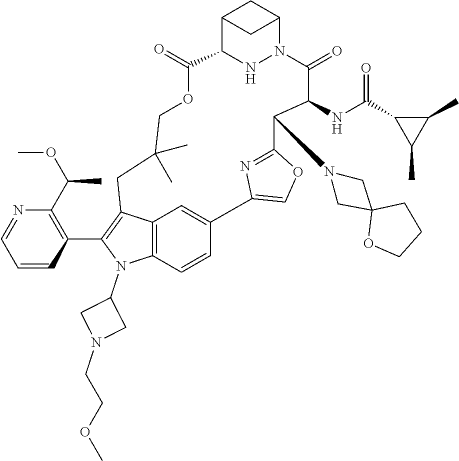 |
| A183 | 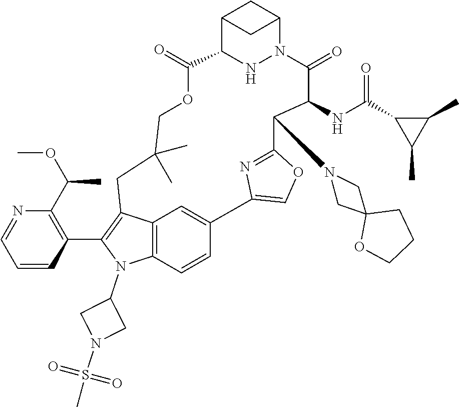 |
| A184 | 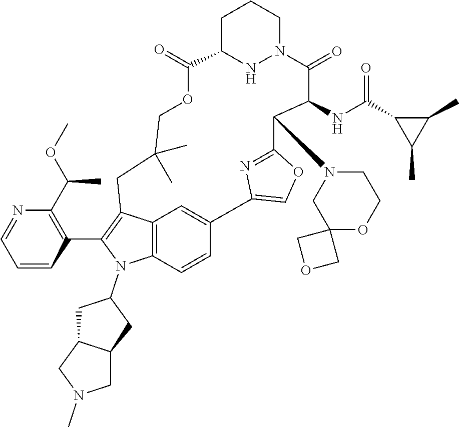 |
| A185 | 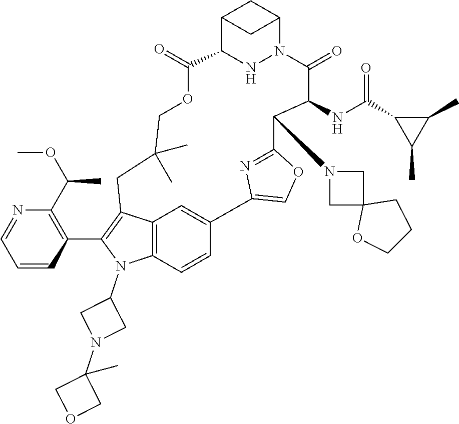 |
| A186 | 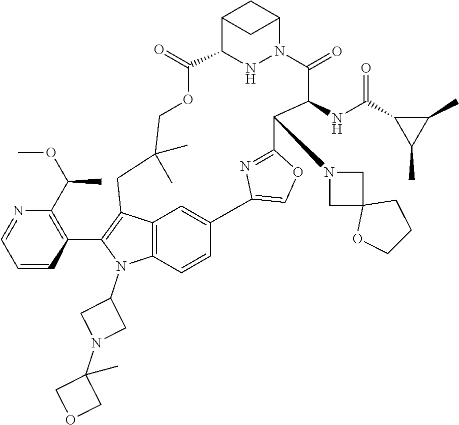 |
| A187 | 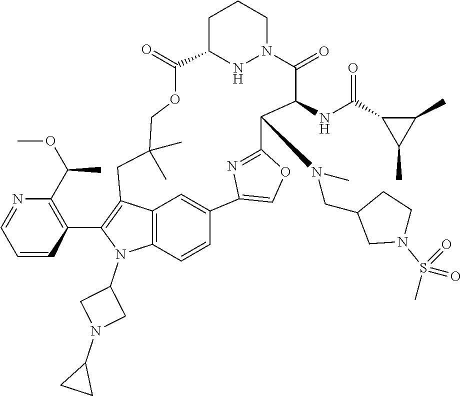 |
| A188 | 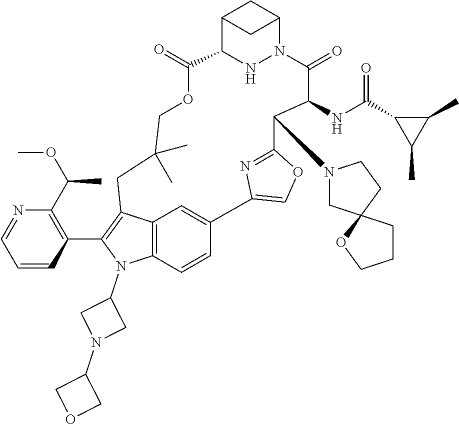 |
| A189 | 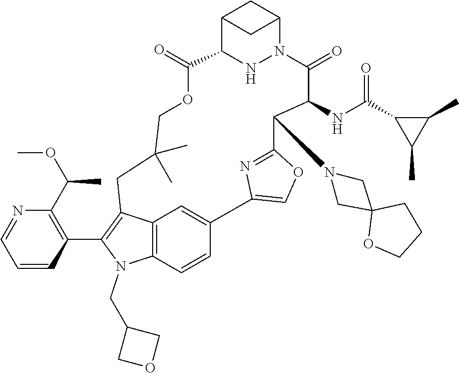 |
| A190 | 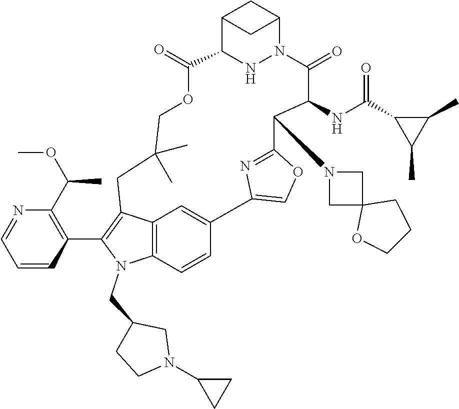 |
| A191 | 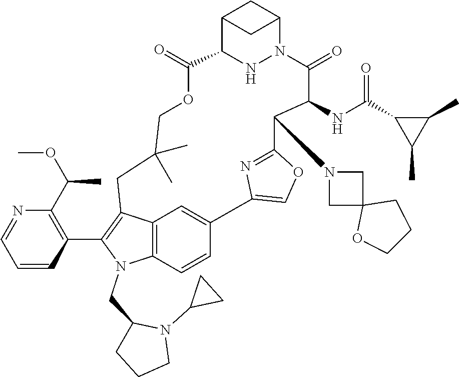 |
| A192 | 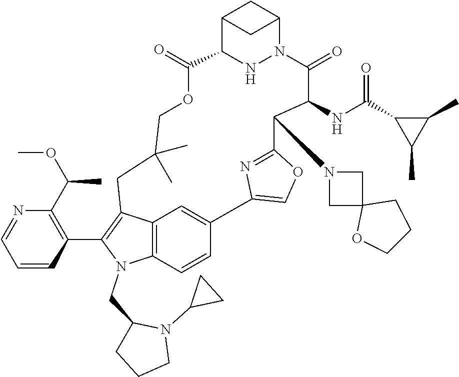 |
| A193 | 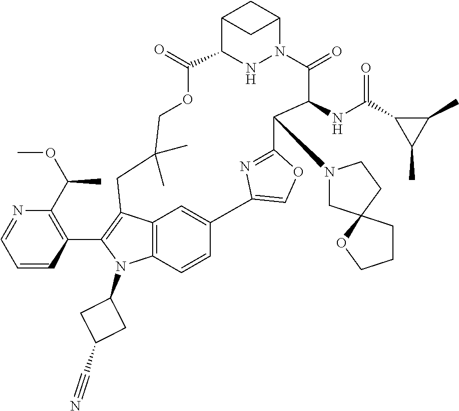 |
| A194 | 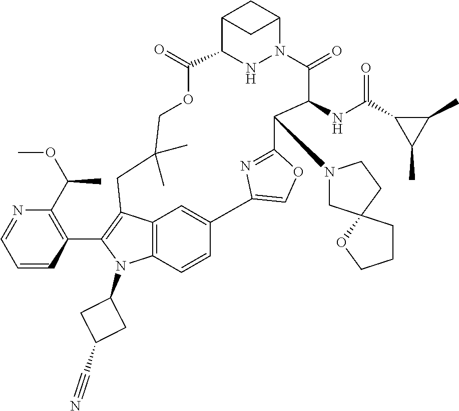 |
| A195 | 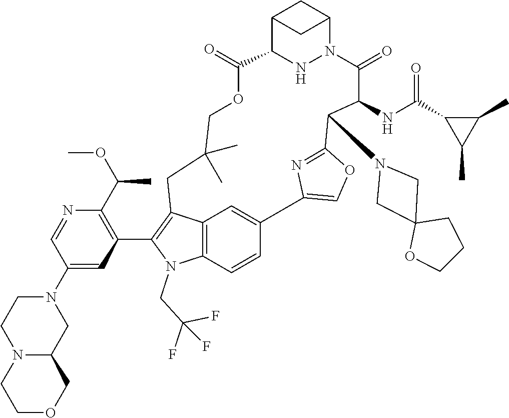 |
| A196 | 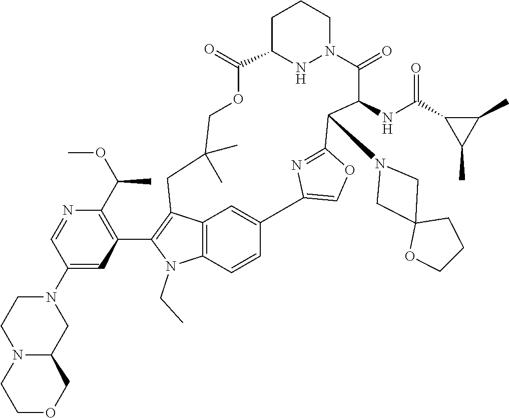 |
| A197 | 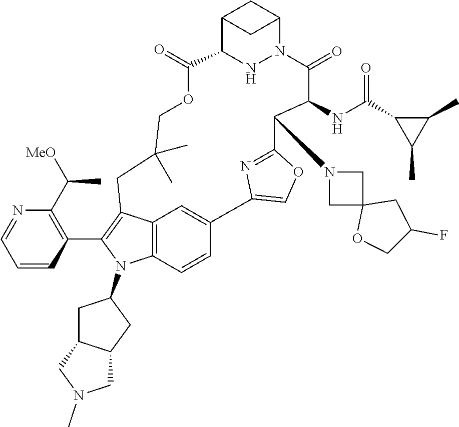 |
| A198 | 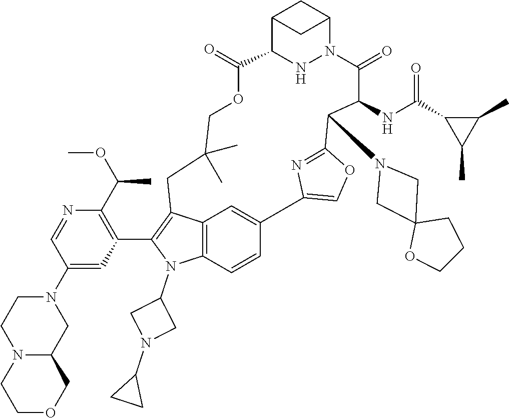 |
| A199 | 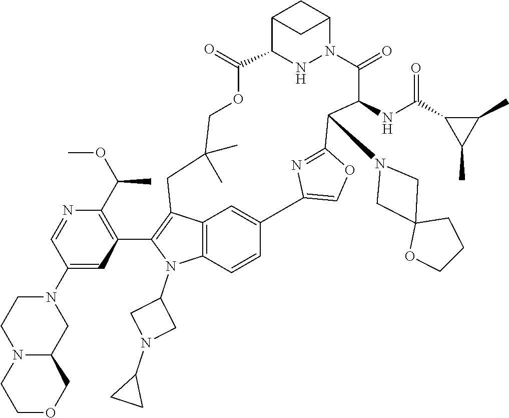 |
| A200 | 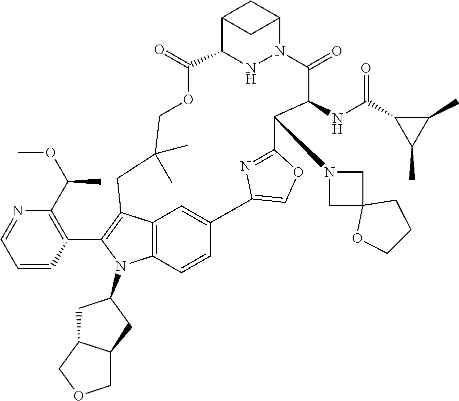 |
| A201 | 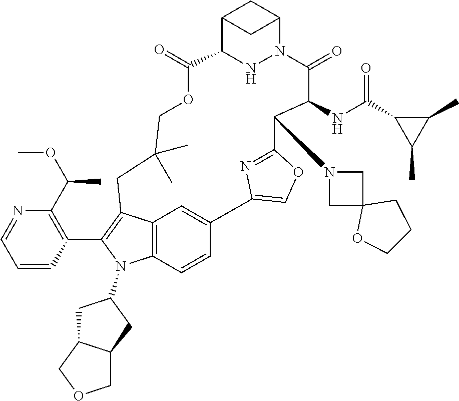 |
| A202 | 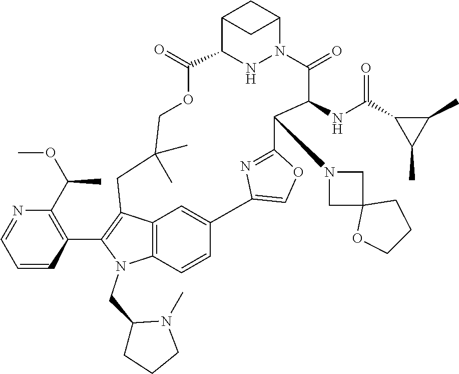 |
| A203 | 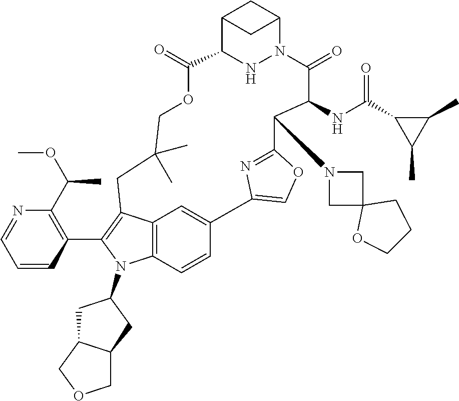 |
| A204 | 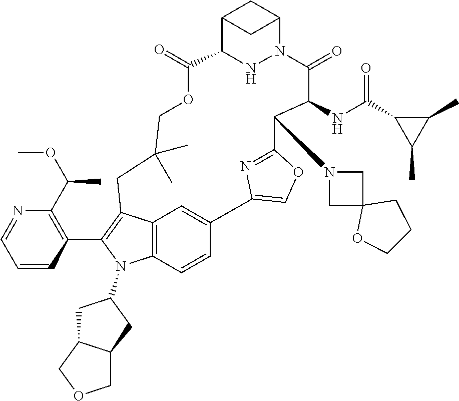 |
| A205 | 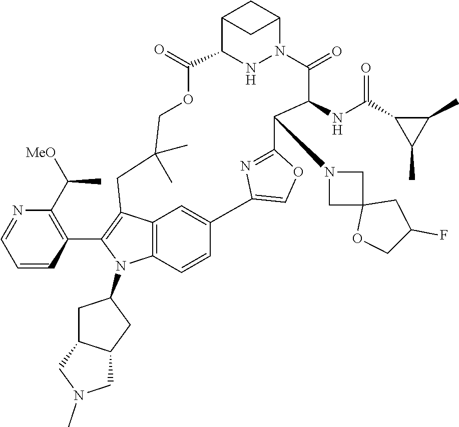 |
| A206 | 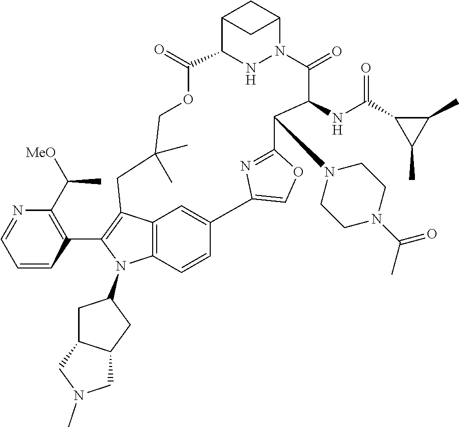 |
| A207 | 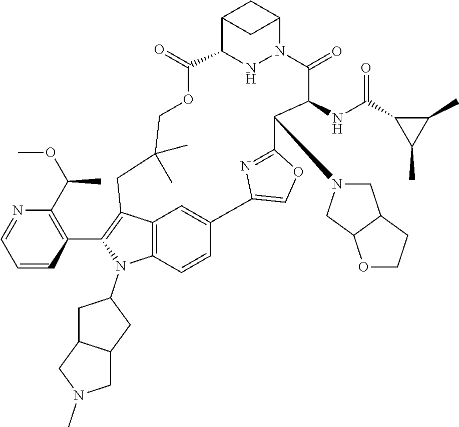 |
| A208 | 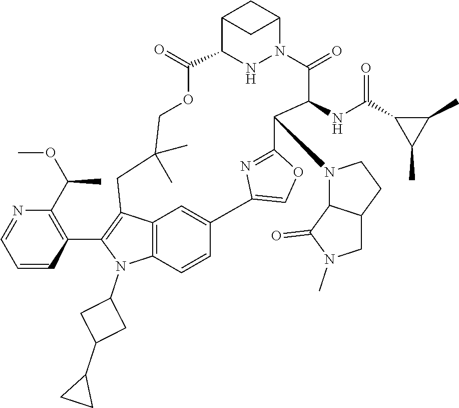 |
| A209 | 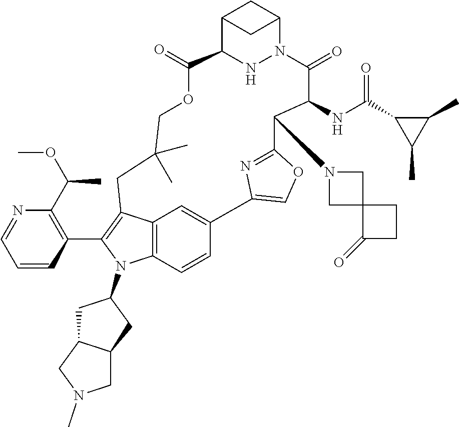 |
| A210 | 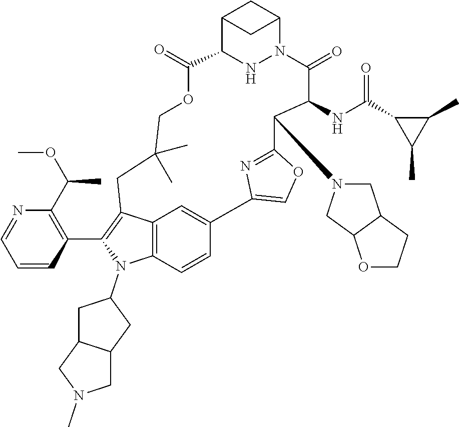 |
| A211 | 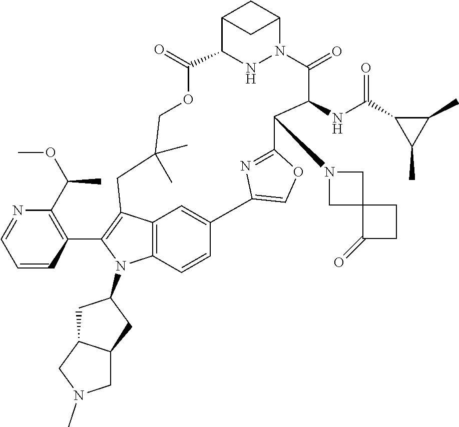 |
| A212 | 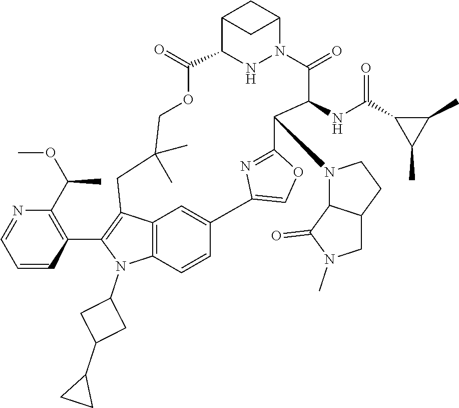 |
| A213 | 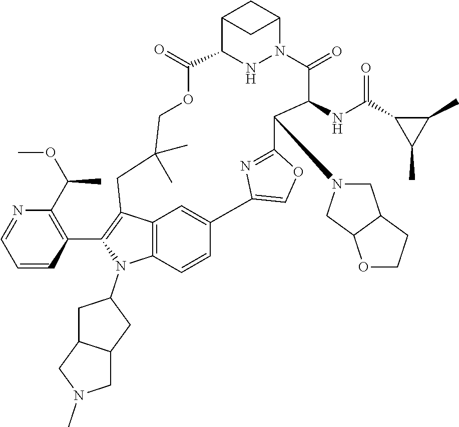 |
| A214 | 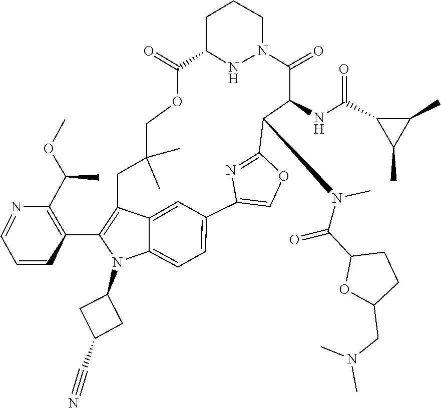 |
| A215 | 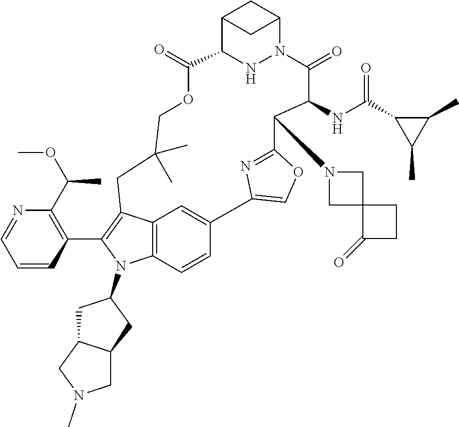 |
| A216 | 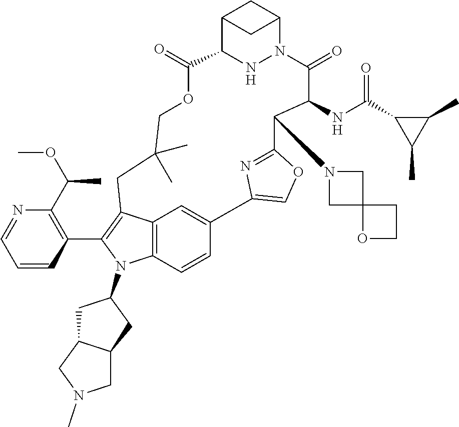 |
| A217 | 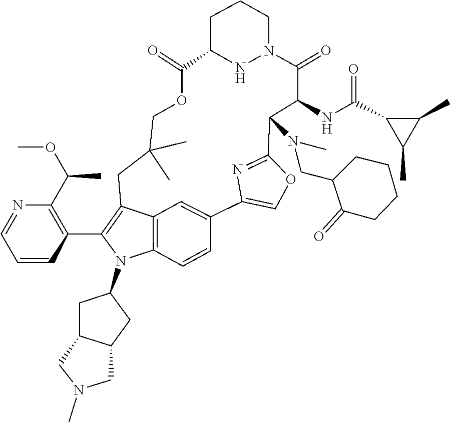 |
| A218 | 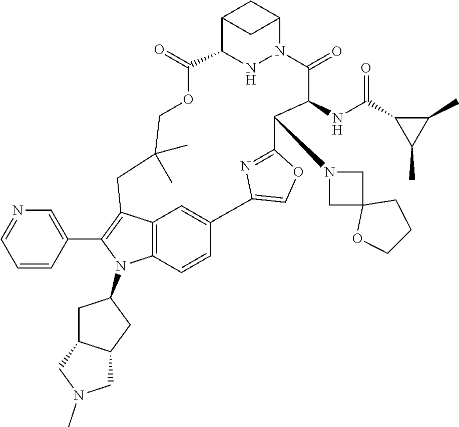 [SMILES: C[C@@H]1[C@H](C)[C@H]1C(=O)N[C@@H]1C(=O)N2N[C@H](C(=O)OCC(C)(C)Cc3c(-c4cccnc4)n([C@H]4C[C@@H]5CN(C)C[C@@H]5C4)c4ccc(cc34)-c3coc(n3)[C@H]1N1CC3(CCCO3)C1)C1CC2C1] |
| A219 | 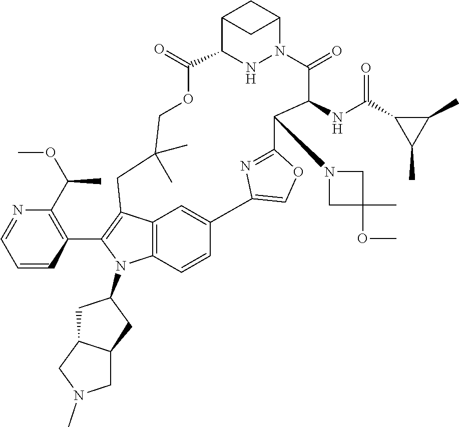 |
| A220 | 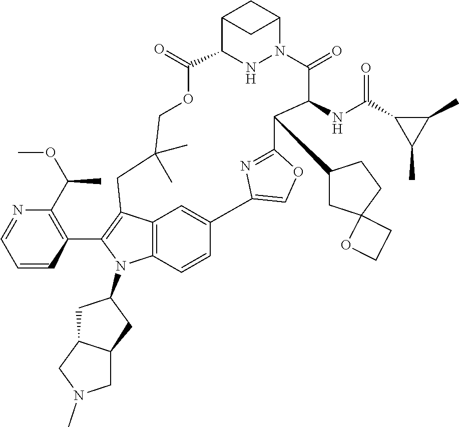 |
| A221 | 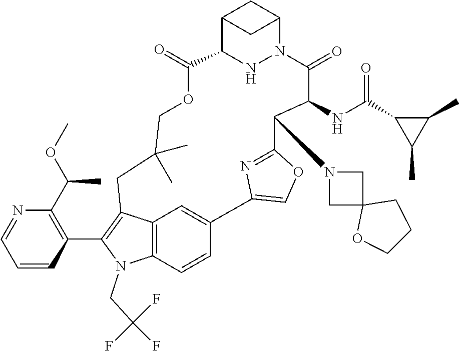 |
| A222 | 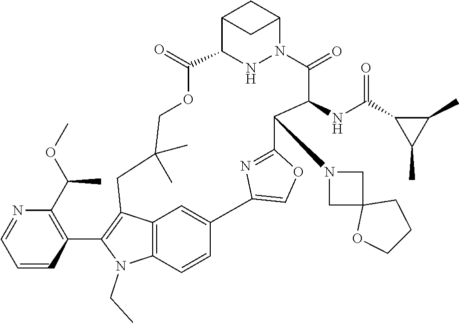 |
| A223 | 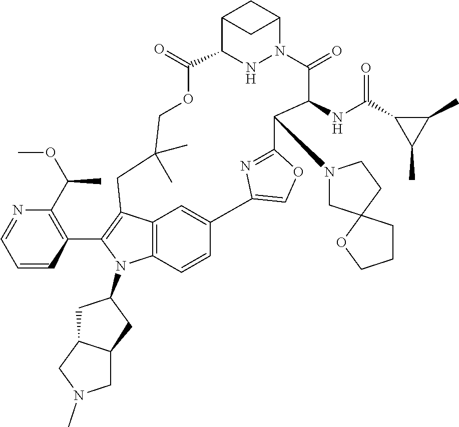 |
| A224 | 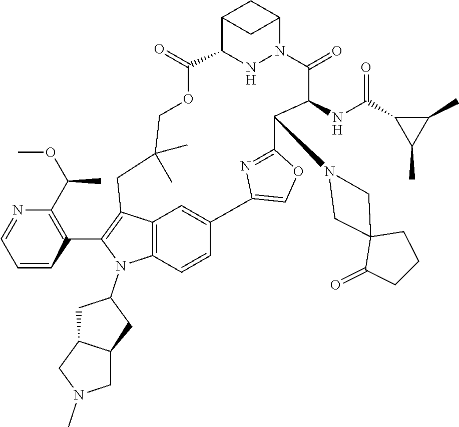 |
| A225 | 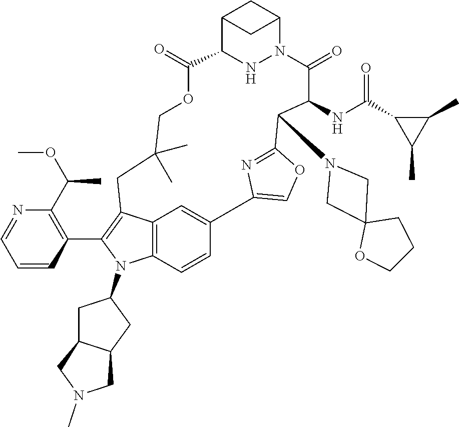 |
| A226 | 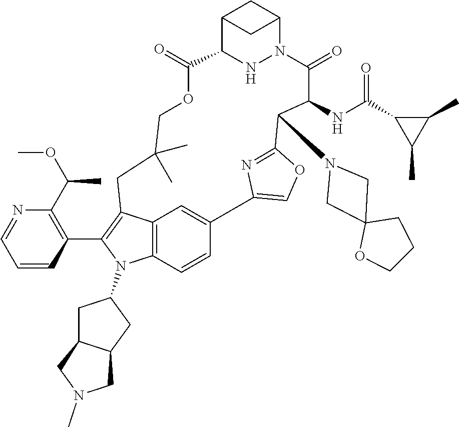 |
| A227 | 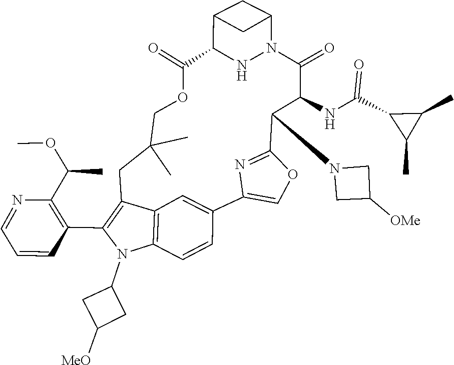 |
| A228 | 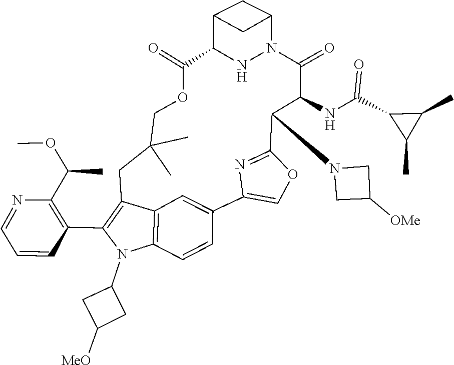 |
| A229 | 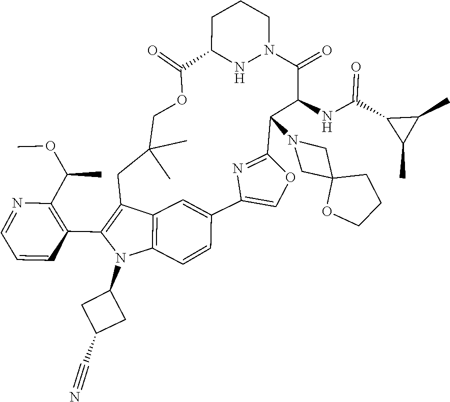 |
| A230 | 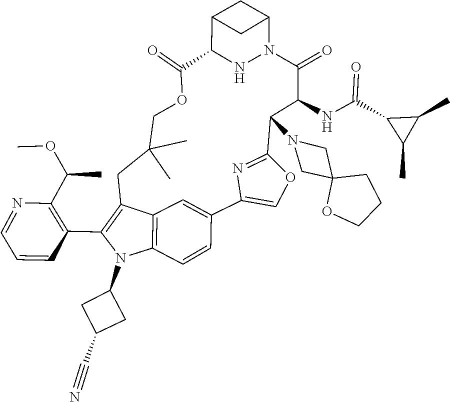 |
| A231 | 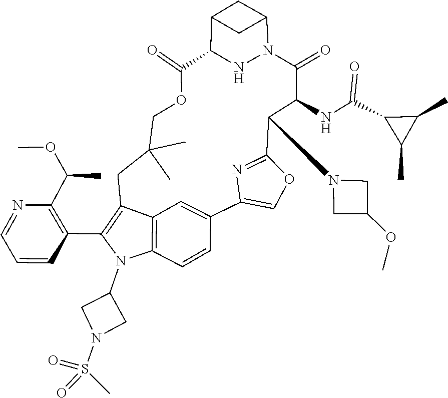 |
| A232 | 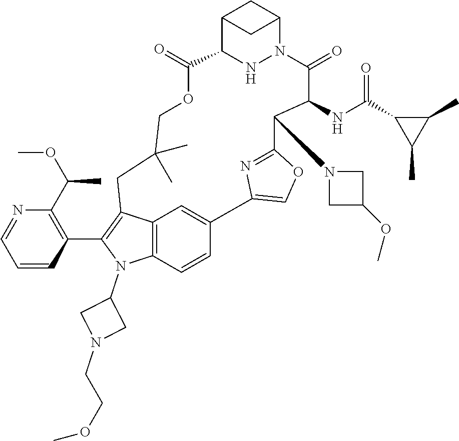 |
| A233 | 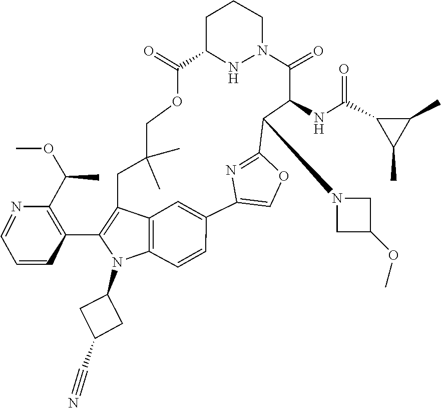 |
| A234 | 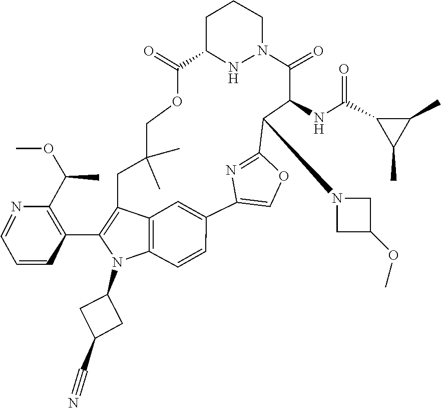 |
| A235 | 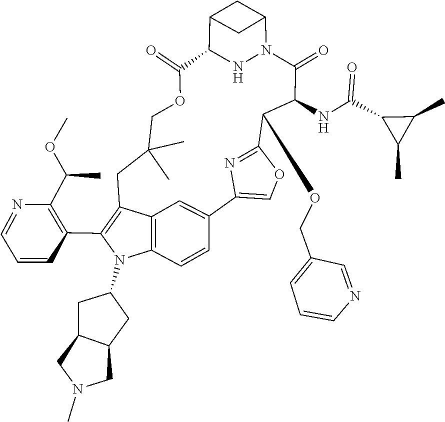 |
| A236 | 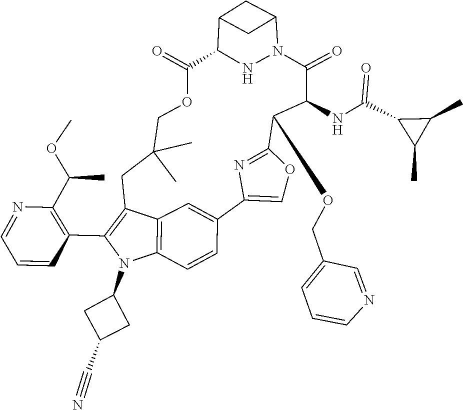 |
| A237 | 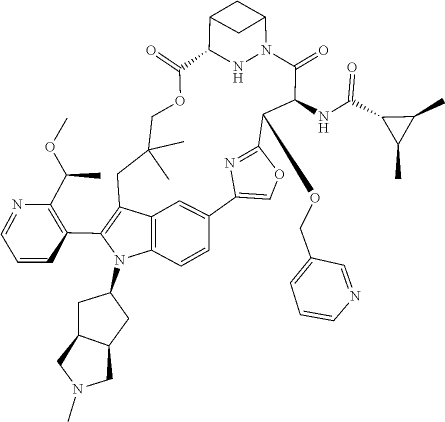 |
| A238 | 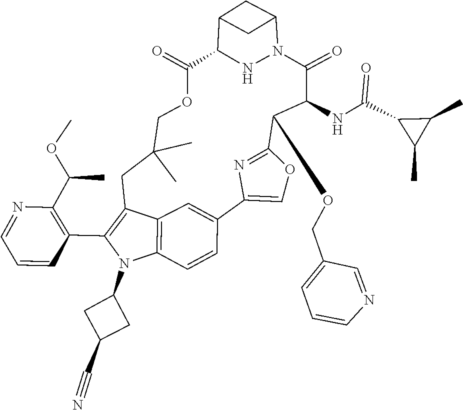 |
| A239 | 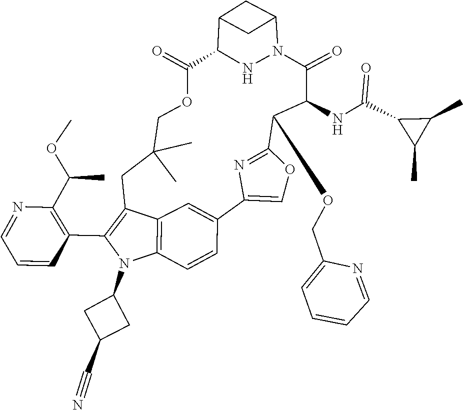 |
| A240 | 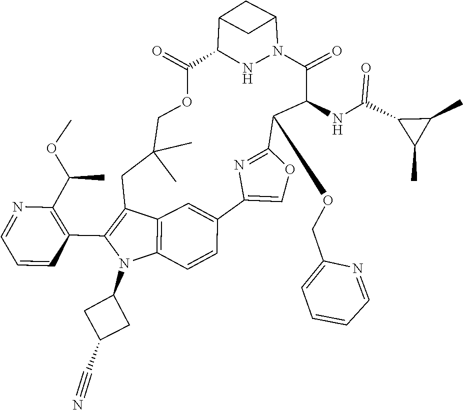 |
| A241 | 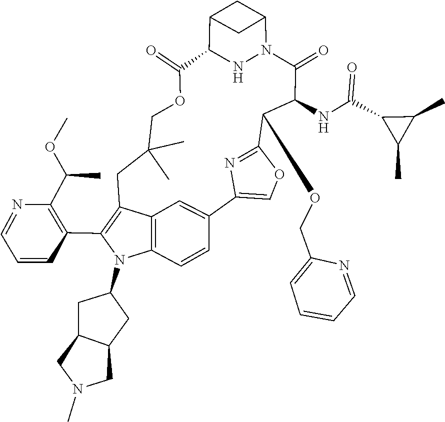 |
| A242 | 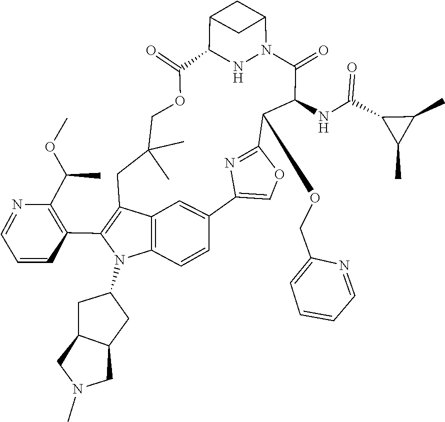 |

Further provided is a pharmaceutical composition comprising a compound of formula I, II, or III, or a compound of Table 1, or a pharmaceutically acceptable salt thereof, and a pharmaceutically acceptable excipient or carrier.

In some embodiments, a compound of the present invention has improved oral bioavailability (% F) compared to what is known in the art. Methods of measuring oral bioavailability are known in the art, and one such method is provided below:

Oral bioavailability may be determined in BALB/c mice. Following intravenous (IV) bolus and oral gavage (PO) administration of a test compound, about 30 μL of whole blood samples are collected at designated time points into tubes containing K₂EDTA. The blood samples are centrifuged at 4600 rpm at 4° C. for about 5 minutes and plasma samples are stored at −80° C. prior to bioanalysis. Plasma samples are extracted by protein precipitation and analyzed by tandem mass spectrometry (LC MS/MS) on, for example, an API 5500 system using electrospray positive ionization.

All PK parameters may be derived from plasma concentration over time data with non-compartment analysis using WinNonlin. The bioavailability (F %, also % F) is estimated using the following equation:

$$F \% = \frac{{AUC}_{inf , PO}}{{AUC}_{inf , IV}} \cdot \frac{{Dose}_{IV}}{{Dose}_{PO}}$$

AUC_{inf,PO} is the area under the plasma concentration over time from time zero to infinity following PO administration.
AUC_{inf,IV} is the area under the plasma concentration overtime from time zero to infinity following IV administration.
Dose_{IV} is the total dose of IV administration
Dose_{PO} is the total dose of PO administration

In general, F % (or % F) values of over 30% are preferred, with values over 50% being more preferred.

In some embodiments, a compound of the present invention is selective for one or more particular Ras mutants over other Ras mutants or wild-type compared to what is known in the art. Methods of measuring such selectivity are known in the art, such as the Ras-Raf binding assay, a protocol for which is provided above. Accordingly, in some embodiments, compounds of the present invention are selective for KRAS having a mutation at G12 over other Ras mutants or over wild-type, or combinations thereof. In some embodiments, compounds of the present invention are selective for KRAS having a mutation at G13 over other Ras mutants or over wild-type, or combinations thereof. In some embodiments, compounds of the present invention are selective for KRAS having a mutation at G12 over other Ras mutants or over wild-type, or combinations thereof.

In some embodiments, a compound of the present invention is more potent for one or more particular Ras mutants over other Ras mutants or wild-type compared to what is known in the art. Methods of measuring such potency are known in the art, such as the pERK assay, a protocol for which is provided in the Examples below. In some embodiments, compounds of the present invention exhibit greater potency with respect to KRAS^{G12V} than what is known in the art. Compounds of the present invention may also exhibit greater potency with respect to other RAS mutants disclosed herein, or combinations thereof. In some embodiments, a compound of the present invention exhibits at least 1.5× greater potency with respect to KRAS^{G12V} versus KRAS^{G12D} using the pERK assay.

In some embodiments, a compound of the present invention exhibits a greater detrimental effect on cell viability with respect to one or more particular Ras mutants over other Ras mutants or wild-type compared to what is known in the art. Methods of measuring cell viability are known in the art, such as the CELLTITER-GLO® Cell Viability Assay, a protocol for which is provided in the Examples below. Accordingly, in some embodiments, compounds of the present invention exhibit a greater decrease in cell viability with respect to KRAS^{G12V} compared to what is known in the art. Compounds of the present invention may also exhibit a greater decrease in cell viability respect to other RAS mutants disclosed herein, or combinations thereof. In some embodiments, compounds of the present invention exhibit a greater decrease in cell viability with respect to KRAS^{G12V} versus wild-type KRAS.

In some embodiments, a compound of the present invention promotes the hydrolysis of RAS-bound GTP to GDP. In some embodiments, the hydrolysis half-life (i.e., the time it takes for half of RAS-bound GTP to be hydrolyzed to GDP) of a compound of the present invention is less than 10 minutes upon administration of the compound at a 1 uM concentration. In some embodiments, the hydrolysis half-life is less than 50 minutes.

In some embodiments, a compound of the present invention may exhibit greater metabolic stability, permeability, or solubility, or a combination thereof, versus what is known in the art. Methods for measuring such properties are known in the art. In some embodiments, a compound of the present invention may exhibit improvements with respect to any of the following properties, or a combination thereof, compared to what is known in the art: selectivity, potency, cell viability, metabolic stability, permeability, oral bioavailability, or solubility.

In some embodiments, a compound of the present invention is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

Compounds of the present invention are also adaptable for uses in antibody-drug conjugates as well as degrader applications.

Further provided is a method of treating cancer in a subject in need thereof, the method comprising administering to the subject a therapeutically effective amount of a compound of the present invention, or a pharmaceutically acceptable salt thereof. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer comprises a Ras mutation, such as K-Ras G12V. Other Ras mutations are described herein.

Further provided is a method of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof. For example, the Ras protein is K-Ras G12V. Other Ras proteins are described herein. The cell may be a cancer cell, such as a pancreatic cancer cell, a colorectal cancer cell, a lung cancer (e.g., non-small cell lung cancer cell), an acute myeloid leukemia cell, a multiple myeloma cell, a thyroid gland adenocarcinoma cell, a myelodysplastic syndrome cell, a melanoma cell, or a squamous cell lung carcinoma cell. Other cancer types are described herein. The cell may be in vivo or in vitro.

With respect to compounds of the present invention, one stereoisomer may exhibit better inhibition than another stereoisomer. For example, one atropisomer may exhibit inhibition, whereas the other atropisomer may exhibit little or no inhibition.

In some embodiments, a method or use described herein further comprises administering an additional anti-cancer therapy. In some embodiments, the additional anti-cancer therapy is a HER2 inhibitor, an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, or a combination thereof. In some embodiments, the additional anticancer therapy is a SHP2 inhibitor. In other embodiments, the additional anticancer agent or therapy is a pan-KRAS inhibitor (e.g., Pan KRAS-IN-1, BI-286, YL-17231). Other additional anti-cancer therapies are described herein.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Scheme below, together with synthetic methods known in the art of synthetic organic chemistry, or variations thereon as appreciated by those skilled in the art. As a further example, synthetic methods described in WO 2020/132597, WO 2021/091982, WO 2021/091967, WO 2021/091956, WO 2022/060836, WO 2022/235864, WO 2022/235870, WO 2023/060253, and WO 2023/133543, the disclosure of each of which is incorporated herein by reference, may be useful in preparing compounds of the invention. These methods include but are not limited to those methods described in the Scheme below.

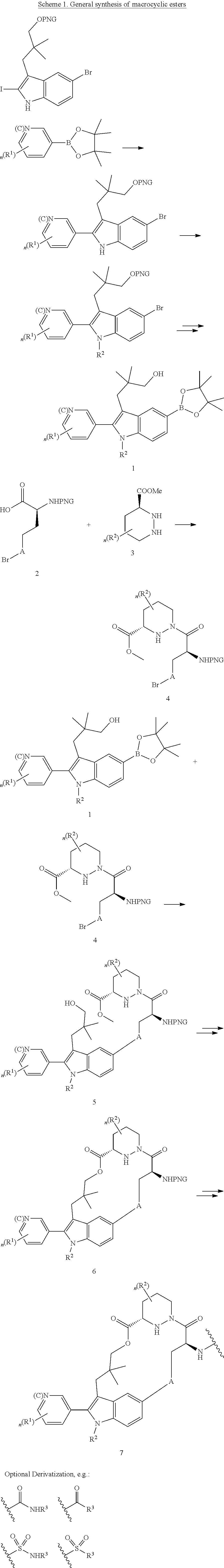

A general synthesis of macrocyclic esters is outlined in Scheme 1. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl (S)-hexahydropyridazine-3-carboxylate analogs (3) can be prepared by a variety of methods highlighted below.

Methyl-amino-3-(4-bromothiazol-2-yl)propanoyl)hexahydropyridazine-3-carboxylate (4) can be prepared via coupling of (S)-2-amino-3-(4-bromothiazol-2-yl)propanoic acid (2) with methyl (S)-hexahydropyridazine-3-carboxylate derivative (3).

The final macrocyclic esters can be made by coupling of methyl-amino-3-(4-bromothiazol-2-yl)propanoyl)hexahydropyridazine-3-carboxylate (4) and an appropriately substituted indolyl boronic ester (1) in the presence of Pd catalyst followed by hydrolysis and macrolactonization steps to result in an appropriately protected macrocyclic intermediate (5). Deprotection and coupling with an appropriately substituted carboxylic acid (or other coupling partner) results in a macrocyclic product. Additional deprotection or functionalization steps could be required to produce a final compound (6).

Further, with respect to Scheme 1, the thiazole may be replaced with an alternative optionally substituted 5 to 6-membered heteroarylene, an optionally substituted 3 to 6-membered cycloalkylene, optionally substituted 3 to 6-membered heterocycloalkylene (e.g., morpholino), or optionally substituted 6-membered arylene (e.g., phenyl). PNG is a hydroxy protecting group when attached to the oxygen atom of the molecule and as an amino protecting group when attached to the nitrogen atom (e.g., in compound 4). The substituents R¹ and R² and moiety A in Scheme 1 are as defined in the specification and claims. The subscript n is 1, 2 or 3. R³ corresponds to variable W, as defined in the specification and claims.

In some embodiments, a general synthesis of macrocyclic esters is outlined in Scheme 1A.

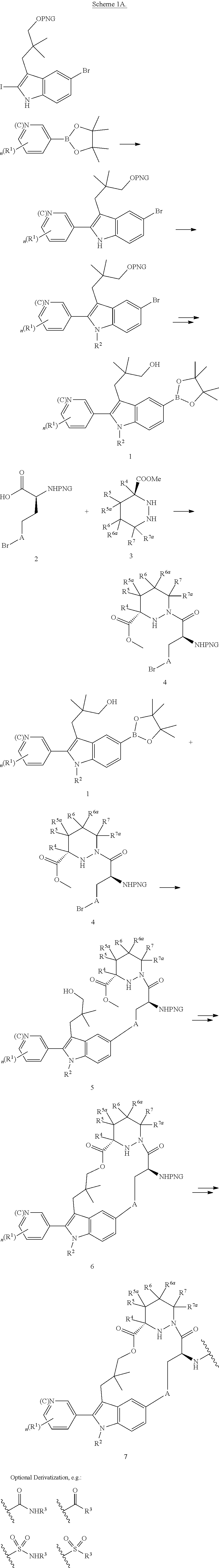

As shown in Schemes 1 and 1A, PNG is a hydroxy protecting group when attached to the oxygen atom of the molecule. PNG is an amino protecting group when attached to the nitrogen atom (e.g., in compound 4). The substituents R¹, R², R⁴, R^{4a}, R⁵, R^{5a}, R⁶, R^{6a}, R⁷, R^{7a} and moiety A are defined in the specification and claims. R³ corresponds to variable W, as defined in the specification and claims.

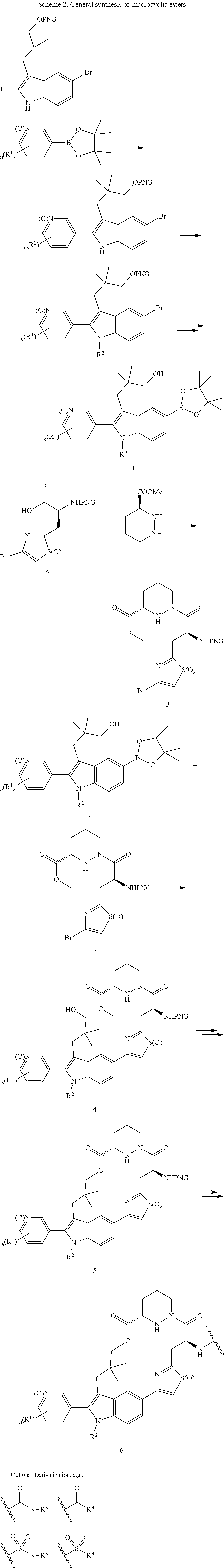

A further general synthesis of macrocyclic esters is outlined in Scheme 2. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl)propanoyl)hexahydropyridazine-3-carboxylate (3) can be prepared via coupling of (S)-2-amino-3-(4-bromothiazol-2-yl)propanoic acid (2) with methyl (S)-hexahydropyridazine-3-carboxylate (3).

The final macrocyclic esters can be made by coupling of methyl-amino-3-(4-bromothiazol-2-yl)propanoyl)hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (4). Deprotection and coupling with an appropriately substituted carboxylic acid (or other coupling partner) results in a macrocyclic product. Additional deprotection or functionalization steps could be required to produce a final compound (6).

Further, with respect to Scheme 1, the thiazole may be replaced with an alternative optionally substituted 5 to 6-membered heteroarylene, an optionally substituted 3 to 6-membered cycloalkylene, optionally substituted 3 to 6-membered heterocycloalkylene (e.g., morpholino), or optionally substituted 6-membered arylene (e.g., phenyl). PNG is a hydroxy protecting group when attached to the oxygen atom of the molecule and as an amino protecting group when attached to the nitrogen atom of the molecule (e.g., in compound 3). The substituents R¹ and R² in Scheme 2 are as defined in the specification and claims. The subscript n is 1, 2 or 3. R³ corresponds to variable W, as defined in the specification and claims.

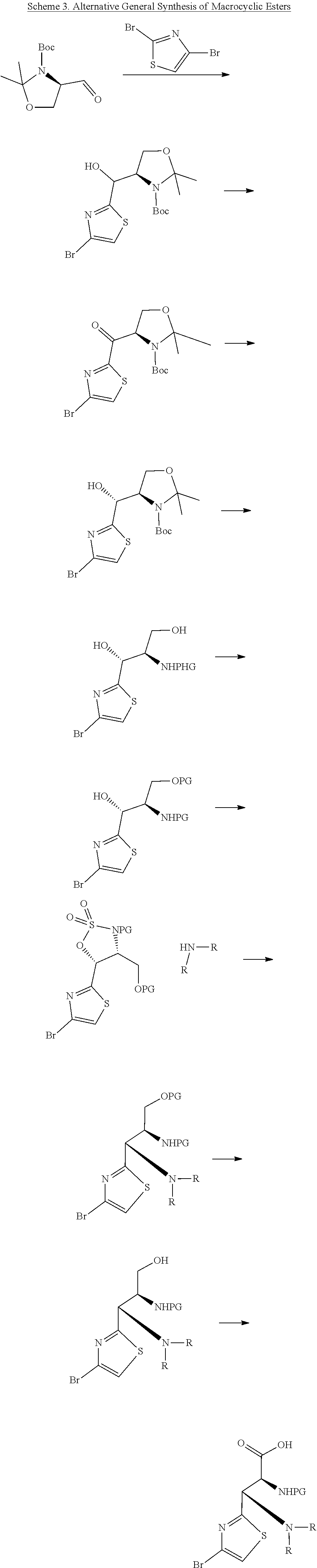

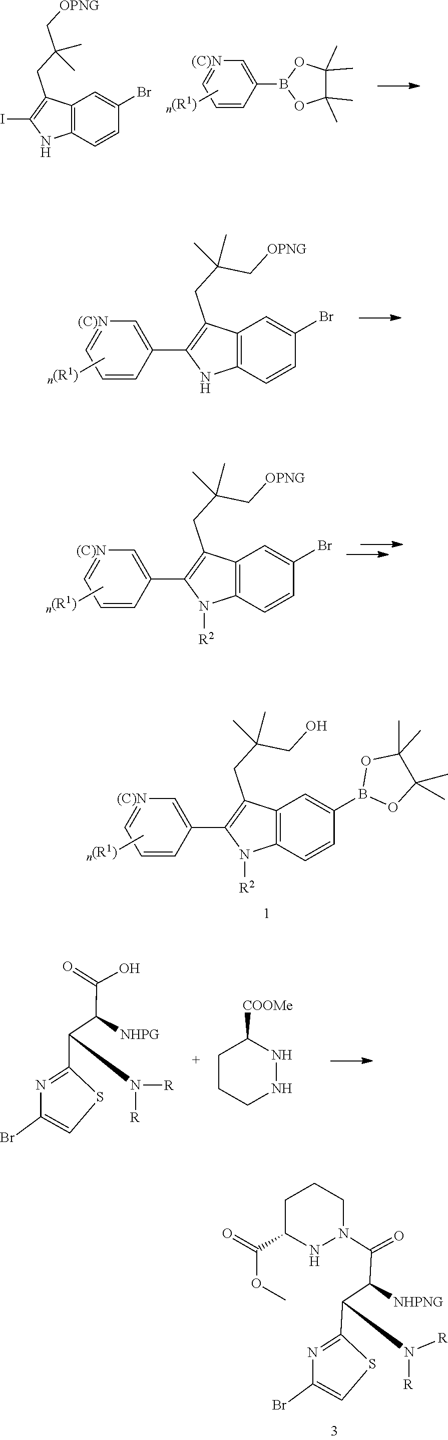

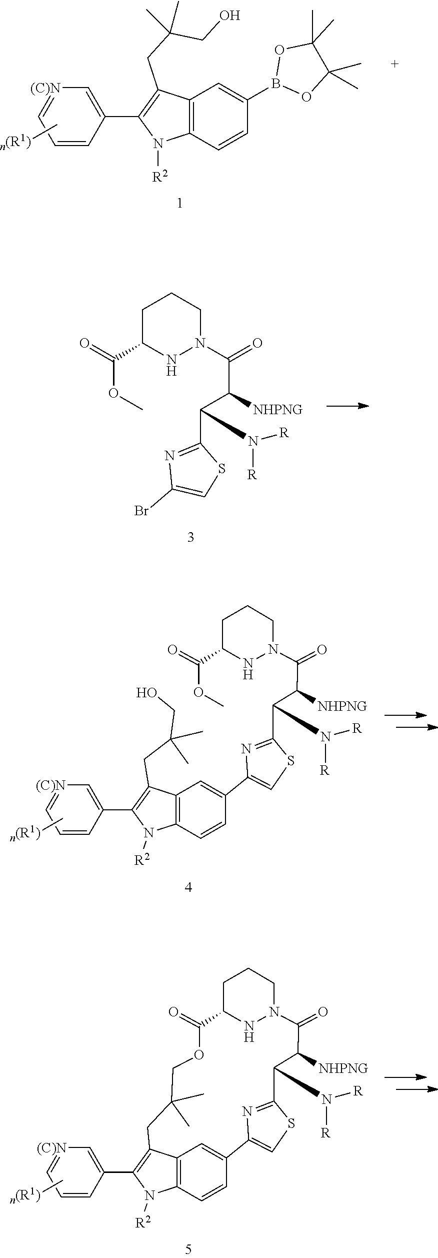

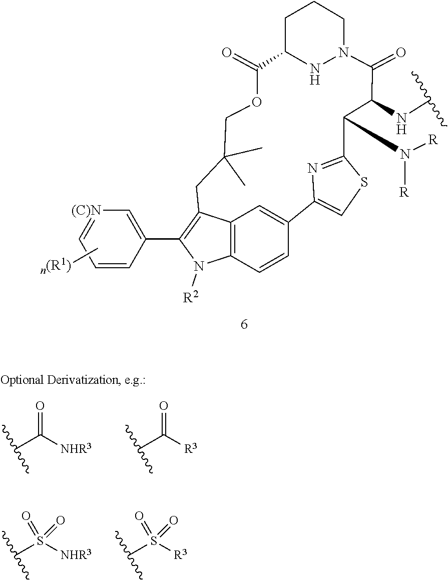

A further general synthesis of macrocyclic esters is outlined in Scheme 3. An appropriately substituted indolyl boronic ester (1) can be prepared as described in Schemes 1 and 2.

Amino-substituted (S)-2-amino-3-(4-bromothiazol-2-yl)propanoic acid can be prepared from 2,4-dibromothiazole as shown in Scheme 3, above.

Amino-substituted methyl-amino-3-(4-bromothiazol-2-yl)propanoyl)hexahydropyridazine-3-carboxylate (3) can be prepared via coupling of amino-substituted (S)-2-amino-3-(4-bromothiazol-2-yl)propanoic acid with methyl (S)-hexahydropyridazine-3-carboxylate.

The final macrocyclic esters can be prepared in the manner described in Schemes 1 and 2. As shown in Scheme 3, PNG is a hydroxy protecting group when attached to the oxygen atom of the molecule and as an amino protecting group when attached to the nitrogen atom of the molecule. PNG is a hydroxy protecting group when attached to oxygen and an amino protecting group when attached to nitrogen. The substituents R¹ and R² are as defined in the specification and claims. The subscript n is 1, 2 or 3. R³ corresponds to variable W, as defined in the specification and claims. The two R substituents on the nitrogen atom in compounds 3, 4, 5 and 6 correspond to substituents R¹² and R¹³, also defined in the specification and claims.

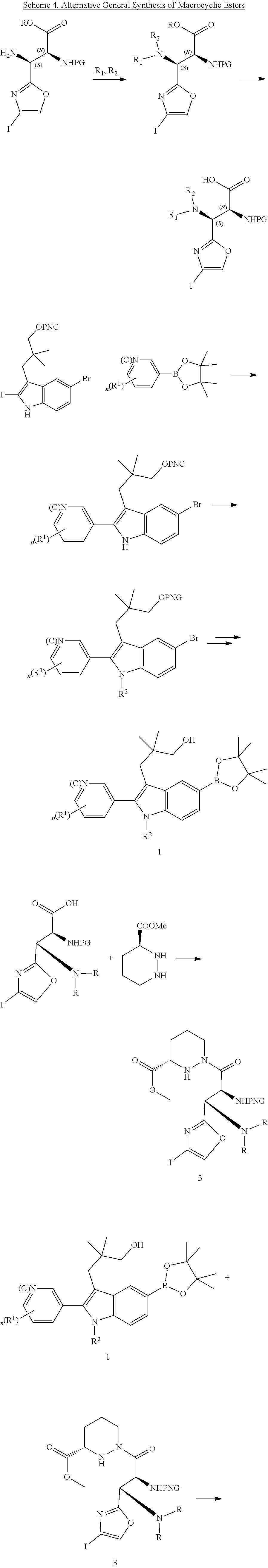

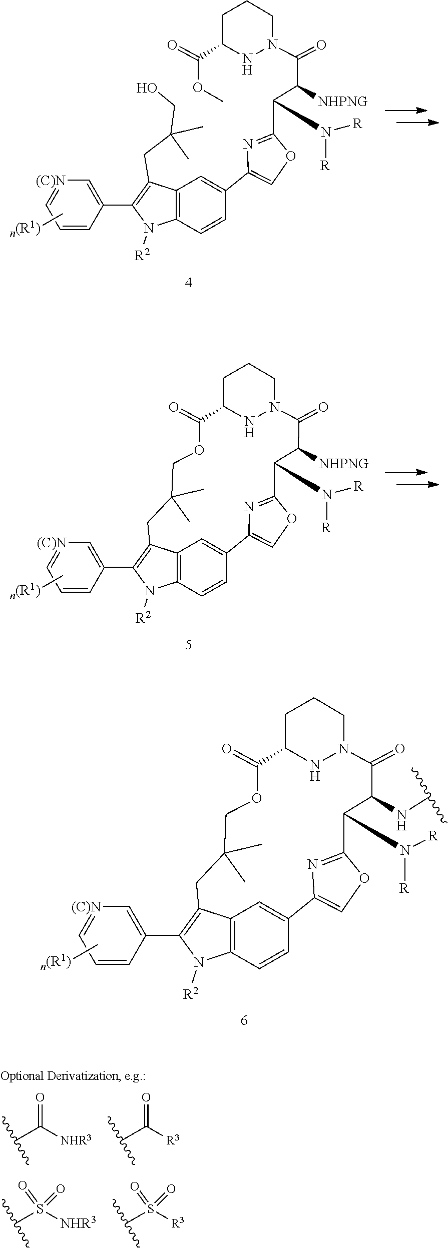

Oxazole-containing compounds can be prepared in an analogous method as shown in Schemes 1-3 but with diamino-substituted iodo-oxazole (3). This compound can be prepared as shown in Scheme 4, above.

The final macrocyclic esters can be prepared in the manner described in Schemes 1 and 2. Compounds with various substitutions on the pyridazine ring, as disclosed and claimed herein, can be prepared using methods known in the art and described in WO2024067857, which is incorporated by reference in its entirety for all purposes.

As shown in Scheme 4, PNG represents an amino protecting group when attached to the nitrogen atom of the molecule. PNG is a hydroxy protecting group when attached to the oxygen atom of the molecule and as an amino protecting group when connected to nitrogen. The substituents R¹ and R² are as defined in the specification and claims. The subscript n is 1, 2 or 3. R³ corresponds to variable W, as defined in the specification and claims. The two R substituents on the nitrogen atom in compounds 3, 4, 5 and 6 correspond to substituents R¹² and R¹³, also defined in the specification and claims. The R¹ and R₂ substituents correspond to substituents R¹² and R¹³, as defined in the specification and claims. In some embodiments, the R group in

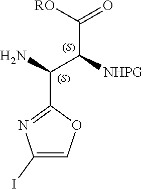

is hydrogen or an optionally substituted alkyl.

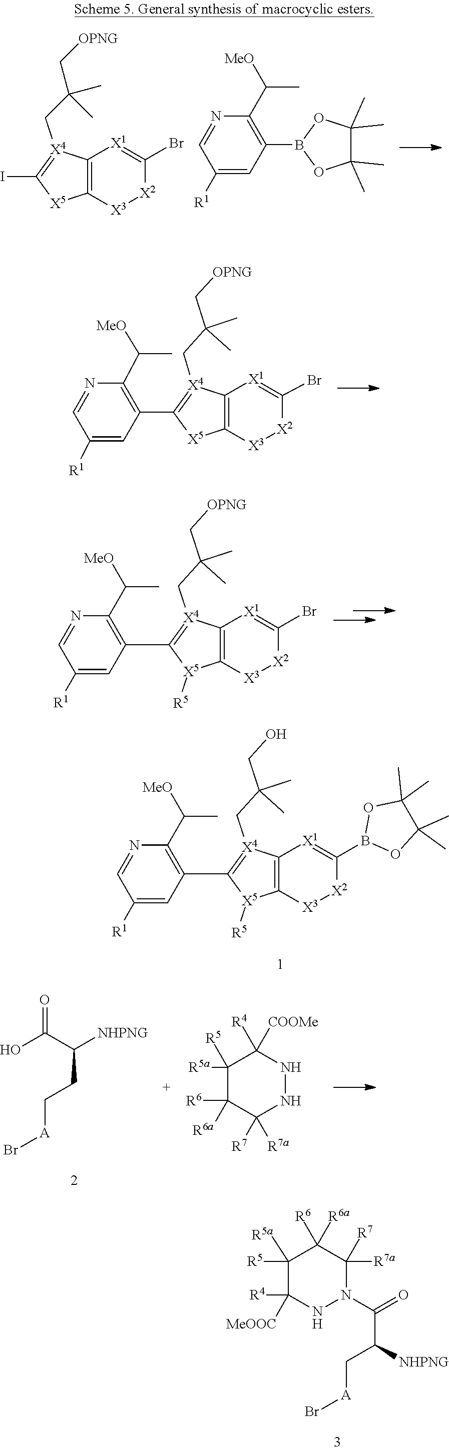

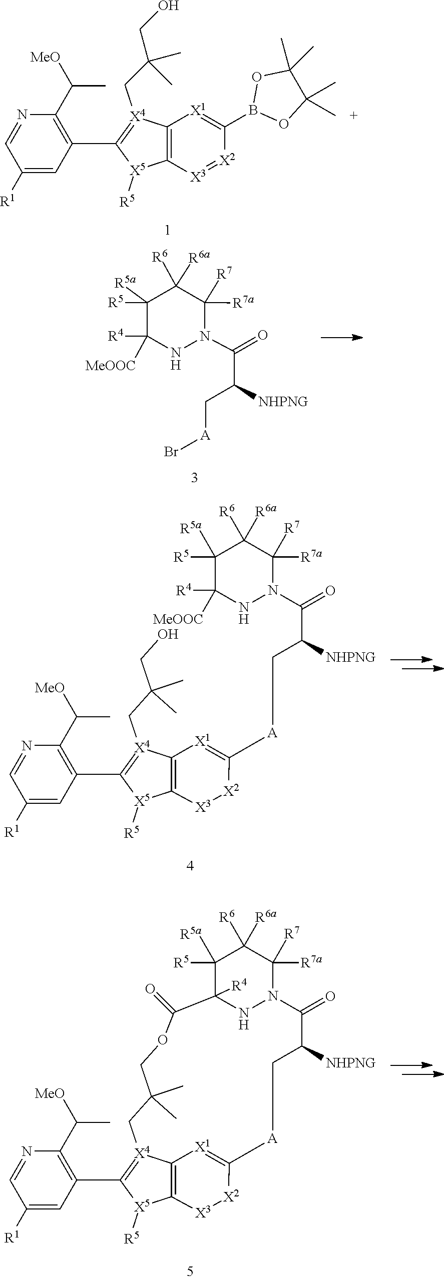

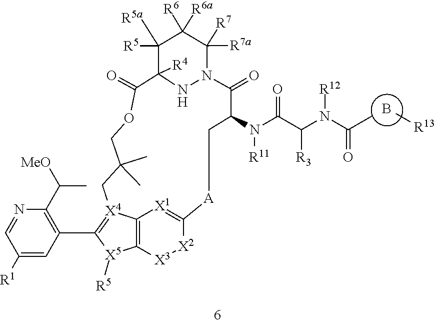

A further general synthesis of macrocyclic esters is outlined in Scheme 5. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl)propanoyl)hexahydropyridazine-3-carboxylate (3) can be prepared via coupling of (S)-2-amino-3-(4-bromothiazol-2-yl)propanoic acid (2) with methyl (S)-hexahydropyridazine-3-carboxylate (3).

The macrocyclic esters can be made by coupling of methyl-amino-3-(4-bromothiazol-2-yl)propanoyl)hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (4). Deprotection and coupling with an appropriately substituted carboxylic acid (or other coupling partner) results in a macrocyclic product. Additional deprotection or functionalization steps could be required to produce a final compound (6).

As shown in Scheme 5, PNG is a hydroxy protecting group when attached to the oxygen atom of the molecule. PNG is an amino protecting group when attached to the nitrogen atom (e.g., in compound 4). The substituents R¹, R², R⁴, R⁵, R^{5a}, R⁶, R^{6a}, R⁷, R^{7a}, R³, R¹¹, R¹² and R¹³; the variables X¹, X², X³, X⁴, X⁵ and A; and ring B are defined in the specification and claims. In some embodiments, X¹, X², and X³ are each CH; X⁴ is C; and X⁵ is N.

The synthetic routes described in the general schemes are suitable for synthesizing compounds in which the hexahydropyridazine moiety:

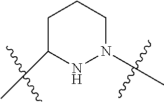 [SMILES: *C1CCCN(*)N1]

is replaced by:

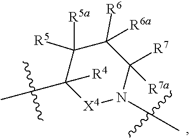

as detailed in the specification and claims.

### Pharmaceutical Compositions and Methods of Use

The compounds with which the invention is concerned are Ras inhibitors and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder, or condition or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating, or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive, or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, intradermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous, or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

In some embodiments, a compound described herein, or a compound of any of formulas I′ or I, or any of the subformulas described herein, or a compound recited in any of the claims, or a pharmaceutically acceptable salt, solvate, tautomer or isomer thereof, can promote, enhance, or increase GTP hydrolysis in a RAS mutant protein. Methods of measuring such hydrolysis are known in the art: see, e.g., WO 2024206858, incorporated herein by reference in its entirety.

### Methods of Use

In some embodiments, the invention discloses a method of treating a disease or disorder that is characterized by aberrant Ras activity due to a Ras mutant. In some embodiments, the disease or disorder is a cancer.

Accordingly, also provided is a method of treating cancer in a subject in need thereof, the method comprising administering to the subject a therapeutically effective amount of a compound of the present invention, or a pharmaceutically acceptable salt thereof, or a pharmaceutical composition comprising such a compound or salt. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a Ras protein-related disorder in a subject in need thereof, the method comprising administering to the subject a therapeutically effective amount of a compound of the present invention, or a pharmaceutically acceptable salt thereof, or a pharmaceutical composition comprising such a compound or salt.

In some embodiments, the compounds of the present invention or pharmaceutically acceptable salts thereof, pharmaceutical compositions comprising such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate, and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma, and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms), multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the Ras protein is wild type (Ras^{WT}). Accordingly, in some embodiments, a compound of the present invention is employed in a method of treating a patient having a cancer comprising a Ras^{WT} (e.g., K-Ras^{WT}, H-Ras^{WT} or N-Ras^{WT}). In some embodiments, the Ras protein is Ras amplification (e.g., K-Ras^{amp}). Accordingly, in some embodiments, a compound of the present invention is employed in a method of treating a patient having a cancer comprising a Ras^{amp} (K-Ras^{amp}, H-Ras^{amp} or N-Ras^{amp}). In some embodiments, the cancer comprises a Ras mutation, such as a Ras mutation described herein. In some embodiments, a mutation is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T, and combinations thereof;
   or a combination of any of the foregoing. In some embodiments, the cancer comprises a K-RasG12V mutation and the cancer is colorectal cancer. In some embodiments, the cancer comprises a K-RasG12V mutation and the cancer is pancreatic cancer. In some embodiments, the cancer comprises a K-RasG12V mutation and the cancer is non-small cell lung cancer.

Methods of detecting Ras mutations are known in the art. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNACIamp; RealQuality; EntroGen; LightMix; StripAssay; Hybcell plexA; Devyser; Surveyor; Cobas; and TheraScreen Pyro. See, also, e.g., WO 2020/106640.

Also provided is a method of inhibiting a Ras protein in a cell, the method comprising contacting the cell with an effective amount of a compound of the present invention, or a pharmaceutically acceptable salt thereof. A method of inhibiting RAF-Ras binding, the method comprising contacting the cell with an effective amount of a compound of the present invention, or a pharmaceutically acceptable salt thereof, is also provided. The cell may be a cancer cell. The cancer cell may be of any type of cancer described herein. The cell may be in vivo or in vitro.

### Combination Therapy

The methods of the invention may include a compound of the present invention used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In certain embodiments, compositions of the disclosure comprise a compound of the present invention and one additional therapeutic agent. In certain embodiments, compositions of the disclosure comprise a compound of the present invention and two additional therapeutic agents. In certain embodiments, compositions of the disclosure comprise a compound of the present invention and three additional therapeutic agents. In certain embodiments, compositions of the disclosure comprise a compound of the present invention and four or more additional therapeutic agents.

Also provided are pharmaceutical compositions including the combinations, or a pharmaceutically acceptable salt thereof, and a pharmaceutically acceptable excipient. Compositions comprising a combination of therapeutic agents may be used in methods of modulating RAS (e.g., in a subject or in a cell) and in methods of treating RAS related diseases and disorders (e.g., cancer), as described herein. The present disclosure provides, inter alia, compositions, methods, and kits for treating or preventing a RAS related disease or disorder.

Exemplary agents that may be used in combination with a compound of the present invention are described below. All references herein are incorporated by reference for the agents described, including compound or molecular structures disclosed therein, whether explicitly stated as such or not.

### a) RAS(ON) Inhibitors

Compositions and methods of the present disclosure may include a compound of the present invention plus a RAS(ON) inhibitor. In some embodiments, the RAS(ON) inhibitor is RMC-6236, RMC-7977, RMC-6291, RMC-4998, RMC-9805, RMC-044, RMC-5127, GFH547, ERAS-0015 or compound 6A of WO 2024/067857. Exemplary RAS(ON) inhibitors useful in combinations according to the present disclosure can be found in any one of the following patent applications: WO 2025201453, WO 2025162395, WO 2025119392, WO2025087431, WO 2025051241, WO 2025045233, WO 2024249299, WO 2024222864, WO 2024206858, WO 2024169914, WO 202453208, WO 2024206858, WO 2024149819, WO 2024104364, WO 2024067857, WO 2024060966, WO 2024017859, WO 2024102421, WO 2024008834, WO 2024008610, WO 2023232776, WO 2023240263, WO 2023086341, WO 2023208005, WO 2023133543, WO 2023060253, WO 2023025832, WO 2023015559, WO 2023060253, WO 2022235870, WO 2022235864, WO 2022060836, WO 2021091982, WO 2021091967, WO 2021091956, WO 2020132597, CN 117903169, CN 117720556, CN 117720555, CN 117720554, CN 117534687, CN 117534685, CN 117534684, PCT/US2024/030993, PCT/US2024/023272, and PCT/US2024/023208, each of which is incorporated herein by reference in its entirety, including the compound structures disclosed therein.

In some embodiments, the RAS(ON) inhibitor is a RAS(ON) multi-selective inhibitor (e.g., daraxonrasib (RMC-6236), AN9025, BPI-572270, compound 6A of WO 2024/067857, ERAS-0015, GFH276, GFH547, HJ-099, RMC-7977, RCZY-680, RCZY-690, RG6505, AUBE00.

In some embodiments, the RAS(ON) inhibitor is the multi-selective inhibitor RMC-6236

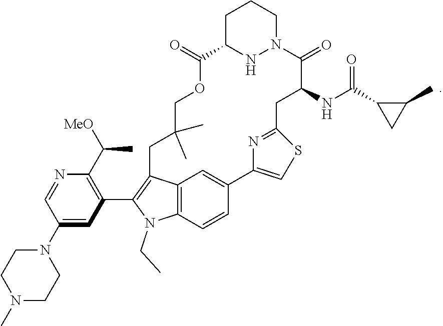

In some embodiments, a RAS(ON) multi-selective inhibitor is compound 6A of WO 2024/067857

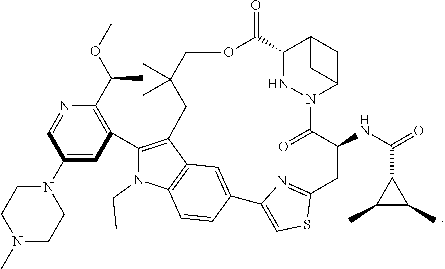

The RAS(ON) multi-selective compounds useful according to the present disclosure exhibit inhibitory activities across a variety of RAS mutants. In some embodiments, a RAS(ON) multi-selective compound inhibits wild type RAS. In some embodiments, a RAS(ON) multi-selective compound inhibits wild type KRAS. In some embodiments, a RAS(ON) multi-selective compound inhibits a RAS mutant with one or more mutations at G12X, G13X, and/or Q61X, wherein X represents any naturally occurring amino acid residue. In certain instances, X is A, C, D, V, S, R, H, K, or L amino acid residue.

In certain embodiments, a RAS(ON) multi-selective compound inhibits a RAS mutant with one or more mutations at G12X, wherein X represents any naturally occurring amino acid residue. In certain instances, X is A, C, D, V, S or R amino acid residue.

In other embodiments, a RAS(ON) multi-selective compound inhibits a RAS mutant with one or more mutations at G13X, wherein X is any naturally occurring amino acid residue. In certain instances, X is A, C, D, V, S or R amino acid residue.

In other embodiments, a RAS(ON) multi-selective compound inhibits a RAS mutant with one or more mutations at Q61X, wherein X is any naturally occurring amino acid residue. In certain instances, X is A, C, D, V, S, R, H, K, or L amino acid residue. In other instances, X is H, K, R, or L amino acid residue.

A variety of RAS proteins may be inhibited by a RAS(ON) multi-selective compound (e.g., KRAS, NRAS, HRAS, and mutants thereof at positions 12, 13 and 61, such as G12A, G12C, G12D, G12V, G12S, G12R, G13C, G13D, Q61H, Q61K, Q61R and Q61L, and others described herein, or a combination thereof). In some embodiments, a RAS(ON) multi-selective compound inhibits a G12A, G12C, G12D, G12R, G12S, G12V, or Q61H mutant of RAS, or a combination thereof.

Compositions and methods described herein may include one or more RAS(ON) mutant-selective inhibitors. Numerous RAS(ON) mutant-selective inhibitors have been disclosed.

Some embodiments of combinations comprising a compound of the present invention include a composition comprising a RAS(ON) mutant-selective inhibitor. In some embodiments, the RAS(ON) mutant-selective inhibitor is a RAS(ON) G12C-selective inhibitor. In some embodiments, the RAS(ON) mutant-selective inhibitor is a RAS(ON) G12D-selective inhibitor. In some embodiments, the RAS(ON) mutant-selective inhibitor is a RAS(ON) G13C-selective inhibitor. In some embodiments, the RAS(ON) mutant-selective inhibitor is a RAS(ON) Q61H-selective inhibitor. In some embodiments, the RAS(ON) mutant-selective inhibitor is a RAS(ON) G13D-selective inhibitor. In some embodiments, the RAS(ON) mutant-selective inhibitor is a RAS(ON) G12R-selective inhibitor.

RAS(ON) mutant-selective inhibitors useful in combinations according to the methods of the present disclosure can be found in any one of the following patent applications: WO 2025104149, WO 2025093625, WO 2025080946, WO 2024249299, WO 2024211663, WO 2024211712, WO 2024208934, WO 2024149819, WO 2024008610, WO 2024102421, WO 2023240263, WO 2023133543, WO 2023015559, WO 2023086341, WO 2023208005, WO 2023232776, WO 2023086341, WO 2023060253, WO 2023015559, WO 2022235870, WO 2022235864, WO 2021091967, WO 2021091982, WO 2021108683, WO 2020132597, each of which is incorporated herein by reference in its entirety, including the compound structures disclosed therein.

In some embodiments, the RAS(ON) mutant-selective inhibitor useful according to the present disclosure is zoldonrasib (RMC-9805) or RMC-9945, a G12D mutant-selective inhibitor

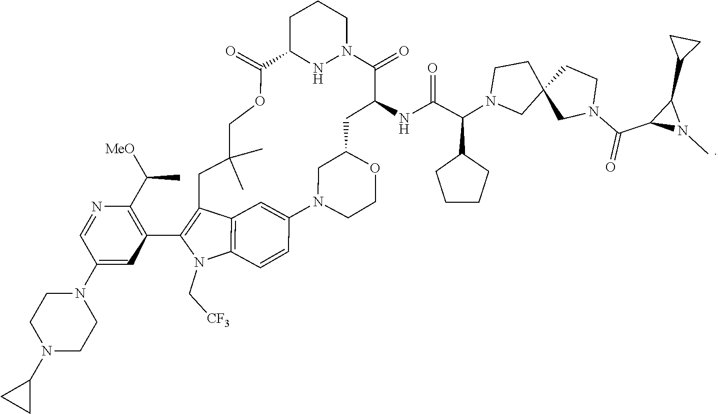

In some embodiments, the RAS(ON) mutant-selective inhibitor is the RAS(ON) G12C-selective tri-complex inhibitor, elironrasib (RMC-6291) or RMC-4998

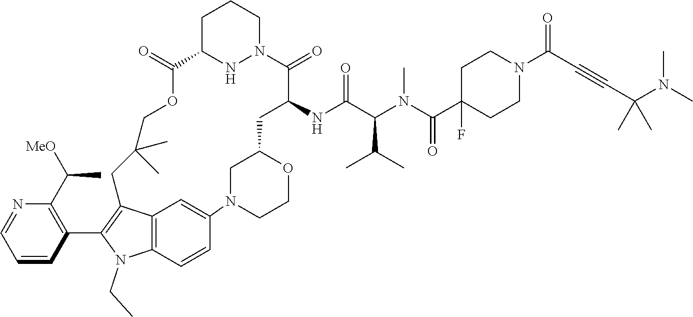

### (Schulze et. al., Science. 2023 Aug. 18; 381(6659): 794-799).

In some embodiments, the RAS(ON) mutant-selective inhibitor is a G12V-selective inhibitor, such as RMC-5127

In some embodiments, the RAS(ON) mutant-selective inhibitor is a G13C-selective inhibitor, such as RMC-8839. In some embodiments, the RAS(ON) mutant-selective inhibitor is a Q61H-selective inhibitor, such as RMC-0708. In some embodiments, the RAS(ON) mutant-selective inhibitor is a G12R-selective inhibitor, such as RMC-8264.

The RAS(ON) inhibitor compounds described herein may be made from commercially available starting materials or synthesized using known organic, inorganic, or enzymatic processes. By way of example, the RAS(ON) compounds can be synthesized using the methods described in WO 2022060836, WO 2021091956, or WO 2021091982, or any of the other RAS(ON) references cited herein, together with synthetic methods known in the art of synthetic organic chemistry, or variations thereon as appreciated by those skilled in the art.

A RAS(ON) inhibitor may be an antibody-drug conjugate, such as WO 2025051241 and WO 2024189481. See also doi.org/10.1021/acs.jmedchem.4c02929. RAS(OFF) inhibitors are also known, such as WO 2025171055.

In some embodiments, the combination therapy comprising a compound of the present invention may include one or more RAS(ON) inhibitors, for example, a compound of the present invention plus one or more RAS(ON) multi-selective inhibitors and/or one or more RAS(ON) mutant-selective inhibitors.

### b) RAS/MAPK Inhibitors

Compositions and methods described herein may include a compound of the present invention in combination with one or more RAS/MAPK pathway inhibitors. The RAS/MAPK pathway is a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions. In some embodiments, a therapeutic agent that may be combined with a RAS(ON) inhibitor is an inhibitor of the MAP kinase (MAPK) pathway (or “MAPK pathway inhibitor”). MAPK pathway inhibitors include, but are not limited to, one or more MAPK pathway inhibitors described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK pathway inhibitor may be PLX8394, LXH254, GDC-5573, or LY3009120. A MAPK pathway inhibitor may be a PI3Kα:RAS breaker, such as BBO-10203.

### i) RAS(OFF) Inhibitors and RAS(OFF) Degraders

Compositions and methods described herein may include a compound of the present invention in combination with one or more RAS(OFF) inhibitors. Numerous mutant-selective and pan-KRAS inhibitors have been disclosed and are known in the art. A RAS(OFF) inhibitor may be administered or formulated in combination with a RAS(ON) inhibitor described herein. RAS(OFF) inhibitors are designed to inhibit RAS activity by targeting different regions of the RAS protein in its inactive state (GDP bound state), preventing its activation and downstream signaling.

In some embodiments, a RAS(OFF) inhibitor is a KRAS(OFF) inhibitor that has a molecular weight of under 700 Da. The term “KRAS(OFF) inhibitor” refers to any RAS(OFF) inhibitor that binds to KRAS in its GDP-bound “OFF” position. In some embodiments, the KRAS(OFF) inhibitor is specific for a KRAS^{G12C} mutation. KRAS^{G12C}(OFF) inhibitors use a covalent binding group that allows them to selectively target the KRAS^{G12C} mutant protein, and many such inhibitors comprise a pyrimidine core. KRAS^{G12C}(OFF) inhibitors all target the same cysteine residue in the KRAS^{G12C} mutant protein, leading to a conformational change that locks the protein in an inactive state. KRAS^{G12C}(OFF) inhibitors include, but are not limited to, adagrasib (MRTX849), divarasib (RG6330/GDC-6036), fulzerasib (IBI351/GFH925), garsorasib (D-1553), glecirasib (JAB-21822), olomorasib (LY3537982), opnurasib (JDQ443), sotorasib (AMG 510), ARS-853, ARS-1620, BI 1823911, BPI-421286, D3S-001, GEC255, HBI-2438, HS-10370, JAB-21000, JAB-21822, JMKX001899, JNJ-74699157 (ARS-3248), MK-1084, HRS-7058 and YL-15293. In some embodiments, the KRAS(OFF) inhibitor is selected from AMG 510 and MRTX849. In some embodiments, the KRAS(OFF) inhibitor is sotorasib (AMG 510). In some embodiments, the KRAS(OFF) inhibitor is adagrasib (MRTX849). In some embodiments, the KRAS(OFF) inhibitor is divarasib (GDC-6036). In some embodiments, the KRAS(OFF) inhibitor is selected from BPI-421286, JNJ-74699157 (ARS-3248), LY3537982, MRTX1257, ARS853, ARS1620, or GDC-6036. A RAS(OFF) inhibitor may be an antibody-drug conjugate. See also C. W. Parry et al. J. Med. Chem. 2025, 68:9, 9129-9161 (doi.org/10.1021/acs.jmedchem.4c02929).

In some embodiments, a KRAS(OFF) inhibitor is specific for a KRAS^{G12D} mutation. Many KRAS^{G12D}(OFF) inhibitors have been developed using KRAS^{G12C}(OFF) inhibitors as a starting point, thus sharing the backbone of G12C inhibitors in combination with other chemical moieties such as piperazine-based compounds. Non-limiting examples of KRAS^{G12D}(OFF) inhibitors include ASP3082, BPI-501836, ERAS-4693, ERAS-5024, HBW-012-D, HBW-012-E, HRS-4642, JAB-22000, KD-8, TSN1611, LY3962673, MRTX282, MRTX1133, Q2a, SHR1127, TH-Z827, TH-Z835, TSN1611, VRTX153, DN022150, HJ-119, JR-6000, NKT-G12D, FWD-K02, JAB-BX600, EB-TM1, ABSK141, BPI-2491, HRS-6093, HRS-7172.

In some embodiments, the small molecule RAS(OFF) inhibitor is specific for a KRAS^{G12V} mutation, such as JAB-23000 or QTX3544. In some embodiments, the small molecule RAS(OFF) inhibitor is specific for a KRAS^{G13D} mutation. In some embodiments, the small molecule RAS(OFF) inhibitor is a pan-KRAS(OFF) inhibitor, such as A2A-03, ABREV01, ABT-200, ADT-030, ADT-1004, AN9025, BBP-454, BGB-53038, BI-2865, BI 3706674, ERAS-4, ERAS-254, ERAS-4001, HB-700 (G12X+G13D), JAB-23400, OC211, PF-07934040, QTX3034, RSC-1255, YL-17231 or ZG2001. In some embodiments, the Ras inhibitor is JAB-23400. In some embodiments, the Ras inhibitor is BI-2852. In some embodiments, the Ras inhibitor targets both the ON and OFF states of Ras, such as BBO-11818, JAB-23E73, JAB-23425, JAB-23E73, BBO-8520, FMC-376, GFH375 (VS-7375), INCB161734, QTX3046, TSN1611, or TH-Z835.

In some embodiments, reference to the term RAS(OFF) inhibitor includes any such RAS(OFF) inhibitor disclosed in any one of the following patent applications: WO 2025202022, WO 2025201480, WO 2025199170, WO 2025194340, WO 2025194134, WO 2025194057, WO 2025194054, WO 2025190342, WO 2025188668, WO 2025179058, WO 2025170938, WO 2025168072, WO 2025167948, WO 2025165972, WO 2025162091, WO 2025157289, WO 2025157260, WO 2025157246, WO 2025157162, WO 2025153038, WO 2025151738, WO 2025151594, WO 2025148979, WO 2025146194, WO 2025136346, WO 2025132549, WO 2025130912, WO 2025129002, WO 2025124415, WO 2025123318, WO 2025123007, WO 2025122619, WO 2025117828, WO 2025111586, WO 2025111582, WO 2025108443, WO 2025106905, WO 2025106901, WO 2025101776, WO 2025096984, WO 2025096957, WO 2025096738, WO 2025092986, WO 2025092798, WO 2025085748, WO 2025085580, WO 2025080653, WO 2025077770, WO 2025077663, WO 2025076523, WO 2025072649, WO 2025072457, WO 2025072451, WO 2025067459, WO 2025067453, WO 2025064848, WO 2025064542, WO 2025061125, WO 2025059366, WO 2025059040, WO 2025054530, WO 2025054347, WO 2025054270, WO 2025053850, WO 2025051242, WO 2025045141, WO 2025049641, WO 2025049619, WO 2025049402, WO 2025049274, WO 2025040767, WO 2025038936, WO 2025036475, WO 2025036470, WO 2025034883, WO 2025034849, WO 2025026903, WO 2025019688, WO 2025018418, WO 2025016899, WO 2025016432, WO 2025011443, WO 2025010415, WO 2025007000, WO 2025006967, WO 2025006962, WO 2025006720, WO 2025006704, WO 2025002430, WO 2025002302, WO 2024259169, WO 2024254404, WO 2024254334, WO 2024255795, WO 2024246099, WO 2024243025, WO 2024238633, WO 2024238343, WO 2024236452, WO 2024235286, WO 2024235225, WO 2024233776, WO 2024230734, WO 2024230707, WO 2024229447, WO 2024229444, WO 2024229442, WO 2024229317, WO 2024227091, WO 2024220645, WO 2024220532, WO 2024218686, WO 2024215862, WO 2024215754, WO 2024213979, WO 2024213122, WO 2024208305, WO 2024209339, WO 2024206766, WO 2024206747, WO 2024197503, WO 2024193698, WO 2024192424, WO 2024179546, WO 2024178313, WO 2024178304, WO 2024173842, WO 2024167922, WO 2024160225, WO 2024159471, WO 2024159470, WO 2024158778, WO 2024158242, WO 2024153119, WO 2024153116, WO 2024138486, WO 2024138206, WO 2024138052, WO 2024131829, WO 2024125642, WO 2024125600, WO 2024123913, WO 2024123102, WO 2024120433, WO 2024120419, WO 2024119277, WO 2024118926, WO 2024109233, WO 2024112654, WO 2024104453, WO 2024104425, WO 2024107686, WO 2024104453, WO 2024103010, WO 2024097559, WO 2024091409, WO 2024088273, WO 2024085661, WO 2024083258, WO 2024083256, WO 2024083246, WO 2024083168, WO 2024078555, WO 2024076674, WO 2024076672, WO 2024076670, WO 2024067714, WO 2024067575, WO 2024064335, WO 2024063578, WO 2024063576, WO 2024061370, WO 2024061333, WO 2024061267, WO 2024056063, WO 2024055112, WO 2024054926, WO 2024054647, WO 2024054625, WO 2024051763, WO 2024051721, WO 2024050742, WO 2024050640, WO 2024046406, WO 2024046370, WO 2024045066, WO 2024044667, WO 2024044649, WO 2024044334, WO 2024041621, WO 2024041606, WO 2024041589, WO 2024041573, WO 2024040131, WO 2024040109, WO 2024040080, WO 2024036270, WO 2024034657, WO 2024034593, WO 2024034591, WO 2024034123, WO 2024032747, WO 2024032704, WO 2024032703, WO 2024032702, WO 2024031088, WO 2024030647, WO 2024030633, WO 2024029613, WO 2024022507, WO 2024022444, WO 2024020159, WO 2024019103, WO 2024017859, WO 2024017392, WO 2024015731, WO 2024015262, WO 2024012456, WO 2024009191, WO 2024008179, WO 2024008178, WO 2024008068, WO 2024006445, WO 2024006424, WO 2024002373, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023246914, WO 2023246903, WO 2023246777, WO 2023244713, WO 2023244615, WO 2023244604, WO 2023244600, WO 2023244599, WO 2023230190, WO 2023226630, WO 2023225302, WO 2023225252, WO 2023220421, WO 2023219941, WO 2023217148, WO 2023215802, WO 2023215801, WO 2023213269, WO 2023212548, WO 2023208005, WO 2023205719, WO 2023199180, WO 2023198191, WO 2023197984, WO 2023190748, WO 2023185864, WO 2023183755, WO 2023183585, WO 2023179703, WO 2023179629, WO 2023173017, WO 2023173016, WO 2023173014, WO 2023172737, WO 2023171781, WO 2023159087, WO 2023159086, WO 2023154766, WO 2023152255, WO 2023151674, WO 2023151621, WO 2023150394, WO 2023150284, WO 2023143623, WO 2023143605, WO 2023143352, WO 2023143352, WO 2023143312, WO 2023141570, WO 2023141300, WO 2023138662, WO 2023138601, WO 2023138589, WO 2023138524, WO 2023133183, WO 2023133181, WO 2023130012, WO 2023125989, WO 2023125627, WO 2023122662, WO 2023122154, WO 2023120742, WO 2023119677, WO 2023117681, WO 2023116934, WO 2023116895, WO 2023114733, WO 2023105491, WO 2023104018, WO 2023103906, WO 2023103523, WO 2023101928, WO 2023099624, WO 2023099624, WO 2023099620, WO 2023099612, WO 2023099608, WO 2023099592, WO 2023098832, WO 2023098425, WO 2023097227, WO 2023081840, WO 2023081476, WO 2023078424, WO 2023077441, WO 2023072297, WO 2023072188, WO 2023066371, WO 2023064857, WO 2023061463, WO 2023061294, WO 2023057985, WO 2023056951, WO 2023056421, WO 2023051586, WO 2023049697, WO 2023046135, WO 2023045960, WO 2023041059, WO 2023041059, WO 2023040989, WO 2023040513, WO 2023039240, WO 2023039020, WO 2023036282, WO 2023034290, WO 2023030517, WO 2023030495, WO 2023030385, WO 2023030495, WO 2023030517, WO 2023030685, WO 2023030687, WO 2023034290, WO 2023036282, WO 2023039240, WO 203020347, WO 2023025116, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023040989, WO 2023039240, WO 2023039020, WO 2023036282, WO 2023034290, WO 2023030517, WO 2023030495, WO 2023030385, WO 2023025116, WO 2023020523, WO 2023020521, WO 2023020519, WO 2023020518, WO 2023020347, WO 2023018812, WO 2023018810, WO 2023018809, WO 2023018699, WO 2023014979, WO 2023014006, WO 2023004102, WO 2023003417, WO 2023001141, WO 2023001123, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762 (e.g., Compound 44 or Compound 66a), WO 2022212894, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021113595, WO 2021107160, WO 2021106231, WO 2021088458, WO 2021086833, WO 2021085653, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, KR 20240101190, KR 20240101189, KR 20240041720, KR 20240041719, CN 118666870, CN 118666869, CN 118580238, CN 118307563, CN 118221700, CN 118221699, CN 118221698, CN 118221685, CN 118126064, CN 118078802, CN 118078801, CN 118005656, CN 117986263, CN 117986263, CN 117946135, CN 117924327, CN 117903117, CN 117800990, CN 117800989, CN 117800976, CN 117736226, CN 117683051, CN 117645627, CN 117624194, CN 117624190, CN 117586280, CN 117486901, CN 117466917, CN 117462688, CN 117362315, CN 117327102, CN 117327094, CN 117327074, CN 117285590, CN 117263959, CN 117247382, CN 117186095, CN 117164605, CN 116969977, CN 116925075, CN 116891489, CN 116731045, CN 116731044, CN 116554208, CN 116514846, CN 116478184, CN 116478141, CN 116410145, CN 116375742, CN 116354988, CN 116332948, CN 116332938, CN 116327956, CN 116262759, CN 116217592, CN 116199703, CN 116162099, CN 116143806, CN 116143805, CN 116120315, CN 116102559, CN 115960105, CN 115894520, CN 115872979, CN 115850267, CN 115785199, CN 115785124, CN 115724842, CN 115724842, CN 115721720, CN 115716840, CN 115703775, CN 115611923, CN 115611898, CN 115583937, CN 115572278, CN 115557949, CN 115521312, CN 115504976, CN 115490709, CN 115466272, CN 115433183, CN 115433179, CN 115403575, CN 115385938, CN 115385937, CN 115385912, CN 115381786, CN 115368383, CN 115368382, CN 115368381, CN 115353506, CN 115322158, CN 115304623, CN 115304602, CN 115197245, CN 115181106, CN 114989195, CN 114989166, CN 114989147, CN 114920741, CN 114920739, CN 114907387, CN 114874234, CN 114874201, CN 114716436, CN 114716435, CN 114685532, CN 114685460, CN 114591319, CN 114539293, CN 114539286, CN 114539246, CN 114437107, CN 114437084, CN 114409653, CN 114380827, CN 114195804, CN 114195788, CN 114437107, CN 114409653, CN 114380827, CN 114195804, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113321654, CN 113248521, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, CN 109574871, US2024270736, or EP 4389751, each of which is incorporated herein by reference in its entirety, including the RAS compound structures disclosed therein which are specifically incorporated herein by reference.

In some embodiments, reference to the term RAS(OFF) inhibitor refers to a pan-KRAS inhibitor, such as selected from one disclosed in any of the following: WO 2024206766, WO 2024206747, WO 2024192424, WO 2024178313, WO 2024178304, WO 2024173842, WO2024153180, WO 2024119277, WO 2024120433, WO 2024115890, WO 2024112654, WO 2024104453, WO 2024104425, WO 2024107686, WO 2024104453, WO 2024103010, WO 2024085661, WO 2024083246, WO 2024083168, WO 2024067575, WO 2024064335, WO 2024063578, WO 2024063576, WO 2024051852, WO 2024051763, WO 2024046370, WO 2024044667, WO 2024041621, WO 2024041606, WO 2024041589, WO 2024040131, WO 2024040109, WO 2024032747, WO 2024032704, WO 2024032703, WO 2024032702, WO 2024031088, WO 2024030647, WO 2024030633, WO 2024015262, WO 2024009191, WO 2024008068, WO 2024002373, WO 2023287896, WO 2023274324, WO 2023246914, WO 2023246777, WO 2023230190, WO 2023215802, WO 2023215801, WO 2023197984, WO 2023190748, WO 2023183585, WO 2023179703, WO 2023173017, WO 2023173016, WO 2023173014, WO 2023172737, WO 2023154766, WO 2023143352, WO 2023143312, WO 2023138589, WO 2023133183, WO 2023122662, WO 2023114733, WO 2023099624, WO 2023099623, WO 2023099612, WO 2023099608, WO 2023099592, WO 2023097227, WO 2023064857, WO 2023056421, WO 2023049697, WO 2023046135, WO 2023039240, WO 2023034290, WO 2023020523, WO 2023020521, WO 2023020519, WO 2023020518, WO 2023001123, WO 2022271823, WO 2022261210, WO 2022258974, WO 2022256459, WO 2022250170, WO 2022248885, WO 2022228543, WO 2022216762, WO 2022072783, WO 2016161361, KR 20240101190, KR 20240101189, KR 20240041720, KR 20240041719, CN 118221700, CN 118126064, CN 117924327, CN 117946135, CN 117800990, CN 117800989, CN 117683051, CN 117486901, CN 117263959, CN 116969977, or CN 116332948, each of which is incorporated herein by reference in its entirety, including the compound structures disclosed therein. In some embodiments, the combination therapy comprising a compound of the present invention may include one or more additional RAS inhibitors, for example, a pan-KRAS inhibitor. In some embodiments, combination comprising a pan-KRAS inhibitor therapy comprises ERAS-4001. In some embodiments, the pan-KRAS inhibitor, is a pan-KRAS inhibitor in a patent application filed in the name of Medshine Discovery, Inc. In some embodiments, combination comprising a pan-KRAS inhibitor therapy includes BGB-53038, BBO-11818, YL-17231, QTX3034, ABT-200, ADT-1004, AN9025, OC211, JAB-23425, BI-2865, BI-2493, ABREV01, A2A-03, or PF-07934040.

In some embodiments, a combination comprising a pan-KRAS inhibitor therapy includes A2A-03, ABREV01, ADT-007, ABT-200, ADT-030, ADT-1004, BBP-454, BGB-53038, BI-2865, BI-2493, BI 3706674, BRSD-143, ERAS-4, ERAS-254, ERAS-4001, HB-700 (G12X+G13D), HZ-V068, ID12241161, JAB-23400, LY4066434, OC211, PF-07985045, PF-07934040, PF-4040, QTX2024, QTX3034, RSC-1255, SIL204, SYNB021225, YL-17231, ZG2001, WEF-001.

In some embodiments, a RAS inhibitor binds to the OFF form as well as the ON form. Non-limiting examples of such inhibitors include, e.g., pan-KRAS: ALTA3263, AMG 410, BBO-11818, HBW-012462, HBW-016-K, HEC211909, JAB-23E73, JAB-23425, JAB-23E73; or a compound disclosed in Zheng, Q.; Shen, T.; Pampel, J.; Shokat, K. M.; Distal Covalent Targeting Suppresses Signaling of Oncogenic KRas-(G13C) in Cancer Cells. ACS Chem. Biol. 2025, 20, 7, 1696-1706; G12C: BBO-8520, FMC-376; G12D: AZD0022, GFH375 (VS-7375), INCB161734, QTX3046, TSN1611, TH-Z835, TLN-372.

In some embodiments, a RAS inhibitor binds to the ON form of RAS but is not a tri-complex inhibitor, such as pan-KRAS inhibitors JTX-102 and JTX-105. Pan-KRAS(ON) inhibitors with a high selectivity for the ON form may be found, for example, in WO 2025193878.

In any embodiment employing a RAS(OFF) inhibitor herein, a RAS(OFF) degrader targeting the OFF state of RAS may be employed. These degraders are known in the art. RAS degraders may be found, for example, in one or more of the following applications: WO 2025190158, WO 2025169901, WO 2025168124, WO 2025168051, WO 2025162250, WO 2025159142, WO 2025151765, WO 2025125630, WO 2025108479, WO 2025107579, WO 2025103476, WO 2025096855, WO 2025085815, WO 2025083472, WO 2025078984, WO 2025076044, WO 2025058008, WO 2025053850, WO 2025024732, WO 2025019823, WO 2025006783, WO 2025006753, WO 2024263586, WO 2024261257, WO 2024261256, WO 2024241248, WO 2024233838, WO 2024199266, WO 2024188281, WO 2024/159164, WO 2024/152247, WO 2024/149214, WO 2024131777, WO 2024120424, WO 2024119278, WO 2024118966, WO 2024118960, WO 2024083258, WO 2024083256, WO 2024055112, WO 2024054625, WO 2024050742, WO 2024044334, WO 2024040080, WO 2024034657, WO 2024034593, WO 2024034591, WO 2024034123, WO 2024029613, WO 2024020159, WO 2024019103, WO 2024017392, WO 2023215906, WO 2023185864, WO 2023171781, WO 2023141570, WO 2023138524, WO 2023130012, WO 2023116934, WO 2023099620, WO 2023081476, WO 2023077441, WO 2022260482, CN 118725012, CN 118496502, CN 118496300, CN 118126040, CN 115785199 or US 2025213706, each of which is incorporated herein by reference in its entirety. Non-limiting examples of RAS degraders include: ASP3082 (KRAS G12D); ASP4396 (KRAS G12D); BPI-585725 (G12X and WT), LT-010366 (G12D); PT0253 (G12D), RD0255359 (KRAS G12C/DV); RP03707 (G12D), JR-9000, 356A, SH1718, IPS-06061, HDB-82. In some embodiments, the RAS(OFF) inhibitor is a peptide-based inhibitor. Peptide-based RAS(OFF) inhibitors have been developed that target specific regions of the RAS protein, such as the Switch II region or the RAS-effector interface. Non-limiting examples include the K-Ras-binding peptide (Krpep-2d), the Ras inhibitory peptide (RasIn) and LUNA18 (NCT05012618). Peptide-based RAS(OFF) inhibitors are a class of compounds that target the RAS protein by disrupting its interaction with its downstream effectors or other signaling proteins. These inhibitors are typically designed to mimic the binding motifs of RAS-interacting proteins or other RAS effectors, such as RAF or PI3K. By binding to RAS at the same site as these effectors, peptide-based inhibitors can effectively compete with these proteins and prevent the activation of downstream signaling pathways. See, e.g., WO 2025162428, WO 2025127968, WO 2025018418, WO 2024219480, WO 2024219446, WO 2024176153, WO 2024101402, WO 2024101386, WO 2023214576, WO 2023140329, WO 2022234853, WO 2022234852, WO 2022234851, and WO 2022234639, each of which is incorporated herein by reference in its entirety.

Peptide-based RAS(OFF) inhibitors can be further classified into two main categories: those that target the RAS-effector interface, and those that target other regions of the RAS protein. Peptide-based inhibitors that target the RAS-effector interface are designed to bind to the switch regions of RAS that are critical for its interaction with downstream effectors, such as RAF or PI3K. These inhibitors typically contain amino acid residues that are similar to those found in the binding motifs of RAS-interacting proteins or effectors and are often designed to form hydrogen bonds or other interactions with key residues on the surface of RAS.

Peptide-based RAS(OFF) inhibitors that target other regions of the RAS protein are typically designed to disrupt other interactions that are critical for the activation or signaling of RAS. For example, some peptide-based inhibitors are designed to bind to the hypervariable region of RAS, which is thought to play a role in membrane localization and anchoring of the protein. By binding to this region, peptide-based inhibitors can prevent the proper localization of RAS to the plasma membrane, which is necessary for its activation and signaling.

Several common motifs have been identified as important for the binding of RAS-interacting proteins and effectors and are often used in the design of peptide-based inhibitors. One example is the RAF-binding domain (RBD), which is found in many RAS-interacting proteins and is important for the interaction of RAS with downstream effectors such as RAF. The RBD contains a conserved amino acid sequence (Arg-Xaa-Arg) that is critical for binding to RAS, and this motif has been incorporated into several peptide-based inhibitors designed to disrupt the RAS-RAF interaction. Another example is the RAS-binding domain (RBD) of PI3K, which is important for the interaction of RAS with this downstream effector. The RBD of PI3K contains several conserved amino acid residues (such as Arg-Arg-Trp) that are critical for binding to RAS, and these motifs have been used in the design of peptide-based inhibitors that target the RAS-PI3K interaction. Other common motifs used in peptide-based RAS(OFF) inhibitors include the Ras-binding domain (RBD) of other RAS-interacting proteins such as RaIGDS and SOS, as well as sequences that mimic the structure of the switch regions of RAS itself. These motifs are typically used to optimize the binding affinity and selectivity of the inhibitor for the desired target protein or interaction.

In some embodiments, the RAS(OFF) inhibitor is an antibody or antigenic binding peptide specific for RAS(OFF). Antibodies have been developed that bind to specific regions of the RAS protein, such as the Switch II region or the RAS-effector interface. For example, some antibodies have been developed that target the switch regions of RAS proteins, which are critical for the activation of these proteins and their interaction with downstream effectors. Binding of these antibodies to the switch regions can prevent the conformational changes required for RAS activation and downstream signaling. Another approach involves the use of antibodies that target RAS-interacting proteins or downstream effectors, such as RAF or PI3K. Binding of these antibodies to their target proteins can disrupt the RAS-dependent signaling pathways and inhibit the growth and survival of cancer cells. Additionally, some antibodies have been developed that can induce the internalization and degradation of RAS proteins, leading to their depletion and inhibition of downstream signaling. For example, some antibodies have been developed that recognize the unique structure of mutant RAS proteins and target them for degradation via the ubiquitin-proteasome pathway. Non-limiting examples of KRAS(OFF)-specific inhibitory antibodies include anti-p21ser, and K27 (DARPin) (see, e.g., Khan et al, Biochim Biophys Acta Mol Cell Res. 2020 February; 1867(2):118570). See also WO 2024136608 and WO 2024111590, each of which is incorporated herein by reference in its entirety.

Antibody-drug conjugates may also be constructed using RAS inhibitors, such as WO 2024189481, which is incorporated herein by reference in its entirety, including the compound structures disclosed therein.

Vaccines may also be used in combination with compounds of the present invention. Non-limiting examples include: AFNT-111 (KRAS G12V), AFNT-211 (KRAS G12V), AFNT-212 (KRAS G12D), ELI-002 (KRAS G12/13X), HB-700, NT-112 (KRAS G12D), and TG01 (pan-KRAS).

ii) Other RAS Modalities Useful in Combination with Compounds of the Present Invention Include: ADGN-123, ADGN-121 (Gene Editing Peptide-RNA Nanoparticles G12D); ADT-030 (Ras/B-Catenin Inhibitor); BBO-10203 (PI3Kα:RAS Breaker); BI 1701963 (Pan-KRAS:SOS1); mRNA-5671 (Nucleic Acid) and RO7673396 (RAS Inhibitor), AZD0240 (TCR-T Cell Product Targeting G12D), MDG2021 (TCR-T Cell Product Targeting G12D), ADGN-121 (Peptide-sgRNA Nanoparticles), BION-302 (Antibody Based) LIB111 (Antibody Based), SIL-204 (ASO/siRNA-Based). SOS1 Inhibitors

In some embodiments, compositions and methods described herein may include a compound of the present invention in combination with one or more SOS1 inhibitors. A SOS1 inhibitor may be administered or formulated in combination with compound of the present invention and/or any additional therapeutic agent described herein. In some embodiments, a SOS1 inhibitor is one or more of RMC-5845, RMC-4948, RMC-0331, BI-1701963, BI-3406, SDR5, MRTX-0902, ZG2001, and BAY-293. In some embodiments, reference to the term SOS1 inhibitor includes any such SOS1 inhibitor disclosed in any one of the following patent applications: WO 2023109929, WO 2023059597, WO 2023029833, WO 2023041049, WO 2023022497, WO 2022157629, WO 2022184116, WO 2022170952, WO 2022170917, WO 2022171184, WO 2022170802, WO 2022161461, WO 2022121813, WO 2022028506, WO 2022139304, WO 2021228028, WO 2019122129, CN 115215847, CN 115028644, CN 114685488, CN 111393519, CN 115677702, and CN 115806560 each of which is incorporated herein by reference in its entirety, including the compound structures disclosed therein which are specifically incorporated herein by reference.

iii) SHP Inhibitors

In some embodiments, compositions and methods described herein may include a compound of the present invention in combination with one or more SHP inhibitors. A SHP inhibitor may be administered or formulated in combination with a compound of the present invention and/or any additional therapeutic agent described herein. In some embodiments, the SHP inhibitor is an inhibitor of SHP1. In some embodiments, the SHP inhibitor is an inhibitor of SHP2. In some embodiments, the SHP1 inhibitor is SB8091 or SB6299 aka DA-4511. In some embodiments, a SHP2 inhibitor is one or more of SHP099, TNO155, RMC-4550, RMC-4630, JAB-3068, JAB-3312, RLY-1971, ERAS-601, SH3809, PF-07284892, or BBP-398. In some embodiments, reference to the term SHP2 inhibitor includes any such SHP2 inhibitor disclosed in any one of the following patent applications: WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2022063190, WO 2022043865, WO 2022042331, WO 2022033430, WO 2022017444, WO 2022007869, WO 2021259077, WO 2021249449, WO 2021249057, WO 2021244659, WO 2021218755, WO 2021176072, WO 2021171261, WO 2021149817, WO 2021148010, WO 2021147879, WO 2021143823, WO 2021143701, WO 2021143680, WO 2021281752, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Ser. No. 11/179,397, U.S. Ser. No. 11/044,675, U.S. Ser. No. 11/034,705, U.S. Ser. No. 11/033,547, U.S. Ser. No. 11/001,561, U.S. Ser. No. 10/988,466, U.S. Ser. No. 10/954,243, U.S. Ser. No. 10/934,302, or U.S. Ser. No. 10/858,359, each of which is incorporated herein by reference in its entirety, including the compound structures disclosed therein which are specifically incorporated herein by reference.

### iv) MEK Inhibitors

In some embodiments, compositions and methods described herein may include a compound of the present invention in combination with one or more MEK inhibitors. A MEK inhibitor may be administered or formulated in combination with a compound of the present invention and/or any additional therapeutic agent described herein. In some embodiments, a MEK inhibitor is one or more of pimasertib, IMM-1-104, selumetinib, cobimetinib (COTELLIC®), trametinib (MEKINIST®), atebimetinib, and binimetinib (MEKTOVI®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N. In some embodiments, reference to the term MEK inhibitor includes any such MEK inhibitor disclosed in any one of the following patent applications: WO 2022221866, WO 2022125941, WO 2022208391, WO 2022015736, WO 2022177557, WO 2021018866, WO 2021069486, WO 2021142144, WO 2021168283, WO 2021234097, WO 2019076947, WO 2018233696, WO 2016188472, WO 2014063024, WO 2013019906, WO 2011047238, WO 2007044515, US 2023032403, and CN 115813930, each of which is incorporated herein by reference in its entirety, including the compound structures disclosed therein which are specifically incorporated herein by reference.

### v) RAF Inhibitors

In some embodiments, compositions and methods described herein may include a compound of the present invention in combination with one or more RAF inhibitors. A RAF inhibitor may be administered or formulated in combination with a compound of the present invention and/or any additional therapeutic agent described herein. In some embodiments, a RAF inhibitor is VS-6766 or BTDX-4933. In some embodiments, a RAF inhibitor is a BRAF inhibitor. BRAF inhibitors that may be used in combination with a compound of the present invention include, for example, Vs6766, IK-595, vemurafenib, dabrafenib, and encorafenib. BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N5811; D594N; D594G; D594A; D594H; F595L; G596D; G596R and A762E. In some embodiments, reference to the term RAF inhibitor includes any such RAF inhibitor disclosed in any one of the following patent applications: WO 2023076991, WO 2022226626, WO 2022226261, WO 2019084459, WO 2018203219, WO 201851306, WO 2017212442, WO 2015075483, WO 2013134243, WO 2013134298, WO 2011047238, WO 2011025965, WO 2011025947, WO 2011025951, WO 2011025940, WO 2011025938, WO 2010065893, WO 2009016460, WO 2009130015, WO 2009111278, WO 2009111279, WO 2008028141, and WO 2006024834, each of which is incorporated herein by reference in its entirety, including the compound structures disclosed therein which are specifically incorporated herein by reference.

### vi) ERK Inhibitors

In some embodiments, compositions and methods described herein may include a compound of the present invention in combination with one or more ERK inhibitors. An ERK inhibitor may be administered or formulated in combination with a compound of the present invention and/or any additional therapeutic agent described herein. In some embodiments, an ERK inhibitor is an ERK1/2 inhibitor, such as ERAS-007. In some embodiments, an ERK inhibitor is an ERK 5 inhibitor. In some embodiments, an ERK inhibitor is one or more of ASTX-029 or 1-75. In some embodiments, reference to the term ERK inhibitor includes any such ERK inhibitor disclosed in any one of the following patent applications: WO 2023076305, WO 2022259222, WO 2022221547, WO 2021110169, WO 2021110168, WO 2021252316, WO 2020102686, WO 2020228817, WO 2020107987, WO 2019233456, WO 2019233457, WO 2016025561, WO 2016192063, WO 2016106029, WO 2016106009, WO 2015051341, WO 2014124230, WO 2014052563, WO 2011041152, WO 200910550, WO 2008153858, CN 114315837, CN 115057860, CN 107973783, each of which is incorporated herein by reference in its entirety, including the compound structures disclosed therein which are specifically incorporated herein by reference.

vii) MAPK Inhibitors

In some embodiments, compositions and methods described herein may include a compound of the present invention in combination with one or more Mitogen-Activated Protein Kinase (MAPK) inhibitors. A MAPK inhibitor may be administered or formulated in combination with a compound of the present invention and/or any additional therapeutic agent described herein. In some embodiments, a MAPK inhibitor is a p38MAPK inhibitor or a MAP3K8 inhibitor. In some embodiments, the MAPK inhibitor is one or more of Tilpisertib (GS-4875) and neflamapidmod (VX-745). In some embodiments, reference to the term MAPK inhibitor includes any such MAPK inhibitor disclosed in any one of the following patent applications: WO 2016029263, CN 114767674, CN 115850179, and CN 1743006, each of which is incorporated herein by reference in its entirety, including the compound structures disclosed therein which are specifically incorporated herein by reference.

In some embodiments, a therapeutic agent that may be combined with a compound of the present invention is an inhibitor of MAP2K4. A non-limiting example of a MAP2K4 inhibitor useful according to the disclosure is HRX-0233.

### C) Kinase Inhibitors

Compositions and methods described herein may include a compound of the present invention in combination with one or more kinase inhibitors. Tyrosine kinases and serine/threonine kinases play a crucial role in various cellular processes such as cell signaling, growth, and differentiation. Kinase inhibitors known in the art have been developed as a treatment for various types of cancer in addition to therapies for conditions such as neurodegenerative diseases, autoimmune disorders, and inflammation.

### i) PKA Inhibitors

In some embodiments, compositions and methods described herein may include one or more Protein Kinase A (PKA) inhibitors. A PKA inhibitor may be administered or formulated in combination with a compound of the present invention and/or any additional therapeutic agent described herein. In some embodiments, a PKA inhibitor is H89. In some embodiments, reference to the term PKA inhibitor includes any such PKA inhibitor disclosed in any one of the following patent applications: CN 106620678 and CN 114632155, each of which is incorporated herein by reference in its entirety, including the compound structures disclosed therein which are specifically incorporated herein by reference.

### ii) FAK Inhibitors

In some embodiments, compositions and methods described herein may include a compound of the present invention in combination with one or more Focal Adhesion Kinase (FAK) inhibitors. A FAK inhibitor may be administered or formulated in combination with a compound of the present invention and/or any additional therapeutic agent described herein. In some embodiments, a FAK inhibitor is one or more of BI853520, defactinib, GSK2256098, PF-00562271, and VS-4718. In some embodiments, reference to the term FAK inhibitor includes any such FAK inhibitor disclosed in any one of the following patent applications: WO 2022152315, WO 2021098679, WO 2020135442, WO 2020191448, WO 2012022408, WO 2013134353, WO 2012110774, WO 2010062578, CN 111072571, and KR 101691536, each of which is incorporated herein by reference in its entirety, including the compound structures disclosed therein which are specifically incorporated herein by reference.

iii) ROCK Inhibitors

In some embodiments, compositions and methods described herein may include a compound of the present invention in combination with one or more Rho-associated, coiled-coil containing protein kinase (ROCK) inhibitors. A ROCK inhibitor may be administered or formulated in combination with a compound of the present invention and/or any additional therapeutic agent described herein. In some embodiments, a ROCK inhibitor is GSK269962A. In some embodiments, reference to the term ROCK inhibitor includes any such ROCK inhibitor disclosed in any one of the following patent applications: WO 2023051753, WO 2022237892, WO 2022012409, WO 2021093795, WO 2021214200, WO 2020177292, WO 202011751, WO 2019014304, WO 2019179525, WO 2019089868, WO 2019014300, WO 2018108156, WO 2018009627, WO 2018009625, WO 2018009622, WO 2017123860, WO 2017205709, WO 2016112236, WO 2014068035, WO 2013030367, WO 2012146724, WO 2012067965, WO 2011107608, CN 108129453, CN 108191821, CN 110917352, CN 108558823, CN 108047193, CN 107973777, CN 108047197, CN 108129448, CN 115869304, and GB202214708, each of which is incorporated herein by reference in its entirety, including the compound structures disclosed therein which are specifically incorporated herein by reference.

### iv) MSK1 Inhibitors

In some embodiments, compositions and methods described herein may include a compound of the present invention in combination with one or more Mitogen- and stress-activated kinase (MSK1) inhibitors. A MSK1 inhibitor may be administered or formulated in combination with a compound of the present invention and/or any additional therapeutic agent described herein. In some embodiments, a MSK1 inhibitor is one or more of SB-747651A, SB 747651A, Ro 320432, CGP 57380, GSK2830371, SR1664, LY-3214996, PFI-4, MSC-2363318A, and AS601245.

### v) RSK Inhibitors

In some embodiments, compositions and methods described herein may include a compound of the present invention in combination with one or more ribosomal S6 kinase (RSK) inhibitors. A RSK1 inhibitor may be administered or formulated in combination with a compound of the present invention and/or any additional therapeutic agent described herein. In some embodiments, a RSK inhibitor is one or more of BI-D1870, LJH685, SL0101-1, FMK, BRD7389, BIX 02565, LJI308, LJI308-S, LJI308-1, and LJH685-S. In some embodiments, a RSK inhibitor is PMD-026. In some embodiments, reference to the term RSK inhibitor includes any such RSK inhibitor disclosed in any one of the following patent applications: WO 2021249558, WO 2020165646, WO 2017141116, and CN 113801139, each of which is incorporated herein by reference in its entirety, including the compound structures disclosed therein which are specifically incorporated herein by reference.

### vi) ALK Inhibitors

In some embodiments, compositions and methods described herein may include a compound of the present invention in combination with one or more Anaplastic Lymphoma Kinase (ALK) inhibitors. An ALK inhibitor may be administered or formulated in combination with a compound of the present invention and/or any additional therapeutic agent described herein. In some embodiments, an ALK inhibitor is one or more of Crizotinib (Xalkori), Ceritinib (Zykadia), Alectinib (Alecensa), Brigatinib (Alunbrig), Lorlatinib (Lorbrena), Ensartinib (X-396), TAE684, ASP3026, TPX-0131, LDK378 (Ceritinib analog), CEP-37440; 4SC-203, TL-398, PLB1003, TSR-011, CT-707, TPX-0005, and AP26113. Additional examples of ALK kinase inhibitors are described in examples 3-39 of WO05016894. In some embodiments, reference to the term ALK inhibitor includes any such ALK inhibitor disclosed in any one of the following patent applications: WO 2019142095, WO 2019179482, WO 2018130928, WO 2018127184, WO 2017101803, WO 2016192132, WO 2014100431, WO 2012082972, CN 111138492, CN 110526914, CN 109836415, CN 105801603, CN 107987056, and CN 105878248, each of which is incorporated herein by reference in its entirety, including the compound structures disclosed therein which are specifically incorporated herein by reference.

### d) Receptor Tyrosine Kinase Inhibitors

Compositions and methods described herein may include a compound of the present invention in combination with one or more receptor tyrosine kinase inhibitors. A receptor tyrosine kinase (RTK) inhibitor is a type of molecule (e.g., small molecule, antibody, and nucleic acid) that binds to and blocks the activity of receptor tyrosine kinases or their ligands. RTKs are proteins found on the surface of cells that play a critical role in cell signaling and growth and have been developed as therapeutics for a range of diseases, including cancer, diabetes, and autoimmune disorders. In some embodiments, a therapeutic agent may be a pan-RTK inhibitor, such as afatinib.

### i) EGFR Inhibitors

In some embodiments, compositions and methods described herein may include a compound of the present invention in combination with one or more EGFR inhibitors. An EGFR inhibitor may be administered or formulated in combination with a compound of the present invention and/or any additional therapeutic agent described herein. EGFR inhibitors include, but are not limited to, small molecule antagonists, antibody inhibitors, or specific antisense nucleotide or siRNA. Useful antibody inhibitors of EGFR include cetuximab (ERBITUX®), panitumumab (VECTIBIX®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (IRESSA®), Lazertinib, erlotinib (TARCEVA®), and lapatinib (TYKERB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (TAGRISSO®). In some embodiments, an EGFR inhibitor is one or more of cetuximab, gefitinib (IRESSA®), erlotinib (TARCEVA®), and afatinib (Gilotrif. Additional non-limiting examples of small molecule EGFR inhibitors include any of the EGFR inhibitors described in Traxler et al., Exp. Opin. Ther. Patents 1998, 8(12):1599-1625. An EGFR inhibitor may be ERAS-801. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4). In some embodiments, the EGFR inhibitor may be bosutinib, crizotinib, dasatinib, erlotinib, gefitinib, lapatinib, pazopanib, ruxolitinib, sunitinib, vemurafenib, abrocitinib, asciminib, futibatinib, ibrutinib, imatinib, pacritinib, or sorafenib. In some embodiments, reference to the term EGFR inhibitor includes any such EGFR inhibitor disclosed in any one of the following patent applications: WO 2023041071, WO 2023049312, WO 2023020600, WO 2023284747, WO 2022206797, WO 2022258977, WO 2022033416, WO 2022033410, WO 2022105908, WO 2022100641, WO 2022014639, WO 2022007841, WO 2021018009, WO 2021057882, WO 2021252661, WO 2021018003, WO 2021073498, WO 2021238827, WO 2020254547, WO 2020216371, WO 2020147838, WO 2020207483, WO 2020254572, WO 2020001350, WO 2021001351, WO 2019164948, WO 2019218958, WO 2019046775, WO 2019015655, WO 2018121758, WO 2018218963, WO 2017220007, WO 2017205459, WO 2017161937, WO 2016192609, WO 199633980, WO 199630347, WO 199730034, WO 199730044, WO 199738994, WO 199749688, WO 199802434, WO 199738983, WO 199519774, WO 199519970, WO 199713771, WO 199802437, WO 199802438, WO 199732881, WO 199833798, WO 199732880, WO 199732880, WO 199702266, WO 199727199, WO 199807726, WO 1997/34895, WO 199631510, WO 199814449, WO 199814450, WO 199814451, WO 199509847, WO 199719065, WO 199817662, WO 199935146, WO 199935132, WO 199907701, WO 199220642, DE 19629652, EP 682027, EP 837063, EP 0787772, EP 0520722, EP 0566226, CN 115960018, CN 110283162, CN 114044774, CN 111973601, CN 111973602, and CN 113896744, each of which is incorporated herein by reference in its entirety, including the compound structures disclosed therein which are specifically incorporated herein by reference.

### ii) HER2 Inhibitors

In some embodiments, compositions and methods described herein may include a compound of the present invention in combination with one or more HER2 inhibitors. A HER2 inhibitor may be administered or formulated in combination with a compound of the present invention and/or any additional therapeutic agent described herein. In some embodiments, an HER2 inhibitor is one or more of tucatinib, rastuzumab (HERCEPTIN®), pertuzumab (Perjeta), lapatinib (TYKERB®), ado-trastuzumab emtansine (Kadcyla), and neratinib (Nerlynx). Non-limiting examples of HER2 inhibitors include monoclonal antibodies such as trastuzumab (HERCEPTIN®) and pertuzumab (PERJETA®); small molecule tyrosine kinase inhibitors such as gefitinib (IRESSA®), erlotinib (TARCEVA®), pilitinib, CP-654577, CP-724714, canertinib (CI 1033), HKI-272, lapatinib (GW-572016; TYKERB®), PKI-166, AEE788, BMS-599626, HKI-357, BIBW 2992, ARRY-334543, and JNJ-26483327. In some embodiments, reference to the term HER2 inhibitor includes any such HER2 inhibitor disclosed in any one of the following patent applications: WO 2021156178, WO 2021156180, WO 2021213800, WO 2021088987, WO 2013561183, and WO 2013056108, each of which is incorporated herein by reference in its entirety, including the compound structures disclosed therein which are specifically incorporated herein by reference.

iii) MET Inhibitors

In some embodiments, compositions and methods described herein may include a compound of the present invention in combination with one or more MET inhibitors. A MET inhibitor may be administered or formulated in combination with a compound of the present invention and/or any additional therapeutic agent described herein. In some embodiments, a MET inhibitor is one or more of Crizotinib (Xalkori), Cabozantinib (Cometriq, Cabometyx), Capmatinib (Tabrecta), Tepotinib (Tepmetko), Savolitinib (Volitinib), Onartuzumab (MetMab), Foretinib (GSK1363089), MGCD-265 (Amuvatinib), SU11274, and SU5416. In some embodiments, reference to the term MET inhibitor includes any such MET inhibitor disclosed in any one of the following patent applications: WO 2022226168, WO 2021222045, WO 2020047184, WO 2020015744, WO 2020244654, WO 2020156453, WO 2019206268, WO 2018077227, WO 2017012539, WO 2016015653, WO 2016012963, WO 2012015677, WO 2011162835, WO 2010089507, WO 2009091374, WO 2009056692, WO 2008051547, WO 2007130468, US 2012237524, CN 103497177, CN 107311983, CN 107382968, CN 110218191, and TW201331206, each of which is incorporated herein by reference in its entirety, including the compound structures disclosed therein which are specifically incorporated herein by reference.

### iv) AXL Inhibitors

In some embodiments, compositions and methods described herein may include a compound of the present invention in combination with one or more AXL inhibitors. An AXL inhibitor may be administered or formulated in combination with a compound of the present invention and/or any additional therapeutic agent described herein. AXL is a receptor tyrosine kinase that belongs to the TAM family of receptors, which also includes TYRO3 and MERTK. In some embodiments, an AXL inhibitor is one or more of bemcentib, BGB324, R428, SGI-7079, TP-0903, BMS-777607, UNC2025, and TP-0903. In some embodiments, reference to the term AXL inhibitor includes any such AXL inhibitor disclosed in any one of the following patent applications: WO 2023045816, WO 2022237843, WO 2022246179, WO 2021012717, WO 2021088787, WO 2021067772, WO 2021239133, WO 2021204713, WO 2020238802, WO 2019039525, WO 2019101178, WO 2019074116, WO 2017146236, WO 2016097918, WO 2015012298, WO 2010005876, WO 2010083465, CN 115073367, and JP 2022171109, each of which is incorporated herein by reference in its entirety, including the compound structures disclosed therein which are specifically incorporated herein by reference.

### v) IGFR Inhibitors

In some embodiments, compositions and methods described herein may include a compound of the present invention in combination with one or more insulin-like growth factor receptor 1 (IGF-1R) inhibitors. An IGFR inhibitor may be administered or formulated in combination with a compound of the present invention and/or any additional therapeutic agent described herein. IGFR inhibitors have been developed to target the IGFR receptor, which plays a critical role in cancer progression and metastasis. In some embodiments, an IGFR inhibitor is one or more of linsitinib, AXL1717, OSI-906 (Linsitinib), BMS-754807, BI 836845, AZ12253801, PQIP (Pyrrolo[1,2-a]quinoxaline), and NVP-AEW541. In some embodiments, reference to the term IGFR inhibitor includes any such IGFR inhibitor disclosed in any one of the following patent applications: WO 2022115946, WO 2022217923, WO 2021203861, WO 2021246413, WO 2020116398, WO 2019046600, WO 2018195250, WO 2018221521, WO 2018204872, WO 2017072196, WO 2016173682, WO 2015162291, WO 2015162292, WO 2010066868, WO 2006069202, and CN 112125916, each of which is incorporated herein by reference in its entirety, including the compound structures disclosed therein which are specifically incorporated herein by reference.

### vi) RET Inhibitors

In some embodiments, compositions and methods described herein may include a compound of the present invention in combination with one or more Rearranged during transfection (RET) inhibitors. An RET inhibitor may be administered or formulated in combination with a compound of the present invention and/or any additional therapeutic agent described herein. RET plays a critical role in various cellular processes, including cell growth, differentiation, survival, and migration. RET is activated by binding of its ligands, such as glial cell line-derived neurotrophic factor (GDNF) family ligands, which leads to the activation of downstream signaling pathways that promote these cellular processes. In some embodiments, a RET inhibitor is one or more of pralsetinib, selpercatinib (LOXO-292), BLU-667, RXDX-105, TPX-0046, GSK3179106, molidustat (BAY 85-3934), and RPI-1 (Retrophin). In some embodiments, reference to the term RET inhibitor includes any such RET inhibitor disclosed in any one of the following patent applications: WO 2021211380, WO 2021057963, WO 2021043209, WO 2021222017, WO 2020035065, WO 2020114487, WO 2020200314, WO 2020200316, WO 2020114494, WO 2018071447, WO 2018213329, WO 2017079140, WO 2014050781, CN 113943285, CN 113683610, CN 113683611, CN 113620944, CN 113620945, CN 113527291, CN 113527292, CN 113527290, CN 113135896, CN 111057075, CN 111233899, and CN 111362923, each of which is incorporated herein by reference in its entirety, including the compound structures disclosed therein which are specifically incorporated herein by reference.

vii) ROS1 Inhibitors

In some embodiments, compositions and methods described herein may include a compound of the present invention in combination with one or more c-ros oncogene 1 (ROS1) inhibitors. A ROS1 inhibitor may be administered or formulated in combination with a a compound of the present invention and/or any additional therapeutic agent described herein. ROS1 is a receptor tyrosine kinase that belongs to the insulin receptor family and plays a role in various cellular processes, including cell growth, differentiation, survival, and migration. In some embodiments, a ROS1 inhibitor is one or more of taletrectinib, DS-6051 b, TPX-0131, GZD824, and PF-06463922. In some embodiments, reference to the term ROS1 inhibitor includes any such ROS1 inhibitor disclosed in any one of the following patent applications: WO 2021098703, WO 2020024825, and US 2017079972, each of which is incorporated herein by reference in its entirety, including the compound structures disclosed therein which are specifically incorporated herein by reference.

viii) PDGFR Inhibitors

In some embodiments, compositions and methods described herein may include a compound of the present invention in combination with one or more platelet-derived growth factor receptor (PDGFR) inhibitors. A PDGFR inhibitor may be administered or formulated in combination with a compound of the present invention and/or any additional therapeutic agent described herein. PDGFR is a family of receptor tyrosine kinases that consists of two members, PDGFRα and PDGFRβ. They are activated by binding to their ligands, such as platelet-derived growth factor (PDGF), which leads to the activation of downstream signaling pathways that promote cell growth, proliferation, and survival. In some embodiments, a PDGFR inhibitor is one or more of CP-673451, imatinib, nintedanib (OFEV®), sunitinib (SUTENT®), pazopanib (VOTRIENT®), regorafenib (STIVARGA®), and dasatinib (SPRYCEL®).

### ix) FGF Inhibitors

In some embodiments, compositions and methods described herein may include a compound of the present invention in combination with fibroblast growth factor (FGF) inhibitors. An FGF inhibitor may be administered or formulated in combination with a compound of the present invention and/or any additional therapeutic agent described herein. FGFRs are a family of receptor tyrosine kinases that consists of four members, FGFR1-4. FGFRs are activated by binding to their ligands, fibroblast growth factors (FGFs), which leads to the activation of downstream signaling pathways that promote cell growth, differentiation, and survival. In some embodiments, the FGFR inhibitor is an inhibitor of FGFR2. In some embodiments, the FGFR inhibitor is an inhibitor of FGFR4. In some embodiments, an FGFR inhibitor is one or more of futibatinib (TAK-659), erdafitinib (BALVERSA®), infigratinib (Truseltiq), Debio 1347, and rogaratinib (BAY 1163877). In some embodiments, reference to the term FGFR inhibitor includes any such FGFR inhibitor disclosed in any one of the following patent applications: WO 2022033472, WO 2022152274, WO 2022166469, WO 2022206939, WO 2021037219, WO 2021089005, WO 2021113462, WO 2020185532, WO 2019213544, WO 2020164603, WO 2019154364, WO 2019034076, WO 2019213506, WO 2019223766, WO 2018028438, WO 2018153373, WO 2018121650, WO 2018010514, WO 2017028816, WO 2017118438, WO 2016134320, WO 2015008844, WO 2014172644, WO 2014007951, WO 2013179033, WO 2013087578, WO 2012047699, CN 105906630, CN 115869315, CN 115141176, CN 115043832, and CN 115028634, each of which is incorporated herein by reference in its entirety. In some embodiments, the FGF pathway inhibitor targets an FGF ligand. Such FGF pathway inhibitors include FGF ligand traps and antibodies. Non-limiting examples include, FP-1039, an FGF ligand trap consisting of the extracellular domain of FGFR1 fused to the Fc portion of human IgG1, designed to sequester FGF ligands and inhibit FGF signaling, and MFGR1877S, a monoclonal antibody targeting FGF ligands, designed to block FGF-mediated signaling, including the compound structures disclosed therein which are specifically incorporated herein by reference.

### x) VEGF Inhibitors

In some embodiments, compositions and methods described herein may include a compound of the present invention in combination with one or more vascular endothelial growth factor (VEGF) signaling inhibitors. VEGF (vascular endothelial growth factor) signaling inhibitors are a class of drugs that target the signaling pathway mediated by VEGF and its receptors. VEGF plays a critical role in angiogenesis, the process of forming new blood vessels from existing ones, and it is overexpressed in many types of cancer, making it an attractive target for cancer therapy. A VEGF inhibitor may be administered or formulated in combination with a compound of the present invention and/or any additional therapeutic agent described herein. In some embodiments, the VEGF inhibitor is an antibody or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto). In some embodiments, the VEGF inhibitor is one or more of bevacizumab, aflibercept, ramucirumab, sorafenib, sunitinib, and pazopanib.

### e) PI3K/mTOR Pathway Inhibitors

Compositions and methods described herein may include a compound of the present invention in combination with one or more inhibitors of the PI3K-AKT-TOR signaling pathway. The PI3K-AKT-mTOR signaling pathway is a critical intracellular pathway that regulates a wide range of cellular processes including cell growth, proliferation, metabolism, and survival. The pathway is initiated when growth factors, such as insulin or IGF-1, bind to cell surface receptors and activate phosphoinositide 3-kinase (PI3K). Activated PI3K then phosphorylates phosphatidylinositol 4,5-bisphosphate (PIP2) to produce phosphatidylinositol 3,4,5-trisphosphate (PIP3), which in turn activates AKT. Activated AKT then phosphorylates a variety of downstream targets including the tuberous sclerosis complex (TSC1/TSC2), leading to the activation of mTOR (mammalian target of rapamycin) complex 1 (mTORC1). Activated mTORC1 promotes protein synthesis and cell growth by phosphorylating key regulators of translation initiation such as S6 kinase (S6K) and eukaryotic initiation factor 4E-binding protein 1 (4E-BP1).

### i) PI3K Inhibitors

In some embodiments, compositions and methods described herein may include a compound of the present invention in combination with one or more PI3K inhibitors. A PI3K inhibitor may be administered or formulated in combination with a compound of the present invention and/or any additional therapeutic agent described herein. PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136. In some embodiments, the PI3K inhibitor is alpelisib or copanlisib.

### ii) AKT Inhibitors

In some embodiments, compositions and methods described herein may include a compound of the present invention in combination with one or more AKT inhibitors. An AKT inhibitor may be administered or formulated in combination with a compound of the present invention and/or any additional therapeutic agent described herein. AKT inhibitors include, but are not limited to, ipatasertib, GSK-2141795, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9). The PI3K/AKT inhibitor may include, but is not limited to, one or more PI3K/AKT inhibitors described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; and GSK2126458.

iii) mTOR Inhibitors

In some embodiments, compositions and methods described herein may include a compound of the present invention in combination with one or more mTOR inhibitors. A mTOR inhibitor may be administered or formulated in combination with a compound of the present invention and/or any additional therapeutic agent described herein. mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (TORISEL®); everolimus (AFINITOR®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

### iv) MNK Inhibitors

In some embodiments, compositions and methods described herein may include a compound of the present invention in combination with one or more mitogen-activated protein kinase-interacting kinase (MNK) inhibitors. A MNK inhibitor may be administered or formulated in combination with a compound of the present invention and/or any additional therapeutic agent described herein. MNK proteins are activated downstream of the mitogen-activated protein kinase (MAPK) signaling pathway, which plays a critical role in the regulation of cellular proliferation, differentiation, and survival. MNKs phosphorylate eIF4E, a key component of the eukaryotic translation initiation complex, which enhances the translation of specific mRNAs, including those encoding proteins involved in cell cycle regulation and oncogenesis. In some embodiments, a MNK inhibitor is one or more tomivosertib (eFT508), CGP57380, and SEL201. In some embodiments, reference to the term MNK inhibitor includes any such MNK inhibitor disclosed in any one of the following patent applications: WO 2021098691, WO 2020108619, WO 2020086713, WO 2018152117, WO 2018228275, WO 2015200481, and CN 115583942, each of which is incorporated herein by reference in its entirety, including the compound structures disclosed therein which are specifically incorporated herein by reference.

v) eIF4 Inhibitors

In some embodiments, compositions and methods described herein may include a compound of the present invention in combination with one or more eukaryotic initiation factor 4A (eIF4A) inhibitors. An eIF4A inhibitor may be administered or formulated in combination with a compound of the present invention and/or any additional therapeutic agent described herein. eIF4A is a critical component of the eukaryotic translation initiation complex, where it functions as an RNA helicase to unwind the secondary structure of mRNA and facilitate ribosome binding. eIF4A is required for the translation of many cancer-associated genes, making it an attractive therapeutic target for cancer treatment. In some embodiments, an eIF4A inhibitor is one or more zotatifin (eFT226), silvestrol, pateamine A, and rocaglates. In some embodiments, reference to the term eIF4A inhibitor includes any such eIF4A inhibitor disclosed in any one of the following patent applications: WO 2023034813, WO 2021195128, and WO 2017091585, each of which is incorporated herein by reference in its entirety, including the compound structures disclosed therein which are specifically incorporated herein by reference.

In some embodiments, compositions and methods described herein may include one or more eukaryotic initiation factor 4G (eIF4G) inhibitors. An eIF4G inhibitor may be administered or formulated in combination with a compound of the present invention and/or any additional therapeutic agent described herein. eIF4G family includes several proteins that are involved in the initiation of protein translation. eIF4G serves as a scaffold for other proteins, including eIF4E and eIF4A, to form the eIF4F complex, which is responsible for binding to the 5′ cap of mRNA and unwinding the secondary structure of the mRNA to allow ribosomal scanning and translation initiation. In some embodiments, an eIF4G inhibitor is one or more pateamine A, and hippuristanol.

### f) DNA Damage Response Inhibitors

Compositions and methods described herein may include a compound of the present invention in combination with one or more DNA damage response (DDR) inhibitors. The DDR pathway is a critical cellular pathway that is activated in response to DNA damage and is essential for maintaining genomic stability, thereby preventing the development of cancer. However, cancer cells often have defects in the DDR pathway, which makes them more sensitive to DDR inhibitors. DDR inhibitors have shown promise in preclinical studies as potential cancer therapeutics, particularly in combination with other agents.

### i) Wee1 Inhibitors

In some embodiments, compositions and methods described herein may include a compound of the present invention in combination with one or more Wee1 inhibitors. Wee1 is a kinase that plays a critical role in regulating the cell cycle by inhibiting the activity of cyclin-dependent kinases (CDKs) and preventing the progression of cells through the G2/M checkpoint. Wee1 is overexpressed in several cancer types and has been implicated in tumor growth and survival. In some embodiments, a Wee1 inhibitor is one or more of imp7068, adavosertib, azenosertib or ZNL-02-096. In some embodiments, reference to the term Wee1 inhibitor includes any such Wee1 inhibitor disclosed in any one of the following patent applications: WO 2022011391, WO 2022247641, WO 2021043152, WO 2020221358, WO 2020083404, WO 2020192581, WO 2019085933, WO 2018133829, WO 2015115355, WO 2015183776, WO 2014085216, and CN 114831993, each of which is incorporated herein by reference in its entirety, including the compound structures disclosed therein which are specifically incorporated herein by reference.

### ii) CHK Inhibitors

In some embodiments, compositions and methods described herein may include a compound of the present invention in combination with one or more checkpoint kinase (CHK) inhibitors. A CHK inhibitor may be administered or formulated in combination with a compound of the present invention and/or any additional therapeutic agent described herein. CHK1 kinase is a critical regulator of the cell cycle and the DNA damage response pathway. In some embodiments, the CHK inhibitor is a CHK1 inhibitor. In some embodiments, a CHK inhibitor is a CHK2 inhibitor. In some embodiments, a CHK1 inhibitor is one or more BBI-355, rabusertib, LY2606368, GDC-0575, and MK-8776. In some embodiments, reference to the term CHK1 inhibitor includes any such CHK1 inhibitor disclosed in any one of the following patent applications: WO 2024196923, WO 2024211271, WO 2024211270, WO 2024118564, WO 2023230477, WO 2022251502, WO 2021113661, WO 2021104461, WO 2019012030, WO 2010118390, WO 2008067027, WO 2002070494, and TW202126818, each of which is incorporated herein by reference in its entirety, including the compound structures disclosed therein which are specifically incorporated herein by reference.

iii) ATM Inhibitors

In some embodiments, compositions and methods described herein may include a compound of the present invention in combination with one or more ataxia telangiectasia mutated (ATM) inhibitors. An ATM inhibitor may be administered or formulated in combination with a compound of the present invention and/or any additional therapeutic agent described herein. ATM plays a role in regulating the replication stress response and maintaining genomic stability. In some embodiments, an ATM inhibitor is one or more M4076, AZD0156, KU-60019, and VE-821. In some embodiments, reference to the term ATM inhibitor includes any such ATM inhibitor disclosed in any one of the following patent applications: WO 2024189299, WO 2022058351, WO 2021197339, WO 2021098734, WO 2021260580, WO 2007026157, WO 2006085067, and US 2016113935, each of which is incorporated herein by reference in its entirety, including the compound structures disclosed therein which are specifically incorporated herein by reference.

### iv) ATR Inhibitors

In some embodiments, compositions and methods described herein may include a compound of the present invention in combination with one or more ataxia telangiectasia and Rad3-related (ATR) inhibitors. An ATR inhibitor may be administered or formulated in combination with a compound of the present invention and/or any additional therapeutic agent described herein. In some embodiments, an ATR inhibitor is one or more ceralaertib, VE-821, RP-350, AZ20, VX-970, abd110, VX-803, and BAY 1895344. In some embodiments, reference to the term ATR inhibitor includes any such ATR inhibitor disclosed in any one of the following patent applications: WO 2025019344, WO 2025019346, WO 2023138343, WO 2023126823, WO 2023109883, WO 2023016529, WO 2022237875, WO 2022268025, WO 2021012049, WO 2021023272, WO 2021260579, WO 2021228758, WO 2019050889, WO 2019154365, WO 2019133711, WO 2017059357, WO 2013049859, WO 2007046426, WO 2007015632, and CN 113797341, each of which is incorporated herein by reference in its entirety, including the compound structures disclosed therein which are specifically incorporated herein by reference.

### v) PARP Inhibitors

In some embodiments, compositions and methods described herein may include a compound of the present invention in combination with one or more Poly(ADP-ribose) polymerase (PARP) inhibitors. A PARP inhibitor may be administered or formulated in combination with a compound of the present invention and/or any additional therapeutic agent described herein. There are 17 PARP (aka tankyrase) family members that have been identified. PARP enzymes play a critical role in DNA damage repair, particularly in the repair of single-strand DNA breaks. PARP inhibitors block the activity of PARP enzymes, leading to the accumulation of DNA damage and ultimately cell death. In some embodiments, a PARP inhibitor is one or more Olaparib, rucaparib, niraparib, and veliparib (ABT-888). In some embodiments, reference to the term PARP inhibitor includes any such PARP inhibitor disclosed in any one of the following patent applications: WO 2025024581, WO 2025037273, WO 2025061057, WO 2024256377, WO 2024255782, WO 2023051812, WO 2023051807, WO 2023051716, WO 2023278592, WO 2022228387, WO 2022022664, WO 2022000946, WO 2022222921, WO 2021163530, WO 2020122034, WO 2020239097, WO 2020142583, WO 2020156577, WO 2020098774, WO 2020196712, WO 2019200382, WO 2018125961, WO 2018205938, WO 2018192576, WO 2018218025, WO 2017032289, WO 2017177838, WO 2017029601, WO 2017088723, WO 2016155655, WO 2015154630, WO 2013097225, WO 2012130166, WO 2011006794, WO 2009046205, WO 2009063244, WO 2008084261, WO 2007138351, WO 2006110816, WO 2005053662, WO 2005012524, CN 113698356, CN 113603647, CN 115073544, CN 108938634, CN 104887680, CN 110343088, CN 108976236, and CN 107629071, each of which is incorporated herein by reference in its entirety, including the compound structures disclosed therein which are specifically incorporated herein by reference.

### vi) DNA-PK Inhibitors

In some embodiments, compositions and methods described herein may include a compound of the present invention in combination with one or more DNA-dependent protein kinase (DNA-PK) inhibitors. An DNA-PK inhibitor may be administered or formulated in combination with a compound of the present invention and/or any additional therapeutic agent described herein. DNA-dependent protein kinase (DNA-PK) is a serine/threonine protein kinase that plays a crucial role in DNA repair and maintenance of genome stability. In some embodiments, a DNA-PK inhibitor is one or more NU7441, AZD7648, VX-984, M3814, and CC-115. In some embodiments, reference to the term DNA-PK inhibitor includes any such DNA-PK inhibitor disclosed in any one of the following patent applications: WO 2025023957, WO 2023220418, WO 2023215991, WO 2023165603, WO 2022187965, WO 2021197159, WO 2021260583, WO 2021204111, WO 2021104277, WO 2021098813, WO 2021022078, WO 2020259613, WO 2019143678, WO 2019143675, WO 2019201283, WO 2015058031, WO 2014159690, WO 2012028233, WO 2009010761, WO 2006032869, WO 2006109084, CN 112574179, CN 112300132, and CN 112300126, each of which is incorporated herein by reference in its entirety, including the compound structures disclosed therein which are specifically incorporated herein by reference.

### g) Cell Cycle Inhibitors

Compositions and methods described herein may include a compound of the present invention in combination with one or more cell cycle inhibitors. Cell cycle inhibitors target specific proteins involved in regulating the cell cycle, which is the process by which a cell divides and replicates its DNA. Non-limiting examples cell cycle proteins include cyclin-dependent kinase (CDK), aurora kinase, and polo-like kinase (PLK). CDKs are a family of kinases that are involved in regulating the cell cycle. CDK inhibitors block the activity of these kinases, leading to cell cycle arrest and/or apoptosis. Aurora kinases are a family of serine/threonine kinases that play a critical role in regulating mitosis. Aurora kinase inhibitors block the activity of these kinases, leading to mitotic arrest and cell death. PLKs are a family of serine/threonine kinases that are involved in regulating multiple stages of the cell cycle. PLK inhibitors block the activity of these kinases, leading to cell cycle arrest and/or apoptosis.

### i) CDK Inhibitors

In some embodiments, compositions and methods described herein may include a compound of the present invention in combination with one or more CDK inhibitors. A CDK inhibitor may be administered or formulated in combination with a compound of the present invention and/or any additional therapeutic agent described herein. Cyclin-dependent kinases are a family of protein kinases that regulate cell division and proliferation. Cell cycle progression is controlled by cyclins and their associated cyclin-dependent kinases, such as CDK1, CDK2, CDK3, CDK4 and CDK6, while other CDKs such as CDK7, CDK8 and CDK9 are critical to transcription. CDK binding to cyclins forms heterodimeric complexes that phosphorylate their substrates on serine and threonine residues, which in turn initiates events required for cell-cycle transcription and progression. In some embodiments, a CDK inhibitor is a CDK2 inhibitor. In some embodiments, a CDK inhibitor is a CDK4/6 inhibitor. In some embodiments, a CDK inhibitor is a CDK7 inhibitor. In some embodiments, a CDK inhibitor is a CDK9 inhibitor. In some embodiments, a CDK inhibitor is one or more palbociclib, ribociclib, abemaciclib, and trilaciclib. In some embodiments, a CDK inhibitor is one or more of tagtociclib (PF-07104091), seliciclib, voruciclib P1446A-05, BLU-222, dinaciclib, AT-7519, RGB286638, and AZD4573.

In some embodiments, reference to the term CDK inhibitor includes any such CDK inhibitor disclosed in any one of the following patent applications: WO 2025040170, WO 2025060620, WO 2024238574, WO 2024027825, WO 2024048541, WO 2022166793, WO 2022187611, WO 2022130304, WO 2021227906, WO 2021057867, WO 2020207260, WO 2020138370, WO 2020125513, WO 2020148635, WO 2020215156, WO 2020052627, WO 2017177837, WO 2017162215, WO 2017177836, WO 2016193939, WO 2016014904, WO 2016015598, WO 2016015605, WO 2015181737, WO 2012061156 A1, WO 2012038411, WO 2010020675, WO 2010125004, WO 2007139732, WO 2006024945, CN 114478529, CN 108794496, CN 105294737, CN 107652284, KR 20180106188, and US 2017152269, each of which is incorporated herein by reference in its entirety, including the compound structures disclosed therein which are specifically incorporated herein by reference.

### ii) Aurora Kinase Inhibitors

In some embodiments, compositions and methods described herein may include a compound of the present invention in combination with one or more aurora kinase inhibitors. An aurora kinase inhibitor may be administered or formulated in combination with a compound of the present invention and/or any additional therapeutic agent described herein. Aurora kinases are a family of serine/threonine kinases that play a critical role in regulating cell division and maintaining genomic stability. The Aurora kinase family consists of three members: Aurora A, Aurora B, and Aurora C. In some embodiments, an aurora kinase inhibitor is one or more palbociclib, ribociclib, and abemaciclib. In some embodiments, an aurora kinase inhibitor is one or more of alisertib, danusertib, barasertib, and MLN8237. In some embodiments, reference to the term aurora kinase inhibitor includes any such aurora kinase inhibitor disclosed in any one of the following patent applications: WO 2021110009, WO 2021008338, WO 2020112514, WO 2019129234, WO 2016077161, WO 2013143466, WO 2011103089, WO 2010081881, WO 2010133794, WO 2009134658, WO 2008001886, WO 2007095124, WO 2007003596, WO 2006129064, CN 114276227, CN 108078991, CN 106543155, CN 104211692, and CN 104098551, each of which is incorporated herein by reference in its entirety, including the compound structures disclosed therein which are specifically incorporated herein by reference.

iii) PLK Inhibitors

In some embodiments, compositions and methods described herein may include a compound of the present invention in combination with one or more polo-like kinase (PLK) inhibitors. A PLK inhibitor may be administered or formulated in combination with a compound of the present invention and/or any additional therapeutic agent described herein. PLKs are a family of serine/threonine kinases that play a crucial role in regulating cell division, DNA damage response, mitotic progression, and consists of four members: PLK1, PLK2, PLK3, and PLK4. In some embodiments, a PLK inhibitor is one or more of volasertib, onvansertib, BI 2536, and GSK461364. In some embodiments, reference to the term PLK inhibitor includes any such PLK inhibitor disclosed in any one of the following patent applications: WO 2011012534 A1, WO 2010065134, WO 2009130453, WO 2009042806, WO 2004043936, WO 2007030361, WO 2006021547, CN 115804777, and EP 2325185, each of which is incorporated herein by reference in its entirety, including the compound structures disclosed therein which are specifically incorporated herein by reference.

### iv) Kinesin Superfamily of Microtubule Motor Protein Inhibitors

In some embodiments, compositions and methods described herein may include a compound of the present invention in combination with one or more Kinesin spindle protein (KSP) inhibitors. In some embodiments, compositions described herein may include one or more Kinesin family (KIF) inhibitors. In some embodiments, a KSP inhibitor may be administered or formulated in combination with a compound of the present invention and/or any additional therapeutic agent described herein. KSP and KIF are a subset of the kinesin superfamily of microtubule motor proteins. KSP, also known as Eg5, is a member of the kinesin superfamily of motor proteins that plays a critical role in mitotic spindle formation and cell division. KSP inhibitors selectively target rapidly dividing cancer cells by disrupting spindle formation and inducing mitotic arrest. In some embodiments, a KSP inhibitor is one or more of SB743921, monastrol, S-Trityl-L-cysteine (STLC), and filanesib (ARRY-520). In some embodiments, a KIF inhibitor is an inhibitor of a Kinesin-8 family microtubule motor protein. In some embodiments, the kinesin-8 family protein is KIF18A. In some embodiments, a KIF inhibitor is one or more of AMG650, BTB-1, K03861, and SJ000291942. In some embodiments, reference to the term kinesin superfamily of microtubule motor protein inhibitor includes any such kinesin superfamily of microtubule motor protein inhibitor disclosed in any one of the following patent applications: WO 2015114854, WO 2015114855, WO 2010084186, WO 2006101761, WO 2006110390, WO 2006044825, WO 2006078574, WO 2005060654, WO 2004092147, WO 2004037171, WO 2004058700, WO 2003050064, WO 2003105855, WO 2022037665, WO 2018114804, WO 2017162663, WO 2016207089, WO 2012073375, JP 2014162787, JP 2019189590, JP2013166713, and KR 20220145566, each of which is incorporated herein by reference in its entirety, including the compound structures disclosed therein which are specifically incorporated herein by reference.

### v) DYRK1 Inhibitors

In some embodiments, compositions and methods described herein may include a compound of the present invention in combination with one or more Dual-specificity tyrosine phosphorylation-regulated kinase 1 (DYRK1) inhibitors. A DYRK1 inhibitor may be administered or formulated in combination with a compound of the present invention and/or any additional therapeutic agent described herein. DYRK1 is a member of the DYRK (dual-specificity tyrosine phosphorylation-regulated kinase) family of protein kinases. It plays essential roles in various cellular processes, including cell cycle regulation, neuronal development, and transcriptional control. In some embodiments, a DYRK1 inhibitor is one or more of harmine, INDY, D4476, and AZ191. In some embodiments, reference to the term DYRK1 inhibitor includes any such DYRK1 inhibitor disclosed in any one of the following patent applications: WO 2023277331 A1, WO 2023140846 A1, WO 2017181087 A1, each of which is incorporated herein by reference in its entirety, including the compound structures disclosed therein which are specifically incorporated herein by reference.

### h) Anti-Apoptotic Protein Inhibitors

Compositions and methods described herein may include a compound of the present invention in combination with one or more anti-apoptotic protein inhibitors. In some embodiments, an anti-apoptotic protein inhibitor may be administered or formulated in combination with a compound of the present invention and/or any additional therapeutic agent described herein. Anti-apoptotic inhibitors target proteins that play a role in preventing apoptosis, a form of programmed cell death. Apoptosis is a critical mechanism for eliminating damaged or unwanted cells. Anti-apoptotic proteins are a family of proteins that inhibit the apoptotic pathway, thereby preventing cell death. There are several known classes of anti-apoptotic inhibitors, including Bcl-2 inhibitors, XIAP inhibitors, survivin inhibitors, Mcl-1 inhibitors, and FLIP inhibitors. These inhibitors work by binding to specific anti-apoptotic proteins and preventing their activity, thereby promoting cell death in cancer cells. In some embodiments, compositions described herein may include one or more anti-apoptotic protein inhibitors. An anti-apoptotic protein inhibitor may be administered or formulated in combination with a RAS(ON) inhibitor and/or any additional therapeutic agent described herein. In some embodiments, the anti-apoptotic protein inhibitor includes a MCL-1 inhibitor. Non-limiting examples of MCL-1 inhibitors include, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263. In some embodiments, the anti-apoptotic protein inhibitor includes a BCL protein inhibitor. Examples of BCL protein inhibitors include but are not limited to Venetoclax (Venclexta), Navitoclax (ABT-263), A-1331852, S63845, and AT-101.

### i) Autophagy Inhibitors

Compositions and methods described herein may include a compound of the present invention in combination with one or more autophagy inhibitors. In some embodiments, an autophagy inhibitor may be administered or formulated in combination with a compound of the present invention and/or any additional therapeutic agent described herein. Autophagy inhibitors include, but are not limited to chloroquine, 3-methyladenine, hydroxychloroquine (PLAQUENIL™), spautin-1, SAR405, bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

### a) ULK Inhibitors

In some embodiments, compositions and methods described herein may include a compound of the present invention in combination with one or more Unc-51-like kinase (ULK) inhibitors. An ULK inhibitor may be administered or formulated in combination with a compound of the present invention and/or any additional therapeutic agent described herein. In some embodiments, a ULK inhibitor is a ULK1/2 inhibitor. In some embodiments, an ULK inhibitor is one or more of ULK-101, MRT68921, SBI-0206965, MRT67307, MRT68920, MRT68922, MRT199665, LY3009120, and Dorsomorphin.

### b) VPS Inhibitors

In some embodiments, compositions and methods described herein may include a compound of the present invention in combination with one or more Vacuolar protein sorting protein (VPS) inhibitors. A VPS inhibitor may be administered or formulated in combination with a compound of the present invention and/or any additional therapeutic agent described herein. VPS (proteins are a family of proteins that play a critical role in the process of autophagy by regulating the formation and function of autophagosomes, structures that engulf and transport cellular components to lysosomes for degradation. Dysregulation of VPS proteins has been implicated in various diseases, including cancer, neurodegenerative disorders, and infectious diseases. In some embodiments, a VPS inhibitor is a VPS34 inhibitor. In some embodiments, a VPS inhibitor is one or more of PIK-III, VPS34-IN1, SAR405, Spautin-1, and NSC185058.

### c) Macropinocytosis Inhibitors

In some embodiments, compositions and methods described herein may include a compound of the present invention in combination with one or more macropinocytosis inhibitors. A macropinocytosis inhibitor may be administered or formulated in combination with a RAS(ON) inhibitor and/or any additional therapeutic agent described herein. Macropinocytosis inhibitors are compounds that can block or reduce the process of macropinocytosis. In some embodiments, a macropinocytosis inhibitor is one or more of EIPA (ethylisopropylamiloride), Wortmannin, Amiloride, Apilimod, Dyngo-4a, and Latrunculin B.

### j) WNT β-Catenin Pathway Inhibitors

Compositions and methods described herein may include a compound of the present invention in combination with one or more WNT/beta-catenin pathway inhibitors. In some embodiments, a WNT/beta-catenin pathway inhibitor may be administered or formulated in combination with a compound of the present invention and/or any additional therapeutic agent described herein. The WNT/beta-catenin pathway is an important signaling pathway that plays a crucial role in development, tissue homeostasis, and disease. Dysregulation of this pathway has been implicated in various cancers, making it an attractive target for cancer therapy. WNT/beta-catenin pathway inhibitors target various components of the pathway, including WNT ligands, receptors, and downstream effectors.

### i) Beta-Catenin Inhibitors

In some embodiments, compositions and methods described herein may include a compound of the present invention and one or more β-catenin inhibitors. A β-catenin inhibitor may be administered or formulated in combination with a compound of the present invention and/or any additional therapeutic agent described herein. Beta-catenin is a protein that plays an important role in the WNT signaling pathway, which regulates various cellular processes including cell proliferation, differentiation, and migration. In normal cells, β-catenin levels are tightly regulated by a destruction complex, which marks beta-catenin for degradation. However, in many cancer cells, the destruction complex is impaired, leading to the accumulation of beta-catenin in the nucleus and the activation of target genes involved in tumor growth and metastasis. In some embodiments, a WNT/β-catenin inhibitor is one or more of FOG-001, OMP-131R10, Foxy-5, LGK974, RXC004, ETC-159, OMP-54F28, Niclosamide, OMP-18R5, OTSA-101, BNC101, DKN-01, Sulindac, Pyrvinium, E7449, BC2059, PRI-724, SM08502, IWP1, IWP2, IWP3, IWP4, IWP12, IWP L6, C59, GNF-6231, GNF-1331, DK-520, DK-419, IgG-2919, Fz7-21, RHPD-P1, SR137892, 1094-0205, 2124-0331, 3235-0367, NSC36784, NSC654259, IgG-2919, Salinomycin, BMD4702, 3289-8625, J01-017a, FJ9, KY-02061, KY-02327, NSC668036, Peptide Pen-N3, SSTC3, CCT031374, TCS 183, XAV939, AZ1366, G007-LK, MSC2504877, G244-LM, IWR-1, JW74, JW55, K-756, NVP-TNKS656, MN-64, RK-287107, WIKI4, KY1220, KYA1797K, MSAB, PKF115-584, CGP049090, AV-65, PNU-74654, Windorphen, IQ-1 tegavivant, foscenvivant, PNPB-29, ZW4864, SAH-BCL9, Carnosic acid, xStAx-VHL, NRX-252114, Septuximab vedotin, PF-06647020, LGR5-mc-vc-PAB-MMAE, LGR5-NMS818, CWP232291, PRI-724 (also known as ICG-001), C-82, and BC2059. In some embodiments, reference to the term β-catenin inhibitor includes any such b-catenin inhibitor disclosed in any one of the following patent applications: CN 104388427 and CN 103830211, each of which is incorporated herein by reference in its entirety, including the compound structures disclosed therein which are specifically incorporated herein by reference.

### ii) PORCN Inhibitors

In some embodiments, compositions and methods described herein may include a compound of the present invention in combination with one or more Porcupine (PORCN) inhibitors. A PORCN inhibitor may be administered or formulated in combination with a compound of the present invention and/or any additional therapeutic agent described herein. PORCN is a membrane-bound O-acyltransferase enzyme that plays a critical role in the WNT signaling pathway by mediating the palmitoylation of WNT ligands. This palmitoylation is essential for the secretion and signaling activity of WMT proteins. Inhibition of PORCN leads to reduced WNT signaling activity. In some embodiments, a PORCN inhibitor is one or more of LGK974 (WNT974), ETC-1922159, CGX1321, and CWP232291.

iii) GSK3 Inhibitors

In some embodiments, compositions and methods described herein may include a compound of the present invention in combination with one or more Glycogen synthase kinase (GSK3) inhibitors. A GSK3 inhibitor may be administered or formulated in combination with a compound of the present invention and/or any additional therapeutic agent described herein. The GSK3 family consists of two closely related serine/threonine kinases: GSK3a and GSK3P. These kinases are involved in numerous cellular processes, including glycogen metabolism, cell cycle regulation, and Wnt signaling. GSK inhibitors have been investigated as potential therapeutics for various diseases, including cancer, diabetes, Alzheimer's disease, and bipolar disorder. In some embodiments, a GSK3 inhibitor is one or more of Tideglusib, laduviglusib, LiCl (Lithium chloride), CHIR99021, SB216763, AZD1080, and LY2090314. In some embodiments, reference to the term GSK3 inhibitor includes any such GSK3 inhibitor disclosed in any one of the following patent applications: WO 2017153834, WO 2014059383, WO 2010012398, WO 2009017455, WO 2003037891, CN 107151235, and CN 102258783, each of which is incorporated herein by reference in its entirety, including the compound structures disclosed therein which are specifically incorporated herein by reference.

### iv) CLK Inhibitors

In some embodiments, compositions and methods described herein may include a compound of the present invention in combination with one or more Cdc2-like kinase (CLK) inhibitors. A CLK inhibitor may be administered or formulated in combination with a compound of the present invention and/or any additional therapeutic agent described herein. LKs (Cdc2-like kinases) are a family of serine/threonine kinases that play a crucial role in pre-mRNA splicing, specifically in the regulation of alternative splicing. There are four members of the CLK family: CLK1, CLK2, CLK3, and CLK4. The CLK family of kinases have been shown to be involved in several diseases, including cancer, neurodegenerative disorders, and viral infections. In some embodiments, a CLK inhibitor is a CLK 2 inhibitor. In some embodiments, a CLK2 inhibitor is one or more of Lorecivivint, SM08502, SM04690, TG003, KH-CB19, Cmpd-1, T3.5, and CX-4945. In some embodiments, reference to the term CLK inhibitor includes any such CLK inhibitor disclosed in WO 2020006115, which is incorporated herein by reference in its entirety, including the compound structures disclosed therein which are specifically incorporated herein by reference.

### k) JAK/STAT Pathway Inhibitors

Compositions and methods described herein may include a compound of the present invention in combination with one or more JAK/STAT pathway inhibitors. In some embodiments, a JAK/STAT pathway inhibitor may be administered or formulated in combination with a compound of the present invention and/or any additional therapeutic agent described herein. The Janus kinase/signal transducer and activator of transcription (JAK/STAT) pathway is a signaling pathway involved in many cellular processes, including immune response, cell growth, and differentiation. Dysregulation of this pathway has been linked to various diseases, including inflammatory disorders, cancer, and autoimmune diseases. Inhibitors of the JAK/STAT pathway can be used for the treatment of these diseases. In some embodiments, a JAK/STAT pathway inhibitor is an inhibitor of JAK1, JAK2 and/or JAK3. In some embodiments, a JAK inhibitor is one or more of Ruxolitinib (JAKAFI®), Pacritinib, Fedratinib, Tofacitinib (XELJANZ®), Abrocitinib, Filgotinib, Oclacitinib, Peficitinib, Upadacitinib, Deucravacitinib, Delgocitinib, and Baricitinib (OLUMIANT®). In some embodiments, reference to the term JAK inhibitor includes any such JAK inhibitor disclosed in any one of the following patent applications: WO 2023011301, WO 2023201044, WO 2022143629, WO 2022251434, WO 2022067106, WO 2022033551, WO 2021244323, WO 2021238817, WO 2021238818, WO 2021178991, WO 2021136345, WO 2021190647, WO 2020219639, WO 2020182159, WO 2020155931, WO 2020038457, WO 2020219524, WO 2020173400, WO 2018204233, WO 2018204238, WO 2018169875, WO 2018117152, WO 2017215630, WO 2016070697, WO 2016027195, CN 117815195, CN 117815367, and CN 115969796, each of which is incorporated herein by reference in its entirety, including the compound structures disclosed therein which are specifically incorporated herein by reference.

In some embodiments, the JAK/STAT pathway inhibitor is a STAT inhibitor. In some embodiments, the STAT inhibitor is an inhibitor of STAT3 and/or STAT5. In some embodiments, the STAT inhibitor is a STAT3 degrader. In some embodiments, the STAT inhibitor is one or more of TTI-101, C-188-9, WP1066, VVD-130850, LLL12B, STA-21, SD-36, Stattic, S31-201, OPB-31121, and Napabucasin (BBI608). In some embodiments, reference to the term STAT inhibitor includes any such STAT inhibitor disclosed in any one of the following patent applications: WO 2024030628, WO 2023164680, WO 2023192960, WO 2023133336, WO2020206424, WO 2023107706, WO 2021150543, WO 2008151037, and CN 109288845, each of which is incorporated herein by reference in its entirety, including the compound structures disclosed therein which are specifically incorporated herein by reference.

### l) Epigenetic Modulators

Compositions and methods described herein may include a compound of the present invention in combination with one or more epigenetic modulators. Epigenetic modulators are a class of therapeutics that target enzymes responsible for modifying the structure and function of chromatin, the complex of DNA and proteins that make up chromosomes. These enzymes, including histone deacetylases (HDACs), histone methyltransferases (HMTs), and DNA methyltransferases (DNMTs), play critical roles in gene expression and regulation by modifying the packaging of DNA and affecting how it is read and transcribed. Epigenetic modulators work by altering the activity of these enzymes, either by inhibiting or enhancing their function, to regulate gene expression in specific ways. By targeting specific epigenetic modifications, such as acetylation, methylation, and DNA methylation, these therapies have the potential to treat a wide range of diseases, including cancer, inflammatory disorders, and neurological disorders.

### i) HDAC Inhibitors

In some embodiments, compositions and methods described herein may include a compound of the present invention in combination with one or more histone deacetylase (HDAC) inhibitors. A HDAC inhibitor may be administered or formulated in combination with a compound of the present invention and/or any additional therapeutic agent described herein. There are several classes of HDACs, including class I, class IIa, class IIb, class III, and class IV. Class I HDACs are further divided into HDAC1, HDAC2, HDAC3, and HDAC8, while class IIa HDACs include HDAC4, HDAC5, HDAC7, and HDAC9. Class IIb HDACs consist of HDAC6 and HDAC10, and class III HDACs are known as sirtuins. HDAC inhibitors can target different classes of HDACs, and their specific effects on gene expression can vary depending on which HDACs they target. In some embodiments, a HDAC inhibitor is one or more of Vorinostat (ZOLINZA®), Romidepsin (ISTODAX®), Belinostat (BELEODAQ®), Panobinostat (FARYDAK®), Entinostat (MS-275), Valproic acid (DEPAKENE®), Trichostatin A (TSA), Sodium butyrate, and Mocetinostat (MGCD0103). Non-limiting examples of HDAC inhibitors include trichostatin, sodium butyrate, apicidan, suberoyl anilide hydroamic acid, vorinostat, LBH 589, romidepsin, ACY-1215, and Panobinostat. In some embodiments, reference to the term HDAC inhibitor includes any such HDAC inhibitor disclosed in any one of the following patent applications: WO 2022110958, WO 2021252628, WO 2019204550, WO 2018178060, WO 2016126724, WO 2014143666, WO 2013041480, and WO 2006120456, each of which is incorporated herein by reference in its entirety, including the compound structures disclosed therein which are specifically incorporated herein by reference.

### ii) BET Inhibitors

In some embodiments, compositions and methods described herein may include a compound of the present invention in combination with one or more bromodomain and extra-terminal protein (BET) inhibitors. A BET inhibitor may be administered or formulated in combination with a compound of the present invention and/or any additional therapeutic agent described herein. BET (bromodomain and extra-terminal) proteins are a family of epigenetic reader proteins that recognize and bind to acetylated lysine residues on histones, leading to chromatin remodeling and gene expression regulation. There are four BET proteins in humans: BRD2, BRD3, BRD4, and BRDT. BET inhibitors specifically target the bromodomains of BET proteins, inhibiting their binding to acetylated lysine residues on histones and leading to alterations in gene expression. BET inhibitors are useful in the treatment of cancer and other diseases characterized by dysregulated gene expression. In some embodiments, a BET inhibitor is one or more of JQ1, I-BET762, OTX015, RVX-208, and CPI-0610. In some embodiments, reference to the term BET inhibitor includes any such BET inhibitor disclosed in any one of the following patent applications: WO 2022046682, WO 2022182857, WO 2021107657, WO 2021107656, WO 2020221006, WO 2020053660, WO 2018097977, WO 2017222977, WO 2017142881, WO 2015075665, WO 2015011084, and CN 113264930, each of which is incorporated herein by reference in its entirety, including the compound structures disclosed therein which are specifically incorporated herein by reference.

iii) EZH2 Inhibitors

In some embodiments, compositions and methods described herein may include a compound of the present invention in combination with one or more Enhancer of Zeste Homolog 2 (EZH2) inhibitors. An EZH2 inhibitor may be administered or formulated in combination with a compound of the present invention and/or any additional therapeutic agent described herein. EZH2 is a histone-lysine N-methyltransferase that is a member of the Polycomb repressive complex 2 (PRC2) family. EZH2 plays a crucial role in gene expression regulation, specifically by catalyzing the trimethylation of histone H3 at lysine 27 (H3K27me3), leading to transcriptional repression of target genes. EZH2 has been found to be overexpressed in several types of cancers and is associated with tumor progression and poor prognosis. In some embodiments, an EZH2 inhibitor is one or more of Tazemetostat, GSK2816126, and CPI-1205 (lirametostat). In some embodiments, reference to the term EZH2 inhibitor includes any such EZH2 inhibitor disclosed in any one of the following patent applications: WO 2023030299, WO 2022179584, WO 2020224607, WO 2021243060, WO 2021086069, WO 2019206155, WO 2018133795, WO 2018137639, WO 2017184999, WO 2017218953, WO 2016201328, WO 2015195848, WO 2013155317, WO 2013138361, and CN 114621191, each of which is incorporated herein by reference in its entirety, including the compound structures disclosed therein which are specifically incorporated herein by reference.

### iv) Co-REST Inhibitors

In some embodiments, compositions and methods described herein may include a compound of the present invention in combination with one or more Co-REST inhibitors. A Co-REST inhibitor may be administered or formulated in combination with a compound of the present invention and/or any additional therapeutic agent described herein. Co-REST is a transcriptional co-repressor protein that interacts with a variety of transcription factors to regulate gene expression. Co-REST acts by recruiting histone deacetylases (HDACs) to chromatin, leading to the repression of gene expression. Inhibition of Co-REST has been proposed as a potential therapeutic strategy for the treatment of various diseases, including neurodegenerative disorders and cancer. In some embodiments, a co-REST inhibitor is one or more of Nocodazole, NSC 1892, and Anacardic acid.

### v) EP300

In some embodiments, compositions and methods described herein may include a compound of the present invention in combination with one or more E1A-binding protein p300 (EP300) inhibitors. An EP300 inhibitor may be administered or formulated in combination with a compound of the present invention and/or any additional therapeutic agent described herein. EP300 is a transcriptional co-activator involved in the regulation of numerous cellular processes, including chromatin remodeling, DNA damage response, and cell cycle progression. EP300 acts as a histone acetyltransferase, catalyzing the transfer of acetyl groups to lysine residues on histone proteins, which leads to changes in chromatin structure and gene expression. EP300 activity has been implicated in diseases, such as cancer, cardiovascular and neurological disorders. In some embodiments, an EP300 inhibitor is one or more of C646, A-485, NU9056, and L002. In some embodiments, reference to the term EP300 inhibitor includes any such EP300 inhibitor disclosed in any one of the following patent applications: WO 2021213521 and WO 2016044694, each of which is incorporated herein by reference in its entirety, including the compound structures disclosed therein which are specifically incorporated herein by reference.

### vi) LSD1

In some embodiments, compositions and methods described herein may include a compound of the present invention in combination with one or more Lysine-specific demethylase 1 (LSD1) inhibitors. A LSD1 inhibitor may be administered or formulated in combination with a compound of the present invention and/or any additional therapeutic agent described herein. LSD1 is an enzyme that plays a crucial role in regulating gene expression through histone modification. It specifically removes the methyl group from lysine 4 on histone 3, leading to gene repression. Dysregulation of LSD1 has been associated with various diseases including cancer and neurodegenerative disorders. In some embodiments, a LSD1 inhibitor is one or more of GSK2879552, IMG-7289, ORY-1001, IMG-8419, SP-2577, CC-90011, HCl-2509, and INCB059872. In some embodiments, reference to the term LSD1 inhibitor includes any such LSD1 inhibitor disclosed in any one of the following patent applications: WO 2021095840, WO 2021175079, WO 2021058024, WO 2020047198, WO 2020052649, WO 2020015745, WO 2020052647, WO 2018137644, WO 2017184934, WO 2017027678, WO 2017116558, WO 2017149463, WO 2016161282, WO 2015123465, WO 2015123424, WO 2013057322, WO 2013057320, WO 2012135113, CN 114805261, CN 111072610 CN 107174584, CN 110478352, CN 106432248, and CN 106045881, each of which is incorporated herein by reference in its entirety, including the compound structures disclosed therein which are specifically incorporated herein by reference.

vii) PRMT5

In some embodiments, compositions and methods described herein may include a compound of the present invention in combination with one or more Protein arginine methyltransferase 5 (PRMT5) inhibitors. A PRMT5 inhibitor may be administered or formulated in combination with a compound of the present invention and/or any additional therapeutic agent described herein. PRMT5 is a member of the PRMT family, which catalyzes the transfer of a methyl group from S-adenosylmethionine (SAM) to the nitrogen atoms of arginine residues in target proteins. PRMT5 is involved in various biological processes, including gene expression regulation, signal transduction, and DNA repair. In some embodiments, a PRMT5 inhibitor is one or more of TNG908, TNG462, AMG193, GSK591, EPZ015666, TC-E 5003, and MS023. In some embodiments, reference to the term PRMT5 inhibitor includes any such PRMT5 inhibitor disclosed in any one of the following patent applications: WO 2023001133, WO 2022206964, WO 2022153161, WO 2021068953, WO 2021088992, WO 2020259478, WO 2020205660, WO 2020250123, WO 2020033288, WO 2019102494, WO 2019112719, WO 2019180631, WO 2018065365, WO 2017153186, WO 2017212385, WO 2017032840, WO 2016022605, WO2014100695, WO 2014145214, WO 2014100719, CN 111825656, CN 114558014, CN 11304554, and CN 112778275, each of which is incorporated herein by reference in its entirety, including the compound structures disclosed therein which are specifically incorporated herein by reference.

viii) MAT2A

In some embodiments, compositions and methods described herein may include a compound of the present invention in combination with one or more methionine adenosyltransferase 2A (MAT2A) inhibitors. A MAT2A inhibitor may be administered or formulated in combination with a compound of the present invention and/or any additional therapeutic agent described herein. MAT2A is an enzyme that catalyzes the production of S-adenosylmethionine (SAM), which is an important cofactor in many biological processes, including DNA methylation, protein methylation, and polyamine synthesis. Elevated MAT2A expression has been associated with various cancers. In some embodiments, a MAT2A inhibitor is one or more of cycloleucine and 2-hydroxy-4-methylthiobutanoic acid. In some embodiments, reference to the term MAT2A inhibitor includes any such MAT2A inhibitor disclosed in any one of the following patent applications: WO 2022256808, WO 2022256806, WO 2019191470, and CN 115716831, each of which is incorporated herein by reference in its entirety, including the compound structures disclosed therein which are specifically incorporated herein by reference.

### ix) DOT1L

In some embodiments, compositions and methods described herein may include a compound of the present invention in combination with one or more Disruptor of Telomeric silencing 1-like (DOT1L) inhibitors. A DOT1L inhibitor may be administered or formulated in combination with a compound of the present invention and/or any additional therapeutic agent described herein. DOT1L is a histone methyltransferase enzyme that catalyzes the methylation of lysine 79 on histone H3. This modification is associated with transcriptional elongation and is important for the maintenance of gene expression programs. The DOT1L family includes enzymes that are involved in epigenetic regulation and transcriptional control, and their dysregulation has been linked to various diseases, including cancer. In some embodiments, a DOT1L inhibitor is one or more of EPZ-5676 (pinometostat) and EPZ-004777. In some embodiments, reference to the term DOT1L inhibitor includes any such DOT1L inhibitor disclosed in any one of the following patent applications: WO 2016090271, WO 2014100662, and CN 108997480, each of which is incorporated herein by reference in its entirety, including the compound structures disclosed therein which are specifically incorporated herein by reference.

iix) UBA1

In some embodiments, compositions and methods described herein may include a compound of the present invention in combination with one or more ubiquitin-activating enzyme inhibitors (e.g., a UBA1 inhibitor). A UBA1 inhibitor may be administered or formulated in combination with a compound of the present invention and/or any additional therapeutic agent described herein. UBA1, also known as ubiquitin-activating enzyme 1, is a key enzyme involved in the ubiquitination process, a fundamental cellular mechanism for protein degradation and regulation. Ubiquitination involves the covalent attachment of ubiquitin molecules to target proteins, marking them for degradation by the proteasome or modulating their activity, localization, or interactions within the cell. Several inhibitors have been developed to modulate UBA1 activity, with the aim of disrupting ubiquitination-mediated processes in diseased cells. These inhibitors include but are not limited to adenosine-based inhibitors which typically compete with ATP for binding to the active site of UBA1, thereby preventing the activation of ubiquitin (e.g., PYR-41 and MLN7243); covalent inhibitors which form irreversible bonds with specific amino acid residues in the active site of UBA1, leading to inhibition of its activity (e.g., TAK-243 (formerly known as MLN4924)); allosteric inhibitors which bind to sites on UBA1 distinct from the active site, inducing conformational changes that inhibit its catalytic activity (e.g., compound 2i); and fragment-based inhibitors which are designed based on smaller molecular fragments that bind to UBA1. In some embodiments, a UBA1 inhibitor is one or more of PYR-41, MLN7243, and TAK-243. In some embodiments, reference to the term UBA1 inhibitor includes any such UBA1 inhibitor disclosed in any one of the following patent applications: WO 2016069393 A1, WO 2016069392 A1, and JP 2013237627 A2, each of which is incorporated herein by reference in its entirety, including the compound structures disclosed therein which are specifically incorporated herein by reference.

### m) Ribonucleotide Reductase Inhibitors

Compositions and methods described herein may include a compound of the present disclosure in combination with one or more ribonucleotide reductase inhibitors (RNRi). RNR inhibitors are a class of compounds that inhibit the enzyme ribonucleotide reductase, which is essential for DNA synthesis and repair. RNR catalyzes the conversion of ribonucleotides (RNA building blocks) into deoxyribonucleotides (DNA building blocks), providing the necessary precursors for DNA replication and repair in proliferating cells. By inhibiting RNR, these compounds effectively limit the production of deoxyribonucleotides, thereby preventing DNA synthesis and halting the proliferation of rapidly dividing cells, such as cancer cells.

RNR is composed of two subunits: the R1 large subunit (containing the catalytic site) and the R2 small subunit (containing a di-iron center critical for enzymatic activity). RRIs typically act by binding to either the active site on the R1 subunit or the iron-oxygen complex in the R2 subunit, leading to the inhibition of the enzyme's activity. In some embodiments, a RNR inhibitor is a nucleoside analog inhibitor, an iron chelator, or an allosteric inhibitor. In some embodiments, a RNR inhibitor useful according to the present disclosure include but are not limited to one or more of hydroxyurea, triapine, didox, GTI-2040, CPI-613 (devimistat), and clofarabine. In some embodiments, reference to the term RNR inhibitor includes any such RNR inhibitor disclosed in any one of the following patent applications: WO 2025049814, WO 2022059691, WO 2022059692, WO 2021034776, WO 2019106579, WO 2014205179, WO 2013105088, WO 199312782, U.S. Pat. Nos. 5,071,835, 5,405,850, 4,814,432, and WO 199518815 each of which is incorporated herein by reference in its entirety, including the compound structures disclosed therein which are specifically incorporated herein by reference.

### n) Additional Therapeutic Agents Useful for Combination Therapy

In some embodiments, compositions and methods described herein may include a compound of the present invention in combination with one or more Farnesyl transferase inhibitors. A farnesyl transferase inhibitor may be administered or formulated in combination with a compound of the present invention and/or any additional therapeutic agent described herein. Farnesyl transferase inhibitors (FTIs) are a class of drugs that target the farnesyl transferase enzyme, which plays a role in a process called protein prenylation. Protein prenylation is an important step in the process of activating certain proteins involved in signal transduction, cell growth, and differentiation. In some embodiments, a farnesyl transferase inhibitor is one or more of tipifarnib, lonafarnib, and rilapladib. In some embodiments, reference to the term farnesyl transferase inhibitor includes any such farnesyl transferase inhibitor disclosed in any one of the following patent applications: WO 2010057028, WO 2007042465, WO 200136395, WO 200064891, WO 200042849, WO 199938862, WO 199928315, WO 199829390, WO 199426723, CN 107312000, CN 107365310, KR 100375421, KR 100388790, each of which is incorporated herein by reference in its entirety, including the compound structures disclosed therein which are specifically incorporated herein by reference.

In some embodiments, compositions and methods described herein may include a compound of the present invention in combination with one or more casein kinase inhibitors. In some embodiments, a casein inhibitor is, SR-3029, a potent and ATP competitive CK16 and CK1E inhibitor.

In some embodiments, compositions and methods described herein may include one or more FLT3 inhibitors in combination with a compound of the present invention disclosed herein. FLT3 (Fms-like tyrosine kinase 3), also known as CD135, is a receptor tyrosine kinase (RTK) that plays a crucial role in regulating hematopoiesis, the process by which blood cells are formed. It is primarily expressed on hematopoietic stem cells (HSCs) and progenitor cells in the bone marrow, where it controls cell proliferation, survival, and differentiation. In some embodiments, a FLT3 inhibitor includes, but are not limited to, midostaurin, gilteritinib, sorafenib, quizartinib, crenolanib, ponatinib and quizartinib.

In some embodiments, compositions and methods described herein may include a compound of the present invention in combination with one or more one or more TGFb pathway inhibitors. In some embodiments, compositions and methods described herein may include one or more TGFb inhibitors. A TGFb inhibitor may be administered or formulated in combination with a compound of the present invention and/or any additional therapeutic agent described herein. TGFb (transforming growth factor beta) is a multifunctional cytokine involved in various cellular processes, including cell growth, differentiation, apoptosis, and immune response. Dysregulation of the TGFb signaling pathway has been implicated in various diseases, including cancer, fibrosis, and autoimmune disorders. In some embodiments, a TGFb inhibitor is one or more of galunisertib (LY2157299), and vactosertib (TEW-7197). In some embodiments, a TGFb inhibitor is one or more of Galunisertib, LY2157299, Fresolimumab, Lerdelimumab, Trabedersen, curcumin, resveratrol and small interfering RNA (siRNA) to silence TGFb receptor expression. In some embodiments, reference to the term TGFb inhibitor includes any such TGFb inhibitor disclosed in any one of the following patent applications: WO 2023043473, WO 2020104648, WO 2020128850, WO 2016140884, WO 2007018818, WO 2004024159, WO 200226935, WO 2002062753, WO 2002062776, and JP 2012087076, each of which is incorporated herein by reference in its entirety, including the compound structures disclosed therein which are specifically incorporated herein by reference.

In some embodiments, compositions and methods described herein may include a compound of the present invention in combination with one or more HSP90 inhibitors. A HSP90 inhibitor may be administered or formulated in combination with a compound of the present invention and/or any additional therapeutic agent described herein. HSP90, also known as heat shock protein 90, is a molecular chaperone that plays a critical role in regulating the folding, stability, and activity of a large number of client proteins involved in various cellular processes, including cell cycle progression, signal transduction, and apoptosis. In some embodiments, a HSP90 inhibitor is one or more of Geldanamycin and its derivatives (e.g., 17-AAG, 17-DMAG), KOS 953, Radicicol and its derivatives (e.g., PU-H71), SNX-2112, Ganetespib, AT13387, Onalespib, Luminespib, and KW-2478. In some embodiments, reference to the term HSP90 inhibitor includes any such HSP90 inhibitor disclosed in any one of the following patent applications: WO 2021137665, WO 2018200534, WO 2017151425, WO 2015200514, WO 2013053833, WO 2013009657, WO 2013119985, WO 2012138894, WO 2011044394, WO 2009097578, WO 2008115719, CN 105237533, and CN 104030904, each of which is incorporated herein by reference in its entirety, including the compound structures disclosed therein which are specifically incorporated herein by reference.

In some embodiments, compositions and methods described herein may include a compound of the present invention in combination with one or more Glutathione peroxidase 4 (GPX4) inhibitors. A GPX4 inhibitor may be administered or formulated in combination with a compound of the present invention and/or any additional therapeutic agent described herein. GPX4 is an antioxidant enzyme that plays a critical role in protecting cells against oxidative stress-induced cell death. GPX4 catalyzes the reduction of lipid hydroperoxides to their corresponding alcohols and acts as a regulator of ferroptosis, a form of regulated cell death driven by lipid peroxidation. In some embodiments, a GPX4 inhibitor is one or more of RSL3, ML162, DPI7, FINO2, MCB-613, CBS9106, ML210, ODSH, and TLN232. In some embodiments, reference to the term GPX4 inhibitor includes any such GPX4 inhibitor disclosed in any one of the following patent applications: WO 2021132592, US 2021244715, and KR 20220115536, each of which is incorporated herein by reference in its entirety, including the compound structures disclosed therein which are specifically incorporated herein by reference.

In some embodiments, compositions and methods described herein may include a compound of the present invention in combination with one or more NRF2 inhibitors. A NRF2 inhibitor may be administered or formulated in combination with a compound of the present invention and/or any additional therapeutic agent described herein. NRF2 is a transcription factor that regulates the expression of genes involved in the cellular antioxidant response, detoxification, and other cytoprotective pathways. It plays a critical role in cellular defense mechanisms against oxidative stress and other forms of cellular damage. In some embodiments, a NRF2 inhibitor is one or more of ML385, Brusatol, CDDO-Im, RTA-408, and trigonelline. In some embodiments, reference to the term NRF2 inhibitor includes any such NRF2 inhibitor disclosed in any one of the following patent applications: WO 2023051088, WO 2021202720, KR 2022013610, and CN 107519168, each of which is incorporated herein by reference in its entirety, including the compound structures disclosed therein which are specifically incorporated herein by reference.

In some embodiments, compositions and methods described herein may include a compound of the present invention in combination with one or more TEA domain (TEAD) inhibitors. A TEAD inhibitor may be administered or formulated in combination with a compound of the present invention and/or any additional therapeutic agent described herein. TEAD is a family of transcription factors that play a key role in regulating gene expression during embryonic development and tissue homeostasis. The four members of the TEAD family (TEAD1-4) are transcriptional co-activators that bind to DNA through their conserved TEA domain and interact with other transcription factors to activate the expression of target genes. In some embodiments, a TEAD inhibitor is one or more of VT-107, a pan-TEAD, VT-104, Verteporfin, CA3, IAG933, K-975, IK-595, and Statins (see, e.g., Chapeau, Emilie and Schmelzle, Tobias (2023) IAG933, an oral selective YAP1-TAZ/pan-TEAD protein-protein interaction inhibitor (PPIi) with pre-clinical activity in monotherapy and combinations with MAPK inhibitors. Nature cancer). In some embodiments, reference to the term TEAD inhibitor includes any such TEAD inhibitor disclosed in any one of the following patent applications: WO 2023280254, WO 2023031781, WO 2022258040, WO 2020070181 WO 2018185266, and WO 2017064277, each of which is incorporated herein by reference in its entirety, including the compound structures disclosed therein which are specifically incorporated herein by reference.

In some embodiments, compositions and methods described herein may include a compound of the present invention in combination with one or more NOTCH/Gamma secretase inhibitors. A NOTCH/Gamma secretase inhibitor may be administered or formulated in combination with a compound of the present invention and/or any additional therapeutic agent described herein. In some embodiments, a NOTCH/Gamma secretase inhibitor is nirogacestat. In some embodiments, reference to the term NOTCH/Gamma secretase inhibitor includes any such NOTCH/Gamma secretase inhibitor disclosed in any one of the following patent applications: WO 2020208572, WO 2017200969, WO 2014047390, WO 2014047372, WO 2011041336, WO 2010090954, WO 2009008980, WO 2009087130, WO 2007110335, CN 103664904, CN 105560244, and KR 20200077480, each of which is incorporated herein by reference in its entirety, including the compound structures disclosed therein which are specifically incorporated herein by reference.

In some embodiments, compositions and methods described herein may include a compound of the present invention in combination with one or more Hedgehog inhibitors. A hedgehog inhibitor may be administered or formulated in combination with a compound of the present invention and/or any additional therapeutic agent described herein. The hedgehog (Hh) family of proteins are secreted signaling molecules that play a crucial role in embryonic development and tissue homeostasis in adults. The Hh signaling pathway is involved in regulating cell growth, differentiation, and survival. In some embodiments, a hedgehog inhibitor is one or more of Vismodegib (ERIVEDGE®), Sonidegib (ODOMZO®), and Glasdegib (DAURISMO™). In some embodiments, reference to the term hedgehog inhibitor includes any such hedgehog inhibitor disclosed in any one of the following patent applications: WO 2011063309, and CN 107163028, each of which is incorporated herein by reference in its entirety, including the compound structures disclosed therein which are specifically incorporated herein by reference.

Compositions and methods described herein may include a compound of the present invention in combination with one or more NFkB pathway inhibitors. An NFkB inhibitor may be administered or formulated in combination with a compound of the present invention and/or any additional therapeutic agent described herein. NF-kappa B (NFκB) is a family of transcription factors involved in regulating various cellular processes, including inflammation, immunity, cell survival, and proliferation. Non-limiting examples of NFkB inhibitors include Bortezomib (VELCADE®), Curcumin, Parthenolide, IKK inhibitors (e.g., IKK-16, BAY 11-7082), Resveratrol, Andrographolide and Proteasome inhibitors (e.g., MG132, lactacystin).

In some embodiments, the additional therapy is the administration of side-effect limiting agents (e.g., agents intended to lessen the occurrence or severity of side effects of treatment. For example, in some embodiments, the compound of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies include a non-drug treatment (e.g., surgery or radiation therapy). In some embodiments, the one or more additional therapies include a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies include a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor).

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, a compound of the present invention may be used as an adjuvant therapy after surgery. In some embodiments, a compound of the present invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, a compound of the present invention can render abnormal cells more sensitive to treatment with radiation for purposes of killing or inhibiting the growth of such cells. Accordingly, this disclosure further relates to a method for sensitizing abnormal cells in a mammal to treatment with radiation which comprises administering to the mammal an amount of a compound of the present disclosure, which amount is effective to sensitize abnormal cells to treatment with radiation. The amount of the compound in this method can be determined according to the means for ascertaining effective amounts of such compounds described herein. In some embodiments, a compound of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

In some embodiments, compositions and methods described herein may include a compound of the present invention in combination with one or more Claudin-18 targeting agents. A Claudin-18 targeting agents may be administered or formulated in combination with a compound of the present invention and/or any additional therapeutic agent described herein. Claudin-18 (e.g., claudin 18.2; CLDN18.2) has become a promising target for the treatment of patients with digestive malignancies, such as gastric cancer (GC), gastroesophageal junction (GEJ) cancer, esophageal cancer, and pancreatic cancer, because of its limited expression in healthy tissues and abnormal overexpression in a range of malignancies. Multiple clinical trials of CLDN18.2-targeted therapies, including monoclonal antibodies, bispecific antibodies, antibody-drug conjugates (ADCs), and chimeric antigen receptor (CAR) T-cell therapies, are ongoing, with some showing promising early results. Malignant transformation of gastric epithelial tissue leads to disruption of cell polarity and then to exposure of CLDN18.2 epitopes on the cell surface. Although targeted monoclonal antibodies are largely unable to access CLDN18.2 located in tight-junction supramolecular complexes in normal tissue, the perturbations in cell polarity that expose CLDN18.2 epitopes may theoretically enable CLDN18.2 targeted agents to bind to CLDN18.2 in malignant tissues with minimal off-target effects, making CLDN18.2 an attractive target for therapy. In some embodiments, a Claudin-18 targeting agent is one or more of Zolbetuximab, ASKB589, Osemitamab (TST001), PT886 (a bispecific antibody that targets CLDN18.2 and CD47), TJ-CD4B, CMG901 (an ADC that is composed of an antiCLDN18.2 monoclonal antibody joined to a cytotoxic payload, monomethyl auristatin E), and CT041 (autologous T cells genetically engineered to express a CLDN18.2-targeted CAR). In some embodiments, reference to the term Claudin-18 targeting agent includes any such Claudin-18 targeting agent disclosed in any one of the following patent applications: WO 2024081544, WO 2024131683, WO 2024137619, WO 2024140670, WO 2024136594, WO 2023034922, WO 2023046202, WO 2022203090, WO 2022133169, WO 2022100613, WO 2022256449, WO 2022136642, WO 2021155380, WO 2021129765, WO 2021011885, WO 2021058000, WO 2021218874, WO 2021027850, WO 2020156554, WO 2020025792, WO 2020114480, WO 2020211792, WO 2020239005, WO 2019219089, WO 2018157147, WO 2018108106, WO 2016166122, WO 2014146778, CN 118290582, CN 118203658, and CN 118286201, each of which is incorporated herein by reference in its entirety, including the compound structures disclosed therein which are specifically incorporated herein by reference.

In some embodiments, a therapeutic agent for combination therapy may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

An additional therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

An additional therapeutic agent may be an immune modulatory agent. For example, an additional therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, which interacts with a checkpoint protein. In some embodiments, the checkpoint inhibitor is an agent, such as an antibody, which interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002. Non-limiting examples of immune modulatory agent includes targets identified in Table 2.

TABLE 2
Exemplary Immune Modulatory Targets

| Target | Biological Function |
|---|---|
| CTLA-4 | Inhibitory Receptor |
| PD-1 | Inhibitory Receptor |
| PD-L1 | Ligand for PD-1 |
| LAG-3 | Inhibitory Receptor |
| B7.1 | Costimulatory Molecule |
| B7-H3 | Inhibitory Ligand |
| B7-H4 | Inhibitory Ligand |
| TIM3 | Inhibitory Receptor |
| VISTA | Inhibitory Receptor |
| CD137 | Costimulatory Molecule |
| OX-40 | Costimulatory Receptor |
| CD40 agonist | Costimulatory Molecule |
| CD40 agonist + FLT3 ligand | Costimulatory Molecule |
| CD27 | Costimulatory Receptor |
| CCR4 | Costimulatory Receptor |
| GITR | Costimulatory Receptor |
| NKG2D | Activating Receptor |
| KIR | Costimulatory Receptor |
| NKG2A | Inhibitory Receptor |
| ENPP1 | Inhibitory Receptor |
| TIGIT | Inhibitory Receptor |
| A2aR | Inhibitory Receptor |
| CD73 | Inhibitory Receptor |
| CD39 | Inhibitory Receptor |
| PVRIG | Inhibitory Receptor |
| IDO | Inhibitory enzyme |
| CSF1R | Inhibitory Receptor |
| LIF | Inhibitory Cytokine |
| CD47 | Inhibitory Receptor |
| SIRPa | Inhibitory Receptor |
| IL-2 | Effector Cytokines |
| IL-15 | Effector Cytokines |
| IL-12 | Effector Cytokines |
| TREM2 | Receptor |
| TGFb | Multifunctional Cytokine |
| CD73/TGFb trap | Multifunctional Cytokine |
| TCR-T cells directed to KRAS^{MUT}, | Cell therapy |
| mesothelin, or PRAME | |
| mRNA cancer vaccines | vaccines |
| BiTEs | Bi-specific T-cell engager |
| Dual EP2/EP4 inhibitor | E-prostanoid receptor |
| Gamma delta T Cells | Cell therapy |
| NK cells | Cell therapy |

Note:
CTLA4, cytotoxic T-lymphocyte-associated antigen 4; LAG3, lymphocyte activation gene 3; PD-1, programmed cell death protein 1; PD-L1, PD-1 ligand; TIM3, T cell membrane protein 3; VISTA, V-domain immunoglobulin (Ig)-containing suppressor of T-cell activation; KIR, killer IgG-like receptor, APC (Antigen Presenting Cells); TREM2 (Triggering receptor expressed on myeloid cells 2); TGF-b (Transforming growth factor beta).

An additional therapeutic agent may be an anti-TIGIT antibody, such as MBSA43, BMS-986207, MK-7684, COM902, AB154, MTIG7192A or OMP-313M32 (etigilimab).

In some embodiments, the combination therapy includes a compound of the present invention and a cancer vaccine composition. In some embodiments, the cancer vaccine composition is HB-700, mRNA-4157, mRNA-5671, BNT111, GVAX Pancreas, IMA901, DCVax, SOT101, Sipuleucel-T, PROSTVAC-VF or TG01.

An additional therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irinotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include GLEEVEC® (Imatinib Mesylate); KYPROLIS® (carfilzomib); VELCADE® (bortezomib); Casodex (bicalutamide); IRESSA® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammaII and calicheamicin omegaII (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel), ABRAXANE® (cremophor-free, albumin-engineered nanoparticle formulation of paclitaxel), and TAXOTERE® (doxetaxel); chloranbucil; tamoxifen (NOLVADEX™); raloxifene; aromatase inhibiting 4(5)-imidazoles; 4-hydroxytamoxifen; trioxifene; keoxifene; LY 117018; onapristone; toremifene (FARESTON®); flutamide, nilutamide, bicalutamide, leuprolide, goserelin; chlorambucil; GEMZAR® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; NAVELBINE® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., XELODA®); and pharmaceutically acceptable salts of any of the above.

Additional non-limiting examples of anti-cancer agents include trastuzumab (HERCEPTIN®), bevacizumab (AVASTIN®), cetuximab (ERBITUX®), rituximab (RITUXAN®), TAXOL®, ARIMIDEX®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, Ianiquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelaamine and thiotepa), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, DNA binding agents (e.g., ZALYPSIS®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as luteinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), cell surface monoclonal antibodies (e.g., anti-CD38 (HUMAX-CD38), anti-CSI (e.g., elotuzumab, PI3K/Akt inhibitors (e.g., perifosine), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), and cFMS inhibitors (e.g., ARRY-382).

In some embodiments, an anti-cancer agent is selected from mechlorethamine, camptothecin, ifosfamide, tamoxifen, raloxifene, gemcitabine, NAVELBINE®, sorafenib, or any analog or derivative variant of the foregoing. In some embodiments, the anti-cancer agent is JAB-3312.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune modulatory therapies, such as an immune checkpoint inhibitor. In some embodiments, a therapeutic agent may be a pan-RTK inhibitor, such as afatinib.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a RAS(ON) inhibitor of the present disclosure is used in combination with a MEK inhibitor and a SOS1 inhibitor. In some embodiments, a RAS(ON) inhibitor of the present disclosure is used in combination with a PD-L1 inhibitor and a SOS1 inhibitor. In some embodiments, a RAS(ON) inhibitor of the present disclosure is used in combination with a PD-L1 inhibitor and a SHP2 inhibitor. In some embodiments, a RAS(ON) inhibitor of the present disclosure is used in combination with a MEK inhibitor and a SHP2 inhibitor. In some embodiments, the cancer is colorectal cancer, and the treatment comprises administration of a Ras inhibitor of the present disclosure in combination with a second or third therapeutic agent.

Proteasome inhibitors include, but are not limited to, carfilzomib (KYPROLIS®), bortezomib (VELCADE®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with a compound of the present invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloproteinase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), EGFR inhibitory agents (e.g., antibodies or antigen binding regions that specifically bind thereto) such as VECTIBIX® (panitumumab), erlotinib (TARCEVA®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with a compound of the present invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with a compound of the present invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-Ia, interferon beta-Ib, interferon gamma, natural interferon gamma-Ia, interferon gamma-Ib, interleukin-1 beta, iobenguane, irinotecan, irsogladine, Ianreotide, LC 9018 (Yakult), leflunomide, lenograstim, lentinan sulfate, letrozole, leukocyte alpha interferon, leuprorelin, levamisole+fluorouracil, liarozole, lobaplatin, Ionidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with a compound of the present invention include ipilimumab (YERVOY®); tremelimumab; galiximab; nivolumab, also known as BMS-936558 (OPDIVO®); pembrolizumab (KEYTRUDA®); avelumab (BAVENCIO®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (IMFINZI®); MSB0010718C; AMP 224; adalimumab (HUMIRA®); ado-trastuzumab emtansine (KADCYLA®); aflibercept (EYLEA®); alemtuzumab (CAMPATH®); basiliximab (SIMULECT®); belimumab (BENLYSTA®); basiliximab (SIMULECT®); belimumab (BENLYSTA®); brentuximab vedotin (ADCETRIS®); canakinumab (ILARIS®); certolizumab pegol (CIMZIA®); daclizumab (ZENAPAX®); daratumumab (DARZALEX®); denosumab (PROLIA®); eculizumab (SOLIRIS®); efalizumab (RAPTIVA®); gemtuzumab ozogamicin (MYLOTARG®); golimumab (SIMPONI®); ibritumomab tiuxetan (ZEVALIN®); infliximab (REMICADE®); motavizumab (NUMAX®); natalizumab (TYSABRI®); obinutuzumab (GAZYVA®); ofatumumab (ARZERRA®); omalizumab (XOLAIR®); palivizumab (SYNAGIS®); pertuzumab (PERJETA®); pertuzumab (PERJETA®); ranibizumab (LUCENTIS®); raxibacumab (ABTHRAX®); tocilizumab (ACTEMRA®); tositumomab; tositumomab-i-131; tositumomab and tositumomab-i-131 (BEXXAR®); ustekinumab (STELARA®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### Enumerated Embodiments

E1. A compound of Formula (I):

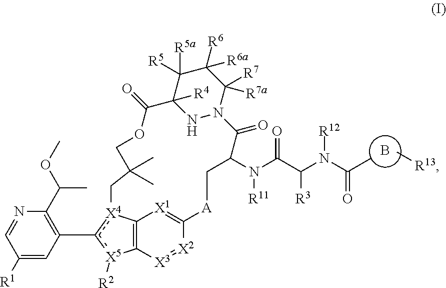

or a pharmaceutically acceptable salt thereof, wherein:
A is optionally substituted 3- to 6-membered cycloalkylene, optionally substituted 4- to 6-membered heterocycloalkylene, optionally substituted C₆₋₁₀ arylene, or optionally substituted 5- to 10-membered heteroarylene;
ring B is optionally substituted 4- to 11-membered heterocycloalkylene, wherein 1 or 2 ring carbon atoms of the 4- to 11-membered heterocycloalkylene are optionally replaced by a member independently selected from C(═O) and SO₂;
X¹, X² and X³ are each independently N or CR⁸, wherein each R⁸ is independently hydrogen, halogen, CN, or C₁₋₆ alkyl;
X⁴ is N and X⁵ is C; or
X⁵ is N and X⁴ is C;
[symbol] is either a single bond or a double bond to maintain the aromaticity of the 5-membered ring containing X⁴ and X⁵;
R¹ is hydrogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted 3- to 6-membered cycloalkyl, optionally substituted 3- to 6-membered cycloalkenyl, optionally substituted 4- to 14-membered heterocycloalkyl, optionally substituted 6- to 10-membered aryl, or optionally substituted 5- to 10-membered heteroaryl;
R² is optionally substituted C₁₋₆ alkyl, optionally substituted C₂₋₆ alkynyl, optionally substituted C₁₋₆ heteroalkyl, optionally substituted 3- to 6-membered cycloalkyl, or optionally substituted 4- to 14-membered heterocycloalkyl;
R³ is hydrogen, optionally substituted C₁₋₆ alkyl, optionally substituted C₁₋₃ heteroalkyl, optionally substituted C₃₋₆ cycloalkyl, or optionally substituted 4- to 6-membered heterocycloalkyl;
or R² and R³ together with the atoms to which they are attached form a fused C₄₋₁₀ cycloalkyl or a fused 4- to 10-membered heterocycloalkyl having 1, 2 or 3 ring heteroatoms selected from N, O, and S, wherein the fused C₄₋₁₀ cycloalkyl and heterocycloalkyl are each optionally substituted;
R⁴, R⁵, R^{5a}, R⁶, R^{6a}, R⁷ and R^{7a} are each independently hydrogen, halogen, or C₁₋₆ alkyl;
or R⁵ and R⁷ are taken together to form methylene or ethylene, which is covalently bound to the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form methylene or ethylene, which is covalently bound to the carbon atoms to which they are attached, and R⁵, R^{5a}, R^{6a}, R⁷ and R^{7a} are each independently hydrogen, halogen, or C₁₋₆ alkyl;
or R⁴ and R⁷ are taken together to form methylene or ethylene, which is covalently bound to the carbon atoms to which they are attached, R⁵, R^{5a}, R⁶, R^{6a}, and R^{7a} are each independently hydrogen, halogen, or C₁₋₆ alkyl;
or R⁵ and R⁶ taken together with the atoms to which they are attached form optionally substituted fused C₃₋₆ cycloalkyl or optionally substituted 4- to 6-membered fused heterocycloalkyl;
or R⁶ and R⁷ taken together with the atoms to which they are attached form optionally substituted fused C₃₋₆ cycloalkyl or optionally substituted 4- to 6-membered fused heterocycloalkyl;
or R⁵ and R^{5a} taken together with the atom to which they are attached form optionally substituted C₃₋₆ cycloalkyl or optionally substituted 4- to 6-membered heterocycloalkyl;
or R⁶ and R^{6a} taken together with the atom to which they are attached form optionally substituted C₃₋₆ cycloalkyl or optionally substituted 4- to 6-membered heterocycloalkyl;
or R⁷ and R^{7a} taken together with the atom to which they are attached form an optionally C₃₋₆ cycloalkyl or optionally substituted 4- to 6-membered heterocycloalkyl;
or R⁵ and R⁶ are taken together to form a bond;
R¹¹ is hydrogen or optionally substituted C₁₋₆ alkyl;
R¹² is hydrogen or optionally substituted C₁₋₆ alkyl; and
R¹³ is H, optionally substituted C₁₋₆ alkyl, —C(O)—R¹⁴, —C(O)OR¹⁴, —C(O)NHR¹⁴, —C(O)NR¹⁴R¹⁴, —SO₂R¹⁴, —SO₂NHR¹⁴, —SO₂NR¹⁴R¹⁴, wherein each R¹⁴ is independently optionally substituted C₁₋₆ alkyl, optionally substituted C₆₋₁₀ aryl, optionally substituted C₃₋₆ cycloalkyl, optionally substituted 5-6 membered heteroaryl, optionally substituted 4-14 membered heterocycloalkyl, optionally substituted C₆₋₁₀ aryl-C₁₋₆ alkyl-, optionally substituted C₃₋₆ cycloalkyl-C₁₋₆ alkyl-, optionally substituted 5-6 membered heteroaryl-C₁₋₆ alkyl-, or optionally substituted 4-14 membered heterocycloalkyl-C₁₋₆ alkyl.

E2. The compound of embodiment E1, or a pharmaceutically acceptable salt thereof, wherein X¹, X² and X³ are each CR⁸; X⁴ is C and X⁵ is N.

E3. The compound of embodiment E1, wherein the compound has the formula I-a:

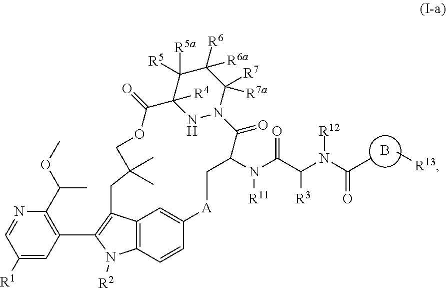

or a pharmaceutically acceptable salt thereof.

E4. The compound of any one of embodiments E1-E3, or a pharmaceutically acceptable salt thereof, wherein:
R⁵ and R⁷ are taken together to form methylene or ethylene, which is covalently bound to the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form methylene or ethylene, which is covalently bound to the carbon atoms to which they are attached, and R⁵, R^{5a}, R^{6a}, R⁷ and R^{7a} are each independently hydrogen, halogen, or C₁₋₆ alkyl;
or R⁴ and R⁷ are taken together to form methylene or ethylene, which is covalently bound to the carbon atoms to which they are attached, R⁵, R^{5a}, R⁶, R^{6a}, and R^{7a} are each independently hydrogen, halogen, or C₁₋₆ alkyl;
or R⁵ and R⁶ taken together with the atoms to which they are attached form optionally substituted fused C₃₋₆ cycloalkyl or optionally substituted 4- to 6-membered fused heterocycloalkyl;
or R⁶ and R⁷ taken together with the atoms to which they are attached form optionally substituted fused C₃₋₆ cycloalkyl or optionally substituted 4- to 6-membered fused heterocycloalkyl;
or R⁵ and R^{5a} taken together with the atom to which they are attached form optionally substituted C₃₋₆ cycloalkyl or optionally substituted 4- to 6-membered heterocycloalkyl;
or R⁶ and R^{6a} taken together with the atom to which they are attached form optionally substituted C₃₋₆ cycloalkyl or optionally substituted 4- to 6-membered heterocycloalkyl;
or R⁷ and R^{7a} taken together with the atom to which they are attached form an optionally C₃₋₆ cycloalkyl or optionally substituted 4- to 6-membered heterocycloalkyl;
or R⁵ and R⁶ are taken together to form a bond.

E5. The compound of any one of embodiments E1-E4, or a pharmaceutically acceptable salt thereof, wherein R¹² is H or methyl.

E6. The compound of any one of embodiments E1-E5, or a pharmaceutically acceptable salt thereof, wherein R³ is isopropyl.

E7. The compound of any one of embodiments E1-E6, or a pharmaceutically acceptable salt thereof, wherein R¹¹ is H.

E8. The compound of any one of embodiments E1-E6, or a pharmaceutically acceptable salt thereof, wherein R¹¹ is C₁₋₆ alkyl.

E9. The compound of any one of embodiments E1-E6, or a pharmaceutically acceptable salt thereof, wherein R¹¹ is methyl.

E10. The compound of embodiment E1, wherein the compound is of formula (II):

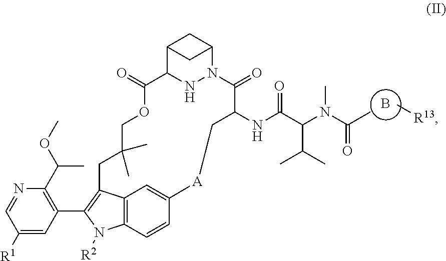

or a pharmaceutically acceptable salt thereof.

E11. The compound of embodiment E1, wherein the compound is of formula (III):

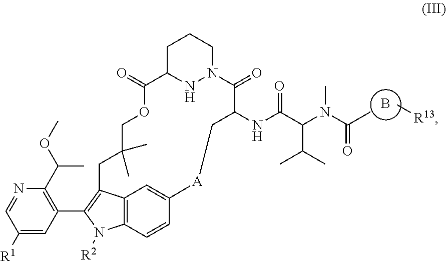

or a pharmaceutically acceptable salt thereof.

E12. The compound of any one of embodiments E1-E11, wherein A is of the formula:

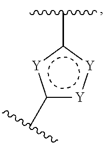

wherein
[symbol] indicates optional double bonds in the ring structure; and
each Y is independently CH, N, O, or S.

E13. The compound of embodiment E12, wherein A is

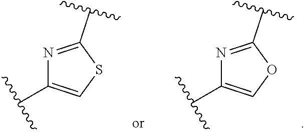 [SMILES: *c1csc(*)n1 ; *c1coc(*)n1]

E14. The compound of any one of embodiment E1-E11, wherein A is of the formula:

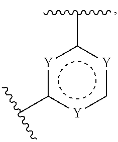

wherein
[symbol] indicates optional double bonds in the ring structure; and
each Y is independently CH or N.

E15. The compound of embodiment E14, wherein A is

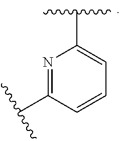 [SMILES: *c1cccc(*)n1]

E16. The compound of any one of embodiment E1-E11, wherein A is of the formula:

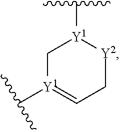

wherein
[symbol] is a single bond or a double bond;
each Y¹ is independently C, CH or N; and
Y² is CH₂, NH or O.

E17. The compound of embodiment E16, wherein A is

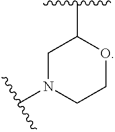 [SMILES: *C1CN(*)CCO1]

E18. The compound of embodiment E16, wherein A is

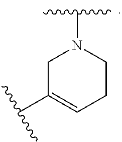 [SMILES: *C1=CCCN(*)C1]

E19. The compound of any one of embodiment E1-E18, or a pharmaceutically acceptable salt thereof, wherein ring B is optionally substituted 4- to 11-membered fused heterocycloalkyl or optionally substituted 4- to 11-membered spiro heterocycloalkyl or optionally substituted 4- to 6-membered heterocycloalkyl.

E20. The compound of any one of embodiments E1-E19, wherein B is of the formula:

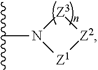

wherein,
n is 1, 2, or 3;
each of Z¹, Z², and Z³ is, independently, C═O, O, NR^{Z}, CH—R^{Z}, C(R^{Z})₂; and
each R^{Z} is independently H, optionally substituted C₁₋₆ alkyl, optionally substituted C₁₋₆ heteroalkyl, optionally substituted 4- to 6-membered cycloalkyl, optionally substituted 4- to 6-membered heterocycloalkyl, or two adjacent Z together form an optionally substituted 4- to 6-membered heterocycloalkyl or optionally substituted 4- to 6-membered cycloalkyl.

E21. The compound of embodiment E20, wherein Z¹ is C(R^{Z})₂ and each R^{Z} combine to form an optionally substituted 4-6 membered heterocycloalkyl.

E22. The compound of embodiments E20 or E21, wherein Z² is C(R^{Z})₂, wherein each R^{Z} is H.

E23. The compound of embodiments E20 or E21, wherein Z² is O.

E24. The compound of embodiments E20 or E21, wherein Z² is C(R^{Z})₂ and each R^{Z} combine to form an optionally substituted 4- to 6-membered heterocycloalkyl.

E25. The compound of embodiment E24, wherein Z¹ is C(R^{Z})₂, wherein each R^{Z} is H.

E26. The compound of embodiment E24, wherein Z¹ is C═O.

E27. The compound of embodiments E21 or E24, wherein said optionally substituted 4- to 6-membered heterocycloalkyl is selected from a group consisting of:

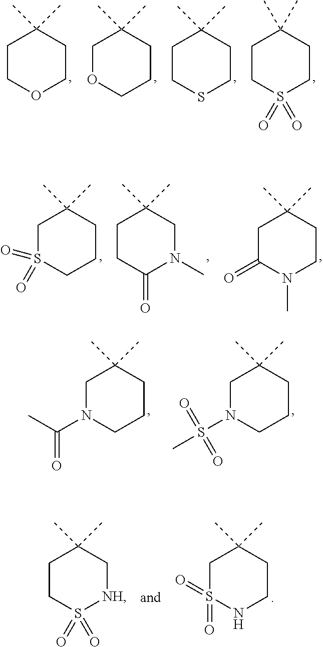

E28. The compound of embodiment E21 or E24, wherein said optionally substituted 4- to 6-heterocycloalkyl is selected from a group consisting of:

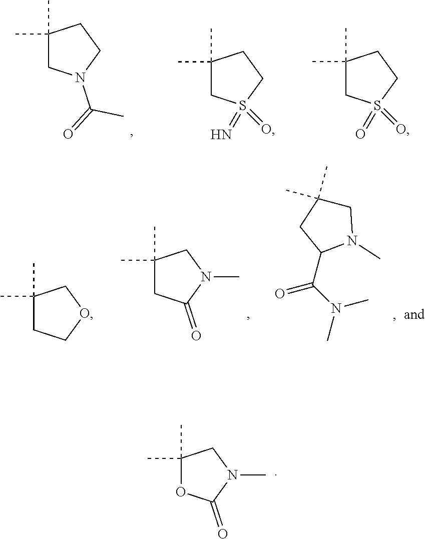

E29. The compound of embodiment E21 or E24, wherein said optionally substituted 4- to 6-membered heterocycloalkyl is selected from a group consisting of:

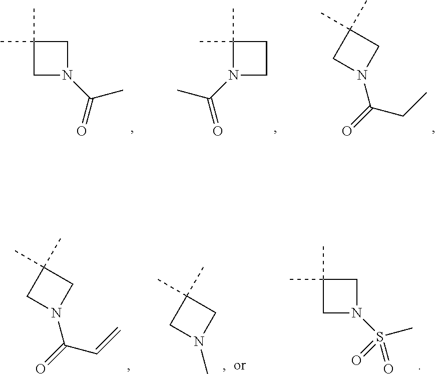

E30. The compound of embodiment E20, wherein Z¹ and Z² are each CH—R^{Z} and the R^{Z} from Z¹ and the R^{Z} from the adjacent Z² combine to form an optionally substituted 4- to 6-membered heterocycloalkyl.

E31. The compound of embodiment E30, wherein said optionally substituted 4- to 6-membered heterocycloalkyl is

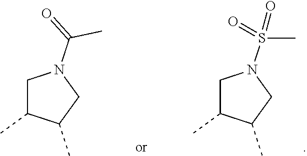

E32. The compound of embodiment E20, wherein Z² is NR^{Z} and the adjacent Z³ is CH—R^{Z}, and the R^{Z} from Z² and the R^{Z} from the adjacent Z³ combine to form an optionally substituted 4- to 6-membered heterocycloalkyl.

E33. The compound of embodiment E32, wherein said optionally substituted 4- to 6-membered heterocycloalkyl is

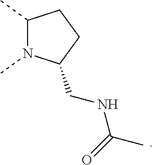

E34. The compound of embodiment E20, wherein Z¹ is C(R^{Z})₂ and each R^{Z} combine to form an optionally substituted 4- to 6-membered cycloalkyl.

E35. The compound of embodiment E34, wherein Z² is C(R^{Z})₂ and each R^{Z} is H.

E36. The compound of embodiment E34, wherein Z² is O.

E37. The compound of embodiment E20, wherein Z² is C(R^{Z})₂ and each R^{Z} combine to form an optionally substituted 4- to 6-membered cycloalkyl.

E38. The compound of embodiment E37, wherein Z¹ is C(R^{Z})₂, wherein each R^{Z} is H.

E39. The compound of embodiment E37, wherein Z¹ is C═O.

E40. The compound of any one of embodiments E34-E39, wherein said optionally substituted 4- to 6-membered cycloalkyl is selected from a group consisting of:

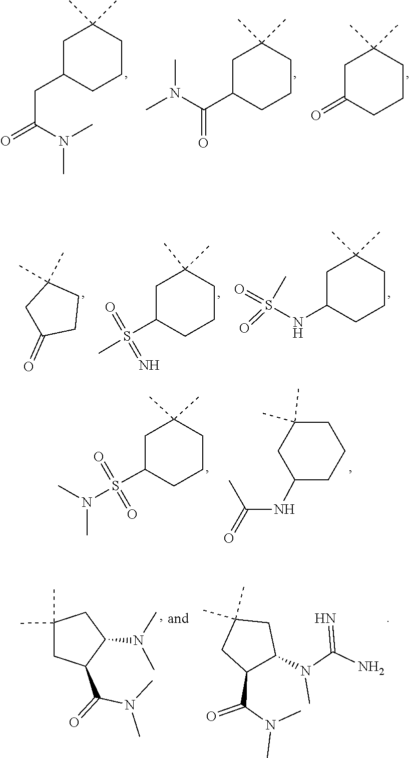

E41. The compound of any one of embodiments E20-E40, wherein each Z³ is CH—R^{Z} and R^{Z} is H.

E42. The compound of any one of embodiments E20-E40, wherein one Z³ is CH—R^{z} and R^{z} is CH₃.

E43. The compound of embodiment E20, wherein Z¹ is CH—R^{Z} and R^{Z} is

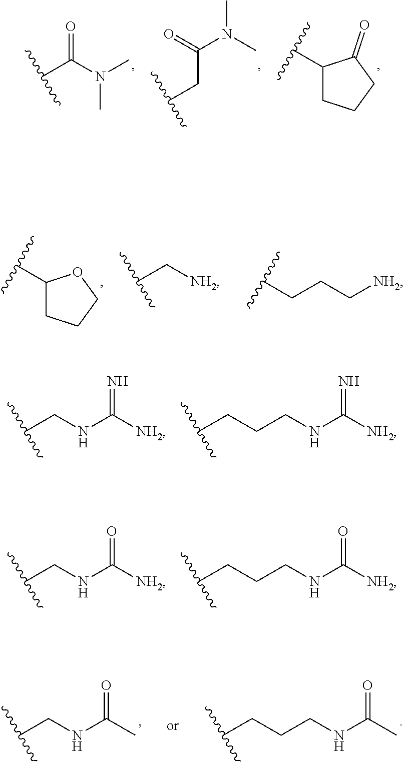

E44. The compound of embodiment E20, wherein Z² is CH—R^{Z} and R^{Z} is

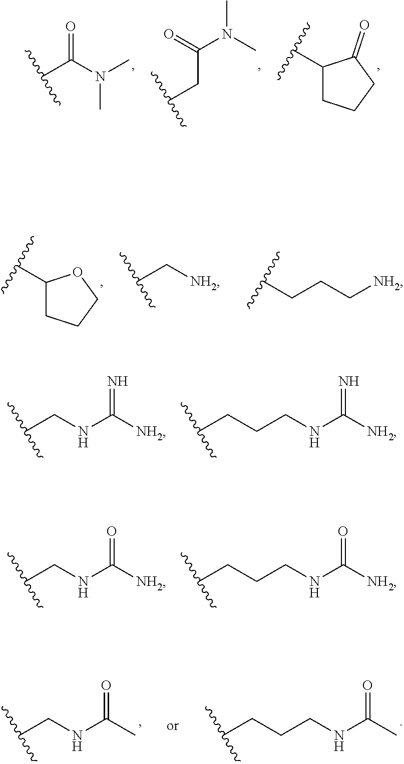

E45. The compound of embodiment E20, wherein Z¹ is C(R^{Z})₂, where one R^{Z} is CH₃ and the other R^{Z} is selected from the group consisting of:

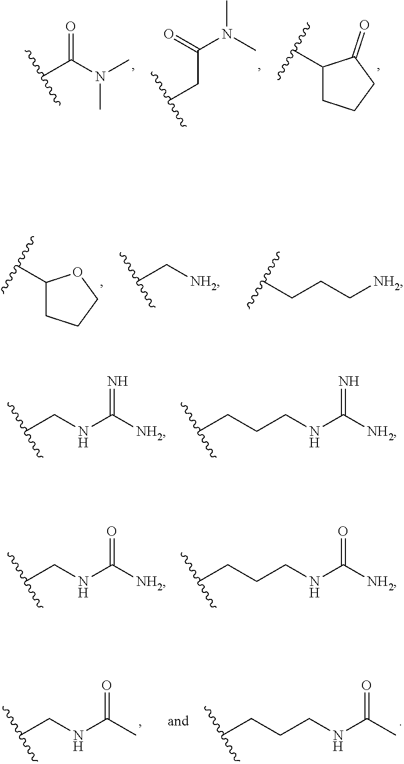

E46. The compound of embodiment E20, wherein Z¹ is C(R^{Z})₂ and one R^{Z} is H or CH₃ and the other R^{Z} is

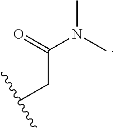 [SMILES: *CC(=O)N(C)C]

E47. The compound of any one of embodiments E1-E19, wherein B—R¹³ is selected from a group consisting of:

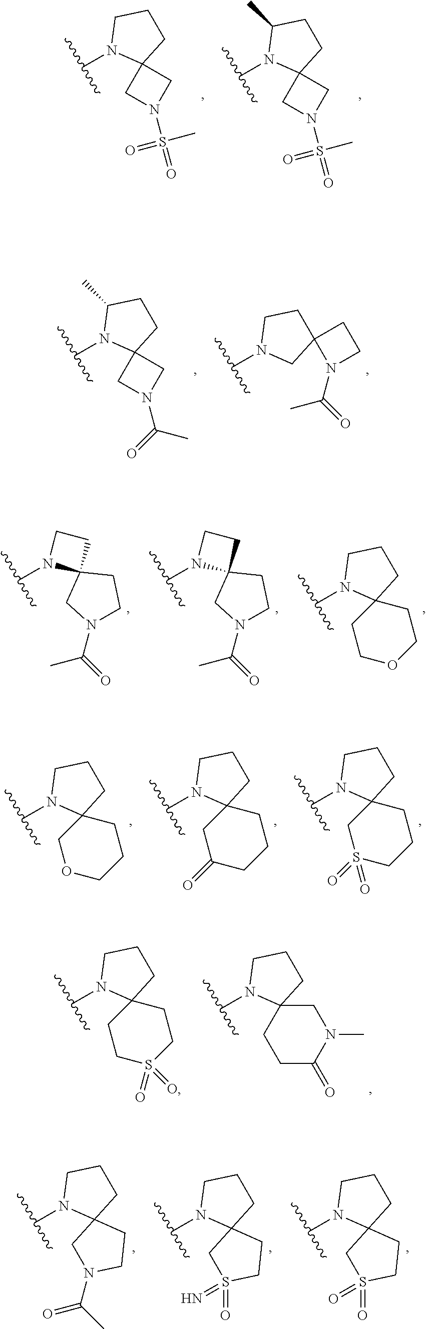

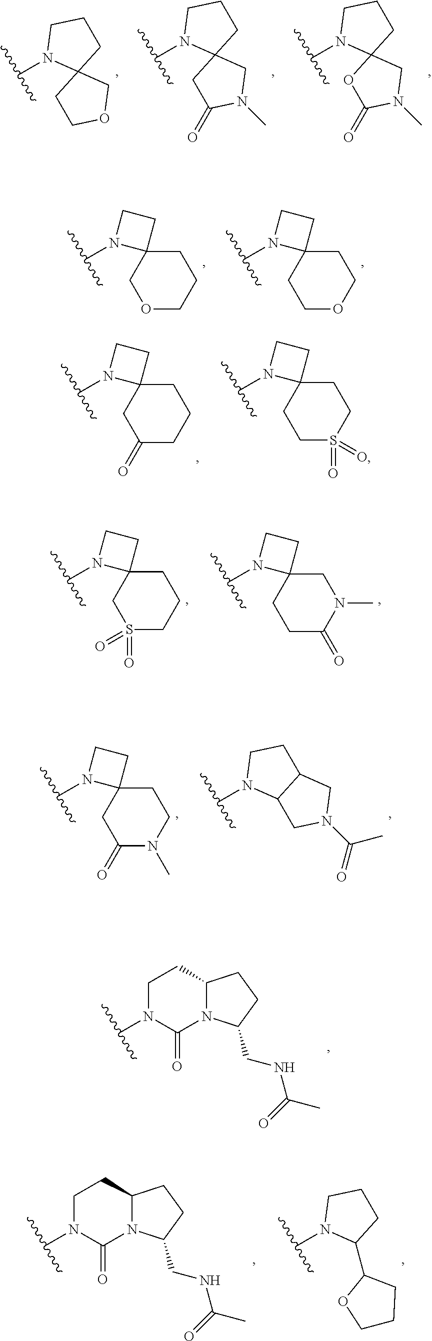

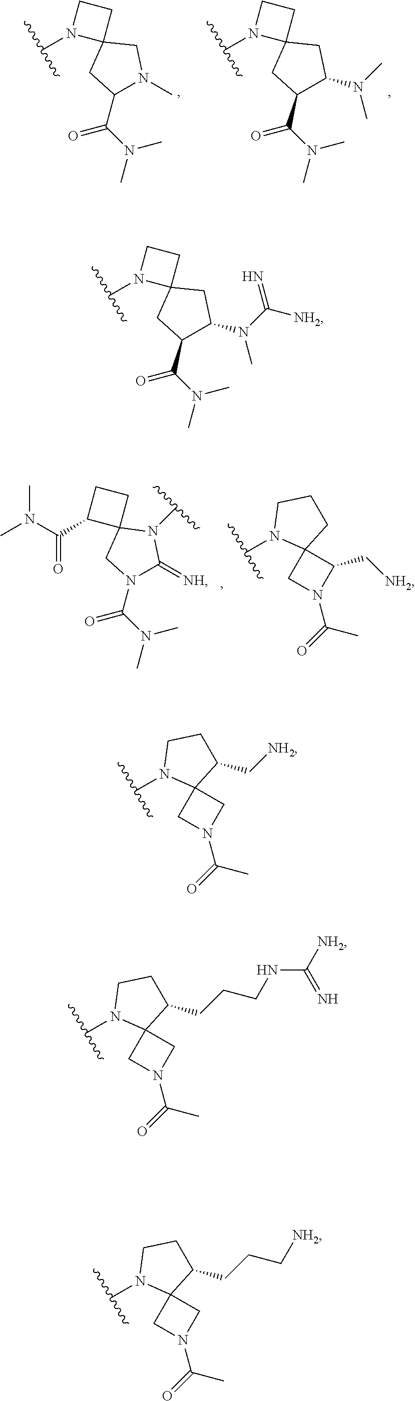

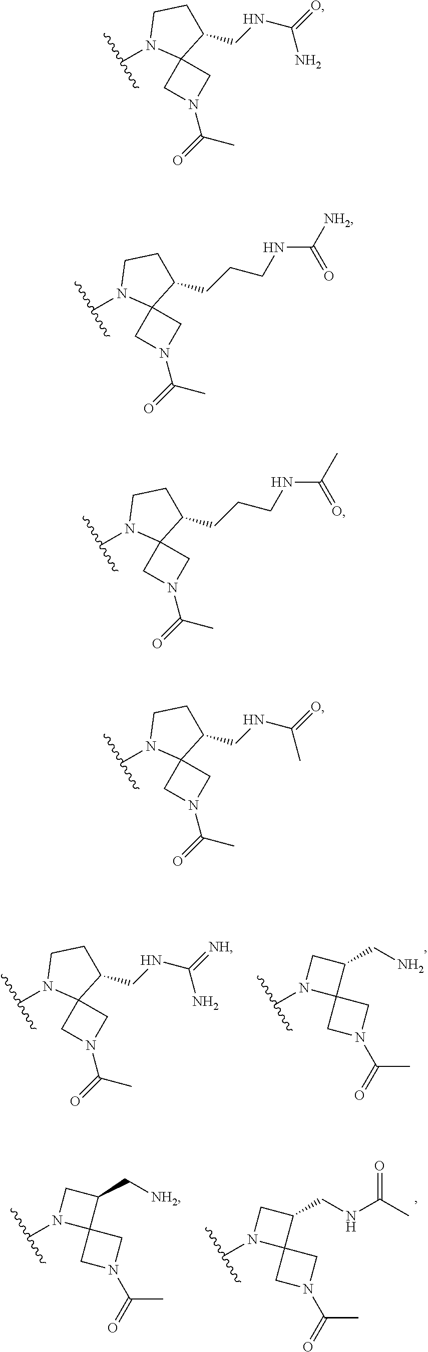

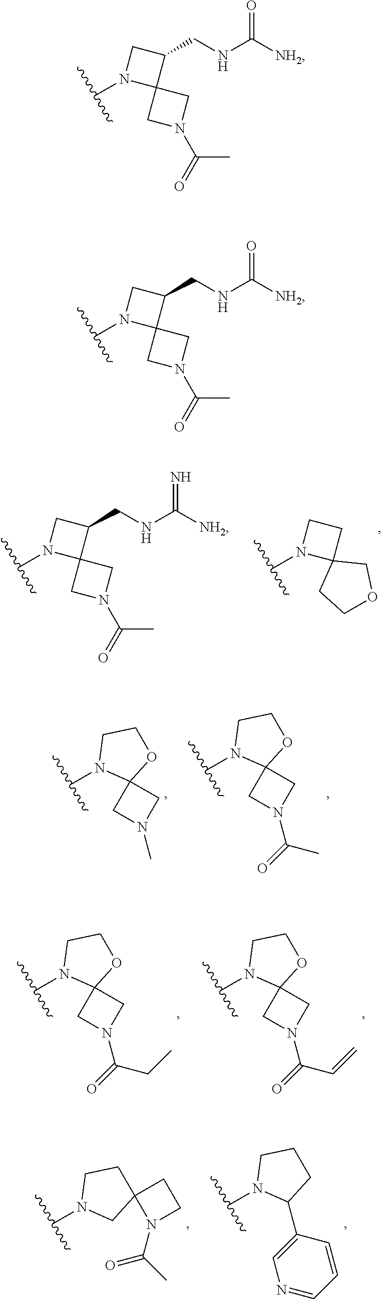

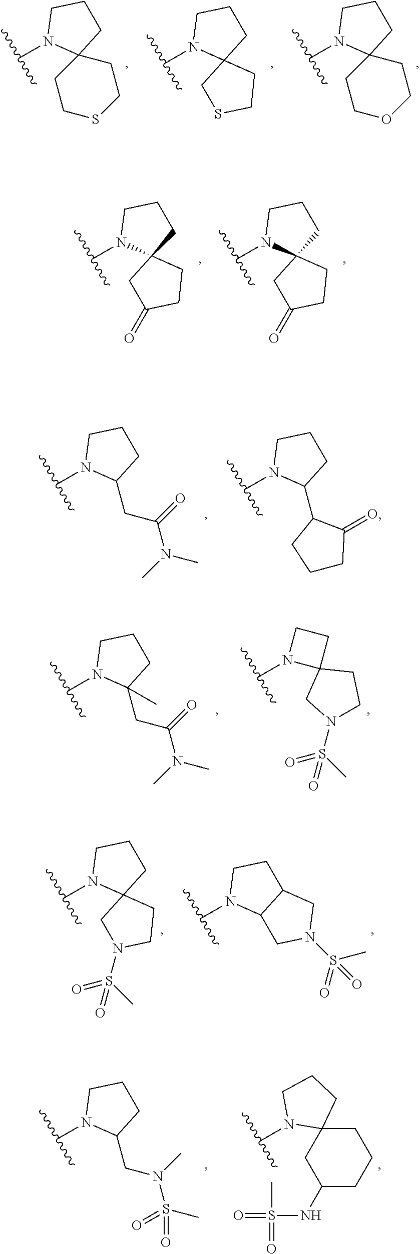

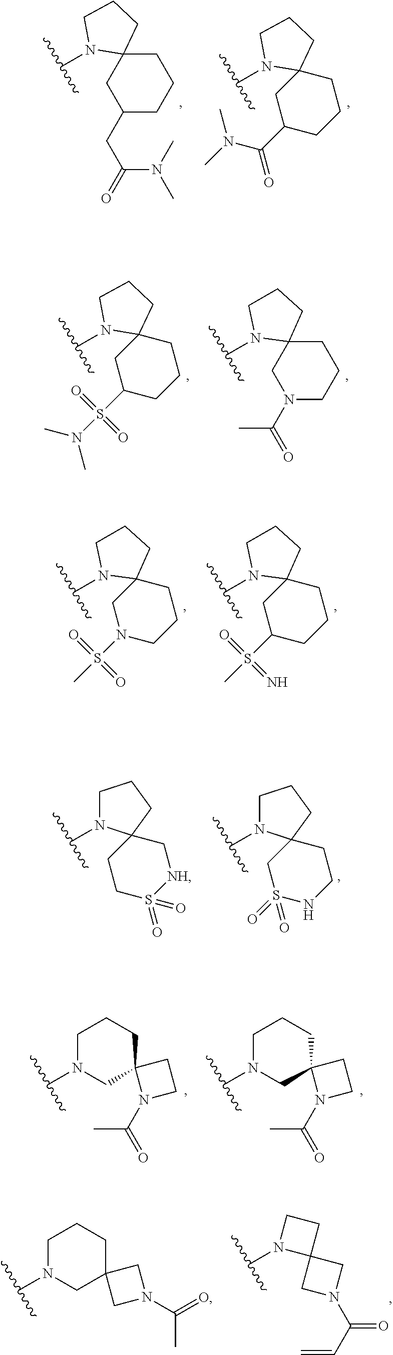

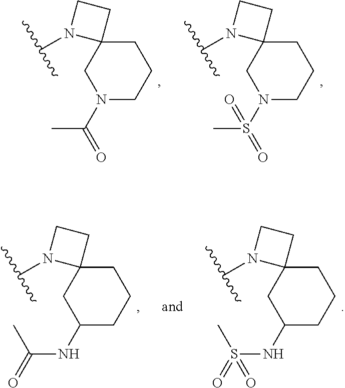 [SMILES: *N1CCC12CCCN(C(C)=O)C2 ; *N1CCC12CCCN(S(C)(=O)=O)C2 ; *N1CCC12CCCC(NC(C)=O)C2 ; *N1CCC12CCCC(NS(C)(=O)=O)C2]

E48. The compound of any one of embodiments E1-19, wherein B—R¹³ is selected from a group consisting of:

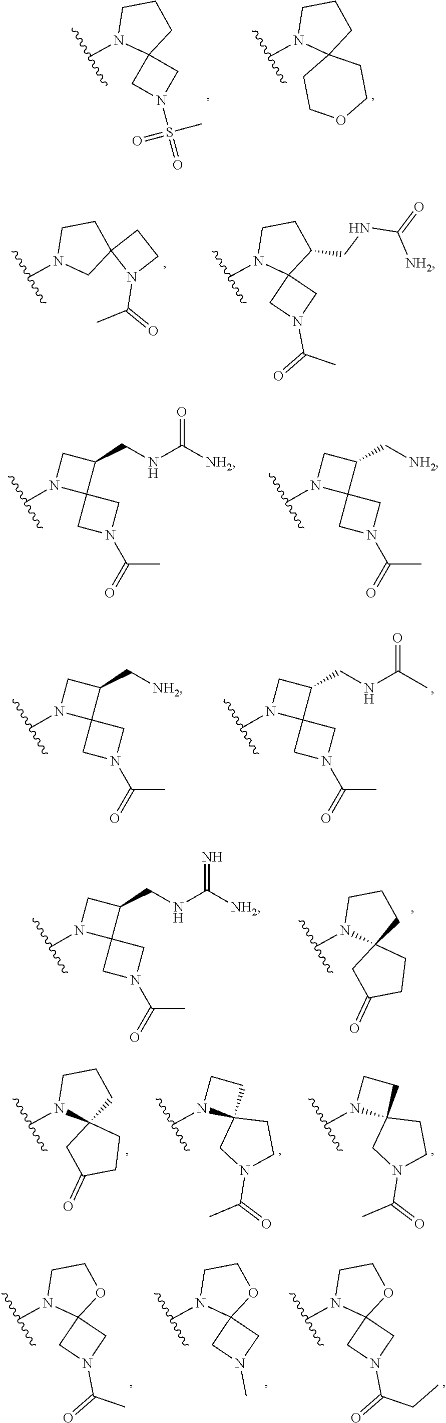

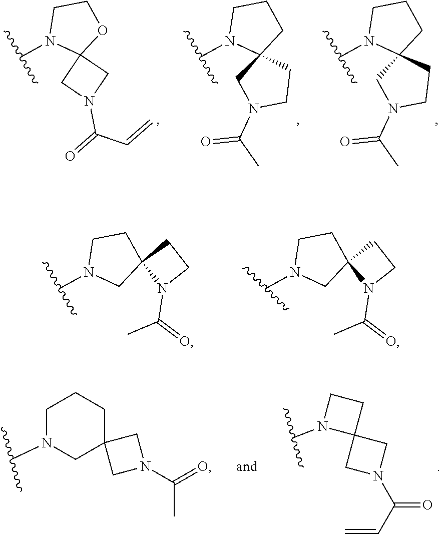

E49. The compound of any one of embodiments E1-E19, wherein B—R¹³ is selected from a group consisting of:

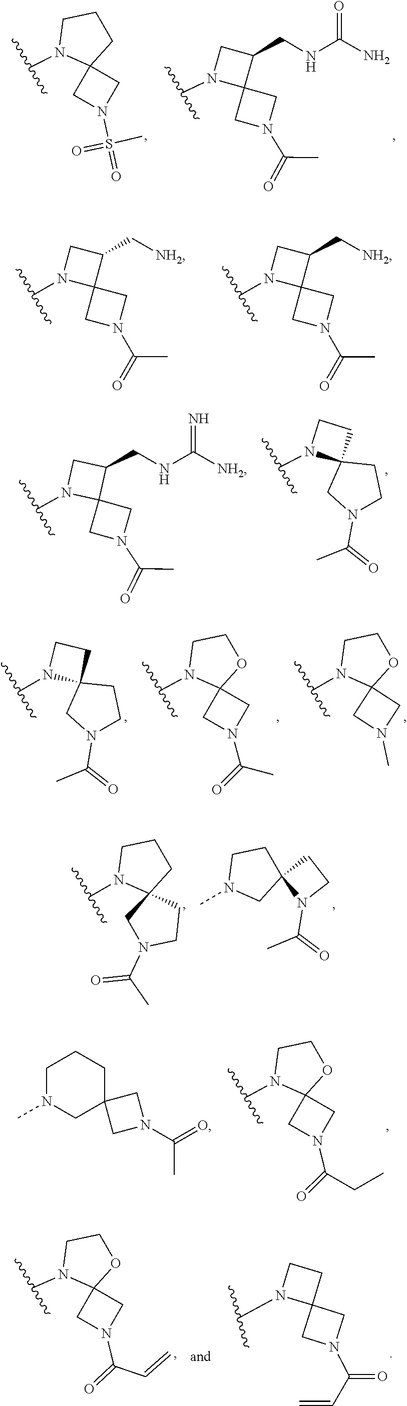

E50. The compound of any one of claims embodiments E1-E49, wherein R² is optionally substituted 4- to 14-membered heterocycloalkyl.

E51. The compound of embodiment E50, wherein R² is of the formula:

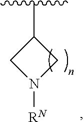

wherein,
N is 1, 2, or 3; and
R^{N} is C₁₋₆ alkyl, 3- to 6-membered cycloalkyl, or 4- to 6-membered heterocycloalkyl.

E52. The compound of embodiment E51, wherein R² is

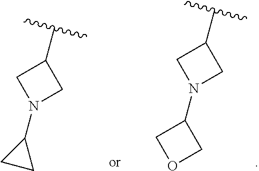 [SMILES: *C1CN(C2CC2)C1 ; *C1CN(C2COC2)C1]

E53. The compound of any one of claims embodiment E1-E49, wherein R² is optionally substituted C₁₋₆ alkyl.

E54. The compound of embodiment E53, wherein R² is

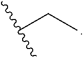 [SMILES: *CC]

E55. The compound of embodiment E53, wherein R² is

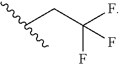 [SMILES: *CC(F)(F)F]

E56. The compound of any one of embodiments E11-E49, wherein R² is optionally substituted 3- to 6-membered cycloalkyl.

E57. The compound of embodiment E56, wherein R² is

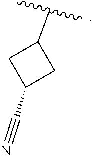

E58. A compound selected from compounds A1-A36 in Table 1 or a pharmaceutically acceptable salt thereof.

E59. A pharmaceutical composition comprising a compound, or a pharmaceutically acceptable salt thereof, of any one of embodiments E1-E58 and a pharmaceutically acceptable excipient or carrier.

E60. A method of treating cancer in a subject in need thereof, the method comprising administering to the subject a therapeutically effective amount of a compound, or a pharmaceutically acceptable salt thereof, of any one of embodiments E1 to E58 or a pharmaceutical composition of embodiment E59.

E61. The method of embodiment E60, wherein the cancer is melanoma.

E62. The method of embodiment E60, wherein the cancer is pancreatic cancer.

E63. The method of embodiment E60, wherein the cancer is colorectal cancer.

E64. The method of embodiment E60, wherein the cancer is non-small cell lung cancer.

E65. The method of embodiment E60, wherein the cancer is gastric cancer.

E66. The method of embodiment E60, wherein the cancer is esophageal cancer.

E67. The method of embodiment E60, wherein the cancer is ovarian cancer.

E68. The method of embodiment E60, wherein the cancer is uterine cancer.

E69. The method of any one of embodiments E60 or E68, wherein the cancer comprises a Ras mutation.

E70. The method of embodiment E69, wherein the Ras mutation is K-Ras G12, G13, Q61, or a combination thereof.

E71. A method of treating a Ras protein-related disorder in a subject in need thereof, the method comprising administering to the subject a therapeutically effective amount of a compound of any one of embodiments E1 to E58, or a pharmaceutically acceptable salt thereof, or a pharmaceutical composition of embodiment E59.

E72. The method of any one of embodiments E60-E71, wherein the method further comprises administering an additional anticancer therapy.

E73. The method of embodiment E72, wherein the additional anticancer therapy is an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CTLA-4 inhibitor, a CDK4/6 inhibitor, a HER2 inhibitor, an immune checkpoint inhibitor or a combination thereof.

E74. A method for inhibiting a RAS mutant in a cell, the method comprising: contacting the cell with a compound of any one of embodiments E1-E58, or pharmaceutically acceptable salt thereof, wherein the RAS mutant has a mutation at position G12, G13, Q61, or a combination thereof.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Instrumentation

Mass spectrometry data collection took place with a Shimadzu LCMS-2020, an Agilent 1260LC-6120/6125MSD, a Shimadzu LCMS-2010EV, or a Waters ACQUITY™ UPLC™, with either a QDa detector or SQ Detector 2. Samples in their liquid phase were injected onto a C-18 reverse phase column. The compounds were eluted from the column using an acetonitrile gradient and fed into the mass analyzer. Initial data analysis took place with either Agilent CHEMSTATION®, Shimadzu LABSOLUTIONS®, or Waters MASSLYNX™. NMR data was collected with either a Bruker AVANCE™ II HD 400 MHz, a Bruker Ascend 500 MHz instrument, or a Varian 400 MHz, and the raw data was analyzed with either TopSpin or Mestrelab Mnova.

### Chemical Syntheses

Definitions used in the following examples and elsewhere herein are:

| | |
|---|---|
| CaCl₂ | calcium chloride |
| CH₂Cl₂, DCM | methylene chloride, dichloromethane |
| CH₃CN, MeCN | acetonitrile |
| COMU | [[(Z)-(1-cyano-2-ethoxy-2-oxoethylidene)amino]oxy-morpholin-4- |
| | ylmethylidene]-dimethylazanium hexafluorophosphate |
| CuI | copper (I) iodide |
| DIPEA | diisopropylethylamine |
| DMA | N,N-dimethylacetamide |
| DMF | N,N-dimethylformamide |
| EDCI | 1-ethyl-3-(3-dimethylaminopropyl)carbodiimide |
| EtOAc | ethyl acetate |
| h | hour |
| H₂O | water |
| HATU | 1-[bis(dimethylamino)methylene]-1H-1,2,3-triazolo[4,5-b]pyridinium 3- |
| | oxide hexafluorophosphate |
| HCl | hydrochloric acid |
| HI | hydroiodic acid |
| HOBt | hydroxybenzotriazole |
| i-PrOH | propan-2-ol |
| K₃PO₄ | potassium phosphate (tribasic) |
| MeOH | methanol |
| MTBE | methyl tert-butyl ether |
| Na₂SO₄ | sodium sulfate |
| NMP | N-methyl pyrrolidone |
| Pd(dppf)Cl₂ | [1,1′-Bis(diphenylphosphino)ferrocene]dichloropalladium(II) |
| Pd(dtbpf)Cl₂ | [1,1′-Bis(di-tert-butylphosphino)ferrocene]dichloropalladium(II) |
| 2-MeTHF | 2-methyloxolane, 2-methyltetrahydrofuran |
| TFA | trifluoroacetic acid |
| THF | tetrahydrofuran, oxolane |

### Synthesis of intermediate (S)-3-(5-bromo-2-(2-(1-methoxyethyl)pyridin-3-yl)-1H-indol-3-yl)-2,2-dimethylpropan-1-ol

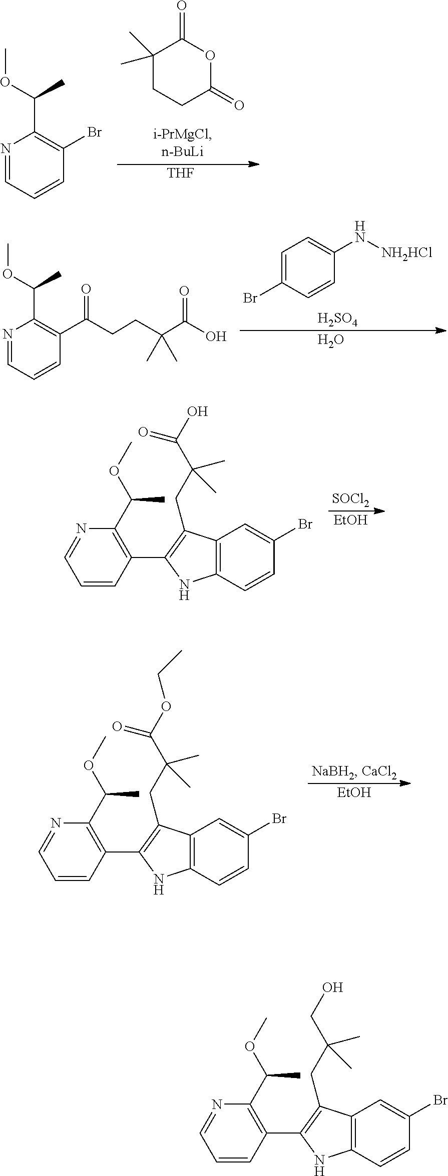

### Step 1

To a stirred mixture of i-PrMgCl (2 M in THF, 0.5 L) at −10° C. under an atmosphere of nitrogen was added n-BuLi (2.5 M in hexane, 333 mL, 833 mmol) dropwise over 15 min. The reaction mixture was stirred for 30 min at −10° C. under an atmosphere of nitrogen, then 3-bromo-2-[(1S)-1-methoxyethyl]pyridine (180 g, 833 mmol) in THF (0.5 L) added dropwise over 30 min at −10° C. The reaction mixture was warmed to −5° C. and stirred for 1 h, then 3,3-dimethyloxane-2,6-dione (118 g, 833 mmol) in THF (1.2 L) was added dropwise over 30 min at −5° C. The reaction mixture was warmed to 0° C. and stirred for 1.5 h. The resulting mixture was quenched by the addition of pre-cooled HCl (4 M in dioxane, 0.6 L) at 0° C. to adjust pH ˜5, diluted with H₂O (3 L) at 0° C., and extracted with EtOAc (3×2.5 L). The combined organic layers were dried over anhydrous Na₂SO₄, filtered, and the filtrate was concentrated under reduced pressure. The residue was purified by silica gel column chromatography to give 5-[2-[(1S)-1-methoxyethyl]pyridin-3-yl]-2,2-dimethyl-5-oxopentanoic acid (87 g, 34% yield) as a solid. LCMS (ESI): m/z [M+H] calcd for C₁₅H₂₁NO₄ 280.15; found 280.1.

### Step 2

To a stirred mixture of 5-[2-[(1 S)-1-methoxyethyl]pyridin-3-yl]-2,2-dimethyl-5-oxopentanoic acid (78 g, 279 mmol) in H₂SO₄ (31 mL, 558 mmol) and H₂O (78 mL) at room temperature under air atmosphere was added (4-bromophenyl)hydrazine HCl salt (68.7 g, 307 mmol) in portions. The resulting mixture was heated to 100° C. and stirred for 18 h. Then, the reaction mixture was cooled to 100° C. and was added NaOH (30% w/w solution, 74 mL, 558 mmol) dropwise. The resulting mixture was heated to 85° C. and stirred for an additional 3 h. The resulting mixture was cooled to room temperature and filtered to give 3-(5-bromo-2-[2-[(1S)-1-methoxyethyl]pyridin-3-yl]-1H-indol-3-yl)-2,2-dimethylpropanoic acid (90 g, 75%). LCMS (ESI): m/z [M+H] calcd for C₂₁H₂₃BrN₂O₃ 431.10; found 431.1.

### Step 3

To a stirred mixture of 3-(5-bromo-2-[2-[(1S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (90 g, 209 mmol) and EtOH (450 mL) at 50-60° C. was added SOCl₂ (22 mL, 307 mmol) under an atmosphere of nitrogen. The resulting mixture was stirred for 18 h at 50-60° C. Then the solution was cooled to room temperature and quenched by aqueous solution of Na₂CO₃ until pH 7-8. Then the mixture was filtered. The filter cake was washed with H₂O (450 mL) and dried for 60 h at 50-60° C. to afford ethyl (S)-3-(5-bromo-2-(2-(1-methoxyethyl)pyridin-3-yl)-1H-indol-3-yl)-2,2-dimethylpropanoate (91 g, 95% yield).

### Step 4

To a stirred mixture of ethyl (S)-3-(5-bromo-2-(2-(1-methoxyethyl)pyridin-3-yl)-1H-indol-3-yl)-2,2-dimethylpropanoate (91 g, 199 mmol) and in EtOH (1000 mL) at 35-45° C. was added NaBH₄ (16 g, 597 mmol) and CaCl₂ (22 g, 199 mmol) under air atmosphere. The resulting mixture was stirred for 12 h at 35-45° C. The mixture was cooled down to 20° C. and the pH was adjusted to pH 1-2 with 3M HCl. Then the mixture was added 30% wt NaOH till pH 4.5-5. Then the mixture was stirred for another 12 h at room temperature and filtered. The filter cake was washed with H₂O (1 L) and dried at 50° C. for 60 h to afford (S)-3-(5-bromo-2-(2-(1-methoxyethyl)pyridin-3-yl)-1H-indol-3-yl)-2,2-dimethylpropan-1-ol.

### Synthesis of intermediate (2S)—N-((6⁴S,4S,Z)-1¹-(1-cyclopropylazetidin-3-yl)-1²-(2-((S)-1-methoxyethyl)pyridin-3-yl)-1⁰,1⁰-dimethyl-5,7-dioxo-1¹H-8-oxa-6²,6³-diaza-2(4,2)-thiazola-1(5,3)-indola-6(2,4)-bicyclo[3.1.1]heptanacycloundecaphane-4-yl)-3-methyl-2-(methylamino)butanamide

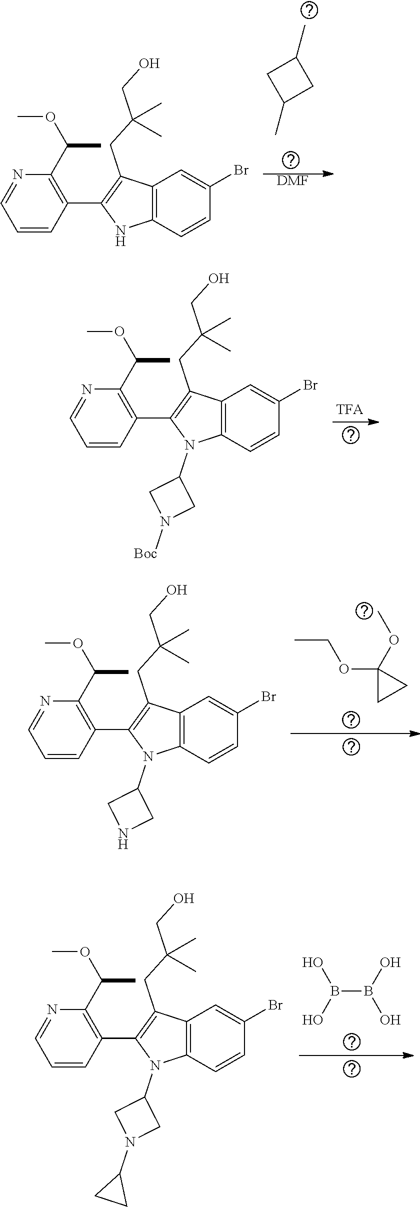

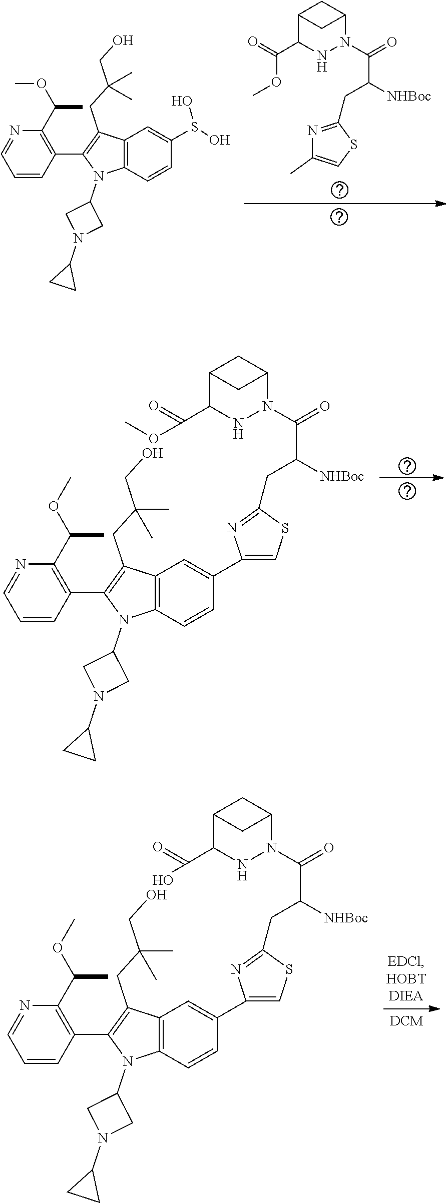

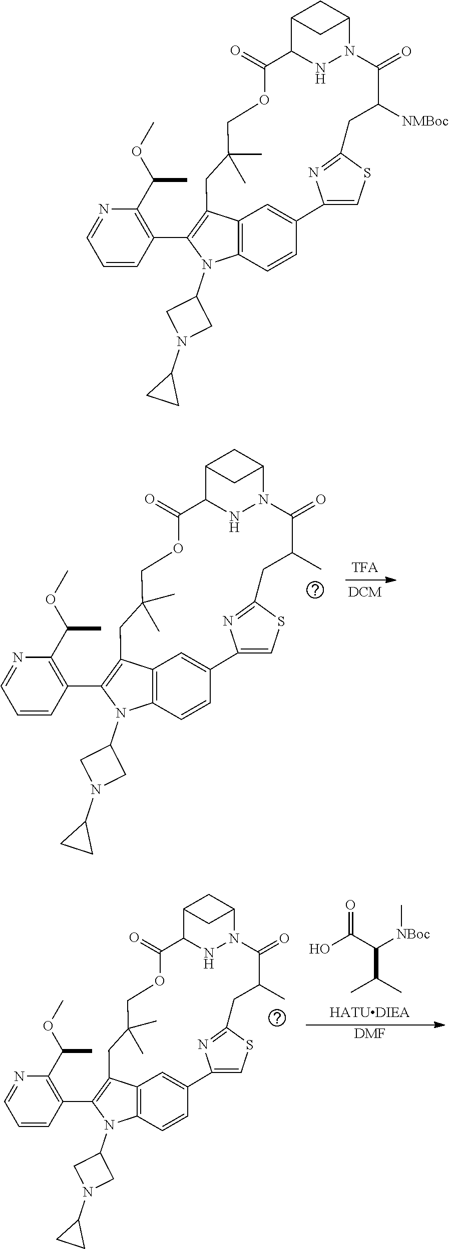

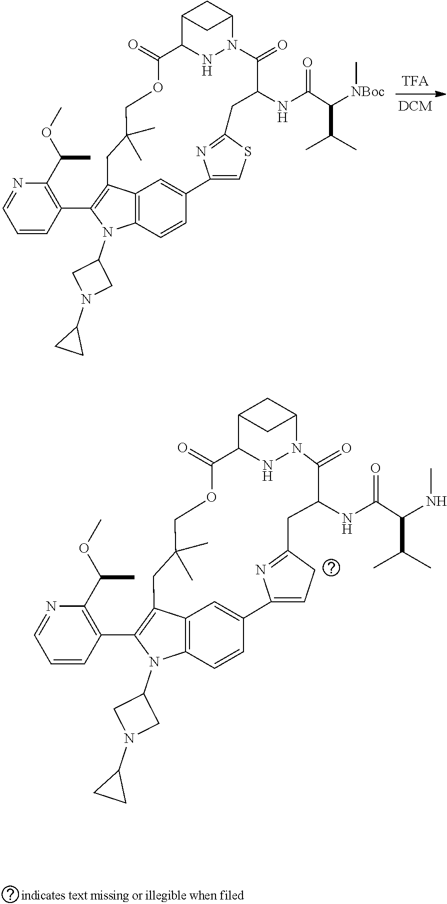 [SMILES: CO[C@@H](C)c1ncccc1-c1c2c3cc(ccc3n1C1CN(C3CC3)C1)-c1csc(n1)CC(NC(=O)[C@H](C(C)C)N(C)C(=O)OC(C)(C)C)C(=O)N1NC(C(=O)OCC(C)(C)C2)C2CC1C2 ; CN[C@H](C(=O)NC1CC2=NC(=CC2)c2ccc3c(c2)c(c(-c2cccnc2[C@H](C)OC)n3C2CN(C3CC3)C2)CC(C)(C)COC(=O)C2NN(C1=O)C1CC2C1)C(C)C]

### Step 1

To a stirred solution of (S)-3-(5-bromo-2-(2-(1-methoxyethyl) pyridin-3-yl)-1H-indol-3-yl)-2,2-dimethylpropan-1-ol (70.0 g, 0.167 mol) and Cs₂CO₃ (382 g, 0.117 mol) in DMF (500 mL) was added tert-butyl 3-iodoazetidine-1-carboxylate (332 g, 1.174 mol) in DMF (200 mL) dropwise at 80° C. under an atmosphere of nitrogen. The resulting mixture was stirred for an additional 12 h at 80° C. The reaction was quenched by addition of saturated NH₄Cl solution (500 mL) at 0° C. with vigorous stirring. The resulting mixture was extracted with EtOAc (3×1 L). The organic layers were washed with brine (3×1 L). The organic layers were combined, dried over Na₂SO₄, filtered and concentrated under reduced pressure. The residue was purified by silica gel column chromatography, eluted with PE/EtOAc (1:1) to afford tert-butyl (S)-3-(5-bromo-3-(3-hydroxy-2,2-dimethylpropyl)-2-(2-(1-methoxyethyl)pyridin-3-yl)-1H-indol-1-yl)azetidine-1-carboxylate (30 g, 30.8% yield) as a yellow solid. LCMS (ESI): m/z [M+H⁺] calc'd for C₂₉H₃₉BrN₃O₄ 572.2, 574.2; found 572.2, 574.2.

### Step 2

A solution of tert-butyl (S)-3-(5-bromo-3-(3-hydroxy-2,2-dimethylpropyl)-2-(2-(1-methoxyethyl)pyridin-3-yl)-1H-indol-1-yl)azetidine-1-carboxylate (39.1 g, 68.1 mmol) and TFA (195 mL) in DCM (390 mL) was stirred at 0° C. for 1 h under air atmosphere. The mixture was basified to pH 8 with saturated NaHCO₃ solution. The aqueous layer was extracted with CH₂Cl₂/MeOH (10:1) (3×300 mL). The resulting mixture was concentrated under reduced pressure. To the above mixture, methanol (390 mL), H₂O (390 mL), and K₂CO₃ (20 g) were added at 0° C. The resulting mixture was stirred at 20° C. for an additional 2 h. The reaction mixture was concentrated under reduced pressure to remove DCM and then extracted with CH₂Cl₂/MeOH (10:1) (3×300 mL). The combined organic layers were washed with brine (3×300 mL), dried over anhydrous Na₂SO₄, filtered and concentrated under reduced pressure. This resulted in (S)-3-(1-(azetidin-3-yl)-5-bromo-2-(2-(1-methoxyethyl)pyridin-3-yl)-1H-indol-3-yl)-2,2-dimethylpropan-1-ol (37 g, crude) as yellow oil. The crude product was used in the next step directly without further purification. LCMS (ESI): m/z [M+H⁺] calc'd for C₂₄H₃₁BrN₃O₂ 472.2, 474.2; found 472.2, 474.2.

### Step 3

To a stirred solution of (S)-3-(1-(azetidin-3-yl)-5-bromo-2-(2-(1-methoxyethyl)pyridin-3-yl)-1H-indol-3-yl)-2,2-dimethylpropan-1-ol (37.0 g, 78.3 mmol) and (1-ethoxycyclopropoxy)trimethylsilane (54.0 g, 313.2 mmol) in isopropanol (370 mL) were added AcOH (18.8 g, 313.2 mmol) and NaBH₃CN (19.6 g, 313.2 mmol) at 0° C. under an atmosphere of argon. The resulting mixture was stirred at 60° C. for 2 h under an atmosphere of argon. The reaction was quenched with ice water (500 mL) at 0° C. with vigorous stirring. The aqueous layer was extracted with EtOAc (3×500 mL). The resulting mixture was concentrated under reduced pressure. The residue was purified by silica gel column chromatography, eluted with CH₂Cl₂/MeOH (12:1) to afford (S)-3-(5-bromo-1-(1-cyclopropylazetidin-3-yl)-2-(2-(1-methoxyethyl)pyridin-3-yl)-1H-indol-3-yl)-2,2-dimethylpropan-1-ol (18 g, 44% yield) as a yellow solid. LCMS (ESI): m/z [M+H⁺] calc'd for C₂₇H₃₅BrN₃O₂ 512.2, 514.2; found 512.2, 514.2.

### Step 4

To a stirred mixture of (S)-3-(5-bromo-1-(1-cyclopropylazetidin-3-yl)-2-(2-(1-methoxyethyl)pyridin-3-yl)-1H-indol-3-yl)-2,2-dimethylpropan-1-ol (30.0 g, 58.5 mmol) and tetrahydroxydiborane (6.3 g, 70.2 mmol) in 2-methyltetrahydrofuran (200 mL) and methanol (100 mL) were added XPhos Pd G3 (4.9 g, 5.8 mmol) and potassium 2,2-dimethylpropanoate (16.4 g, 117.0 mmol) at 0° C. under argon atmosphere. The resulting mixture was stirred at 40° C. for additional 2 h. The reaction was poured into NH₄Cl solution (200 mL) at 0° C. The resulting mixture was extracted with EtOAc (2×200 mL). The combined organic layers were washed with brine (2×50 mL), dried over anhydrous Na₂SO₄, filtered and concentrated under reduced pressure. This resulted in (S)-(1-(1-cyclopropylazetidin-3-yl)-3-(3-hydroxy-2,2-dimethylpropyl)-2-(2-(1-methoxyethyl)pyridin-3-yl)-1H-indol-5-yl)boronic acid (30 g, crude) as a brown yellow solid. LCMS (ESI): m/z [M+H⁺] calc'd for C₂₇H₃₇BN₃O₄ 478.3; found 478.2.

### Step 5

To a stirred mixture of (S)-(1-(1-cyclopropylazetidin-3-yl)-3-(3-hydroxy-2,2-dimethylpropyl)-2-(2-(1-methoxyethyl)pyridin-3-yl)-1H-indol-5-yl)boronic acid (30.0 g, 62.8 mmol) and methyl (S)-2-((S)-3-(4-bromothiazol-2-yl)-2-((tert-butoxycarbonyl)amino)propanoyl)-2,3-diazabicyclo[3.1.1]heptane-4-carboxylate (33.8 g, 69.1 mmol) in toluene (300 mL), H₂O (100 mL) and 1,4-dioxane (100 mL) were added K₃PO₄ (33.3 g, 157.0 mmol) and Pd(dppf)Cl₂·CH₂Cl₂ (4.1 g, 5.0 mmol) at 0° C. under argon atmosphere. The resulting mixture was stirred at 80° C. for an additional 2 h. Desired product could be detected by LCMS. The reaction was poured into NH₄Cl solution (100 mL) at 0° C. The resulting mixture was extracted with EtOAc (2×100 mL). The combined organic layers were washed with brine (2×30 mL), dried over anhydrous Na₂SO₄, filtered and concentrated under reduced pressure. The residue was purified by silica gel column chromatography, eluted with CHCl₃/MeOH (10:1) to afford methyl (S)-2-((S)-2-((tert-butoxycarbonyl)amino)-3-(4-(1-(1-cyclopropylazetidin-3-yl)-3-(3-hydroxy-2,2-dimethylpropyl)-2-(2-((S)-1-methoxyethyl)pyridin-3-yl)-1H-indol-5-yl)thiazol-2-yl)propanoyl)-2,3-diazabicyclo[3.1.1]heptane-4-carboxylate (30 g, 56% yield) as a yellow solid. LCMS (ESI): m/z [M+H⁺] calc'd for C₄₅H₆₀N₇O₇S 842.4; found 842.4

### Step 6

To a stirred mixture of methyl (S)-2-((S)-2-((tert-butoxycarbonyl)amino)-3-(4-(1-(1-cyclopropylazetidin-3-yl)-3-(3-hydroxy-2,2-dimethylpropyl)-2-(2-((S)-1-methoxyethyl)pyridin-3-yl)-1H-indol-5-yl)thiazol-2-yl)propanoyl)-2,3-diazabicyclo[3.1.1]heptane-4-carboxylate (30.0 g, 35.6 mmol) in H₂O (50 mL) and THF (200 mL) were added LiOH·H₂O (4.2 g, 178.1 mmol) at 0° C. under air atmosphere. The resulting mixture was stirred at 20° C. for an additional 12 h. Desired product could be detected by LCMS. The residue was acidified to pH 6 with citric acid. The resulting mixture was extracted with EtOAc (2×200 mL). The combined organic layers were washed with H₂O (2×50 mL), dried over anhydrous Na₂SO₄, filtered and concentrated under reduced pressure. This resulted in (S)-2-((S)-2-((tert-butoxycarbonyl)amino)-3-(4-(1-(1-cyclopropylazetidin-3-yl)-3-(3-hydroxy-2,2-dimethylpropyl)-2-(2-((S)-1-methoxyethyl)pyridin-3-yl)-1H-indol-5-yl)thiazol-2-yl)propanoyl)-2,3-diazabicyclo[3.1.1]heptane-4-carboxylic acid (28 g, crude) as a light yellow solid. LCMS (ESI): m/z [M+H⁺] calc'd for C₄₄H₅₈N₇O₇S 828.4; found 828.5.

### Step 7

To a stirred mixture of (S)-2-((S)-2-((tert-butoxycarbonyl)amino)-3-(4-(1-(1-cyclopropylazetidin-3-yl)-3-(3-hydroxy-2,2-dimethylpropyl)-2-(2-((S)-1-methoxyethyl)pyridin-3-yl)-1H-indol-5-yl)thiazol-2-yl)propanoyl)-2,3-diazabicyclo[3.1.1]heptane-4-carboxylic acid (28.0 g, 33.8 mmol) and DIPEA (131.1 g, 1014.4 mmol) in DCM (1000 mL) were added EDCl (157.4 g, 1014.4 mmol) and HOBT (45.6 g, 338.1 mmol) at 0° C. under air atmosphere. The resulting mixture was stirred at 20° C. for an additional 2 h. The reaction was poured into NH₄Cl solution (100 mL) at 0° C. The resulting mixture was extracted with EtOAc (2×200 mL). The combined organic layers were washed with brine (2×100 mL), dried over anhydrous Na₂SO₄, filtered and concentrated under reduced pressure. The residue was purified by silica gel column chromatography, eluted with PE/EtOAc (1:4) to afford tert-butyl ((6⁴S,4S,Z)-1¹-(1-cyclopropylazetidin-3-yl)-1²-(2-((S)-1-methoxyethyl)pyridin-3-yl)-1⁰,1⁰-dimethyl-5,7-dioxo-1¹H-8-oxa-6²,6³-diaza-2(4,2)-thiazola-1(5,3)-indola-6(2,4)-bicyclo[3.1.1]heptanacycloundecaphane-4-yl)carbamate (15 g, 54% yield) as a yellow solid. LCMS (ESI): m/z [M+H⁺] calc'd for C₄₄H₅₆N₇O₆S 810.4; found 810.5.

### Step 8

To a stirred mixture of tert-butyl ((6⁴S,4S,Z)-1¹-(1-cyclopropylazetidin-3-yl)-1²-(2-((S)-1-methoxyethyl)pyridin-3-yl)-1⁰,1⁰-dimethyl-5,7-dioxo-1¹H-8-oxa-62,63-diaza-2(4,2)-thiazola-1(5,3)-indola-6(2,4)-bicyclo[3.1.1]heptanacycloundecaphane-4-yl)carbamate (13 g, 16.0 mmol) in DCM (150 mL) were added TFA (75 mL) at 0° C. under nitrogen atmosphere. The resulting mixture was concentrated under reduced pressure. The mixture was acidified to pH 8 with saturated NaHCO₃ solution (200 mL). The resulting mixture was extracted with EtOAc (2×100 mL). The combined organic layers were washed with brine (2×20 mL), dried over anhydrous Na₂SO₄, filtered and concentrated under reduced pressure. This resulted in (6⁴S,4S,2)-4-amino-1¹-(1-cyclopropylazetidin-3-yl)-1²-(2-((S)-1-methoxyethyl)pyridin-3-yl)-1⁰,1⁰-dimethyl-1¹H-8-oxa-6²,6³-diaza-2(4,2)-thiazola-1(5,3)-indola-6(2,4)-bicyclo[3.1.1]heptanacycloundecaphane-5,7-dione (13 g, crude) as a white solid. LCMS (ESI): m/z [M+H⁺] calc'd for C₃₉H₄₈N₇O₄S 710.3; found 710.5.

### Step 9

To a stirred mixture of (6⁴S,4S,2)-4-amino-1¹-(1-cyclopropylazetidin-3-yl)-1²-(2-((S)-1-methoxyethyl)pyridin-3-yl)-1⁰,1⁰-dimethyl-1¹H-8-oxa-6²,6³-diaza-2(4,2)-thiazola-1(5,3)-indola-6(2,4)-bicyclo[3.1.1]heptanacycloundecaphane-5,7-dione (13 g, 18.3 mmol) and DIPEA (23.6 g, 183.1 mmol) in DMF (100 mL) were added (2S)-2-[(tert-butoxycarbonyl)(methyl)amino]-3-methylbutanoic acid (8.4 g, 36.6 mmol) and HATU (10.4 g, 27.4 mmol) at 0° C. under nitrogen atmosphere. The resulting mixture was stirred at 20° C. for an additional 2 h. The reaction was poured into NH₄Cl solution (100 mL) at 0° C. The resulting mixture was extracted with EtOAc (2×100 mL). The combined organic layers were washed with brine (2×50 mL), dried over anhydrous Na₂SO₄, filtered and concentrated under reduced pressure. The residue was purified by silica gel column chromatography, eluted with PE/EtOAc (1:4) to afford tert-butyl ((2S)-1-(((6⁴S,4S,Z)-1¹-(1-cyclopropylazetidin-3-yl)-1²-(2-((S)-1-methoxyethyl)pyridin-3-yl)-1⁰,1⁰-dimethyl-5,7-dioxo-1¹H-8-oxa-6²,6³-diaza-2(4,2)-thiazola-1 (5,3)-indola-6(2,4)-bicyclo[3.1.1]heptanacycloundecaphane-4-yl)amino)-3-methyl-1-oxobutan-2-yl)(methyl)carbamate (12 g, 70% yield) as a white solid. LCMS (ESI): m/z [M+H⁺] calc'd for C₅₀H₆₇N₈O₇S 923.5; found 923.5.

### Step 10

To a stirred mixture of tert-butyl ((2S)-1-(((6⁴S,4S,Z)-1¹-(1-cyclopropylazetidin-3-yl)-1²-(2-((i)-1-methoxyethyl)pyridin-3-yl)-1⁰,1⁰-dimethyl-5,7-dioxo-1¹H-8-oxa-6²,6³-diaza-2(4,2)-thiazola-1(5,3)-indola-6(2,4)-bicyclo[3.1.1]heptanacycloundecaphane-4-yl)amino)-3-methyl-1-oxobutan-2-yl)(methyl)carbamate (6.0 g, 6.4 mmol) in DCM (60 mL) were added TFA (50 mL) in portions at 0° C. under nitrogen atmosphere. The resulting mixture was stirred at 20° C. for an additional 2 h. The resulting mixture was concentrated under reduced pressure. The mixture was basified to pH 8 with saturated NaHCO₃ solution (100 mL). The resulting mixture was extracted with EtOAc (2×50 mL). The combined organic layers were washed with NaCl (2×30 mL), dried over anhydrous Na₂SO₄, filtered and concentrated under reduced pressure. The residue was purified by reversed-phase flash chromatography with the following conditions (column, C18 silica gel; mobile phase, MeCN in Water (10 mmol/L NH₄HCO₃), 10% to 50% gradient in 10 min; detector, UV 254 nm) to afford (2S)—N-((6⁴S,4S,2)-1¹-(1-cyclopropylazetidin-3-yl)-1²-(2-((S)-1-methoxyethyl)pyridin-3-yl)-1⁰,1⁰-dimethyl-5,7-dioxo-1¹H-8-oxa-6²,6³-diaza-2(4,2)-thiazola-1(5,3)-indola-6(2,4)-bicyclo[3.1.1]heptanacycloundecaphane-4-yl)-3-methyl-2-(methylamino)butanamide (3.1 g, 58% yield, 98.4% purity at 254 nm, 98.0% purity at 220 nm) as a white solid. LCMS (ESI): m/z [M+H⁺] calc'd for C₄₅H₆₀N₈O₅S 823.4; found 823.4

### Synthesis of N-methyl-N—[(S)-2-methyl-1-[N-(7S,13S)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]-7,7-dioxo-7λ⁶-thia-1-aza-1-spiro[4.4]nonanecarboxamide

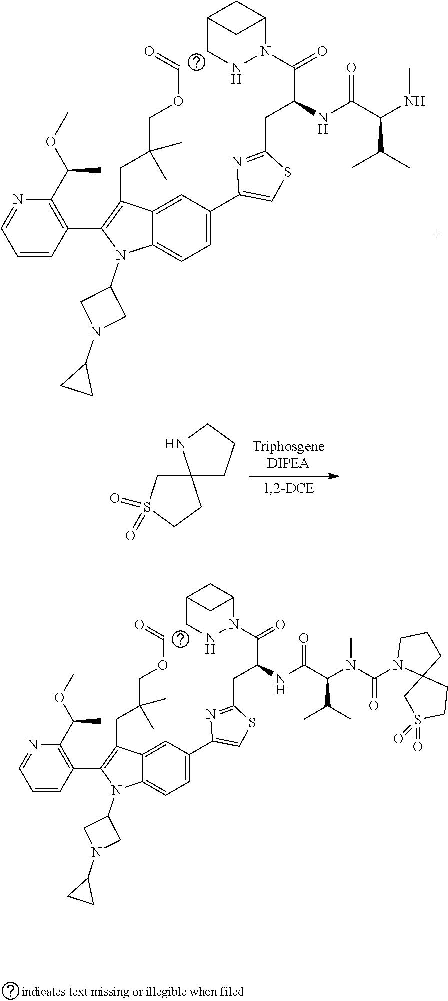 [SMILES: CN[C@H](C(=O)N[C@@H](Cc1nc(-c2ccc3c(c2)c(CC(C)(C)COC=O)c(-c2cccnc2[C@H](C)OC)n3C2CN(C3CC3)C2)cs1)C(=O)N1NCC2CC1C2)C(C)C ; O=S1(=O)CCC2(CCCN2)C1 ; CO[C@@H](C)c1ncccc1-c1c(CC(C)(C)COC=O)c2cc(-c3csc(C[C@H](NC(=O)[C@H](C(C)C)N(C)C(=O)N4CCCC45CCS(=O)(=O)C5)C(=O)N4NCC5CC4C5)n3)ccc2n1C1CN(C2CC2)C1]

To a stirred mixture of 7-thia-1-azaspiro[4.4]nonane 7,7-dioxide (63.9 mg, 364 μmol) and N-DIPEA (317 μL, 1.82 mmol) in 1,2-Dichloroethane (1.5 mL, 19 mmol) was added ditrichloromethyl carbonate (27 mg, 91.1 μmol) at room temperature under air atmosphere. The reaction was stirred at room temperature for 30 minutes and then added (2S)—N-((6⁴S,4S,Z)-1¹-(1-cyclopropylazetidin-3-yl)-1²-(2-((S)-1-methoxyethyl)pyridin-3-yl)-1⁰,1⁰-dimethyl-5,7-dioxo-1¹H-8-oxa-6²,6³-diaza-2(4,2)-thiazola-1(5,3)-indola-6(2,4)-bicyclo[3.1.1]heptanacycloundecaphane-4-yl)-3-methyl-2-(methylamino)butanamide (150 mg, 182 μmol). The resulting mixture was heated to 45° C. and stirred for 16 hours. The reaction was quenched by addition of MeOH (5 mL) and the reaction solution was concentrated under reduced pressure. The residue was dissolved in DMSO (2 mL) and acidified with formic acid (0.2 mL). The mixture was purified via Prep-HPLC (30-70% MeCN in H₂O with 0.1% formic acid) to afford N-methyl-N—[(S)-2-methyl-1-[N-(7S,13S)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]-7,7-dioxo-7λ⁶-thia-1-aza-1-spiro[4.4]nonanecarboxamide (50.02 mg, 25.35% yield) as a white powder. LCMS (ESI): m/z [M+K] calc'd for C₅₃H₆₉KN₉O₈S₂, 1062.4; found 1061.1.

### Synthesis of N-methyl-N—[(S)-2-methyl-1-[N-(7S,13S,20M)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]-(1R,5R)-7-acetyl-2,7-diazabicyclo[3.3.0]octane-2-carboxamide and N-methyl-N—[(S)-2-methyl-1-[N-(7S,13S,20M)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]-(1S,5S)-7-acetyl-2,7-diazabicyclo[3.3.0]octane-2-carboxamide

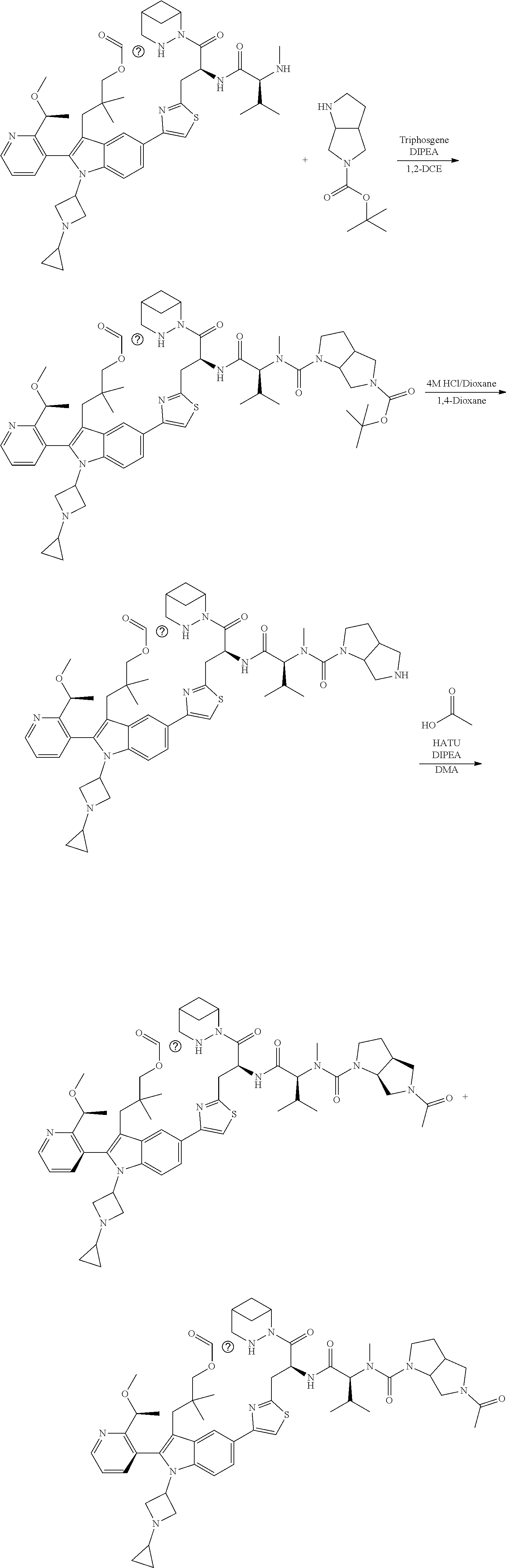

### Step 1

To a stirred mixture of tert-butyl 2,7-diazabicyclo[3.3.0]octane-7-carboxylate (103 mg, 486 μmol), and DIPEA (423 μL, 2.43 mmol) in 1,2-Dichloroethane (2 mL, 25.3 mmol) was added ditrichloromethyl carbonate (36.1 mg, 121 μmol) at room temperature under air. The resulting mixture was stirred for 30 minutes at room temperature and N-[(7S,13S)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-yl](S)-3-methyl-2-(methylamino)butyramide (0.2 g, 243 μmol) was then added. The reaction solution was heated to 45° C. and stirred for 16 hours, then quenched through addition of MeOH (5 mL). The mixture was concentrated under reduced pressure and the residue was purified via Prep-HPLC (20-60% MeCN in H₂O with 0.1% formic acid) to provide tert-butyl 2-{N-methyl[(S)-2-methyl-1-[N-(7S,13S)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]carbamoyl}-2,7-diazabicyclo[3.3.0]octane-7-carboxylate (55 mg, 31.8% yield) as a white solid. LCMS (ESI): m/z [M+K+] calc'd for C₅₇H₇₆KN₁₀O₈S 1099.5; found 1098.8

### Step 2

To a stirred mixture of tert-butyl 2-{N-methyl[(S)-2-methyl-1-[N-(7S,13S)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]carbamoyl}-2,7-diazabicyclo[3.3.0]octane-7-carboxylate (55 mg, 51.8 μmol) in 1,4-dioxane (0.5 mL, 5.86 mmol) was added 4M HCl in 1,4-dioxane (0.260 mL, 1.04 mmol) at room temperature under air. The reaction solution was stirred for 1 h at room temperature. The resulting mixture was quenched by addition of saturated NaHCO₃ solution (5 mL) at room temperature. The mixture was extracted with DCM (3×3 mL) and the combined organics were dried over MgSO₄ and concentrated under reduced pressure. The crude material N-methyl-N—[(S)-2-methyl-1-[N-(7S,13S)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]-2,7-diazabicyclo[3.3.0]octane-2-carboxamide (35.6 mg, 71.47% yield) was used directly with no further purification. LCMS (ESI): m/z [M+H⁺] calc'd for C₅₂H₆₈KN₁₀O₆S 999.5; found 998.1

### Step 3

To a stirred solution of N-methyl-N—[(S)-2-methyl-1-[N-(7S,13S)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]-2,7-diazabicyclo[3.3.0]octane-2-carboxamide (30 mg, 31.2 μmol), acetic acid (3.57 μL, 62.4 μmol), and DIPEA (27.2 μL, 156 μmol) in DMA (0.3 mL, 3.23 mmol) was added HATU (17.8 mg, 46.8 μmol) at room temperature under air atmosphere. The resulting mixture was stirred for 16 h at room temperature. The solution was acidified with formic acid (0.1 mL) and injected directly onto Prep-HPLC for purification (40-80% MeCN in H₂O with 0.1% formic acid) to afford N-methyl-N—[(S)-2-methyl-1-[N-(7S,13S,20M)-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]-(1R,5R)-7-acetyl-2,7-diazabicyclo[3.3.0]octane-2-carboxamide (2.05 mg, 6.55% yield) and N-methyl-N—[(S)-2-methyl-1-[N-(7S,13S,20A4-21-(1-cyclopropyl-3-azetidinyl)-20-{2-[(S)-1-methoxyethyl]-3-pyridyl}-17,17-dimethyl-8,14-dioxo-15-oxa-4-thia-9,21,27,29-tetraazahexacyclo[17.5.2.1²,⁵.1⁹,¹³.1¹⁰,¹².0²²,²⁶]nonacosa-1(24),2,5(29),19,22,25-hexaen-7-ylcarbamoyl]propyl]-(1S,5S)-7-acetyl-2,7-diazabicyclo[3.3.0]octane-2-carboxamide (2.1 mg, 6.71% yield) as white solids. LCMS (ESI): m/z [M+DMSO] calc'd for C₅₆H₇₆N₁₀O₈S₂ 1080.5; found 1082.0

TABLE 3
Exemplary Compounds Prepared by
Methods of the Present Invention

| | Exact Mass | MS Found (M + H⁺), except |
|---|---|---|
| Ex# | (g/mol) | where indicated |
| A1 | 1038.48 | 1061.2 [M + Na]⁺ |
| A2 | 989.52 | 990.4 |
| A3 | 1002.52 | 1003.3 |
| A4 | 1060.53 | 1061.6 |
| A5 | 1060.53 | 1061.5 |
| A6 | 1017.53 | 1018.6 |
| A7 | 1059.54 | 1060.5 |
| A8 | 1017.53 | 1018.6 |
| A9 | 1059.54 | 1060.6 |
| A10 | 1059.55 | 1060.6 |
| A11 | 1002.52 | 1003.7 |
| A12 | 1002.52 | 1003.7 |
| A13 | 987.50 | 988.5 |
| A14 | 987.50 | 988.4 |
| A15 | 1002.52 | 1003.7 |
| A16 | 1016.53 | 1017.7 |
| A17 | 1016.53 | 1017.6 |
| A18 | 998.54 | 999.7 |
| A19 | 1016.53 | 1017.7 |
| A20 | 1016.53 | 1017.7 |
| A21 | 1002.52 | 1003.7 |
| A22 | 1002.52 | 1003.7 |
| A23 | 1002.52 | 1003.7 |
| A24 | 1006.56 | 1007.7 |
| A25 | 988.46 | 989.7 |
| A26 | 1016.53 | 1017.7 |
| A27 | 960.52 | 961.6 |
| A28 | 988.52 | 989.7 |
| A29 | 1002.53 | 1003.5 |
| A30 | 1000.52 | 1001.7 |
| A31 | 1003.42 | 1004.4 |
| A32 | 1127.52 | 1128.5 |
| A33 | 1126.53 | 1127.5 |
| A34 | 1073.55 | 1074.7 |
| A35 | 1004.49 | 1005.6 |
| A36 | 988.50 | 989.5 |
| A37 | 881.4588 | 882.6 |
| A38 | 881.4588 | 882.6 |
| A39 | 882.4541 | 883.7 |
| A40 | 882.4541 | 883.6 |
| A41 | 897.4537 | 898.6 |
| A42 | 882.4541 | 883.6 |
| A43 | 847.4381 | 848.8 |
| A44 | 861.4537 | 862.7 |
| A45 | 903.4643 | 904.8 |
| A46 | 889.4487 | 890.8 |
| A47 | 889.4599 | 890.8 |
| A48 | 903.4755 | 904.8 |
| A49 | 926.5167 | 927.2 |
| A50 | 926.5167 | 927.2 |
| A51 | 925.5214 | 926.3 |
| A52 | 926.5167 | 927.3 |
| A53 | 926.5167 | 927.3 |
| A54 | 926.5167 | 927.2 |
| A55 | 929.5276 | 930.3 |
| A56 | 915.5007 | 916.3 |
| A57 | 926.5167 | 927.2 |
| A58 | 875.4694 | 876.8 |
| A59 | 917.48 | 918.8 |
| A60 | 917.4912 | 918.9 |
| A61 | 888.4534 | 889.9 |
| A62 | 901.3945 | 902.8 |
| A63 | 900.4534 | 901.9 |
| A64 | 928.4847 | 930 |
| A65 | 926.4439 | 927.9 |
| A66 | 896.4333 | 897.9 |
| A67 | 925.5214 | 926.2 |
| A68 | 954.5367 | 955.3 |
| A69 | 925.5214 | 926.2 |
| A70 | 931.4779 | 932.2 |
| A71 | 931.4779 | 932.2 |
| A72 | 926.5167 | 927.3 |
| A73 | 926.5167 | 927.3 |
| A74 | 929.5276 | 930.2 |
| A75 | 928.5323 | 929.2 |
| A76 | 915.5007 | 916.3 |
| A77 | 915.5007 | 916.2 |
| A78 | 888.4534 | 889.9 |
| A79 | 888.4534 | 889.9 |
| A80 | 918.444 | 919.9 |
| A81 | 914.4691 | 915.9 |
| A82 | 968.4408 | 969.8 |
| A83 | 900.4534 | 901.9 |
| A84 | 936.4346 | 937.9 |
| A85 | 936.4346 | 938 |
| A86 | 941.5163 | 942.1 |
| A87 | 941.5163 | 942.1 |
| A88 | 931.4779 | 932.2 |
| A89 | 928.5323 | 929.2 |
| A90 | 925.5214 | 926.2 |
| A91 | 926.5167 | 927.2 |
| A92 | 954.5367 | 955.2 |
| A94 | 895.4381 | 986.95 |
| A95 | 946.5065 | 947.9 |
| A96 | 960.5222 | 961.9 |
| A97 | 896.4333 | 897.94 |
| A98 | 924.4534 | 925.9 |
| A99 | 925.4487 | 926.96 |
| A100 | 925.4487 | 926.89 |
| A101 | 920.4333 | 921.8 |
| A102 | 963.4255 | 964.7 |
| A103 | 908.4585 | 909.9 |
| A104 | 944.5524 | 967.2 |
| A105 | 944.5524 | 967.2 |
| A106 | 931.4592 | 932.8 |
| A107 | 945.5113 | 946.9 |
| A108 | 965.4469 | 966.8 |
| A109 | 950.4502 | 951.8 |
| A110 | 902.4691 | 903.7 |
| A111 | 888.4898 | 889.8 |
| A112 | 888.4898 | 889.8 |
| A113 | 954.4752 | 955.9 |
| A114 | 924.4346 | 925.8 |
| A115 | 924.4346 | 925.8 |
| A116 | 940.4117 | 941.7 |
| A117 | 904.4306 | 905.8 |
| A118 | 936.4204 | 937.8 |
| A119 | 904.4306 | 905.8 |
| A120 | 936.4204 | 937.8 |
| A121 | 896.4333 | 897.8 |
| A122 | 964.4207 | 965.8 |
| A123 | 964.4207 | 965.8 |
| A124 | 925.4487 | 926.8 |
| A125 | 974.563 | 975.2 |
| A126 | 960.5837 | 961.1 |
| A127 | 974.563 | 975.1 |
| A128 | 910.4741 | 911.8 |
| A129 | 911.4694 | 912.8 |
| A130 | 874.4741 | 875.8 |
| A131 | 874.4741 | 875.8 |
| A132 | 886.4741 | 887.8 |
| A133 | 900.4898 | 901.9 |
| A134 | 895.4381 | 897 |
| A135 | 898.449 | 900 |
| A136 | 938.4803 | 940 |
| A137 | 952.4596 | 953.9 |
| A138 | 925.4487 | 927.1 |
| A139 | 939.4755 | 941 |
| A140 | 898.449 | 899.9 |
| A141 | 888.4898 | 889.9 |
| A142 | 902.4691 | 904 |
| A143 | 902.4691 | 904 |
| A144 | 914.4691 | 916.1 |
| A145 | 900.4898 | 902.1 |
| A146 | 904.4647 | 906.1 |
| A147 | 910.4553 | 912 |
| A148 | 888.4898 | 890.1 |
| A149 | 922.4775 | 922.9 |
| A150 | 922.4775 | 922.9 |
| A151 | 930.5367 | 931.5 |
| A152 | 930.5367 | 931.4 |
| A153 | 922.4775 | 923 |
| A154 | 900.4898 | 902.1 |
| A155 | 900.4898 | 902.1 |
| A156 | 928.5211 | 929.3 |
| A157 | 1027.59 | 1028.5 |
| A158 | 1068.616 | 1069.6 |
| A159 | 1084.611 | 1085.6 |
| A160 | 942.5367 | 943.4 |
| A161 | 942.5367 | 943.3 |
| A162 | 973.5426 | 974.4 |
| A163 | 942.5367 | 943.4 |
| A164 | 942.5367 | 943.4 |
| A165 | 915.4895 | 916.3 |
| A166 | 928.5211 | 929.4 |
| A167 | 928.5211 | 929.3 |
| A168 | 914.5054 | 915.5 |
| A169 | 888.4898 | 889.6 |
| A170 | 1024.554 | 1025.6 |
| A171 | 1024.554 | 1026.6 |
| A172 | 1038.606 | 1039.45 |
| A173 | 925.485 | 926.55 |
| A174 | 874.4741 | 875.4 |
| A175 | 888.4898 | 889.55 |
| A176 | 902.5054 | 903.45 |
| A177 | 1024.554 | 1025.1 |
| A178 | 870.4428 | 871.3 |
| A179 | 930.5004 | 931.6 |
| A180 | 902.4691 | 903.5 |
| A181 | 902.4691 | 903.5 |
| A182 | 918.5004 | 919.55 |
| A183 | 938.436 | 939.4 |
| A184 | 932.516 | 933.5 |
| A185 | 930.5004 | 931.1 |
| A186 | 930.5004 | 931.1 |
| A187 | 967.499 | 968.1 |
| A188 | 930.5004 | 931.55 |
| A189 | 875.4582 | 876.35 |
| A190 | 928.5211 | 929.1 |
| A191 | 928.5211 | 929 |
| A192 | 928.5211 | 929 |
| A193 | 898.4741 | 899.5 |
| A194 | 898.4741 | 899.55 |
| A195 | 1027.514 | 1028.6 |
| A196 | 961.5426 | 962.5 |
| A197 | 946.5117 | 987.4 |
| A198 | 1040.585 | 1041.1 |
| A199 | 1040.585 | 1041.1 |
| A200 | 915.4895 | 916.6 |
| A201 | 915.4895 | 916.6 |
| A202 | 902.5054 | 903.6 |
| A203 | 915.4895 | 916 |
| A204 | 915.4895 | 916.2 |
| A205 | 946.5117 | 987.4 |
| A206 | 943.532 | 944.4 |
| A207 | 928.5211 | 929.4 |
| A208 | 927.5007 | 928.4 |
| A209 | 926.5054 | 927.4 |
| A210 | 928.5211 | 929.4 |
| A211 | 926.5054 | 927.4 |
| A212 | 927.5007 | 928.4 |
| A213 | 928.5211 | 929.4 |
| A215 | 927.5007 | 928.4 |
| A216 | 914.5054 | 915.5 |
| A217 | 944.5524 | 967.2 |
| A218 | 870.4792 | 871 |
| A219 | 916.5211 | 917.4 |
| A220 | 928.5211 | 929.4 |
| A221 | 888.001 | 888.55 |
| A222 | 833.4476 | 834.55 |
| A223 | 942.5367 | 943.4 |
| A224 | 940.5211 | 941.4 |
| A225 | 928.5211 | 929.5 |
| A226 | 928.5211 | 929.55 |
| A227 | 863.4582 | 864.2 |
| A228 | 863.4582 | 864.2 |
| A229 | 872.4585 | 873.45 |
| A230 | 884.4585 | 885.55 |
| A231 | 912.4204 | 913.5 |
| A232 | 892.4847 | 893.55 |
| A233 | 858.4428 | 859.45 |
| A234 | 858.4428 | 859.45 |
| A235 | 924.4898 | 925.5 |
| A236 | 880.4272 | 881.55 |
| A237 | 924.4898 | 925.5 |
| A238 | 880.4272 | 881.45 |
| A239 | 880.4272 | 881.4 |
| A240 | 880.4272 | 881.45 |
| A241 | 924.4898 | 925.5 |
| A242 | 924.4898 | 925.55 |

Note:
values may differ slightly from values found elsewhere in this application due to different measurements and rounding.

### Example 1. Characterization of RAS(ON) GTP Hydrolysis-Promoting Compounds

The rate of RAS GTP hydrolysis was assayed in the presence of various KRAS mutations. To assess GTP hydrolysis activity, recombinant KRAS proteins (residue 1-169 of KRAS4B) were expressed in E. coli and purified using a TEV protease-cleavable His6-tag and Ni²⁺ affinity chromatography. The His6 tag was removed by treatment with TEV protease, and the KRAS protein was isolated by passing through a second Ni²⁺ column followed by size exclusion chromatography. The purified KRAS proteins were loaded with GTP by incubation for 2 hours on ice with 2 mM GTP and 10 mM Ethylenediaminetetraacetic Acid, followed by addition of 10 mM MgCl₂ and incubation on ice for 1 additional hour. The excess GTP was removed by overnight dialysis against buffer (12.5 mM HEPES, 75 mM NaCl, pH 7.5) at 4° C. The GTP-loaded KRAS protein were flash frozen in liquid nitrogen, then stored at −80° C. until use.

GTP-loaded KRAS proteins (1 μM) were combined with 25 μM of recombinant human cyclophilin A and 10 μM of compound in reaction buffer (12.5 mM HEPES, 75 mM NaCl, 1 mM MgCl₂, 1 mM DTT, 1% DMSO, pH 7.5) pre-warmed to 37° C. At fixed time points, aliquots were removed and quenched by heating to 80° C. to denature the proteins, then centrifuged to pellet the protein precipitate. The supernatant was assessed for GTP levels using Promega GTPase-Glo™ according to manufacturer instructions. The levels of GTP as a function of incubation time were fit to a single phase exponential decay to determine the rate constant for hydrolysis activity.

### Hydrolysis Half-Life

The hydrolysis activity of compounds A1-A36 against KRAS was characterized by their hydrolysis half-life measured at 1 uM against KRAS G12V. The hydrolysis activity of each compound was designated as “strong” if the half-life measured was less than 10 minutes, “medium” if the half-life measured was 10 minutes to 50 minutes, and “weak” if the half-life measured was greater than 50 minutes (Table 4).

TABLE 4
Hydrolysis Activity of Table 1 Compounds Against KRAS

| Compound | Hydrolysis Activity Against KRASG12V |
|---|---|
| A1 | strong |
| A2 | medium |
| A3 | medium |
| A4 | medium |
| A5 | strong |
| A6 | strong |
| A7 | medium |
| A8 | strong |
| A9 | strong |
| A10 | strong |
| A11 | weak |
| A12 | weak |
| A13 | medium |
| A14 | medium |
| A15 | strong |
| A16 | strong |
| A17 | strong |
| A18 | strong |
| A19 | strong |
| A20 | medium |
| A21 | strong |
| A22 | strong |
| A23 | medium |
| A24 | weak |
| A25 | strong |
| A26 | strong |
| A27 | medium |
| A28 | strong |
| A29 | strong |
| A30 | strong |
| A31 | strong |
| A32 | strong |
| A33 | strong |
| A34 | strong |
| A35 | strong |
| A36 | strong |
| A37 | medium |
| A38 | medium |
| A39 | weak |
| A40 | medium |
| A41 | medium |
| A42 | weak |
| A43 | weak |
| A44 | weak |
| A45 | weak |
| A46 | weak |
| A47 | weak |
| A48 | weak |
| A49 | weak |
| A50 | weak |
| A51 | weak |
| A52 | weak |
| A53 | weak |
| A54 | weak |
| A55 | weak |
| A56 | weak |
| A57 | weak |
| A58 | weak |
| A59 | weak |
| A60 | weak |
| A61 | weak |
| A62 | weak |
| A63 | medium |
| A64 | weak |
| A65 | strong |
| A66 | weak |
| A67 | weak |
| A68 | weak |
| A69 | weak |
| A70 | weak |
| A71 | weak |
| A72 | weak |
| A73 | weak |
| A74 | weak |
| A75 | weak |
| A76 | weak |
| A77 | weak |
| A78 | weak |
| A79 | weak |
| A80 | medium |
| A81 | medium |
| A82 | weak |
| A83 | weak |
| A84 | weak |
| A85 | weak |
| A86 | weak |
| A87 | weak |
| A88 | weak |
| A89 | weak |
| A90 | weak |
| A91 | weak |
| A92 | weak |
| A93 | weak |
| A94 | weak |
| A95 | weak |
| A96 | weak |
| A97 | strong |
| A98 | weak |
| A99 | weak |
| A100 | weak |
| A101 | weak |
| A102 | weak |
| A103 | weak |
| A104 | weak |
| A105 | weak |
| A106 | weak |
| A107 | weak |
| A108 | weak |
| A109 | weak |
| A110 | medium |
| A111 | medium |
| A112 | strong |
| A113 | medium |
| A114 | weak |
| A115 | weak |
| A116 | weak |
| A117 | weak |
| A118 | medium |
| A119 | weak |
| A120 | weak |
| A121 | weak |
| A122 | weak |
| A123 | strong |
| A124 | weak |
| A125 | weak |
| A126 | weak |
| A127 | weak |
| A128 | strong |
| A129 | weak |
| A130 | medium |
| A131 | medium |
| A132 | strong |
| A133 | strong |
| A134 | medium |
| A135 | weak |
| A136 | weak |
| A137 | weak |
| A138 | weak |
| A139 | strong |
| A140 | weak |
| A141 | strong |
| A142 | weak |
| A143 | weak |
| A144 | medium |
| A145 | strong |
| A146 | strong |
| A147 | medium |
| A148 | weak |
| A149 | weak |
| A150 | strong |
| A151 | strong |
| A152 | strong |
| A153 | weak |
| A154 | strong |
| A155 | weak |
| A156 | medium |
| A157 | strong |
| A158 | strong |
| A159 | strong |
| A160 | medium |
| A161 | strong |
| A162 | strong |
| A163 | strong |
| A164 | strong |
| A165 | strong |
| A166 | weak |
| A167 | strong |
| A168 | strong |
| A169 | strong |
| A170 | strong |
| A171 | weak |
| A172 | strong |
| A173 | strong |
| A174 | strong |
| A175 | strong |
| A176 | strong |
| A177 | strong |
| A178 | strong |
| A179 | strong |
| A180 | strong |
| A181 | strong |
| A182 | strong |
| A183 | weak |
| A184 | weak |
| A185 | strong |
| A186 | weak |
| A187 | strong |
| A188 | weak |
| A189 | strong |
| A190 | weak |
| A191 | strong |
| A192 | strong |
| A193 | strong |
| A194 | strong |
| A195 | medium |
| A196 | weak |
| A197 | strong |
| A198 | strong |
| A199 | strong |
| A200 | weak |
| A201 | weak |
| A202 | medium |
| A203 | strong |
| A204 | strong |
| A205 | medium |
| A206 | strong |
| A207 | weak |
| A208 | strong |
| A209 | strong |
| A210 | strong |
| A211 | weak |
| A212 | medium |
| A213 | weak |
| A215 | strong |
| A216 | strong |
| A217 | strong |
| A218 | weak |
| A219 | strong |
| A220 | strong |
| A221 | medium |
| A222 | strong |
| A223 | strong |
| A224 | strong |
| A225 | strong |
| A226 | strong |
| A227 | strong |
| A228 | strong |
| A229 | strong |
| A230 | strong |
| A231 | strong |
| A232 | strong |
| A233 | strong |
| A234 | strong |
| A235 | strong |
| A236 | strong |
| A237 | weak |
| A238 | medium |
| A239 | strong |
| A240 | strong |
| A241 | strong |
| A242 | weak |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

1. A compound of Formula (I): 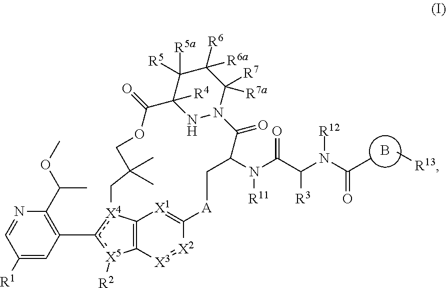
or a pharmaceutically acceptable salt thereof, wherein:
A is optionally substituted 3- to 6-membered cycloalkylene, optionally substituted 4- to 6-membered heterocycloalkylene, optionally substituted C₆₋₁₀ arylene, or optionally substituted 5- to 10-membered heteroarylene;
ring B is optionally substituted 4- to 11-membered heterocycloalkylene, wherein 1 or 2 ring carbon atoms of the 4- to 11-membered heterocycloalkylene are optionally replaced by a member independently selected from C(═O) and SO₂;
X¹, X² and X³ are each independently N or CR⁸, wherein each R⁸ is independently hydrogen, halogen, CN, or C₁₋₆ alkyl;
X⁴ is N and X⁵ is C; or
X⁵ is N and X⁴ is C; [symbol]
is either a single bond or a double bond to maintain the aromaticity of the 5-membered ring containing X⁴ and X⁵;
R¹ is hydrogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted 3- to 6-membered cycloalkyl, optionally substituted 3- to 6-membered cycloalkenyl, optionally substituted 4- to 14-membered heterocycloalkyl, optionally substituted 6- to 10-membered aryl, or optionally substituted 5- to 10-membered heteroaryl;
R² is optionally substituted C₁₋₆ alkyl, optionally substituted C₂₋₆ alkynyl, optionally substituted C₁₋₆ heteroalkyl, optionally substituted 3- to 6-membered cycloalkyl, or optionally substituted 4- to 14-membered heterocycloalkyl;
R³ is hydrogen, optionally substituted C₁₋₆ alkyl, optionally substituted C₁₋₃ heteroalkyl, optionally substituted C₃₋₆ cycloalkyl, or optionally substituted 4- to 6-membered heterocycloalkyl;
or R² and R³ together with the atoms to which they are attached form a fused C₄₋₁₀ cycloalkyl or a fused 4- to 10-membered heterocycloalkyl having 1, 2 or 3 ring heteroatoms selected from N, O, and S, wherein the fused C₄₋₁₀ cycloalkyl and heterocycloalkyl are each optionally substituted;
R⁴, R⁵, R^{5a}, R⁶, R^{6a}, R⁷ and R^{7a} are each independently hydrogen, halogen, or C₁₋₆ alkyl;
or R⁵ and R⁷ are taken together to form methylene or ethylene, which is covalently bound to the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form methylene or ethylene, which is covalently bound to the carbon atoms to which they are attached, and R⁵, R^{5a}, R^{6a}, R⁷ and R^{7a} are each independently hydrogen, halogen, or C₁₋₆ alkyl;
or R⁴ and R⁷ are taken together to form methylene or ethylene, which is covalently bound to the carbon atoms to which they are attached, R⁵, R^{5a}, R⁶, R^{6a}, and R^{7a} are each independently hydrogen, halogen, or C₁₋₆ alkyl;
or R⁵ and R⁶ taken together with the atoms to which they are attached form optionally substituted fused C₃₋₆ cycloalkyl or optionally substituted 4- to 6-membered fused heterocycloalkyl;
or R⁶ and R⁷ taken together with the atoms to which they are attached form optionally substituted fused C₃₋₆ cycloalkyl or optionally substituted 4- to 6-membered fused heterocycloalkyl;
or R⁵ and R^{5a} taken together with the atom to which they are attached form optionally substituted C₃₋₆ cycloalkyl or optionally substituted 4- to 6-membered heterocycloalkyl;
or R⁶ and R^{6a} taken together with the atom to which they are attached form optionally substituted C₃₋₆ cycloalkyl or optionally substituted 4- to 6-membered heterocycloalkyl;
or R⁷ and R^{7a} taken together with the atom to which they are attached form an optionally C₃₋₆ cycloalkyl or optionally substituted 4- to 6-membered heterocycloalkyl;
or R⁵ and R⁶ are taken together to form a bond;
R¹¹ is hydrogen or optionally substituted C₁₋₆ alkyl;
R¹² is hydrogen or optionally substituted C₁₋₆ alkyl; and
R¹³ is H, optionally substituted C₁₋₆ alkyl, —C(O)—R¹⁴, —C(O)OR¹⁴, —C(O)NHR¹⁴, —C(O)NR¹⁴R¹⁴, —SO₂R¹⁴, —SO₂NHR¹⁴, —SO₂NR¹⁴R¹⁴, wherein each R¹⁴ is independently optionally substituted C₁₋₆ alkyl, optionally substituted C₆₋₁₀ aryl, optionally substituted C₃₋₆ cycloalkyl, optionally substituted 5-6 membered heteroaryl, optionally substituted 4-14 membered heterocycloalkyl, optionally substituted C₆₋₁₀ aryl-C₁₋₆ alkyl-, optionally substituted C₃₋₆ cycloalkyl-C₁₋₆ alkyl-, optionally substituted 5-6 membered heteroaryl-C₁₋₆ alkyl-, or optionally substituted 4-14 membered heterocycloalkyl-C₁₋₆ alkyl.

2. The compound of claim 1, wherein the compound has the formula I-a: 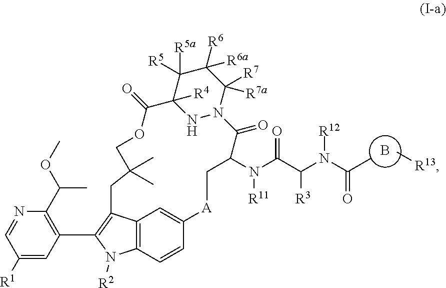
or a pharmaceutically acceptable salt thereof.

3. The compound of claim 1 or 2, or a pharmaceutically acceptable salt thereof, wherein:
R⁵ and R⁷ are taken together to form methylene or ethylene, which is covalently bound to the carbon atoms to which they are attached, and R⁴, R^{5a}, R⁶, R^{6a}, and R^{7a} are each independently hydrogen, halogen, or C₁₋₆ alkyl;
or R⁴ and R⁶ are taken together to form methylene or ethylene, which is covalently bound to the carbon atoms to which they are attached, and R⁵, R^{5a}, R^{6a}, R⁷ and R^{7a} are each independently hydrogen, halogen, or C₁₋₆ alkyl;
or R⁴ and R⁷ are taken together to form methylene or ethylene, which is covalently bound to the carbon atoms to which they are attached, R⁵, R^{5a}, R⁶, R^{6a}, and R^{7a} are each independently hydrogen, halogen, or C₁₋₆ alkyl;
or R⁵ and R⁶ taken together with the atoms to which they are attached form optionally substituted fused C₃₋₆ cycloalkyl or optionally substituted 4- to 6-membered fused heterocycloalkyl;
or R⁶ and R⁷ taken together with the atoms to which they are attached form optionally substituted fused C₃₋₆ cycloalkyl or optionally substituted 4- to 6-membered fused heterocycloalkyl;
or R⁵ and R^{5a} taken together with the atom to which they are attached form optionally substituted C₃₋₆ cycloalkyl or optionally substituted 4- to 6-membered heterocycloalkyl;
or R⁶ and R^{6a} taken together with the atom to which they are attached form optionally substituted C₃₋₆ cycloalkyl or optionally substituted 4- to 6-membered heterocycloalkyl;
or R⁷ and R^{7a} taken together with the atom to which they are attached form an optionally C₃₋₆ cycloalkyl or optionally substituted 4- to 6-membered heterocycloalkyl;
or R⁵ and R⁶ are taken together to form a bond.

4. The compound of claim 1, wherein the compound is of formula (II): 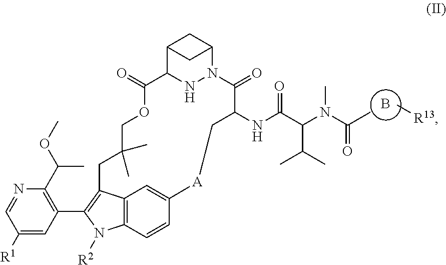
or a pharmaceutically acceptable salt thereof.

5. The compound of claim 1, wherein the compound is of formula (III): 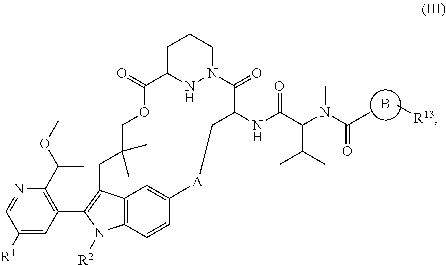
or a pharmaceutically acceptable salt thereof.

6. The compound of any one of claims 1-5, wherein A is of the formula: 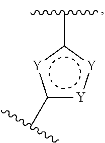
wherein [symbol]
indicates optional double bonds in the ring structure; and
each Y is independently CH, N, O, or S.

7. The compound of any one of claims 1-5, wherein A is of the formula: 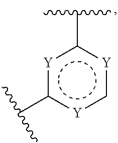
wherein [symbol]
indicates optional double bonds in the ring structure; and
each Y is independently CH or N.

8. The compound of any one of claims 1-5, wherein A is of the formula: 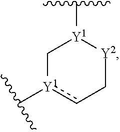
wherein [symbol]
is a single bond or a double bond;
each Y¹ is independently C, CH or N; and
Y² is CH₂, NH or O.

9. The compound of any one of claims 1-8, or a pharmaceutically acceptable salt thereof, wherein ring B is optionally substituted 4- to 11-membered fused heterocycloalkyl or optionally substituted 4- to 11-membered spiro heterocycloalkyl or optionally substituted 4- to 6-membered heterocycloalkyl.

10. The compound of any one of claims 1-9, wherein B is of the formula: 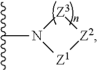
wherein,
n is 1, 2, or 3;
each of Z¹, Z², and Z³ is, independently, C═O, O, NR^{Z}, CH—R^{Z}, C(R^{Z})₂; and
each R^{Z} is independently H, optionally substituted C₁₋₆ alkyl, optionally substituted C₁₋₆ heteroalkyl, optionally substituted 4- to 6-membered cycloalkyl, optionally substituted 4- to 6-membered heterocycloalkyl, or two adjacent Z together form an optionally substituted 4- to 6-membered heterocycloalkyl or optionally substituted 4- to 6-membered cycloalkyl.

11. The compound of claim 10, wherein Z¹ is C(R^{Z})₂ and each R^{Z} combine to form an optionally substituted 4-6 membered heterocycloalkyl.

12. The compound of claim 10 or 11, wherein Z² is C(R^{Z})₂, wherein each R^{Z} is H.

13. The compound of claim 10 or 11, wherein Z² is O.

14. The compound of claim 10 or 11, wherein Z² is C(R^{Z})₂ and each R^{Z} combine to form an optionally substituted 4- to 6-membered heterocycloalkyl.

15. The compound of claim 14, wherein Z¹ is C(R^{Z})₂, wherein each R^{Z} is H.

16. The compound of claim 14, wherein Z¹ is C═O.

17. The compound of claim 10, wherein Z¹ and Z² are each CH—R^{Z} and the R^{Z} from Z¹ and the R^{Z} from the adjacent Z² combine to form an optionally substituted 4- to 6-membered heterocycloalkyl.

18. The compound of claim 10, wherein Z² is NR^{Z} and the adjacent Z³ is CH—R^{Z}, and the R^{Z} from Z² and the R^{Z} from the an adjacent Z³ combine to form an optionally substituted 4- to 6-membered heterocycloalkyl.

19. The compound of claim 10, wherein Z¹ is C(R^{Z})₂ and each R^{Z} combine to form an optionally substituted 4- to 6-membered cycloalkyl.

20. The compound of claim 19, wherein Z² is C(R^{Z})₂ and each R^{Z} is H.

21. The compound of claim 19, wherein Z² is O.

22. The compound of claim 10, wherein Z² is C(R^{Z})₂ and each R^{Z} combine to form an optionally substituted 4- to 6-membered cycloalkyl.

23. The compound of claim 22, wherein Z¹ is C(R^{Z})₂, wherein each R^{Z} is H.

24. The compound of claim 22, wherein Z¹ is C═O.

25. The compound of any one of claims 10-24, wherein each Z³ is CH—R^{Z} and R^{Z} is H.

26. The compound of any one of claims 10-24, wherein one Z³ is CH—R^{z} and R^{z} is CH₃.

27. The compound of claim 10, wherein Z¹ or Z² is CH—R^{Z} and R^{Z} is

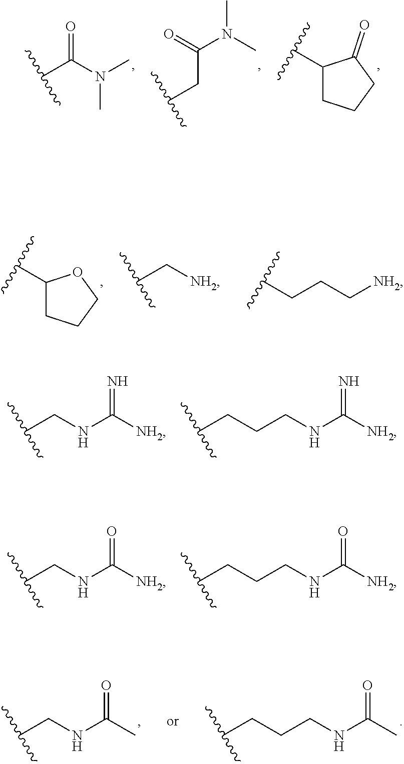

28. The compound of claim 10, wherein Z¹ is C(R^{Z})₂, where one R^{Z} is CH₃ and the other R^{Z} is selected from the group consisting of:

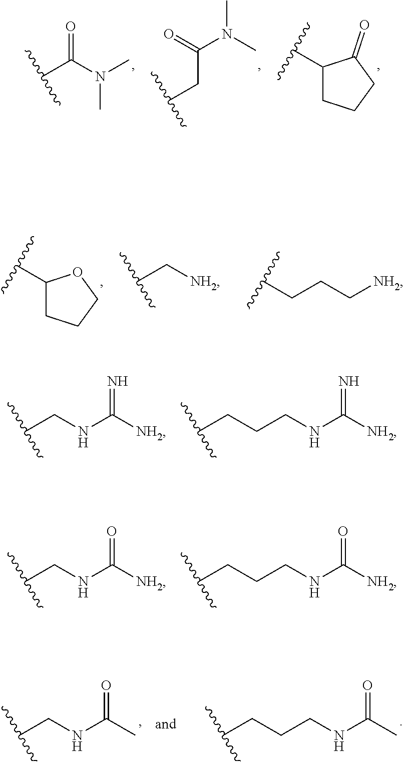

29. The compound of any one of claims 1-9, wherein B—R¹³ is selected from a group consisting of:

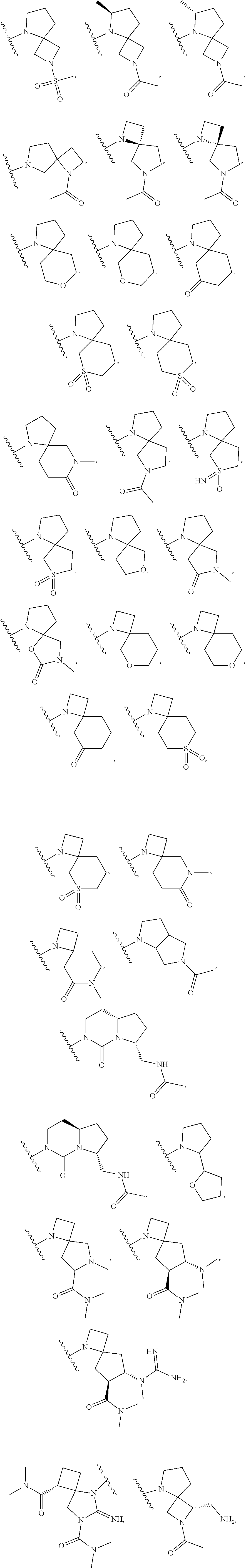

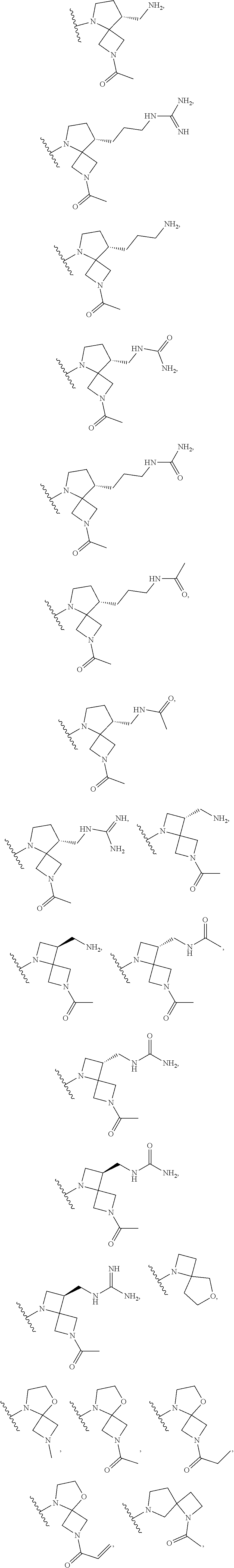

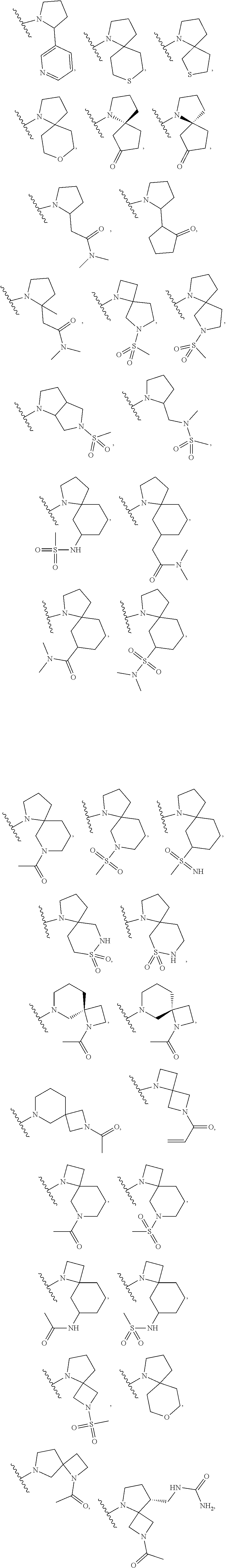

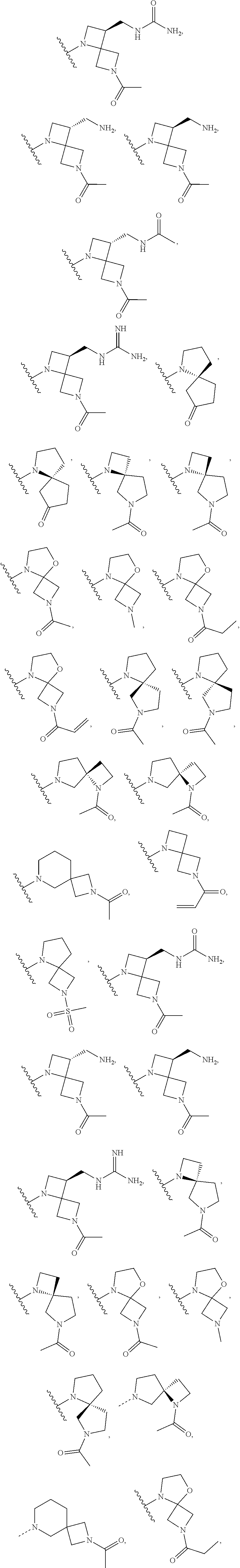

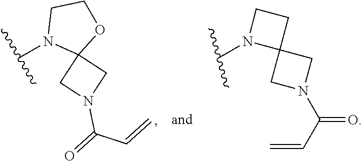 [SMILES: *N1CCOC12CN(C(=O)C=C)C2 ; *N1CCC12CN(C(=O)C=C)C2]

30. The compound of any one of claims 1-29, wherein R² is optionally substituted 4- to 14-membered heterocycloalkyl.

31. The compound of claim 30, wherein R² is of the formula: 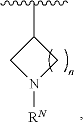
wherein,
N is 1, 2, or 3; and
R^{N} is C₁₋₆ alkyl, 3- to 6-membered cycloalkyl, or 4- to 6-membered heterocycloalkyl.

32. The compound of any one of claims 1-29, wherein R² is optionally substituted C₁₋₆ alkyl.

33. The compound of any one of claims 1-29, wherein R² is optionally substituted 3- to 6-membered cycloalkyl.

34. A compound selected from compounds A1-A36 in Table 1 or a pharmaceutically acceptable salt thereof.

35. A pharmaceutical composition comprising a compound, or a pharmaceutically acceptable salt thereof, of any one of claims 1-34 and a pharmaceutically acceptable excipient or carrier.

36. A method of treating cancer in a subject in need thereof, the method comprising administering to the subject a therapeutically effective amount of a compound, or a pharmaceutically acceptable salt thereof, of any one of claims 1 to 34 or a pharmaceutical composition of claim 35.

37. The method of claim 36, wherein the cancer is melanoma, pancreatic cancer, colorectal cancer, non-small cell lung cancer, gastric cancer, esophageal cancer, ovarian cancer, or uterine cancer.

38. The method of claim 36 or 37, wherein the cancer comprises a Ras mutation.

39. A method of treating a Ras protein-related disorder in a subject in need thereof, the method comprising administering to the subject a therapeutically effective amount of a compound of any one of claims 1 to 34, or a pharmaceutically acceptable salt thereof, or a pharmaceutical composition of claim 35.

40. The method of any one of claims 36-39, wherein the method further comprises administering an additional anticancer therapy.

41. The method of claim 40, wherein the additional anticancer therapy is an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CTLA-4 inhibitor, a CDK4/6 inhibitor, a HER2 inhibitor, an immune checkpoint inhibitor or a combination thereof.

42. A method for inhibiting a RAS mutant in a cell, the method comprising: contacting the cell with a compound of any one of claims 1-34, or pharmaceutically acceptable salt thereof, wherein the RAS mutant has a mutation at position G12, G13, Q61, or a combination thereof.
